(19) United States
(12) Patent Application Publication
(10) Pub. No.: US 2022/0380742 A1
(43) Pub. Date: Dec. 1, 2022

(54) SIALIDASE-CD20-ANTIBODY FUSION PROTEINS AND METHODS OF USE THEREOF

(71) Applicant: Palleon Pharmaceuticals Inc., Waltham, MA (US)

(72) Inventors: Li Peng, Lexington, MA (US); Sujata B. Nerle, Somerville, MA (US); Wayne C. Gatlin, Wakefield, MA (US)

(21) Appl. No.: 17/624,118

(22) PCT Filed: Jul. 3, 2020
(86) PCT No.: PCT/US2020/040814

§ 371 Date: Dec. 30, 2021

Related U.S. Application Data
(60) Provisional application No. 62/956,977, filed on Jan. 3, 2020.
(60) Provisional application No. 62/870,348, filed on Jul. 3, 2019.

(51) Int. Cl. C12N 9/24; C07K 16/28; A61K 47/68; A61P 35/00

(52) U.S. Cl. CPC C12N 9/2402; C12Y 302/01018; C07K 16/2887; A61K 47/6849; A61P 35/00; C07K 2319/30; A61K 2039/505

(57) ABSTRACT

The invention relates generally to recombinant sialidase and anti-CD20 immunoglobulin antigen-binding domain fusion proteins. The invention also provides antibody conjugates including a sialidase and an anti-CD20 antibody or a portion thereof. The invention further relates to methods of using the sialidase fusion proteins or antibody conjugates for treating cancer.

### CROSS REFERENCE TO RELATED APPLICATIONS

This application claims the benefit of, and priority to, U.S. Provisional Patent Application Ser. No. 62/870,348, filed Jul. 3, 2019, and U.S. Provisional Patent Application Ser. No. 62/956,977, filed Jan. 3, 2020, the entire disclosure of each of which is hereby incorporated by reference in its entirety.

### FIELD OF THE INVENTION

The invention relates generally to recombinant sialidase fusion proteins and antibody conjugates, and their use in the treatment of cancer.

### BACKGROUND

A growing body of evidence supports roles for glycans, and sialoglycans in particular, at various pathophysiological steps of tumor progression. Glycans regulate tumor proliferation, invasion, hematogenous metastasis and angiogenesis (Fuster et al. (2005) NAT. REV. CANCER 5(7): 526-42). The sialylation of cell surface glycoconjugates is frequently altered in cancers, resulting in the expression of sialylated tumor-associated carbohydrate antigens. The expression of sialylated glycans by tumor cells is often associated with increased aggressiveness and metastatic potential of a tumor.

It has recently become apparent that Siglecs (sialic acid-binding immunoglobulin-like lectins), a family of sialic acid binding lectins, play a role in cancer immune suppression by binding to hypersialylated cancer cells and mediating the suppression of signals from activating NK cell receptors, thereby inhibiting NK cell-mediated killing of tumor cells (Jandus et al. (2014) J. CLIN. INVEST. 124: 1810-1820; Läubli et al. (2014) PROC. NATL. ACAD. SCI. USA 111: 14211-14216; Hudak et al. (2014) NAT. CHEM. BIOL. 10: 69-75). Likewise, enzymatic removal of sialic acids by treatment with sialidase can enhance NK cell-mediated killing of tumor cells (Jandus, supra; Hudak, supra; Xiao et al. (2016) PROC. NATL. ACAD. SCI. USA 113(37): 10304-9).

CD20 is expressed in a vast majority of mature B-cell neoplasms and some cases of B-lymphoblastic leukemia/lymphomas, plasma-cell myelomas, Hodgkin lymphomas, T-cell neoplasms, and acute myeloid leukemias (AMLs). For example, 90% of B cell lymphomas stain positive for CD20 (Chu et al. (2006) AM J CLIN PATHOL 126:534), 40% of pre-B acute lymphoblastic leukemias (ALLs) and lymphoblastic lymphoma (LBLs) stain positive for CD20, 80% of nodular lymphocyte predominant Hodgkin lymphomas stain positive for CD20, and 20% of classic Hodgkin lymphomas stain positive for CD20. Anti-CD20 antibodies include obinutuzumab, approved for use in, for example, chronic lymphocytic leukemias and follicular lymphomas, ofatumumab, approved for use in, for example, chronic lymphocytic leukemias, and rituximab, approved for use in, for example, non-Hodgkin's lymphomas and chronic lymphocytic leukemias.

Cancer immunotherapy with immune checkpoint inhibitors, including antibodies blocking the PD-1/PD-L1 pathway, has improved the outcome of many cancer patients. However, despite advances that have been made to date, many patients do not respond to currently available immune checkpoint inhibitors. Accordingly, there is still a need for effective interventions that overcome the immune suppressive tumor microenvironment and for treating cancers associated with hypersialylated cancer cells.

### SUMMARY OF THE INVENTION

The invention is based, in part, upon the discovery that it is possible to produce fusion proteins containing a sialidase enzyme and an anti-CD20 immunoglobulin antigen-binding and/or antibody conjugates including a sialidase enzyme and an anti-CD20 antibody or a portion thereof. The fusion proteins and/or antibody conjugates have suitable substrate specificities and activities to be useful in removing sialic acid and/or sialic acid containing molecules from the surface of cancer cells, e.g., CD20-expressing cancer cells, and/or removing sialic acid and/or sialic acid containing molecules from the tumor microenvironment, and/or reducing the concentration of sialic acid and/or sialic acid containing molecules in the tumor microenvironment.

Accordingly, in one aspect, the invention provides a fusion protein comprising (or consisting essentially of): (a) sialidase enzyme; and (b) an anti-CD20 immunoglobulin antigen-binding domain.

In certain embodiments, the sialidase is a human sialidase, e.g., a recombinant mutant human sialidase. In certain embodiments, the sialidase comprises: (a) a substitution of a proline residue at a position corresponding to position 5 of wild-type human Neu2 (P5); (b) a substitution of a lysine residue at a position corresponding to position 9 of wild-type human Neu2 (K9); (c) a substitution of a lysine residue at a position corresponding to position 44 of wild-type human Neu2 (K44); (d) a substitution of a lysine residue at a position corresponding to position 45 of wild-type human Neu2 (K45); (e) a substitution of a leucine residue at a position corresponding to position 54 of wild-type human Neu2 (L54); (f) a substitution of a proline residue at a position corresponding to position 62 of wild-type human Neu2 (P62); (g) a substitution of a glutamine residue at a position corresponding to position 69 of wild-type human Neu2 (Q69); (h) a substitution of an arginine residue at a position corresponding to position 78 of wild-type human Neu2 (R78); (i) a substitution of an aspartic acid residue at a position corresponding to position 80 of wild-type human Neu2 (D80); (j) a substitution of an alanine residue at a position corresponding to position 93 of wild-type human Neu2 (A93); (k) a substitution of a glycine residue at a position corresponding to position 107 of wild-type human Neu2 (G107); (l) a substitution of a glutamine residue at a position corresponding to position 108 of wild-type human Neu2 (Q108); (m) a substitution of a glutamine residue at a position corresponding to position 112 of wild-type human Neu2 (Q112); (n) a substitution of a cysteine residue at a position corresponding to position 125 of wild-type human Neu2 (C125); (o) a substitution of a glutamine residue at a position corresponding to position 126 of wild-type human Neu2 (Q126); (p) a substitution of an alanine residue at a position corresponding to position 150 of wild-type human Neu2 (A150); (q) a substitution of a cysteine residue at a position corresponding to position 164 of wild-type human Neu2 (C164); (r) a substitution of an arginine residue at a position corresponding to position 170 of wild-type human Neu2 (R170); (s) a substitution of an alanine residue at a position corresponding to position 171 of wild-type human Neu2 (A171); (t) a substitution of a glutamine residue at a position corresponding to position 188 of wild-type human Neu2 (Q188); (u) a substitution of an arginine residue at a position corresponding to position 189 of wild-type human Neu2 (R189); (v) a substitution of an alanine residue at a position corresponding to position 213 of wild-type human Neu2 (A213); (w) a substitution of a leucine residue at a position corresponding to position 217 of wild-type human Neu2 (L217); (x) a substitution of a glutamic acid residue at a position corresponding to position 225 of wild-type human Neu2 (E225); (y) a substitution of a histidine residue at a position corresponding to position 239 of wild-type human Neu2 (H239); (z) a substitution of a leucine residue at a position corresponding to position 240 of wild-type human Neu2 (L240); (aa) a substitution of an arginine residue at a position corresponding to position 241 of wild-type human Neu2 (R241); (bb) a substitution of an alanine residue at a position corresponding to position 242 of wild-type human Neu2 (A242); (cc) a substitution of a valine residue at a position corresponding to position 244 of wild-type human Neu2 (V244); (dd) a substitution of a threonine residue at a position corresponding to position 249 of wild-type human Neu2 (T249); (ee) a substitution of an aspartic acid residue at a position corresponding to position 251 of wild-type human Neu2 (D251); (ff) a substitution of a glutamic acid residue at a position corresponding to position 257 of wild-type human Neu2 (E257); (gg) a substitution of a serine residue at a position corresponding to position 258 of wild-type human Neu2 (S258); (hh) a substitution of a leucine residue at a position corresponding to position 260 of wild-type human Neu2 (L260); (ii) a substitution of a valine residue at a position corresponding to position 265 of wild-type human Neu2 (V265); (jj) a substitution of a glutamine residue at a position corresponding to position 270 of wild-type human Neu2 (Q270); (kk) a substitution of a tryptophan residue at a position corresponding to position 292 of wild-type human Neu2 (W292); (ll) a substitution of a serine residue at a position corresponding to position 301 of wild-type human Neu2 (S301); (mm) a substitution of a tryptophan residue at a position corresponding to position 302 of wild-type human Neu2 (W302); (nn) a substitution of a valine residue at a position corresponding to position 363 of wild-type human Neu2 (V363); or (oo) a substitution of a leucine residue at a position corresponding to position 365 of wild-type human Neu2 (L365); or a combination of any of the foregoing substitutions. For example, the sialidase may comprise a substitution of K9, P62, A93, Q216, A242, Q270, S301, W302, V363, or L365, or a combination of any of the foregoing substitutions.

In certain embodiments, in the sialidase: (a) the proline residue at a position corresponding to position 5 of wild-type human Neu2 is substituted by histidine (P5H); (b) the lysine residue at a position corresponding to position 9 of wild-type human Neu2 is substituted by aspartic acid (K9D); (c) the lysine residue at a position corresponding to position 44 of wild-type human Neu2 is substituted by arginine (K44R) or glutamic acid (K44E); (d) the lysine residue at a position corresponding to position 45 of wild-type human Neu2 is substituted by alanine (K45A), arginine (K45R), or glutamic acid (K45E); (e) the leucine residue at a position corresponding to position 54 of wild-type human Neu2 is substituted by methionine (L54M); (f) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T); (g) the glutamine residue at a position corresponding to position 69 of wild-type human Neu2 is substituted by histidine (Q69H); (h) the arginine residue at a position corresponding to position 78 of wild-type human Neu2 is substituted by lysine (R78K); (i) the aspartic acid residue at a position corresponding to position 80 of wild-type human Neu2 is substituted by proline (D80P); (j) the alanine residue at a position corresponding to position 93 of wild-type human Neu2 is substituted by glutamic acid (A93E) or lysine (A93K); (k) the glycine residue at a position corresponding to position 107 of wild-type human Neu2 is substituted by aspartic acid (G107D); (l) the glutamine residue at a position corresponding to position 108 of wild-type human Neu2 is substituted by histidine (Q108H); (m) the glutamine residue at a position corresponding to position 112 of wild-type human Neu2 is substituted by arginine (Q112R) or lysine (Q112K); (n) the cysteine residue at a position corresponding to position 125 of wild-type human Neu2 is substituted by leucine (C125L); (o) the glutamine residue at a position corresponding to position 126 of wild-type human Neu2 is substituted by leucine (Q126L), glutamic acid (Q126E), phenylalanine (Q126F), histidine (Q126H), isoleucine (Q126I), or tyrosine (Q126Y); (p) the alanine residue at a position corresponding to position 150 of wild-type human Neu2 is substituted by valine (A150V); (q) the cysteine residue at a position corresponding to position 164 of wild-type human Neu2 is substituted by glycine (C164G); (r) the arginine residue at a position corresponding to position 170 of wild-type human Neu2 is substituted by proline (R170P); (s) the alanine residue at a position corresponding to position 171 of wild-type human Neu2 is substituted by glycine (A171G); (t) the glutamine residue at a position corresponding to position 188 of wild-type human Neu2 is substituted by proline (Q188P); (u) the arginine residue at a position corresponding to position 189 of wild-type human Neu2 is substituted by proline (R189P); (v) the alanine residue at a position corresponding to position 213 of wild-type human Neu2 is substituted by cysteine (A213C), asparagine (A213N), serine (A213S), or threonine (A213T); (w) the leucine residue at a position corresponding to position 217 of wild-type human Neu2 is substituted by alanine (L217A) or valine (L217V); (x) the threonine residue at a position corresponding to position 249 of wild-type human Neu2 is substituted by alanine (T249A); (y) the aspartic acid residue at a position corresponding to position 251 of wild-type human Neu2 is substituted by glycine (D251G); (z) the glutamic acid residue at a position corresponding to position 225 of wild-type human Neu2 is substituted by proline (E225P); (aa) the histidine residue at a position corresponding to position 239 of wild-type human Neu2 is substituted by proline (H239P); (bb) the leucine residue at a position corresponding to position 240 of wild-type human Neu2 is substituted by aspartic acid (L240D), asparagine (L240N), or tyrosine (L240Y); (cc) the arginine residue at a position corresponding to position 241 of wild-type human Neu2 is substituted by alanine (R241A), aspartic acid (R241D), leucine (R241L), glutamine (R241Q). or tyrosine (R241Y); (dd) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (ee) the valine residue at a position corresponding to position 244 of wild-type human Neu2 is substituted by isoleucine (V244I), lysine (V244K), or proline (V244P); (ff) the glutamic acid residue at a position corresponding to position 257 of wild-type human Neu2 is substituted by proline (E257P); (gg) the serine residue at a position corresponding to position 258 is substituted by cysteine (S258C); (hh) the leucine residue at a position corresponding to position 260 of wild-type human Neu2 is substituted by aspartic acid (L260D), phenylalanine (L260F), glutamine (L260Q), or threonine (L260T); (ii) the valine residue at a position corresponding to position 265 of wild-type human Neu2 is substituted by phenylalanine (V265F); (jj) the glutamine residue at a position corresponding to position 270 of wild-type human Neu2 is substituted by alanine (Q270A), histidine (Q270H), phenylalanine (Q270F), proline (Q270P), serine (Q270S), or threonine (Q270T); (kk) the tryptophan residue at a position corresponding to position 292 of wild-type human Neu2 is substituted by arginine (W292R); (ll) the serine residue at a position corresponding to position 301 of wild-type human Neu2 is substituted by alanine (S301A), aspartic acid (S301D), glutamic acid (S301E), phenylalanine (S301F), histidine (S301H), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y); (mm) the tryptophan residue at a position corresponding to position 302 of wild-type human Neu2 is substituted by alanine (W302A), aspartic acid (W302D), phenylalanine (W302F), glycine (W302G), histidine (W302H), isoleucine (W302I), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y); (nn) the valine residue at a position corresponding to position 363 of wild-type human Neu2 is substituted by arginine (V363R); or (oo) the leucine residue at a position corresponding to position 365 of wild-type human Neu2 is substituted by glutamine (L365Q), histidine (L365H), isoleucine (L365I), lysine (L365K) or serine (L365S); or the sialidase comprises a combination of any of the foregoing substitutions. For example, the sialidase may comprise a substitution selected from K9D, P62G, P62N, P62S, P62T, A93E, Q126Y, A242F, A242W, A242Y, Q270A, Q270T, S301A, S301R, W302K, W302R, V363R, and L365I, or a combination of any of the foregoing substitutions.

In certain embodiments, the sialidase comprises: (a) a substitution or deletion of a methionine residue at a position corresponding to position 1 of wild-type human Neu2 (M1); (b) a substitution of a valine residue at a position corresponding to position 6 of wild-type human Neu2 (V6); (c) a substitution of an isoleucine residue at a position corresponding to position 187 of wild-type human Neu2 (I187); or (d) a substitution of a cysteine residue at a position corresponding to position 332 of wild-type human Neu2 (C332); or a combination of any of the foregoing substitutions.

In certain embodiments, in the sialidase: (a) the methionine residue at a position corresponding to position 1 of wild-type human Neu2 is deleted (ΔM1), is substituted by alanine (M1A), or is substituted by aspartic acid (M1D); (b) the valine residue at a position corresponding to position 6 of wild-type human Neu2 is substituted by tyrosine (V6Y); (c) the isoleucine residue at a position corresponding to position 187 of wild-type human Neu2 is substituted by lysine (I187K); or (d) the cysteine residue at a position corresponding to position 332 of wild-type human Neu2 is substituted by alanine (C332A); or the sialidase comprises a combination of any of the foregoing substitutions.

In certain embodiments, the sialidase comprises: (a) the M1D, V6Y, P62G, A93E, I187K, and C332A substitutions; (b) the M1D, V6Y, K9D, A93E, I187K, C332A, V363R, and L365I substitutions; (c) the M1D, V6Y, P62N, I187K, and C332A substitutions; (d) the M1D, V6Y, I187K, Q270A, S301R, W302K, and C332A substitutions; (e) the M1D, V6Y, P62S, I187K, Q270A, S301R, W302K, and C332A substitutions; (f) the M1D, V6Y, P62T, I187K, Q270A, S301R, W302K, and C332A substitutions; (g) the M1D, V6Y, P62N, I187K, Q270A, S301R, W302K, and C332A substitutions; (h) the M1D, V6Y, P62G, A93E, I187K, S301A, W302R, and C332A substitutions; (i) the M1D, V6Y, P62G, A93E, Q126Y, I187K, Q270T, and C332A substitutions; (j) the M1D, V6Y, P62G, A93E, Q126Y, I187K, and C332A substitutions; or (k) the M1D, V6Y, P62G, A93E, Q126Y, I187K, A242F, Q270T, and C332A substitutions.

In certain embodiments, the sialidase is selected from Neu1, Neu2, Neu3, and Neu4, e.g., the sialidase is Neu2.

In certain embodiments, the sialidase has a different substrate specificity than the corresponding wild-type sialidase. For example, in certain embodiments the sialidase can cleave α2,3, α2,6, and/or α2,8 linkages. In certain embodiments the sialidase can cleave α2,3 and α2,8 linkages.

In certain embodiments, the sialidase comprises any one of SEQ ID NOs: 48-62, 102, 105, 108, or 133, or an amino acid sequence that has at least 85%, 90%, 95%, 96%, 97%, 98%, or 99% sequence identity to any one of SEQ ID NOs: 48-62, 102, 105, 108, or 133.

In certain embodiments, the sialidase comprises mutation or combination of mutations set forth in any one of Tables 5-12 or 14-27. In certain embodiments, the sialidase comprises a mutation or combination of mutations set forth in any one of Tables 1-4.

In certain embodiments, the fusion protein further comprises an immunoglobulin Fc domain. In certain embodiments, the immunoglobulin Fc domain is derived from a human IgG1, IgG2, IgG3, IgG4, IgA1, IgA2, IgD, IgE, or IgM Fc domain, e.g., the immunoglobulin Fc domain is derived from a human IgG1, IgG2, IgG3, or IgG4 Fc domain, e.g., the immunoglobulin Fc domain is derived from a human IgG1 Fc domain.

In certain embodiments, the anti-CD20 immunoglobulin antigen-binding domain is associated (for example, covalently or non-covalently associated) with a second anti-CD20 immunoglobulin antigen-binding domain to produce an anti-CD20 antigen-binding site.

In certain embodiments, the anti-CD20 immunoglobulin antigen-binding domain is derived from an antibody selected from ofatumumab, rituximab, ocrelizumab, obinutuzumab, ibritumomab, veltuzumab, reditux, PRO131921, TRU-015, ubituximab, NCD1.2, blontuvetmab, FBTA05, mosunetuzumab, ocaratuzumab, ocrelizumab, and tositumomab, e.g., the anti-CD20 immunoglobulin antigen-binding domain is derived from ofatumumab or rituximab.

In certain embodiments, the sialidase and the immunoglobulin Fc domain and/or the anti-CD20 immunoglobulin antigen-binding domain are linked by a peptide bond or an amino acid linker.

In certain embodiments, the fusion protein comprises any one of SEQ ID NOs: 66-74, 78, 88-97, 103, 104, 106, 107, 109, 110, 113, 115, 117, 119, 121, 127, 128, 134, or 135.

In another aspect, the invention provides an antibody conjugate comprising any of the foregoing fusion proteins. In certain embodiments, the antibody conjugate comprises a single sialidase. In other embodiments, the antibody conjugate comprises two sialidases, which can be the same or different. In certain embodiments the antibody conjugate comprises two identical sialidases. In certain embodiments, the antibody conjugate comprises a single anti-CD20 antigen-binding site. In other embodiments, the antibody conjugate comprises two anti-CD20 antigen-binding sites, which can be the same or different. In certain embodiments, the antibody conjugate comprises two identical anti-CD20 antigen-binding sites.

In certain embodiments, the antibody conjugate has a molecular weight from about 135 kDa to about 165 kDa, or the antibody conjugate has a molecular weight from about 215 kDa to about 245 kDa.

In certain embodiments, the antibody conjugate comprises: (a) a first polypeptide comprising an immunoglobulin light chain; (b) a second polypeptide comprising an immunoglobulin heavy chain; and (c) a third polypeptide comprising an immunoglobulin Fc domain and a sialidase; wherein the first and second polypeptides are covalently linked together and the second and third polypeptides are covalently linked together, and wherein the first polypeptide and the second polypeptide together define an anti-CD20 antigen-binding site. The third polypeptide may, for example, comprise the sialidase and the immunoglobulin Fc domain in an N- to C-terminal orientation. The first polypeptide may, for example, comprise any one of SEQ ID NOs: 64 or 66, the second polypeptide may, for example, comprise any one of SEQ ID NOs: 67 or 129, and/or the third polypeptide may, for example, comprise any one of SEQ ID NOs: 68-74, 78, 88-97, 103, 104, 106, 107, 109, 110, 113, 115, 117, 127, 128, 134, or 135. The first polypeptide may, for example, comprise any one of SEQ ID NOs: 77, 80, or 111, the second polypeptide may, for example, comprise any one of SEQ ID NOs: 81 or 130, and/or the third polypeptide may, for example, comprise any one of SEQ ID NOs: 68-74, 78, 88-97, 103, 104, 106, 107, 109, 110, 113, 115, 117, 127, 128, 134, or 135.

In certain embodiments, the antibody conjugate comprises: (a) a first polypeptide comprising a first immunoglobulin light chain; (b) a second polypeptide comprising a first immunoglobulin heavy chain and a first sialidase; (c) a third polypeptide comprising a second immunoglobulin heavy chain and a second sialidase; and (d) a fourth polypeptide comprising a second immunoglobulin light chain; wherein the first and second polypeptides are covalently linked together, the third and fourth polypeptides are covalently linked together, and the second and third polypeptides are covalently linked together, and wherein the first polypeptide and the second polypeptide together define a first anti-CD20 antigen-binding site, and the third polypeptide and the fourth polypeptide together define a second anti-CD20 antigen-binding site. The second and third polypeptides may, for example, comprise the first and second immunoglobulin heavy chain and the first and second sialidase, respectively, in an N- to C-terminal orientation. The first and/or fourth polypeptide may, for example, comprise any one of SEQ ID NOs: 77, 80, or 111. The second and/or third polypeptide may, for example, comprise any one of SEQ ID NOs: 119 or 121.

In certain embodiments, the antibody conjugate comprises: (a) a first polypeptide comprising a first sialidase, a first immunoglobulin Fc domain, and a first single chain variable fragment (scFv); and (b) a second polypeptide comprising a second sialidase, a second immunoglobulin Fc domain, and an optional second single chain variable fragment (scFv); wherein the first and second polypeptides are covalently linked together, and wherein the first scFv defines a first anti-CD20 antigen-binding site, and the second scFv, when present, defines a second anti-CD20 antigen-binding site. The first polypeptide may, for example comprise the first sialidase, the first immunoglobulin Fc domain, and the first scFv in an N- to C-terminal orientation. The second polypeptide may, for example, comprise the second sialidase, the second immunoglobulin Fc domain, and the optional second scFv in an N- to C-terminal orientation.

In certain embodiments, the antibody conjugate comprises: (a) a first polypeptide comprising an immunoglobulin light chain; (b) a second polypeptide comprising an immunoglobulin heavy chain and a single chain variable fragment (scFv); and (c) a third polypeptide comprising an immunoglobulin Fc domain and a sialidase, wherein the first and second polypeptides are covalently linked together and the second and third polypeptides are covalently linked together, and wherein the immunoglobulin light chain and immunoglobulin heavy chain together define a first anti-CD20 antigen-binding site and the scFv defines a second anti-CD20 antigen-binding site. The second polypeptide may, for example comprise the immunoglobulin heavy chain and the scFv in an N- to C-terminal orientation. The third polypeptide may, for example, comprise the sialidase and the immunoglobulin Fc domain in an N- to C-terminal orientation.

In another aspect, the invention provides an isolated nucleic acid comprising a nucleotide sequence encoding any of the foregoing fusion proteins or at least a portion of any of the foregoing antibody conjugates. In another aspect, the invention provides an expression vector comprising any of the foregoing nucleic acids. In another aspect, the invention provides a host cell comprising any of the foregoing expression vectors.

In another aspect, the invention provides a pharmaceutical composition comprising any of the foregoing fusion proteins or any of the foregoing antibody conjugates.

In another aspect, the invention provides a method of treating cancer in a subject in need thereof. The method comprises administering to the subject an effective amount of any of the foregoing fusion proteins, any of the foregoing antibody conjugates, or any of the foregoing pharmaceutical compositions.

In certain embodiments, the cancer is selected from a lymphoma (e.g., a B-cell lymphoma, Hodgkin lymphoma, lymphoblastic lymphoma), a leukemia (e.g., hairy cell leukemia, B-cell chronic lymphocytic leukemia, acute myeloid leukemia (AML), acute lymphoblastic leukemia), and a melanoma.

These and other aspects and features of the invention are described in the following detailed description and claims.

### DESCRIPTION OF THE DRAWINGS

The invention can be more completely understood with reference to the following drawings.

FIG. 1 depicts an SDS-PAGE gel showing recombinant human Neu1, Neu2, Neu3, and Salmonella typhimurium (St-sialidase) under non-reducing and reducing conditions. Monomer and dimer species are indicated.

FIG. 2 is a bar graph showing the enzymatic activity of recombinant human Neu1, Neu2, and Neu3.

FIG. 3 is a line graph showing enzymatic activity as a function of substrate concentration for recombinant human Neu2 and Neu3 at the indicated pH.

FIG. 4 depicts a schematic representation of an exemplary sialic acid biotinylated probe that can be used in phage display or yeast display screening for Neu2 variants.

FIG. 5 depicts an exemplary protocol that facilitates phage display screening of Neu2 variants.

FIG. 6 depicts an exemplary protocol that facilitates yeast display screening of Neu2 variants.

FIG. 7A depicts an SDS-PAGE gel showing recombinant Neu2-Fc (wildtype) and Neu2-M106-Fc under non-reducing and reducing conditions. FIG. 7B is an SEC-HPLC trace of Neu2-Fc (wildtype) and Neu2-M106-Fc. The monomer species has a retention time of 21 minutes.

FIG. 8 is a line graph depicting the enzymatic activity of Neu2 variant M106.

FIG. 9A depicts an SDS-PAGE gel showing Neu2-M173-Fc under non-reducing and reducing conditions. FIG. 9B is an SEC-HPLC trace of Neu2-M173-Fc. The monomer species has a retention time of 6.367 minutes. The monomer species has a purity of approximately 90% after purification by Protein A and CHT chromatography.

FIG. 10 depicts the enzyme activity of Neu2-M173-Fc, using 4-MU-Neu5Ac as the substrate, and fixing enzyme concentration to 2 μg/well.

FIG. 11A depicts an SDS-PAGE gel showing Neu2-M106 under non-reducing (NR) and reducing (R) conditions. FIG. 11B depicts a schematic representation of the Neu2 structure with the position of the R243 cleavage site indicated.

FIG. 12 depicts a reducing SDS-PAGE gel showing Neu2-M106 produced by a large or small scale expression with (+) or without (−) trypsin treatment.

FIG. 13 depicts an SDS-PAGE gel showing Neu2-M106 following incubation with trypsin and one of the protease inhibitors iron citrate (Fe Cit), aprotinin, AEBSF, leupeptin, or E-64 at the indicated concentrations.

FIG. 14 is a table depicting different mutations and combinations of mutations surrounding the trypsin cleavage site in Neu2.

FIG. 15A depicts a reducing SDS-PAGE analysis of Neu2 variants with the indicated mutation at position A242 with or without trypsin treatment. Trypsin digestion was for 5 minutes at 4° C. using a 5,000% dilution of trypsin. The digestion was quenched by addition of SDS, and 2 μg of protein was loaded on the gel. FIG. 15B depicts the enzymatic activity of Neu2 variants with the indicated mutation at position A242. FIG. 15C is an SEC-HPLC trace of Neu2 variants with the indicated mutation at position A242. Neu2-M106 (the mutational background in which the mutations at position A242 were tested) is shown as a control.

FIG. 16 depicts a reducing SDS-PAGE analysis of the indicated Neu2 variants with or without trypsin treatment. Neu2-M106 is shown as a control. For example, Neu2-M255 was shown to have a greater than 10 fold improved trypsin resistance relative to Neu2-M106.

FIGS. 17A-17I depict schematic representations of certain antibody conjugate constructs containing a sialidase enzyme, e.g., a human sialidase enzyme, and an anti-CD20 antigen binding site. For each antibody conjugate construct that contains more than one (e.g., two) sialidase, each sialidase may be the same or different. For each antibody conjugate construct that contains more than one (e.g., two) anti-CD20 antigen binding site, each anti-CD20 antigen binding site may be the same or different. For each antibody conjugate construct that contains an Fc domain, it is understood that the Fc domain can be a wild type Fc domain or can be an engineered Fc domain. For example, the Fc domain may be engineered to contain either a “knob” mutation, e.g., T366Y, or a “hole” mutation, e.g., Y407T, or both, to promote heterodimerization, or the Fc domain may be engineered to contain one or more modifications, e.g., point mutations, to provide any other modified Fc domain functionality.

FIG. 18 depicts schematic representations of certain antibody conjugate constructs containing a sialidase enzyme, e.g., a human sialidase enzyme, and an antigen binding site. For each antibody conjugate construct that contains more than one (e.g., two) antigen binding site, each antigen binding site may be the same or different. For each antibody conjugate construct that contains an Fc domain, it is understood that the Fc domain can be a wild type Fc domain or can be an engineered Fc domain. For example, the Fc domain may be engineered to contain either a “knob” mutation, e.g., T366Y, or a “hole” mutation, e.g., Y407T, or both, to promote heterodimerization, or the Fc domain may be engineered to contain one or more modifications, e.g., point mutations, to provide any other modified Fc domain functionality.

FIGS. 19A-19D are schematic representations of exemplary fusion protein conjugates referred to as a Raptor antibody sialidase conjugate (FIG. 19A), a Janus antibody sialidase conjugate (FIG. 19B), a Lobster antibody sialidase conjugate (FIG. 19C), and a Bunk antibody sialidase conjugate (FIG. 19D).

FIG. 20 depicts an SDS-PAGE gel showing purified recombinant human Janus Rituximab under non-reducing and reducing conditions.

FIG. 21 shows an SEC-HPLC trace of purified Janus Rituximab, showing good monomer purity.

FIG. 22 is a line graph showing enzyme activity of Janus Rituximab using 4-MU-Neu5Ac as the substrate and measured as the level of fluorescence (AU) generated. Specifically, when Janus Rituximab is incubated with the fluorogenic substrate 4-MU-Neu5Ac, it cleaves the substrate, generating fluorescence which is indicative of the sialidase activity of the construct.

FIG. 23 depicts an SDS-PAGE gel showing purified recombinant human Janus Ofatumumab under non-reducing and reducing conditions.

FIG. 24 shows an SEC-HPLC trace of purified Janus Ofatumumab, showing good monomer purity.

FIG. 25 is a line graph showing enzyme activity of Janus Ofatumumab using 4-MU-Neu5Ac as the substrate and measured as the level of fluorescence (AU) generated. Specifically, when Janus Ofatumumab is incubated with the fluorogenic substrate 4-MU-Neu5Ac, it cleaves the substrate, generating fluorescence which is indicative of the sialidase activity of the construct.

FIGS. 26A and 26B are bar graphs showing levels of PNA staining (geometric mean fluorescence intensity, gMFI) in transgenic cell lines EL4-CD20 and A20-CD20, respectively. Increased PNA staining in cells treated with Janus Rituximab and Janus Ofatumumab correlates with a decrease sialylation, demonstrating that Janus ASCs can remove sialic acid present on cells.

FIGS. 27A and 27B are bar graphs showing levels of Annexin V staining, a marker of apoptosis, in human B-cell lymphoma cell lines Raji and Ramos. Treatment with Janus Rituximab and Janus Ofatumumab resulted in increased Annexin-V staining in both cell lines, at levels comparable to or slightly higher than Rituximab or Ofatumumab. The increased Annexin-V staining indicates that Janus Rituximab and Janus Ofatumumab induce apoptosis in cancer cells.

FIG. 28A shows an SEC-HPLC trace of purified Janus Ofatumumab #2, with a large peak at ˜6.5 min indicating good monomer purity. FIG. 28B is a line graph showing enzyme activity of Janus Ofatumumab #2 using 4-MU-Neu5Ac as the substrate and measured as the level of fluorescence (AU) generated.

FIG. 29A shows an SEC-HPLC trace of purified Janus Ofatumumab #3, with a large peak at ˜6.5 min indicating good monomer purity. FIG. 29B is a line graph showing enzyme activity of Janus Ofatumumab #3 using 4-MU-Neu5Ac as the substrate and measured as the level of fluorescence (AU) generated.

FIG. 30A shows an SEC-HPLC trace of purified Janus Ofatumumab LOF, with a large peak at ˜20.8 min indicating acceptable monomer purity. FIG. 30B is a line graph showing enzyme activity of Janus Ofatumumab LOF using 4-MU-Neu5Ac as the substrate and measured as the level of fluorescence (AU) generated.

FIG. 31A shows an SEC-HPLC trace of purified Raptor Ofatumumab #1, with a large peak at ˜6 min indicating good monomer purity. FIG. 31B is a line graph showing enzyme activity of Raptor Ofatumumab #1 using 4-MU-Neu5Ac as the substrate and measured as the level of fluorescence (AU) generated.

FIG. 32A shows an SEC-HPLC trace of purified Raptor Ofatumumab #2, with a large peak at ˜6 min indicating good monomer purity. FIG. 32B is a line graph showing enzyme activity of Raptor Ofatumumab #2 using 4-MU-Neu5Ac as the substrate and measured as the level of fluorescence (AU) generated.

FIG. 33 shows the removal of cell surface sialic acids from either Raji (FIG. 33A) or Ramos cells (FIG. 33B) by Janus Ofatumumab #2 (“Janus #2”), Janus Ofatumumab #3 (“Janus #3”), Raptor Ofatumumab #1 (“Raptor #1”), Raptor Ofatumumab #2 (“Raptor #2”), or Janus Ofatumumab LOF (“Janus LOF”). Removal of cell surface sialic acids was measured using a hexameric version of the extracellular domain of human Siglec9 (Hydra9). Removal of cell surface sialic acid leads to a decrease in Hydra9 binding.

FIG. 34 shows the removal of cell surface sialic acids from either Raji (FIG. 34A) or Ramos cells (FIG. 34B) by Janus Ofatumumab #2 (“Janus #2”), Janus Ofatumumab #3 (“Janus #3”), Raptor Ofatumumab #1 (“Raptor #1”), Raptor Ofatumumab #2 (“Raptor #2”), or Janus Ofatumumab LOF (“Janus LOF”). Removal of cell surface sialic acids was measured using PNA to detect exposed galactose residues. Removal of cell surface sialic acid leads to an increase in PNA binding.

FIG. 35 depicts the ability of Janus Ofatumumab #2 (“Janus #2”), Janus Ofatumumab #3 (“Janus #3”), Raptor Ofatumumab #1 (“Raptor #1”), Raptor Ofatumumab #2 (“Raptor #2”), or Janus Ofatumumab LOF (“Janus LOF”) to bind cell surface CD20 on either Raji (FIG. 35A) or Ramos cells (FIG. 35B).

FIG. 36 depicts the in vivo activity of ofatumumab (“ofatumumab” or “ofa”), Janus Ofatumumab #2 (“Janus” 2), or Janus Ofatumumab LOF (“Janus LOF”) in a syngeneic EL4-CD20 lymphoma intravenous dissemination model as of day 28 (FIG. 36A) or at the end of in-life as of day 41 (FIG. 36B). Triangles indicate dosing of various test articles. The 50% survival point for each treatment group is shown.

### DETAILED DESCRIPTION

Various features and aspects of the invention are discussed in more detail below.

The invention relates to fusion proteins and/or antibody conjugates comprising a sialidase enzyme and an anti-CD20 antibody or portion thereof, e.g., an immunoglobulin Fc domain and/or an antigen-binding domain. The sialidase enzyme portion of the fusion protein and/or antibody conjugate may comprise at least one mutation relative to a wild-type sialidase. The mutations, or combination of mutations, can improve the expression, activity or both the expression and activity of the sialidase to improve its use in cancer diagnosis and/or treatment.

The invention further relates to pharmaceutical compositions and methods of using fusion proteins and/or antibody conjugates to treat cancer.

### I. Sialidase Anti-CD20 Fusion Proteins

To promote the selective removal of sialic acids on hypersialylated cancer cells, e.g., CD20 expressing cancer cells, and/or in the tumor microenvironment, it may be helpful to target a sialidase as described herein to such a cell or to such a tumor microenvironment. Additionally, in order to promote the removal of sialic acid by a sialidase in a subject, it may be helpful to extend the plasma half-life of the sialidase in the subject. These can be achieved by including the sialidase in a fusion protein and/or antibody conjugate (e.g., a chemically conjugated conjugate).

Accordingly, the invention further provides fusion proteins comprising a sialidase enzyme, or a functional fragment thereof, and a portion or fragment of an anti-CD20 antibody, such as an immunoglobulin Fc domain (also referred to herein as an Fc domain), or an immunoglobulin antigen-binding domain (also referred to herein as an antigen-binding domain). In certain embodiments, the sialidase and anti-CD20 antibody or portion thereof (e.g., immunoglobulin Fc domain or antigen-binding domain) are linked by a peptide bond or an amino acid linker.

As used herein, unless otherwise indicated, the term “fusion protein” is understood to refer to a single polypeptide chain comprising amino acid sequences based upon two or more separate proteins or polypeptide chains, where the two amino acid sequences may be fused together directly or via an intervening linker sequence, e.g., via an intervening amino acid linker. A nucleotide sequence encoding such a fusion protein can, for example, be created using conventional recombinant DNA technologies.

In certain embodiments, a fusion protein comprises a tag, such as a Strep tag (e.g., a Strep II tag), a His tag (e.g., a 10×His tag), a myc tag, or a FLAG tag. The tag can be located on the C-terminus or the N-terminus of the fusion protein.

a. Sialidase Portion

As used herein, the term “sialidase” refers to any enzyme, or a functional fragment thereof, that cleaves a terminal sialic acid residue from a substrate, for example, a glycoprotein or a glycolipid. The term sialidase includes variants having one or more amino acid substitutions, deletions, or insertions relative to a wild-type sialidase sequence, and/or fusion proteins or conjugates including a sialidase. Sialidases are also called neuraminidases, and, unless indicated otherwise, the two terms are used interchangeably herein. As used herein, the term “functional fragment” of a sialidase refers to fragment of a full-length sialidase that retains, for example, at least 10%, at least 20%, at least 30%, at least 40%, at least 50%, at least 60%, at least 70%, at least 80%, at least 90%, at least 95%, or 100% of the enzymatic activity of the corresponding full-length, naturally occurring sialidase. Sialidase enzymatic activity may be assayed by any method known in the art, including, for example, by measuring the release of sialic acid from the fluorogenic substrate 4-methylumbelliferyl-N-acetylneuraminic acid (4MU-NeuAc). In certain embodiments, the functional fragment comprises at least 100, 150, 200, 250, 300, 310, 320, 330, 340, 350, 360, or 370 consecutive amino acids present in a full-length, naturally occurring sialidase.

In certain embodiments, a sialidase portion of a sialidase-anti-CD20 fusion protein is derived from a eukaryotic sialidase, e.g., a mammalian sialidase, e.g., a human or mouse sialidase.

Four sialidases are encoded in the human genome: Neu1, Neu2, Neu3 and Neu4. Human Neu1 is a lysosomal neuraminidase enzyme which functions in a complex with beta-galactosidase and cathepsin A. The amino acid sequence of human Neu1 is depicted in SEQ ID NO: 7, and a nucleotide sequence encoding human Neu1 is depicted in SEQ ID NO: 23.

Human Neu2 is a cytosolic sialidase enzyme. The amino acid sequence of human Neu2 is depicted in SEQ ID NO: 1, and a nucleotide sequence encoding human Neu2 is depicted in SEQ ID NO: 24. Unless stated otherwise, as used herein, wild-type human Neu2 refers to human Neu2 having the amino acid sequence of SEQ ID NO: 1.

Human Neu3 is a plasma membrane sialidase with an activity specific for gangliosides. Human Neu3 has two isoforms: isoform 1 and isoform 2. The amino acid sequence of human Neu3, isoform 1 is depicted in SEQ ID NO: 8, and a nucleotide sequence encoding human Neu3, isoform 1 is depicted in SEQ ID NO: 25. The amino acid sequence of human Neu3, isoform 2 is depicted in SEQ ID NO: 9, and a nucleotide sequence encoding human Neu3, isoform 2 is depicted in SEQ ID NO: 34.

Human Neu4 has two isoforms: isoform 1 is a peripheral membrane protein and isoform 2 localizes to the lysosome lumen. The amino acid sequence of human Neu4, isoform 1 is depicted in SEQ ID NO: 10, and a nucleotide sequence encoding human Neu4, isoform 1 is depicted in SEQ ID NO: 26. The amino acid sequence of human Neu4, isoform 2 is depicted in SEQ ID NO: 11, and a nucleotide sequence encoding human Neu4, isoform 2 is depicted in SEQ ID NO: 35.

Four sialidases have also been found in the mouse genome and are referred to as Neu1, Neu2, Neu3 and Neu4. The amino acid sequence of mouse Neu1 is depicted in SEQ ID NO: 38, and a nucleotide sequence encoding mouse Neu1 is depicted in SEQ ID NO: 42. The amino acid sequence of mouse Neu2 is depicted in SEQ ID NO: 39 and a nucleotide sequence encoding mouse Neu2 is depicted in SEQ ID NO: 43. The amino acid sequence of mouse Neu3 is depicted in SEQ ID NO: 40, and a nucleotide sequence encoding mouse Neu3 is depicted in SEQ ID NO: 44. The amino acid sequence of mouse Neu4 is depicted in SEQ ID NO: 41, and a nucleotide sequence encoding mouse Neu4 is depicted in SEQ ID NO: 45.

In certain embodiments, a sialidase portion of a sialidase-anti-CD20 fusion protein is derived from a prokaryotic sialidase. Exemplary prokaryotic sialidases include sialidases from Salmonella typhimurium and Vibrio cholera. The amino acid sequence of Salmonella typhimurium sialidase (St-sialidase) is depicted in SEQ ID NO: 30, and a nucleotide sequence encoding Salmonella typhimurium sialidase is depicted in SEQ ID NO: 6. The amino acid sequence of Vibrio cholera sialidase is depicted in SEQ ID NO: 36, and a nucleotide sequence encoding Vibrio cholera sialidase is depicted in SEQ ID NO: 37.

In certain embodiments, the sialidase portion of a sialidase-anti-CD20 fusion protein is a mutant sialidase, e.g., a recombinant mutant human sialidase. In certain embodiments, the recombinant mutant human sialidase has about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more than 100% of the enzymatic activity of a corresponding (or template) wild-type human sialidase.

In certain embodiments, the recombinant mutant human sialidase has the same substrate specificity as the corresponding wild-type human sialidase. In other embodiments, the recombinant mutant human sialidase has a different substrate specificity than the corresponding wild-type human sialidase. For example, in certain embodiments the recombinant mutant human sialidase can cleave α2,3, α2,6, and/or α2,8 linkages. In certain embodiments the sialidase can cleave α2,3 and α2,8 linkages.

In certain embodiments, the expression yield of the recombinant mutant human sialidase in mammalian cells, e.g., HEK293 cells, CHO cells, murine myeloma cells (NS0, Sp2/0), or human fibrosarcoma cells (HT-1080), e.g., HEK293 cells, is greater than about 10%, about 20%, about 50%, about 75%, about 100%, about 150%, about 200%, about 250%, about 300%, about 400%, about 500%, about 600%, about 700%, about 800%, about 900%, or about 1,000% of the expression yield of the corresponding wild-type human sialidase.

In certain embodiments, the recombinant mutant human sialidase has about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more than 100% of the enzymatic activity of a corresponding wild-type human sialidase, and the expression yield of the recombinant mutant human sialidase in mammalian cells, e.g., HEK293 cells, is greater than about 10%, about 20%, about 50%, about 75%, about 100%, about 150%, about 200%, about 250%, about 300%, about 400%, about 500%, about 600%, about 700%, about 800%, about 900%, or about 1,000% of the expression yield of a corresponding wild-type human sialidase.

In certain embodiments, the amino acid sequence of the recombinant mutant human sialidase has at least 50%, 55%, 60%, 65%, 70%, 75%, 80%, 85%, 90%, 95%, 96%, 97%, 98%, or 99% sequence identity to the amino acid sequence of a corresponding wild-type human sialidase.

1. Substitution of Cysteine Residues

In certain embodiments, the recombinant mutant human sialidase comprises a substitution of at least one cysteine (cys, C) residue. It has been discovered that certain cysteine residues in sialidases may inhibit expression of functional protein as a result of protein aggregation. Accordingly, in certain embodiments, the recombinant mutant human sialidase contains at least one mutation to remove a free cysteine (e.g., for Neu1 (SEQ ID NO: 7), a mutation of, for example, one or more of C111, C117, C171, C183, C218, C240, C242, and C252; for Neu2 (SEQ ID NO: 1), a mutation of, for example, one or more of C125, C196, C219, C272, C332, and C352; for Neu3 (SEQ ID NO: 8), a mutation of, for example, one or more of C7, C90, C99, C106, C127, C136, C189, C194, C226, C242, C250, C273, C279, C295, C356, C365, C368, C384, C383, C394, and C415; and for Neu4 (SEQ ID NO: 10), a mutation of, for example, one or more of C88, C125, C126, C186, C191, C211, C223, C239, C276, C437, C453, C480, and C481). Free cysteines can be substituted with any amino acid. In certain embodiments, the free cysteine is substituted with serine (ser, S), isoleucine (iso, I), valine (val, V), phenylalanine (phe, F), leucine (leu, L), or alanine (ala, A). Exemplary cysteine substitutions in Neu2 include C125A, C1251, C125S, C125V, C196A, C196L, C196V, C272S, C272V, C332A, C332S, C332V, C352L, and C352V.

In certain embodiments, the recombinant mutant human sialidase comprises two or more cysteine substitutions. Exemplary double or triple cysteine substitutions in Neu2 include: C125S and C332S; C272V and C332A; C272V and C332S; C332A and C352L; C125S and C196L; C196L and C352L; C196L and C332A; C332A and C352L; and C196L, C332A and C352L.

In certain embodiments, the recombinant mutant human sialidase is a Neu2 sialidase and comprises the substitutions C322A and C352L (SEQ ID NO: 5).

In certain embodiments, the sialidase contains an amino acid substitution at 2, 3, 4, 5, or 6 cysteines typically present in a human sialidase, e.g., Neu2 or Neu3.

In certain embodiments, the recombinant mutant human sialidase comprises a substitution or combination of substitutions corresponding to a substitution or combination of substitutions listed in TABLE 1 (amino acid positions corresponding to wild-type human Neu2 (SEQ ID NO: 1)).

TABLE 1
Substitution(s)

C125A
C125I
C125S
C125V
C196A
C196L
C196V
C272S
C272V
C332A
C332S
C332V
C352L
C352V
C125S + C332S
C272V + C332A
C272V + C332S
C332A + C352L
C125S + C196L
C196L + C352L
C196L + C332A
C196L + C332A + C352L

2. Substitutions of Residues to Increase pI and/or Decrease Hydrophobicity

The isoelectric point (pI) of a protein is the pH at which the net charge is zero. The pI also generally indicates the pH at which the protein is least soluble, which may affect the ability to express and purify the protein. Generally, a protein has good solubility if its pI is greater than 2 units above the pH of the solution. Human Neu2 has a predicted pI of 7.5. Thus, human Neu2 is least soluble around neutral pH, which is undesirable because expression and physiological systems are at neutral pH. In contrast, the sialidase from Salmonella typhimurium (St-sialidase), which exhibits good solubility and recombinant expression, has a pI of 9.6. Accordingly, to increase expression of human Neu2 or the other human sialidases, a recombinant mutant human sialidase may be designed to contain one or more amino acid substitution(s) wherein the substitution(s) increase(s) the pI of the sialidase relative to a sialidase without the substitution. Additionally, decreasing the number of hydrophobic amino acids on the surface of a sialidase may improve expression of sialidase by, for example, reducing aggregation. Accordingly, to increase expression of human Neu2 or the other human sialidases, a recombinant mutant human sialidase may be designed to contain one or more amino acid substitution(s) wherein the substitution(s) decrease(s) the hydrophobicity of a surface of the sialidase relative to a sialidase without the substitution(s).

Accordingly, in certain embodiments, the recombinant mutant human sialidase comprises at least one amino acid substitution, wherein the substitution increases the isoelectric point (pI) of the sialidase and/or decreases the hydrophobicity of the sialidase relative to a sialidase without the substitution. This may be achieved by introducing one or more charged amino acids, for example, positively or negatively charged amino acids, into the recombinant sialidase. In certain embodiments, the amino acid substitution is to a charged amino acid, for example, a positively charged amino acid such as lysine (lys, K), histidine (his, H), or arginine (arg, R), or a negatively charged amino acid such as aspartic acid (asp, D) or glutamic acid (glu, E). In certain embodiments, the amino acid substitution is to a lysine residue. In certain embodiments, the substitution increases the pI of the sialidase to about 7.75, about 8, about 8.25, about 8.5, about 8.75, about 9, about 9.25, about 9.5, or about 9.75.

In certain embodiments, the amino acid substitution occurs at a surface exposed D or E amino acid, in a helix or loop, or in a position that has a K or R in the corresponding position of St-sialidase. In certain embodiments, the amino acid substitution occurs at an amino acid that is remote from the catalytic site or otherwise not involved in catalysis, an amino acid that is not conserved with the other human Neu proteins or with St-Sialidase or Clostridium NanH, or an amino acid that is not located in a domain important for function (e.g., an Asp-box or beta strand).

Exemplary amino acid substitutions in Neu2 that increase the isoelectric point (pI) of the sialidase and/or decrease the hydrophobicity of the sialidase relative to a sialidase without the substitution include A2E, A2K, D215K, V325E, V325K, E257K, and E319K. In certain embodiments, the recombinant mutant human sialidase comprises two or more amino acid substitutions, including, for example, A2K and V325E, A2K and V325K, E257K and V325K, A2K and E257K, and E257K and A2K and V325K.

In certain embodiments, the recombinant mutant human sialidase comprises a substitution or combination of substitutions corresponding to a substitution or combination of substitutions listed in TABLE 2 (amino acid positions corresponding to wild-type human Neu2 (SEQ ID NO: 1)).

TABLE 2
Substitution(s)

A2K
E72K
D215K
E257K
V325K
A2K + E257K
A2K + V325E
A2K + V325K
E257K + V325K

3. Addition of N-Terminal Peptides and N- or C-Terminal Substitutions

It has been discovered that the addition of a peptide sequence of two or more amino acids to the N-terminus of a human sialidase can improve expression and/or activity of the sialidase. In certain embodiments, the peptide is at least 2 amino acids in length, for example, from 2 to 20, from 2 to 10, from 2 to 5, or 2, 3, 4, 5, 6, 7, 8, 9, 10, 11, 12, 13, 14, 15, 16, 17, 18, 19, or 20 amino acids in length. In certain embodiments, the peptide may form, or have a propensity to form, an α-helix.

In mice, a Neu2 isoform (type B) found in thymus contains six amino acids not present in the canonical isoform of Neu2 found in skeletal muscle. In certain embodiments herein, the N-terminal six amino acids of the mouse thymus Neu2 isoform, MEDLRP (SEQ ID NO: 4), or variations thereof, can be added onto a human Neu, e.g., human Neu2. In certain embodiments, the recombinant mutant human sialidase comprises a peptide at least two amino acid residues in length covalently associated with an N-terminal amino acid of the sialidase. In certain embodiments the recombinant mutant human sialidase comprises the peptide MEDLRP (SEQ ID NO: 4) or EDLRP (SEQ ID NO: 3) covalently associated with an N-terminal amino acid of the sialidase. In certain embodiments, the sialidase may further comprise a cleavage site, e.g., a proteolytic cleavage site, located between the peptide, e.g., MEDLRP (SEQ ID NO: 4) or EDLRP (SEQ ID NO: 3), and the remainder of the sialidase. In certain embodiments, the peptide, e.g., MEDLRP (SEQ ID NO: 4) or EDLRP (SEQ ID NO: 3), may be post-translationally cleaved from the remainder of the sialidase.

Alternatively to, or in combination with, the N-terminal addition, 1-5 amino acids of the 12 amino acid N-terminal region of the recombinant mutant human sialidase may be removed, e.g., the N-terminal methionine can be removed. In certain embodiments, if the recombinant mutant human sialidase is Neu2, the N-terminal methionine can be removed, the first five amino acids (MASLP; SEQ ID NO: 12) can be removed, or the second through fourth amino acids (ASLP; SEQ ID NO: 13) can be removed.

In certain embodiments, 1-5 amino acids of the 12 amino acid N-terminal region of the recombinant mutant human sialidase are substituted with MEDLRP (SEQ ID NO: 4), EDLRP (SEQ ID NO: 3), or TVEKSVVF (SEQ ID NO: 14). For example, in certain embodiments, if the recombinant mutant human sialidase is Neu2, the amino acids MASLP (SEQ ID NO: 12), ASLP (SEQ ID NO: 13) or M are substituted with MEDLRP (SEQ ID NO: 4), EDLRP (SEQ ID NO: 3) or TVEKSVVF (SEQ ID NO: 14).

Human sialidases have a β-propeller structure, characterized by 6 blade-shaped β-sheets arranged toroidally around a central axis. Generally, hydrophobic interactions between the blades of a β-propeller, including between the N- and C-terminal blades, enhance stability. Accordingly, in order to increase expression of human Neu2 or the other human sialidases, a recombinant mutant human sialidase can be designed comprising an amino acid substitution that increases hydrophobic interactions and/or hydrogen bonding between the N- and C-terminal β-propeller blades of the sialidase.

Accordingly, in certain embodiments, the recombinant mutant human sialidase comprises a substitution of at least one wild-type amino acid residue, wherein the substitution increases hydrophobic interactions and/or hydrogen bonding between the N- and C-termini of the sialidase relative to a sialidase without the substitution. In certain embodiments, the wild-type amino acid is substituted with asparagine (asn, N), lysine (lys, K), tyrosine (tyr, Y), phenylalanine (phe, F), or tryptophan (trp, W). Exemplary substitutions in Neu2 that increase hydrophobic interactions and/or hydrogen bonding between the N- and C-termini include L4N, L4K, V6Y, L7N, L4N and L7N, L4N and V6Y and L7N, V12N, V12Y, V12L, V6Y, V6F, or V6W. In certain embodiments, the sialidase comprises the V6Y substitution.

In certain embodiments, the recombinant mutant human sialidase comprises a combination of the above substitutions. For example, a recombinant mutant human Neu2 sialidase can comprise the additional amino acids MEDLRP (SEQ ID NO: 4), EDLRP (SEQ ID NO: 3), or TVEKSVVF (SEQ ID NO: 14) at the N-terminus and, in combination, can comprise at least one L4N, L4K, V6Y, L7N, L4N and L7N, L4N and V6Y and L7N, V12N, V12Y, V12L, V6Y, V6F, or V6W substitution. In certain embodiments, the amino acids MASLP (SEQ ID NO: 12), ASLP (SEQ ID NO: 13) or M of a recombinant mutant human Neu2 sialidase are replaced with MEDLRP (SEQ ID NO: 4), EDLRP (SEQ ID NO: 3) or TVEKSVVF (SEQ ID NO: 14) and the recombinant mutant human Neu2 sialidase also comprises at least one L4N, L4K, V6Y, L7N, L4N and L7N, L4N and V6Y and L7N, V12N, V12Y, V12L, V6Y, V6F, or V6W substitution.

In certain embodiments, the recombinant mutant human sialidase comprises a mutation or combination of mutations corresponding to a mutation or combination of mutations listed in TABLE 3 (amino acid positions corresponding to wild-type human Neu2 (SEQ ID NO: 1)).

TABLE 3
Mutation(s)

Substitute M at the N-terminus with EDLRP
(SEQ ID NO: 3)
Substitute M at the N-terminus with MEDLRP
(SEQ ID NO: 4)
Insert MEDLRP (SEQ ID NO: 4) at the N-terminus
Substitute MASLP (SEQ ID NO: 12)
at the N-terminus with
MEDLRP (SEQ ID NO: 4)
L4N
V6Y
L7N
V6F
V6W

Additionally, in certain embodiments, the sialidase comprises a substitution or deletion of an N-terminal methionine at the N-terminus of the sialidase. For example, in certain embodiments, the sialidase comprises a substitution of a methionine residue at a position corresponding to position 1 of wild-type human Neu2 (SEQ ID NO: 1), e.g., the methionine at a position corresponding to position 1 of wild-type human Neu2 is substituted by alanine (M1A) or aspartic acid (M1D). In other embodiments, the sialidase comprises a deletion of a methionine residue at a position corresponding to position 1 (ΔM1) of wild-type human Neu2 (SEQ ID NO: 1).

In certain embodiments, the recombinant mutant human sialidase comprises a substitution or combination of substitutions corresponding to a substitution or combination of substitutions listed in TABLE 4 (amino acid positions corresponding to wild-type human Neu2 (SEQ ID NO: 1)).

TABLE 4
Mutation(s)

Deletion of M1, V6Y, I187K
M1R, V6Y, I187K
M1H, V6Y, I187K
M1K, V6Y, I187K
M1D, V6Y, I187K
M1T, V6Y, I187K
M1N, V6Y, I187K
M1Q, V6Y, I187K
M1G, V6Y, I187K
M1A, V6Y, I187K
M1V, V6Y, I187K
M1L, V6Y, I187K
M1F, V6Y, I187K
M1Y, V6Y, I187K

4. Substitutions of Residues to Decrease Proteolytic Cleavage

It has been discovered that certain sialidases (e.g., human Neu2) are susceptible to cleavage by a protease (e.g., trypsin). As a result, proteolytic cleavage of the sialidase may occur during recombinant protein production, harvesting, purification, or formulation, during administration to a subject, or after administration to a subject. Accordingly, in certain embodiments, the recombinant mutant human sialidase comprises a substitution of at least one wild-type amino acid residue, wherein the substitution decreases cleavage of the sialidase by a protease (e.g., trypsin) relative to a sialidase without the substitution.

In certain embodiments, incubation of the recombinant mutant human sialidase with a protease (e.g., trypsin) results in from about 1% to about 50%, from about 1% to about 40%, from about 1%, to about 30%, from about 1% to about 20%, from about 1% to about 10%, from about 1% to about 5%, from about 5% to about 50%, from about 5% to about 40%, from about 5% to about 30%, from about 5% to about 20%, from about 5% to about 10%, from about 10% to about 50%, from about 10% to about 40%, from about 10% to about 30%, from about 10% to about 20%, from about 20% to about 50%, from about 20% to about 40%, from about 20% to about 30%, from about 30% to about 50%, from about 30% to about 40%, or from about 40% to about 50% of the proteolytic cleavage of a corresponding wild-type sialidase when incubated with the protease under the same conditions. In certain embodiments, incubation of the recombinant mutant human sialidase with a protease (e.g., trypsin) results in less than 50%, less than 40%, less than 30%, less than 10%, less than 5%, less than 3%, less than 1%, or less than 0.5% of the proteolytic cleavage of a corresponding wild-type sialidase when incubated with the protease under the same conditions. Proteolytic cleavage can be assayed by any method known in the art, including for example, by SDS-PAGE as described in Example 4 herein.

Exemplary substitutions that increase resistance to proteolytic cleavage include: (i) a substitution of an alanine residue at a position corresponding to position 242 of wild-type human Neu2 (SEQ ID NO: 1), e.g., a substitution by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (ii) a substitution of an arginine residue at a position corresponding to position 243 of wild-type human Neu2 (SEQ ID NO: 1), e.g., a substitution by glutamic acid (R243E), histidine (R243H), asparagine (R243N), glutamine (R243Q), or lysine (R243K); (iii) a substitution of a valine residue at a position corresponding to position 244 of wild-type human Neu2 (SEQ ID NO: 1), e.g., a substitution by isoleucine (V244I), lysine (V244K), or proline (V244P); or (iv) a combination of any of the foregoing. In certain embodiments, the recombinant mutant human sialidase comprises a substitution selected from A242C, A242F, A242Y, and A242W. In certain embodiments, the recombinant mutant human sialidase comprises a substitution or a combination of substitutions corresponding to a substitution or a combination of substitutions listed in TABLE 5 (amino acid positions corresponding to wild-type human Neu2 (SEQ ID NO: 1)).

TABLE 5

| Wild Type | |
|---|---|
| Human Neu2 | |
| (SEQ ID NO: 1) | |
| Amino Acid | Exemplary Substitution(s) at Specified Position(s) |
| A242 | C, F, G, H, I, K, L, M, N, P, Q, R, S, V, W, Y |
| R243 | E, H, N, Q, K |
| V244 | I, K, P |

Additional exemplary substitutions that increase resistance to proteolytic cleavage (and/or increase expression yield and/or enzymatic activity) include: (i) a substitution of a leucine residue at a position corresponding to position 240 of wild-type human Neu2 (SEQ ID NO: 1), e.g., a substitution by aspartic acid (L240D), asparagine (L240N), or tyrosine (L240Y); (ii) a substitution of an alanine residue at a position corresponding to position 213 of wild-type human Neu2 (SEQ ID NO: 1), e.g., a substitution by cysteine (A213C), asparagine (A213N), serine (A213S), or threonine (A213T); (iii) a substitution of an arginine residue at a position corresponding to position 241 of wild-type human Neu2 (SEQ ID NO: 1), e.g., a substitution by alanine (R241A), aspartic acid (R241D), leucine (R241L), glutamine (R241Q). or tyrosine (R241Y); (iv) a substitution of a serine residue at a position corresponding to position 258 of wild-type human Neu2 (SEQ ID NO: 1), e.g., a substitution by cysteine (S258C); (v) a substitution of a leucine residue at a position corresponding to position 260 of wild-type human Neu2 (SEQ ID NO: 1), e.g., a substitution by aspartic acid (L260D), phenylalanine (L260F), glutamine (L260Q), or threonine (L260T); (vi) a substitution of a valine residue at a position corresponding to position 265 of wild-type human Neu2 (SEQ ID NO: 1), e.g., a substitution by phenylalanine (V265F); or (vii) a combination of any of the foregoing. It is contemplated that, in certain embodiments, a substitution or a combination of substitutions at these positions may improve hydrophobic and/or aromatic interaction between secondary structure elements in the sialidase (e.g., between an α-helix and the nearest β-sheet) thereby stabilizing the structure and improving resistance to proteolytic cleavage.

In certain embodiments, the recombinant mutant sialidase comprises a mutation at position L240. In certain embodiments, the recombinant mutant sialidase comprises a combination of mutations at positions (i) A213 and A242, (ii) A213, A242, and S258, (iii) L240 and L260, (iv) R241 and A242, (v) A242 and L260, (vi) A242 and V265, or (vii) L240 and A242. In certain embodiments, the recombinant mutant human sialidase comprises a combination of substitutions selected from (i) A213C, A242F, and S258C, (ii) A213C and A242F, (iii) A213T and A242F, (iv) R241Y and A242F, and (v) L240Y and A242F. In certain embodiments, the recombinant mutant human sialidase comprises a substitution or combination of substitutions corresponding to a substitution or combination of substitutions listed in TABLE 6 (amino acid positions corresponding to wild-type human Neu2 (SEQ ID NO: 1)).

TABLE 6
Substitution(s)

A242C, V244P
A242R, V244R
A242R, V244H
A242Y, V244P
A242T, V244P
A242N, V244P
A213C, A242F
A213S, A242F
A213T, A242F
A213N, A242F
A213C, A242F, S258C
A242F, L260F
A242F, V265F
L240Y
L240Y, L260F
L240D, L260T
L240N, L260T
L240N, L260D
L240N, L260Q
L240Y, A242F
R241A, A242F
R241Y, A242F

5. Other Substitutions

In certain embodiments, the recombinant mutant human sialidase comprises at least one of the following substitutions: I187K, A328E, K370N, or H210N. In certain embodiments, a recombinant mutant human Neu2 comprises the substitution of the amino acids GDYDAPTHQVQW (SEQ ID NO: 15) with the amino acids SMDQGSTW (SEQ ID NO: 16) or STDGGKTW (SEQ ID NO: 17). In certain embodiments, a recombinant mutant human Neu2 comprises the substitution of the amino acids PRPPAPEA (SEQ ID NO: 18) with the amino acids QTPLEAAC (SEQ ID NO: 19). In certain embodiments, a recombinant mutant human Neu2 comprises the substitution of the amino acids NPRPPAPEA (SEQ ID NO: 20) with the amino acids SQNDGES (SEQ ID NO: 21).

In certain embodiments, the recombinant mutant human sialidase comprises at least one substitution at a position corresponding to V212, A213, Q214, D215, T216, L217, E218, C219, Q220, V221, A222, E223, V224, E225, or T225.

In certain embodiments, the recombinant mutant human sialidase comprises an amino acid substitution at a position identified in TABLE 7 (amino acid positions corresponding to wild-type human Neu2 (SEQ ID NO: 1). In certain embodiments, the sialidase comprises an amino acid substitution identified in TABLE 7. In certain embodiments, the sialidase comprises a combination of any amino acid substitutions identified in TABLE 7.

TABLE 7

| Wild Type | |
|---|---|
| Human Neu2 | |
| (SEQ ID NO: 1) | |
| Amino Acid | Exemplary Substitution(s) at Specified Position(s) |
| M1 | D |
| L4 | S, T, Y, L, F, A, P, V, I, N, D, or H |
| P5 | G |
| V6 | Y |
| L7 | F, Y, S, I, T, or N |
| K9 | D |
| V12 | L, A, P, V, N, D, or H |
| F13 | S, N, R, K, T, G, D, E, or A |
| I22 | S, N, R, K, T, G, D, E, A, Y, L, F, P, V, I, or H |
| A24 | S, N, R, K, T, G, D, E, A, Y, L, F, P, V, I, or H |
| L34 | S, T, Y, L, F, A, P, V, I, N, D, or H |
| A36 | S, T, Y, L, F, A, P, V, I, N, D, or H |
| K44 | R or E |
| K45 | A, E, or R |
| L54 | M |
| P62 | H, G, N, T, S, F, I, D, or E |
| H64 | F, Y, S, I, T, or N |
| Q69 | H |
| R78 | K |
| D80 | P |
| P89 | S, T, Y, L, F, A, P, V, I, N, D, H, or M |
| A93 | EorK |
| G107 | D |
| Q108 | H |
| Q112 | R or K |
| C125 | Y, F, or L |
| Q126 | E, F, H, I, L, or Y |
| A150 | V |
| T156 | R, N, D, C, G, H, I, L, F, S, Y, V, A, P, or T |
| F157 | R, N, D, C, G, H, I, L, F, S, Y, V, A, or P |
| A158 | R, N, D, C, G, H, I, L, F, S, Y, V, A, P, or T |
| V159 | R, N, D, C, G, H, I, L, F, S, Y, V, A, or P |
| G160 | R, N, D, C, G, H, I, L, F, S, Y, V, A, P, or T |
| P161 | R, N, D, C, G, H, I, L, F, S, Y, V, A, or P |
| G162 | R, N, D, C, G, H, I, L, F, S, Y, V, A, P, or T |
| H163 | R, N, D, C, G, H, I, L, F, S, Y, V, A, or P |
| C164 | R, N, D, C, G, H, I, L, F, S, Y, V, A, P, or T |
| L165 | R, N, D, C, G, H, I, L, F, S, Y, V, A, or P |
| R170 | P |
| A171 | G |
| V176 | R, N, D, C, G, H, I, L, F, S, Y, V, P, or A |
| P177 | S, T, Y, L, F, A, P, V, I, N, D, or H |
| A178 | S, T, Y, L, F, A, P, V, I, N, D, or H |
| L184 | S, N, R, K, T, G, D, E, A, F, H, I, L, P, V, or Y |
| H185 | S, N, R, K, T, G, D, E, or A |
| P186 | S, N, R, K, T, G, D, E, A, F, H, I, L, P, V, or Y |
| I187 | S, N, R, K, T, G, D, E, or A |
| Q188 | P, S, N, R, K, T, G, D, E, or A |
| R189 | P |
| P190 | F, M, A, D, G, H, N, P, R, S, or T |
| I191 | M, A, D, F, H, I, L, N, P, S, T, V, Y, E, G, K, or R |
| A194 | S, T, Y, L, F, A, P, V, I, N, D, or H |
| A213 | C, N, S, or T |
| L217 | R, N, D, C, G, H, I, L, F, S, Y, or V |
| C219 | R, N, D, C, G, H, I, L, F, S, Y, or V |
| A222 | D |
| E225 | P |
| H239 | P |
| L240 | D, N, or Y |
| R241 | A, D, L, Q, or Y |
| A242 | C, F, G, H, I, K, L, M, N, Q, R, S, V, W, or Y |
| V244 | I or P |
| T249 | A |
| D251 | G |
| E257 | P |
| S258 | C |
| L260 | D, F, Q, or T |
| V265 | F |
| Q270 | S, T, A, H, P, or F |
| G271 | S, N, R, K, T, G, D, E, or A |
| C272 | S, N, R, K, T, G, D, E, A, C, H, Y, F, H, L, P, or V |
| W292 | R |
| S301 | A, D, E, F, G, H, I, K, L, M, N, P, Q, T, V, W, Y, |
| | C, or R |
| W302 | A, D, E, F, G, H, I, L, M, N, P, Q, R, S, T, V, Y, or |
| | K |
| E319 | D |
| V325 | F, Y, S, I, T, N, A, D, H, L, P, or V |
| L326 | F, Y, S, I, T, N, A, D, H, L, P, or V |
| L327 | F, Y, S, I, T, N, A, D, H, L, P, or V |
| C332 | A, D, G, H, N, P, R, S, or T |
| Y359 | A or S |
| V363 | R, S, T, Y, L, F, A, P, V, I, N, D, or H |
| L365 | K, Q, F, Y, S, I, T, N, A, D, H, L, P, or V |

For example, in certain embodiments, the recombinant mutant human sialidase comprises: (a) a substitution of a proline residue at a position corresponding to position 5 of wild-type human Neu2 (P5); (b) a substitution of a lysine residue at a position corresponding to position 9 of wild-type human Neu2 (K9); (c) a substitution of a lysine residue at a position corresponding to position 44 of wild-type human Neu2 (K44); (d) a substitution of a lysine residue at a position corresponding to position 45 of wild-type human Neu2 (K45); (e) a substitution of a leucine residue at a position corresponding to position 54 of wild-type human Neu2 (L54); (f) a substitution of a proline residue at a position corresponding to position 62 of wild-type human Neu2 (P62); (g) a substitution of a glutamine residue at a position corresponding to position 69 of wild-type human Neu2 (Q69); (h) a substitution of an arginine residue at a position corresponding to position 78 of wild-type human Neu2 (R78); (i) a substitution of an aspartic acid residue at a position corresponding to position 80 of wild-type human Neu2 (D80); (j) a substitution of an alanine residue at a position corresponding to position 93 of wild-type human Neu2 (A93); (k) a substitution of a glycine residue at a position corresponding to position 107 of wild-type human Neu2 (G107); (l) a substitution of a glutamine residue at a position corresponding to position 108 of wild-type human Neu2 (Q108); (m) a substitution of a glutamine residue at a position corresponding to position 112 of wild-type human Neu2 (Q112); (n) a substitution of a cysteine residue at a position corresponding to position 125 of wild-type human Neu2 (C125); (o) a substitution of a glutamine residue at a position corresponding to position 126 of wild-type human Neu2 (Q126); (p) a substitution of an alanine residue at a position corresponding to position 150 of wild-type human Neu2 (A150); (q) a substitution of a cysteine residue at a position corresponding to position 164 of wild-type human Neu2 (C164); (r) a substitution of an arginine residue at a position corresponding to position 170 of wild-type human Neu2 (R170); (s) a substitution of an alanine residue at a position corresponding to position 171 of wild-type human Neu2 (A171); (t) a substitution of a glutamine residue at a position corresponding to position 188 of wild-type human Neu2 (Q188); (u) a substitution of an arginine residue at a position corresponding to position 189 of wild-type human Neu2 (R189); (v) a substitution of an alanine residue at a position corresponding to position 213 of wild-type human Neu2 (A213); (w) a substitution of a leucine residue at a position corresponding to position 217 of wild-type human Neu2 (L217); (x) a substitution of a glutamic acid residue at a position corresponding to position 225 of wild-type human Neu2 (E225); (y) a substitution of a histidine residue at a position corresponding to position 239 of wild-type human Neu2 (H239); (z) a substitution of a leucine residue at a position corresponding to position 240 of wild-type human Neu2 (L240); (aa) a substitution of an arginine residue at a position corresponding to position 241 of wild-type human Neu2 (R241); (bb) a substitution of an alanine residue at a position corresponding to position 242 of wild-type human Neu2 (A242); (cc) a substitution of a valine residue at a position corresponding to position 244 of wild-type human Neu2 (V244); (dd) a substitution of a threonine residue at a position corresponding to position 249 of wild-type human Neu2 (T249); (ee) a substitution of an aspartic acid residue at a position corresponding to position 251 of wild-type human Neu2 (D251); (ff) a substitution of a glutamic acid residue at a position corresponding to position 257 of wild-type human Neu2 (E257); (gg) a substitution of a serine residue at a position corresponding to position 258 of wild-type human Neu2 (S258); (hh) a substitution of a leucine residue at a position corresponding to position 260 of wild-type human Neu2 (L260); (ii) a substitution of a valine residue at a position corresponding to position 265 of wild-type human Neu2 (V265); (jj) a substitution of a glutamine residue at a position corresponding to position 270 of wild-type human Neu2 (Q270); (kk) a substitution of a tryptophan residue at a position corresponding to position 292 of wild-type human Neu2 (W292); (ll) a substitution of a serine residue at a position corresponding to position 301 of wild-type human Neu2 (S301); (mm) a substitution of a tryptophan residue at a position corresponding to position 302 of wild-type human Neu2 (W302); (nn) a substitution of a valine residue at a position corresponding to position 363 of wild-type human Neu2 (V363); or (oo) a substitution of a leucine residue at a position corresponding to position 365 of wild-type human Neu2 (L365); or a combination of any of the foregoing substitutions. For example, the sialidase may comprise a substitution of K9, P62, A93, Q216, A242, Q270, 5301, W302, V363, or L365, or a combination of any of the foregoing substitutions.

In certain embodiments, in the sialidase: (a) the proline residue at a position corresponding to position 5 of wild-type human Neu2 is substituted by histidine (P5H); (b) the lysine residue at a position corresponding to position 9 of wild-type human Neu2 is substituted by aspartic acid (K9D); (c) the lysine residue at a position corresponding to position 44 of wild-type human Neu2 is substituted by arginine (K44R) or glutamic acid (K44E); (d) the lysine residue at a position corresponding to position 45 of wild-type human Neu2 is substituted by alanine (K45A), arginine (K45R), or glutamic acid (K45E); (e) the leucine residue at a position corresponding to position 54 of wild-type human Neu2 is substituted by methionine (L54M); (f) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T); (g) the glutamine residue at a position corresponding to position 69 of wild-type human Neu2 is substituted by histidine (Q69H); (h) the arginine residue at a position corresponding to position 78 of wild-type human Neu2 is substituted by lysine (R78K); (i) the aspartic acid residue at a position corresponding to position 80 of wild-type human Neu2 is substituted by proline (D80P); (j) the alanine residue at a position corresponding to position 93 of wild-type human Neu2 is substituted by glutamic acid (A93E) or lysine (A93K); (k) the glycine residue at a position corresponding to position 107 of wild-type human Neu2 is substituted by aspartic acid (G107D); (l) the glutamine residue at a position corresponding to position 108 of wild-type human Neu2 is substituted by histidine (Q108H); (m) the glutamine residue at a position corresponding to position 112 of wild-type human Neu2 is substituted by arginine (Q112R) or lysine (Q112K); (n) the cysteine residue at a position corresponding to position 125 of wild-type human Neu2 is substituted by leucine (C125L); (o) the glutamine residue at a position corresponding to position 126 of wild-type human Neu2 is substituted by leucine (Q126L), glutamic acid (Q126E), phenylalanine (Q126F), histidine (Q126H), isoleucine (Q126I), or tyrosine (Q126Y); (p) the alanine residue at a position corresponding to position 150 of wild-type human Neu2 is substituted by valine (A150V); (q) the cysteine residue at a position corresponding to position 164 of wild-type human Neu2 is substituted by glycine (C164G); (r) the arginine residue at a position corresponding to position 170 of wild-type human Neu2 is substituted by proline (R170P); (s) the alanine residue at a position corresponding to position 171 of wild-type human Neu2 is substituted by glycine (A171G); (t) the glutamine residue at a position corresponding to position 188 of wild-type human Neu2 is substituted by proline (Q188P); (u) the arginine residue at a position corresponding to position 189 of wild-type human Neu2 is substituted by proline (R189P); (v) the alanine residue at a position corresponding to position 213 of wild-type human Neu2 is substituted by cysteine (A213C), asparagine (A213N), serine (A213S), or threonine (A213T); (w) the leucine residue at a position corresponding to position 217 of wild-type human Neu2 is substituted by alanine (L217A) or valine (L217V); (x) the threonine residue at a position corresponding to position 249 of wild-type human Neu2 is substituted by alanine (T249A); (y) the aspartic acid residue at a position corresponding to position 251 of wild-type human Neu2 is substituted by glycine (D251G); (z) the glutamic acid residue at a position corresponding to position 225 of wild-type human Neu2 is substituted by proline (E225P); (aa) the histidine residue at a position corresponding to position 239 of wild-type human Neu2 is substituted by proline (H239P); (bb) the leucine residue at a position corresponding to position 240 of wild-type human Neu2 is substituted by aspartic acid (L240D), asparagine (L240N), or tyrosine (L240Y); (cc) the arginine residue at a position corresponding to position 241 of wild-type human Neu2 is substituted by alanine (R241A), aspartic acid (R241D), leucine (R241L), glutamine (R241Q). or tyrosine (R241Y); (dd) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (ee) the valine residue at a position corresponding to position 244 of wild-type human Neu2 is substituted by isoleucine (V244I), lysine (V244K), or proline (V244P); (ff) the glutamic acid residue at a position corresponding to position 257 of wild-type human Neu2 is substituted by proline (E257P); (gg) the serine residue at a position corresponding to position 258 is substituted by cysteine (S258C); (hh) the leucine residue at a position corresponding to position 260 of wild-type human Neu2 is substituted by aspartic acid (L260D), phenylalanine (L260F), glutamine (L260Q), or threonine (L260T); (ii) the valine residue at a position corresponding to position 265 of wild-type human Neu2 is substituted by phenylalanine (V265F); (jj) the glutamine residue at a position corresponding to position 270 of wild-type human Neu2 is substituted by alanine (Q270A), histidine (Q270H), phenylalanine (Q270F), proline (Q270P), serine (Q270S), or threonine (Q270T); (kk) the tryptophan residue at a position corresponding to position 292 of wild-type human Neu2 is substituted by arginine (W292R); (ll) the serine residue at a position corresponding to position 301 of wild-type human Neu2 is substituted by alanine (S301A), aspartic acid (S301D), glutamic acid (S301E), phenylalanine (S301F), glycine (S301G), histidine (S301H), isoleucine (S301I), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y); (mm) the tryptophan residue at a position corresponding to position 302 of wild-type human Neu2 is substituted by alanine (W302A), aspartic acid (W302D), glutamic acid (W302E), phenylalanine (W302F), glycine (W302G), histidine (W302H), isoleucine (W302I), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y); (nn) the valine residue at a position corresponding to position 363 of wild-type human Neu2 is substituted by arginine (V363R); or (oo) the leucine residue at a position corresponding to position 365 of wild-type human Neu2 is substituted by glutamine (L365Q), histidine (L365H), isoleucine (L365I), lysine (L365K) or serine (L365S); or the sialidase comprises a combination of any of the foregoing substitutions. For example, the sialidase may comprise a substitution selected from K9D, P62G, P62N, P62S, P62T, D80P, A93E, Q126H, Q126Y, R189P, H239P, A242T, Q270A, Q270S, Q270T, S301A, S301R, W302K, W302R, V363R, and L365I, or a combination of any of the foregoing substitutions.

In certain embodiments, the recombinant mutant human sialidase comprises a deletion of a leucine residue at a position corresponding to position 184 of wild-type human Neu2 (ΔL184), a deletion of a histidine residue at a position corresponding to position 185 of wild-type human Neu2 (ΔH185), a deletion of a proline residue at a position corresponding to position 186 of wild-type human Neu2 (ΔP186), a deletion of an isoleucine residue at a position corresponding to position 187 of wild-type human Neu2 (ΔI187), and a deletion of a glutamine residue at a position corresponding to position 184 of wild-type human Neu2 (ΔQ188), or a combination of any of the foregoing deletions.

In certain embodiments, the recombinant mutant human sialidase comprises an insertion between a threonine residue at a position corresponding to position 216 of wild-type human Neu2 and a leucine residue at a position corresponding to position 217 of wild-type human Neu2, for example, an insertion of an amino acid selected from S, T, Y, L, F, A, P, V, I, N, D, and H.

Additional exemplary sialidase mutations, and combinations of sialidase mutations, are described in International (PCT) Patent Application No. PCT/US2019/012207, filed Jan. 3, 2019, including in the Detailed Description in the section entitled “I. Recombinant Human Sialidases,” and in the Examples in Examples 1, 2, 3, 4, 5, and 6.

6. Combinations of Substitutions

In certain embodiments, the recombinant mutant human sialidase comprises a combination of any of the mutations contemplated herein. For example, the recombinant mutant sialidase enzyme may comprise a combination of 2, 3, 4, 5, 6, 7, 8, 9, 10, 11, 12, 13, 14, 15 or more of the mutations contemplated herein. It is contemplated that the recombinant mutant sialidase enzyme may comprise 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 3-4 of the mutations contemplated herein.

For example, the recombinant mutant sialidase enzyme may comprise a M1 deletion (ΔM1), M1A substitution, M1D substitution, V6Y substitution, K9D substitution, P62G substitution, P62N substitution, P62S substitution, P62T substitution, A93E substitution, I187K substitution, Q270A substitution, S301R substitution, W302K substitution, C332A substitution, V363R substitution, L365I substitution, or a combination of any of the foregoing.

In certain embodiments, the recombinant mutant sialidase enzyme comprises a M1 deletion (ΔM1), M1A substitution, M1D substitution, V6Y substitution, I187K substitution, C332A substitution, or a combination of any of the foregoing. For example, the recombinant mutant sialidase enzyme may comprise a combination of mutations selected from: M1A and V6Y; M1A and I187K; M1A and C332A; M1D and V6Y; M1D and I187K; M1D and C332A; ΔM1 and V6Y; ΔM1 and I187K; ΔM1 and C332A; V6Y and I187K; V6Y and C332A; I187K and C332A; M1A, V6Y, and I187K; M1A, V6Y, and C332A; M1A, I187K, and C332A; M1D, V6Y, and I187K; M1D, V6Y, and C332A; M1D, I187K, and C332A; ΔM1, V6Y, and I187K; ΔM1, V6Y, and C332A; ΔM1, I187K, and C332A; V6Y, I187K, and C332A; M1A, V6Y, I187K, and C332A; M1D, V6Y, I187K, and C332A; and ΔM1, V6Y, I187K, and C332A.

In certain embodiments, the recombinant mutant sialidase enzyme comprises (i) an amino acid substitution identified in TABLE 7, or a combination of any amino acid substitutions identified in TABLE 7, and (ii) an M1 deletion (ΔM1), M1A substitution, M1D substitution, V6Y substitution, I187K substitution, C332A substitution, or a combination of any of the foregoing. For example, the recombinant mutant sialidase enzyme may comprise (i) an amino acid substitution identified in TABLE 7, or a combination of any amino acid substitutions identified in TABLE 7, and (ii) a combination of mutations selected from: M1A and V6Y; M1A and I187K; M1A and C332A; M1D and V6Y; M1D and I187K; M1D and C332A; ΔM1 and V6Y; ΔM1 and I187K; ΔM1 and C332A; V6Y and I187K; V6Y and C332A; I187K and C332A; M1A, V6Y, and I187K; M1A, V6Y, and C332A; M1A, I187K, and C332A; M1D, V6Y, and I187K; M1D, V6Y, and C332A; M1D, I187K, and C332A; ΔM1, V6Y, and I187K; ΔM1, V6Y, and C332A; ΔM1, I187K, and C332A; V6Y, I187K, and C332A; M1A, V6Y, I187K, and C332A; M1D, V6Y, I187K, and C332A; and ΔM1, V6Y, I187K, and C332A.

In certain embodiments, the recombinant mutant sialidase enzyme comprises: (a) the M1D, V6Y, P62G, A93E, I187K, and C332A substitutions; (b) the M1D, V6Y, K9D, A93E, I187K, C332A, V363R, and L365I substitutions; (c) the M1D, V6Y, P62N, I187K, and C332A substitutions; (d) the M1D, V6Y, I187K, Q270A, S301R, W302K, and C332A substitutions; (e) the M1D, V6Y, P62S, I187K, Q270A, S301R, W302K, and C332A substitutions; (f) the M1D, V6Y, P62T, I187K, Q270A, S301R, W302K, and C332A substitutions; (g) the M1D, V6Y, P62N, I187K, Q270A, S301R, W302K, and C332A substitutions; (h) the M1D, V6Y, P62G, A93E, I187K, S301A, W302R, and C332A substitutions; (i) the M1D, V6Y, P62G, A93E, Q126Y, I187K, Q270T, and C332A substitutions; (j) the M1D, V6Y, P62G, A93E, Q126Y, I187K, and C332A substitutions; or (k) the M1D, V6Y, P62G, A93E, Q126Y, I187K, A242F, Q270T, and C332A substitutions.

In certain embodiments, the recombinant mutant human sialidase comprises a substitution of a serine residue at a position corresponding to position 301 of wild-type human Neu2 (S301) in combination with a substitution of a tryptophan residue at a position corresponding to position 302 of wild-type human Neu2 (W302). For example, the recombinant mutant human sialidase may comprise a combination of substitutions corresponding to a combination of substitutions listed in a row of TABLE 8 (amino acid positions corresponding to wild-type human Neu2 (SEQ ID NO: 1)). For example, the recombinant mutant human sialidase may comprise: the S301K and W302R substitutions; the S301K and W302K substitutions; or the S301A and W302S substitutions.

TABLE 8
Substitutions

S301A, W302R
S301A, W302S
S301A, W302T
S301K, W302S
S301N, W302S
S301T, W302S
S301T, W302T
S301T, W302R
S301A, W302A
S301K, W302R
S301K, W302T
S301N, W302T
S301K, W302K
S301P, W302R
S301P, W302S
S301P, W302T

In certain embodiments, the recombinant mutant human sialidase comprises a combination of substitutions corresponding to a combination of substitutions listed in a row of TABLE 9 (amino acid positions corresponding to wild-type human Neu2 (SEQ ID NO: 1)).

TABLE 9
Substitutions

M1D, V6Y, P62G, I187K, C332A
M1D, V6Y, K9D, I187K, C332A, V363R, L365I
M1D, V6Y, P62G, A93E, I187K, C332A
M1D, V6Y, K9D, I187K, C332A, V363R, L365K
M1D, V6Y, K9D, I187K, C332A, V363R, L365S
M1D, V6Y, K9D, I187K, C332A, V363R, L365Q
M1D, V6Y, K9D, I187K, C332A, V363R, L365H
M1D, V6Y, A93K, I187K, C332A
M1D, V6Y, A93E, I187K, C332A
V6Y, I187K, W292R
V6Y, G107D, I187K
V6Y, C125L
C125L, I187K
V6Y, C125L, I187K
M1D, V6Y, K45A, I187K, C332A
M1D, V6Y, Q270A, I187K, C332A
M1D, V6Y, K44R, K45R, I187K, C332A
M1D, V6Y, Q112R, I187K, C332A
M1D, V6Y, Q270F, I187K, C332A
M1D, V6Y, I187K, S301R, W302K, C332A
M1D, V6Y, K44E, K45E, I187K, C332A
M1D, V6Y, I187K, L217V, C332A
M1D, V6Y, I187K, L217A, C332A
M1D, V6Y, K44E, K45E, I187K, S301R, W302K, C332A
M1D, V6Y, Q112R, I187K, S301R, W302K, C332A
M1D, V6Y, I187K, Q270A, S301R, W302K, C332A
M1D, V6Y, K44E, K45E, Q112R, I187K, C332A
M1D, V6Y, K44E, K45E, I187K, Q270A, C332A
M1D, V6Y, K45A, I187K, Q270A, C332A
M1D, V6Y, I187K, Q270H, C332A
M1D, V6Y, I187K, Q270P, C332A
M1D, V6Y, Q112K, I187K, C332A
M1D, V6Y, P62S, I187K, Q270A, S301R, W302K, C332A
M1D, V6Y, P62T, I187K, Q270A, S301R, W302K, C332A
M1D, V6Y, P62N, I187K, Q270A, S301R, W302K, C332A
V6Y, P62H, I187K
V6Y, Q108H, I187K
M1D, V6Y, P62H, I187K, C332A
M1D, V6Y, P62G, I187K, C332A
V6Y, P62G, I187K
M1D, V6Y, P62H, H87K
M1D, V6Y, Q108H, I187K
M1D, V6Y, P62N, I187K, C332A
M1D, V6Y, P62D, I187K, C332A
M1D, V6Y, P62E, I187K, C332A
V6Y, C164G, I187K, T249A
V6Y, C164G, I187K
V6Y, Q126L, I187K D251G
V6Y, L54M, Q69H, R78K, A171G, I187K
V6Y, P62T, I187K
V6Y, A150V, I187K
P5H, V6Y, P62S, I187K
V6Y, C164G, I187K
Q126Y, Q170T
Q126Y, A242F, Q270T
M1D, V6Y, P62G, A93E, Q126E, I187K, C332A
M1D, V6Y, P62G, A93E, Q126I, I187K, C332A
M1D, V6Y, P62G, A93E, Q126L, I187K, C332A
M1D, V6Y, P62G, A93E, Q126Y, I187K, C332A
M1D, V6Y, P62G, A93E, Q126F, I187K, C332A
M1D, V6Y, P62G, A93E, Q126H, I187K, C332A
M1D, V6Y, P62G, A93E, I187K, Q270S, C332A
M1D, V6Y, P62G, A93E, I187K, Q270T, C332A
M1D, V6Y, P62G, A93E, Q126Y, I187K, Q270T, C332A
M1D, V6Y, P62G, A93E, Q126Y, I187K, A242F, Q270T, C332A
M1D, V6Y, P62G, D80P, A93E, I187K, C332A
M1D, V6Y, P62G, A93E, R170P, I187K, C332A
M1D, V6Y, P62G, A93E, I187K, Q188P, C332A
M1D, V6Y, P62G, A93E, I187K, R189P, C332A
M1D, V6Y, P62G, A93E, I187K, E225P, C332A
M1D, V6Y, P62G, A93E, I187K, H239P, C332A
M1D, V6Y, P62G, A93E, I187K, E257P, C332A
M1D, V6Y, P62G, A93E, I187K, S301A, C332A
M1D, V6Y, P62G, A93E, I187K, S301D, C332A
M1D, V6Y, P62G, A93E, I187K, S301E, C332A
M1D, V6Y, P62G, A93E, I187K, S301F, C332A
M1D, V6Y, P62G, A93E, I187K, S301H, C332A
M1D, V6Y, P62G, A93E, I187K, S301K, C332A
M1D, V6Y, P62G, A93E, I187K, S301L, C332A
M1D, V6Y, P62G, A93E, I187K, S301M, C332A
M1D, V6Y, P62G, A93E, I187K, S301N, C332A
M1D, V6Y, P62G, A93E, I187K, S301P, C332A
M1D, V6Y, P62G, A93E, I187K, S301Q, C332A
M1D, V6Y, P62G, A93E, I187K, S301R, C332A
M1D, V6Y, P62G, A93E, I187K, S301T, C332A
M1D, V6Y, P62G, A93E, I187K, S301V, C332A
M1D, V6Y, P62G, A93E, I187K, S301W, C332A
M1D, V6Y, P62G, A93E, I187K, S301Y, C332A
M1D, V6Y, P62G, A93E, I187K, W302A, C332A
M1D, V6Y, P62G, A93E, I187K, W302D, C332A
M1D, V6Y, P62G, A93E, I187K, W302F, C332A
M1D, V6Y, P62G, A93E, I187K, W302G, C332A
M1D, V6Y, P62G, A93E, I187K, W302H, C332A
M1D, V6Y, P62G, A93E, I187K, W302I, C332A
M1D, V6Y, P62G, A93E, I187K, W302L, C332A
M1D, V6Y, P62G, A93E, I187K, W302M, C332A
M1D, V6Y, P62G, A93E, I187K, W302N, C332A
M1D, V6Y, P62G, A93E, I187K, W302P, C332A
M1D, V6Y, P62G, A93E, I187K, W302Q, C332A
M1D, V6Y, P62G, A93E, I187K, W302R, C332A
M1D, V6Y, P62G, A93E, I187K, W302S, C332A
M1D, V6Y, P62G, A93E, I187K, W302T, C332A
M1D, V6Y, P62G, A93E, I187K, W302V, C332A
M1D, V6Y, P62G, A93E, I187K, W302Y, C332A
M1D, V6Y, P62G, A93E, I187K, S301A, W302A, C332A
M1D, V6Y, P62G, A93E, I187K, S301A, W302R, C332A
M1D, V6Y, P62G, A93E, I187K, S301A, W302S, C332A
M1D, V6Y, P62G, A93E, I187K, S301A, W302T, C332A
M1D, V6Y, P62G, A93E, I187K, S301K, W302S, C332A
M1D, V6Y, P62G, A93E, I187K, S301K, W302R, C332A
M1D, V6Y, P62G, A93E, I187K, S301K, W302T, C332A
M1D, V6Y, P62G, A93E, I187K, S301N, W302S, C332A
M1D, V6Y, P62G, A93E, I187K, S301N, W302T, C332A
M1D, V6Y, P62G, A93E, I187K, S301T, W302R, C332A
Q126Y, Q270T
Q126Y, A242F, Q270T

In certain embodiments, the recombinant mutant human sialidase comprises the amino acid sequence of any one of SEQ ID NOs: 48-62, 102, 105, 108, or 133, or an amino acid sequence that has at least 85%, 90%, 95%, 96%, 97%, 98%, or 99% sequence identity to any one of SEQ ID NOs: 48-62, 102, 105, 108, or 133.

In certain embodiments, the recombinant mutant human sialidase comprises the amino acid sequence of X₁X₂SX₃X₄X₅LQX₆ESVFQSGAHAYRIPALLYLPGQQSLLAFAEQRASX₇X₈DEHAELIVX₉RRGD YDAX₁₀THQVQWX₁₁AQEVVAQAX₁₂LDGHRSMNPCPLYDX₁₃QTGTLFLFFIAIPX₁₄X₁₅VTEX₁₆Q QLQTRANVTRLX₁₇X₁₈VTSTDHGRTWSSPRDLTDAAIGPX₁₉YREWSTFAVGPGHX₂₀LQLHDRX₂₁RSLVVPAYAYRKLHPX₂₂QRPIPSAFX₂₃FLSHDHGRTWARGHFVAQDTX₂₄ECQVAEVETGEQRV VTLNARSHLRARVQAQSX₂₅NX₂₆GLDFQX₂₇SQLVKKLVEPPPX₂₈GX₂₉QGSVISFPSPRSGPGSP AQX₃₀LLYTHPTHX₃₁X₃₂QRADLGAYLNPRPPAPEAWSEPX₃₃LLAKGSX₃₄AYSDLQSMGTGPDGS PLFGX₃₅LYEANDYEEIX₃₆FX₃₇MFTLKQAFPAEYLPQ

(SEQ ID NO: 47), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Lys, Arg, or Glu. X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Ala, Glu or Lys, X₁₄ is Gly or Asp, X₁₅ is Gln or His, X₁₆ is Gln, Arg, or Lys, X₁₇ is Ala, Cys, Ile, Ser, Val, or Leu, X₁₈ is Gln or Leu, X₁₉ is Ala or Val, X₂₀ is Cys or Gly, X₂₁ is Ala or Gly, X₂₂ is Arg, Ile, or Lys, X₂₃ is Ala, Cys, Leu, or Val, X₂₄ is Leu, Ala, or Val, X₂₅ is Thr or Ala, X₂₆ is Asp or Gly, X₂₇ is Glu or Lys, X₂₈ is Gln, Ala, His, Phe, or Pro, X₂₉ is Cys or Val, X₃₀ is Trp or Arg, X₃₁ is Ser or Arg, X₃₂ is Trp or Lys, X₃₃ is Lys or Val, X₃₄ is Ala, Cys, Ser, or Val, X₃₅ is Cys, Leu, or Val, X₃₆ is Val or Arg, and X₃₇ is Leu, Gln, His, Ile, Lys, or Ser, and the sialidase comprises at least one mutation relative to wild-type human Neu2 (SEQ ID NO: 1).

In certain embodiments, the recombinant mutant human sialidase comprises the amino acid sequence of X₁ASLPX₂LQX₃ESVFQSGAHAYRIPALLYLPGQQSLLAFAEQRASKKDEHAELIVLRRGDYDAX₄THQVQWQAQEVVAQARLDGHRSMNPCPLYDX₅QTGTLFLFFIAIPGQVTEQQQLQTRANVTRLCQ VTSTDHGRTWSSPRDLTDAAIGPAYREWSTFAVGPGHCLQLHDRARSLVVPAYAYRKLHPX₆QRP IPSAFCFLSHDHGRTWARGHFVAQDTLECQVAEVETGEQRVVTLNARSHLRARVQAQSTNDGLD FQESQLVKKLVEPPPX₇GCQGSVISFPSPRSGPGSPAQWLLYTHPTHX₈X₉QRADLGAYLNPRPP APEAWSEPVLLAKGSX₁₀AYSDLQSMGTGPDGSPLFGCLYEANDYEEIX₁₁FX₁₂MFTLKQAFPAE YLPQ

(SEQ ID NO: 46), wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Arg, Ile, or Lys, X₇ is Gln, Ala, His, Phe, or Pro, X₈ is Ser or Arg, X₉ is Trp or Lys, X₁₀ is Ala, Cys, Ser, or Val, X₁₁ is Val or Arg, and X₁₂ is Leu, Gln, His, Ile, Lys, or Ser, and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Ile or Lys, X₇ is Gln or Ala, X₈ is Ser or Arg, X₉ is Trp or Lys, X₁₀ is Ala or Cys, X₁₁ is Val or Arg, and X₁₂ is Leu or Ile.

In certain embodiments, the recombinant mutant human sialidase comprises the amino acid sequence of X₁X₂SX₃X₄X₅LQX₆ESVFQSGAHAYRIPALLYLPGQQSLLAFAEQRASX₇X₈DEHAELIVX₉RRGD YDAX₁₀THQVQWX₁₁AQEVVAQAX₁₂LX₁₃GHRSMNPCPLYDX₁₄QTGTLFLFFIAIPX₁₃X₁₆VTEX₁₇QQLQTRANVTRLX₁₈X₁₉VTSTDHGRTWSSPRDLTDAAIGPX₂₀YREWSTFAVGPGHX₂₁LQLHDX₂₂X₂₃RSLVVPAYAYRKLHPX₂₄X₂₃X₂₆PIPSAFX₂₇FLSHDHGRTWARGHFVX₂₈QDTX₂₉ECQVAEVX₃₀TGEQRVVTLNARSX₃₁X₃₂X₃₃X₃₄RX₃₅QAQSX₃₆NX₃₇GLDFQX₃₈X₃₉QX₄₀VKKL X₄₁EPPPX₄₂GX₄₃QGSVISFPSPRSGPGSPAQX₄₄LLYTHPTHX₄₅X₄₆QRADLGAYLNPRPPAPEA WSEPX₄₇LLAKGSX₄₈AYSDLQSMGTGPDGSPLFGX₄₉LYEANDYEEIX₅₀FX₅₁MFTLKQAFPAEYL PQ

(SEQ ID NO: 125), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Lys, Arg, or Glu, X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Asp or Pro, X₁₄ is Ala, Glu or Lys, X₁₅ is Gly or Asp, X₁₆ is Gln or His, X₁₇ is Gln, Arg, or Lys, X₁₈ is Ala, Cys, Ile, Ser, Val, or Leu, X₁₉ is Gln, Leu, Glu, Phe, His, Ile, Leu, or Tyr, X₂₀ is Ala or Val, X₂₁ is Cys or Gly, X₂₂ is Arg or Pro, X₂₃ is Ala or Gly, X₂₄ is Arg, Ile, or Lys, X₂₅ is Gln or Pro, X₂₆ is Arg or Pro, X₂₇ is Ala, Cys, Leu, or Val, X₂₈ is Ala, Cys, Asn, Ser, or Thr, X₂₉ is Leu, Ala, or Val, X₃₀ is Glu or Pro, X₃₁ is His or Pro, X₃₂ is Leu, Asp, Asn, or Tyr, X₃₃ is Arg, Ala, Asp, Leu, Gln, or Tyr, X₃₄ is Ala, Cys, Phe, Gly, His, Ile, Lys, Leu, Met, Asn, Gln, Arg, Ser, Val, Trp, or Tyr, X₃₅ is Val, Ile, or Lys, X₃₆ is Thr or Ala, X₃₇ is Asp or Gly, X₃₈ is Glu, Lys, or Pro, X₃₉ is Ser or Cys, X₄₀ is Leu, Asp, Phe, Gln, or Thr, X₄₁ is Val or Phe, X₄₂ is Gln, Ala, His, Phe, Pro, Ser, or Thr, X₄₃ is Cys or Val, X₄₄ is Trp or Arg, X₄₅ is Ser, Arg, Ala, Asp, Glu, Phe, Gly, His, Ile, Lys, Leu, Met, Asn, Pro, Gln, Thr, Val, Trp, or Tyr, X₄₆ is Trp, Lys, Ala, Asp, Glu, Phe, Gly, His, Ile, Lys, Leu, Met, Asn, Pro, Gln, Arg, Ser, Thr, Val, or Tyr, X₄₇ is Lys or Val, X₄₈ is Ala, Cys, Ser, or Val, X₄₉ is Cys, Leu, or Val, X₅₀ is Val or Arg, and X₅₁ is Leu, Gln, His, Ile, Lys, or Ser, and the sialidase comprises at least one mutation relative to wild-type human Neu2 (SEQ ID NO: 1).

In certain embodiments, the recombinant mutant human sialidase comprises the amino acid sequence of X₁ASLPX₂LQX₃ESVFQSGAHAYRIPALLYLPGQQSLLAFAEQRASKKDEHAELIVLRRGDYDAX₄THQVQWQAQEVVAQARLDGHRSMNPCPLYDX₃QTGTLFLFFIAIPGQVTEQQQLQTRANVTRLCX₆VTSTDHGRTWSSPRDLTDAAIGPAYREWSTFAVGPGHCLQLHDRARSLVVPAYAYRKLHPX₇QR PIPSAFCFLSHDHGRTWARGHFVAQDTLECQVAEVETGEQRVVTLNARSHLRX₈RVQAQSTNDGL DFQESQLVKKLVEPPPX₉GCQGSVISFPSPRSGPGSPAQWLLYTHPTHX₁₀X₁₁QRADLGAYLNPR PPAPEAWSEPVLLAKGSX₁₂AYSDLQSMGTGPDGSPLFGCLYEANDYEEIX₁₃FX₁₄MFTLKQAFP AEYLPQ

(SEQ ID NO: 124), wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Gln, Leu, Glu, Phe, His, Ile, Leu, or Tyr, X₇ is Arg, Ile, or Lys, X₈ is Ala, Cys, Phe, Gly, His, Ile, Lys, Leu, Met, Asn, Gln, Arg, Ser, Val, Trp, or Tyr, X₉ is Gln, Ala, His, Phe, Pro, Ser, or Thr, X₁₀ is Ser, Arg, Ala, Asp, Glu, Phe, Gly, His, Ile, Lys, Leu, Met, Asn, Pro, Gln, Thr, Val, Trp, or Tyr, X₁₁ is Trp, Lys, Ala, Asp, Glu, Phe, Gly, His, Ile, Lys, Leu, Met, Asn, Pro, Gln, Arg, Ser, Thr, Val, or Tyr, X₁₂ is Ala, Cys, Ser, or Val, X₁₃ is Val or Arg, and X₁₄ is Leu, Gln, His, Ile, Lys, or Ser, and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Gln or Tyr, X₇ is Ile or Lys, X₈ is Ala or Thr, X₉ is Gln, Ala, or Thr, X₁₀ is Ser, Arg, or Ala, X₁₁ is Trp, Lys, or Arg, X₁₂ is Ala or Cys, X₁₃ is Val or Arg, and X₁₄ is Leu or Ile.

In certain embodiments, the recombinant mutant human sialidase comprises a conservative substitution relative to a recombinant mutant human sialidase sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution: p matrix (e.g., the PAM 250 matrix).

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; -W, wordsize [Integer]: default=11 for nucleotides/28 for megablast/3 for proteins; -y, Dropoff (X) for blast extensions in bits: default=20 for blastn/7 for others; —X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

b. Antibody Portion

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

In certain embodiments, the fusion protein comprises an immunoglobulin Fc domain. As used herein, unless otherwise indicated, the term “immunoglobulin Fc domain” refers to a fragment of an immunoglobulin heavy chain constant region which, either alone or in combination with a second immunoglobulin Fc domain, is capable of binding to an Fc receptor. An immunoglobulin Fc domain may include, e.g., immunoglobulin CH2 and CH3 domains. An immunoglobulin Fc domain may include, e.g., immunoglobulin CH2 and CH3 domains and an immunoglobulin hinge region. Boundaries between immunoglobulin hinge regions, CH2, and CH3 domains are well known in the art, and can be found, e.g., in the PROSITE database (available on the world wide web at prosite.expasy.org).

In certain embodiments, the immunoglobulin Fc domain is derived from a human IgG1, IgG2, IgG3, IgG4, IgA1, IgA2, IgD, IgE, and IgM Fc domain. A single amino acid substitution (S228P according to Kabat numbering; designated IgG4Pro) may be introduced to abolish the heterogeneity observed in recombinant IgG4 antibody. See Angal, S. et al. (1993) MOL. IMMUNOL. 30:105-108.

In certain embodiments, the immunoglobulin Fc domain is derived from a human IgG1 isotype or another isotype that elicits antibody-dependent cell-mediated cytotoxicity (ADCC) and/or complement mediated cytotoxicity (CDC). In certain embodiments, the immunoglobulin Fc domain is derived from a human IgG1 isotype (e.g., SEQ ID NO: 31 or SEQ ID NO: 5).

In certain embodiments, the immunoglobulin Fc domain is derived from a human IgG4 isotype or another isotype that elicits little or no antibody-dependent cell-mediated cytotoxicity (ADCC) and/or complement mediated cytotoxicity (CDC). In certain embodiments, the immunoglobulin Fc domain is derived from a human IgG4 isotype.

In certain embodiments, the immunoglobulin Fc domain comprises either a “knob” mutation, e.g., T366Y, or a “hole” mutation, e.g., Y407T, for heterodimerization with a second polypeptide (residue numbers according to EU numbering, Kabat, E. A., et al. (1991) SEQUENCES OF PROTEINS OF IMMUNOLOGICAL INTEREST, FIFTH EDITION, U.S. Department of Health and Human Services, NIH Publication No. 91-3242).

In certain embodiments, the fusion protein comprises an immunoglobulin antigen-binding domain. The inclusion of such a domain may improve targeting of a fusion protein to a sialylated cancer cell, e.g., a CD20 expressing cancer cell, and/or to the tumor microenvironment. As used herein, unless otherwise indicated, the term “immunoglobulin antigen-binding domain” refers to a polypeptide that, alone or in combination with another immunoglobulin antigen-binding domain, defines an antigen-binding site. Exemplary immunoglobulin antigen-binding domains include, for example, immunoglobulin heavy chain variable region and an immunoglobulin light chain variable region, where the variable regions together define an antigen binding site, e.g., an anti-CD20 antigen binding site.

In certain embodiments, the immunoglobulin antigen-binding domain is derived from an anti-CD20 antibody. Exemplary anti-CD20 antibodies include ofatumumab, rituximab, ocrelizumab, obinutuzumab, ibritumomab, veltuzumab, reditux, PRO131921, TRU-015, ubituximab, NCD1.2, blontuvetmab, FBTA05, mosunetuzumab, ocaratuzumab, ocrelizumab, and tositumomab.

In certain embodiments, the immunoglobulin antigen-binding domain is derived from rituximab. The rituximab heavy chain amino acid sequence is depicted in SEQ ID NO: 63, and the rituximab light chain amino acid sequence is depicted in SEQ ID NO: 64. In certain embodiments, the immunoglobulin antigen-binding domain is derived from ofatumumab. The ofatumumab heavy chain antigen binding fragment amino acid sequence is depicted in SEQ ID NO: 65, and the ofatumumab light chain amino acid sequence is depicted in SEQ ID NO: 77.

c. Linker

In certain embodiments, the sialidase portion of the fusion protein can be linked or fused directly to the anti-CD20 antibody portion (e.g., immunoglobulin Fc domain and/or immunoglobulin antigen-binding domain) of the fusion protein. In other embodiments, the sialidase portion can be covalently bound to the anti-CD20 antibody portion by a linker.

The linker may couple, with one or more natural amino acids, the sialidase, or functional fragment thereof, and the antibody portions or fragments, where the amino acid (for example, a cysteine amino acid) may be introduced by site-directed mutagenesis. The linker may include one or more unnatural amino acids. It is contemplated that, in certain circumstances, a linker containing for example, one or more sulfhydryl reactive groups (e.g., a maleimide) may covalently link a cysteine in the sialidase portion or the antibody portion that is a naturally occurring cysteine residue or is the product of site-specific mutagenesis.

The linker may be a cleavable linker or a non-cleavable linker. Optionally or in addition, the linker may be a flexible linker or an inflexible linker.

The linker should be a length sufficiently long to allow the sialidase and the antibody portions to be linked without steric hindrance from one another and sufficiently short to retain the intended activity of the fusion protein. The linker preferably is sufficiently hydrophilic to avoid or minimize instability of the fusion protein. The linker preferably is sufficiently hydrophilic to avoid or minimize insolubility of the fusion protein. The linker should be sufficiently stable in vivo (e.g., it is not cleaved by serum, enzymes, etc.) to permit the fusion protein to be operative in vivo.

The linker may be from about 1 angstroms (Å) to about 150 Å in length, or from about 1 Å to about 120 Å in length, or from about 5 Å to about 110 Å in length, or from about 10 Å to about 100 Å in length. The linker may be greater than about 2, 3, 4, 5, 6, 7, 8, 9, 10, 11, 12, 13, 14, 15, 16, 17, 18, 19, 20, 25, 27, 30 or greater angstroms in length and/or less than about 110, 100, 90, 85, 80, 75, 70, 65, 60, 55, 50, 45, 43, 42, 41, 40, 39, 38, 37, 36, 35, 34, 33, 32, 31, or fewer A in length. Furthermore, the linker may be about 5, 10, 15, 20, 25, 30, 35, 40, 45, 50, 55, 60, 65, 70, 75, 80, 85, 90, 95, 100, 110, and 120 Å in length.

In certain embodiments, the linker comprises a polypeptide linker that connects or fuses the sialidase portion of the fusion protein to the anti-CD20 antibody portion (e.g., immunoglobulin Fc domain and/or immunoglobulin antigen-binding domain) of the fusion protein. For example, it is contemplated that a gene encoding a sialidase portion linked directly or indirectly (for example, via an amino acid containing linker) to an antibody portion can be created and expressed using conventional recombinant DNA technologies. For example, the amino terminus of a sialidase portion can be linked to the carboxy terminus of either the light or the heavy chain of an antibody portion. For example, for a Fab fragment, the amino terminus or carboxy terminus of the sialidase can be linked to the first constant domain of the heavy antibody chain (CH1). When a linker is employed, the linker may comprise hydrophilic amino acid residues, such as Gln, Ser, Gly, Glu, Pro, His and Arg. In certain embodiments, the linker is a peptide containing 1-25 amino acid residues, 1-20 amino acid residues, 2-15 amino acid residues, 3-10 amino acid residues, 3-7 amino acid residues, 4-25 amino acid residues, 4-20 amino acid residues, 4-15 amino acid residues, 4-10 amino acid residues, 5-25 amino acid residues, 5-20 amino acid residues, 5-15 amino acid residues, or 5-10 amino acid residues. Exemplary linkers include glycine and serine-rich linkers, e.g., (GlyGlyPro)ₙ, or (GlyGlyGlyGlySer)ₙ, where n is 1-5. In certain embodiments, the linker comprises, consists, or consists essentially of GGGGS (SEQ ID NO: 126). In certain embodiments, the linker comprises, consists, or consists essentially of GGGGSGGGGS (SEQ ID NO: 98). In certain embodiments, the linker comprises, consists, or consists essentially of EPKSS (SEQ ID NO: 99). Additional exemplary linker sequences are disclosed, e.g., in George et al. (2003) PROTEIN ENGINEERING 15:871-879, and U.S. Pat. Nos. 5,482,858 and 5,525,491.

In certain embodiments, the fusion protein comprises the amino acid sequence of any one of SEQ ID NOs: 66-74, 78, 88-97, 103, 104, 106, 107, 109, 110, 113, 115, 117, 119, 121, 127, 128, 134, or 135, or an amino acid sequence that has at least 85%, 90%, 95%, 96%, 97%, 98%, or 99% sequence identity to any one of SEQ ID NOs: 66-74, 78, 88-97, 103, 104, 106, 107, 109, 110, 113, 115, 117, 119, 121, 127, 128, 134, or 135.

d. Antibody Conjugates

The invention further provides antibody conjugates containing one or more of the fusion proteins disclosed herein. As used herein, unless otherwise indicated, the term “antibody conjugate” is understood to refer to an antibody, or a functional fragment thereof, that comprises antigen-binding activity (e.g., anti-CD20 antigen-binding activity) and/or Fc receptor-binding activity, conjugated (e.g., covalently coupled) to an additional functional moiety. In certain embodiments, the antibody or functional antibody fragment is conjugated to a sialidase enzyme, e.g., a recombinant mutant human sialidase enzyme disclosed herein. In certain embodiments, an antibody conjugate comprises a single polypeptide chain. In certain embodiments, an antibody conjugate comprises two, three, four, or more polypeptide chains that are covalently or non-covalently associated together to produce a multimeric complex, e.g., a dimeric, trimeric or tetrameric complex. For example, an antibody conjugate may comprise a first polypeptide (fusion protein) comprising a recombinant mutant human sialidase enzyme and an immunoglobulin heavy chain, and a second polypeptide comprising an immunoglobulin light chain, where, for example, the immunoglobulin heavy and light chains together define a single antigen-binding site, e.g., an anti-CD20 antigen-binding site.

In certain embodiments, the antibody conjugate can include a single sialidase. In other embodiments, the antibody conjugate can include more than one (e.g., two) sialidases. If more than one sialidase is included, the sialidases can be the same or different. In certain embodiments, the antibody conjugate can include a single anti-CD20 antigen-binding site. In other embodiments, the antibody conjugate can include more than one (e.g., two) anti-CD20 antigen-binding sites. If two antigen-binding sites are used, they can be the same or different. In certain embodiments, the antibody conjugate comprises an immunoglobulin Fc fragment.

In certain embodiments, the antibody conjugate comprises one or two immunoglobulin heavy chains, or a functional fragment thereof. In certain embodiments, the antibody conjugate comprises one or two immunoglobulin light chains, or a functional fragment thereof. In certain embodiments, the antibody conjugate comprises a sialidase fused to the N- or C-terminus of an immunoglobulin heavy chain or an immunoglobulin light chain.

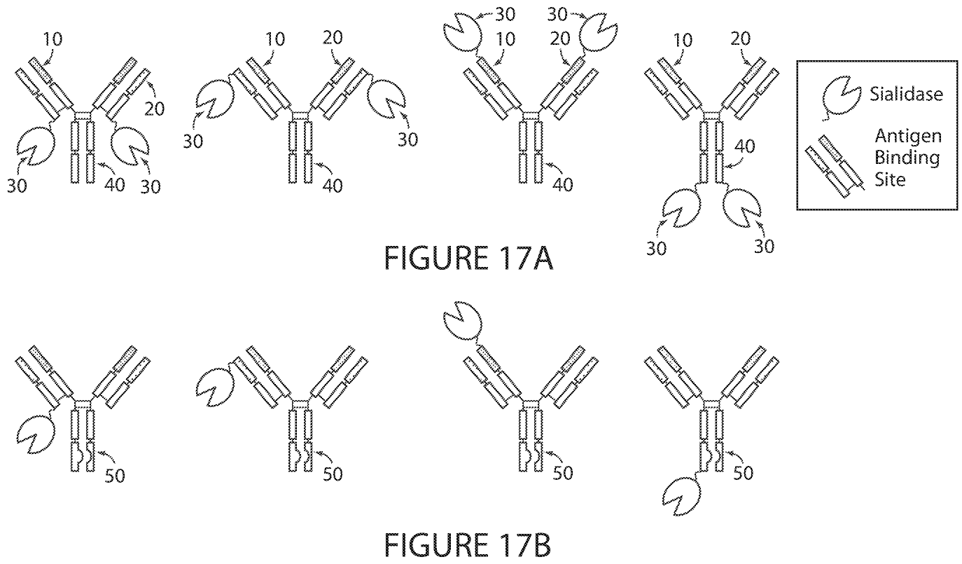

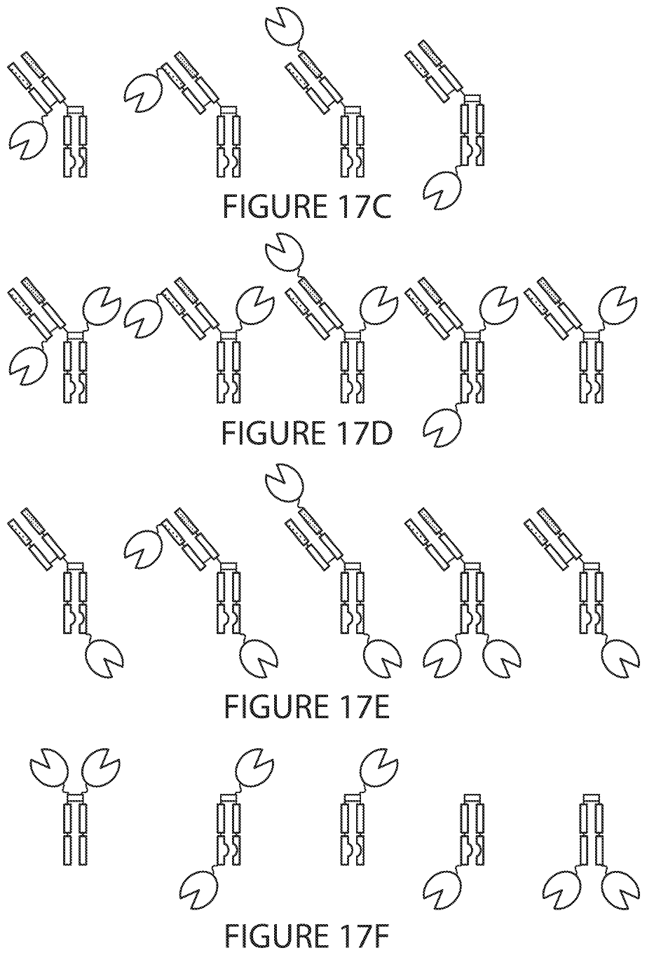

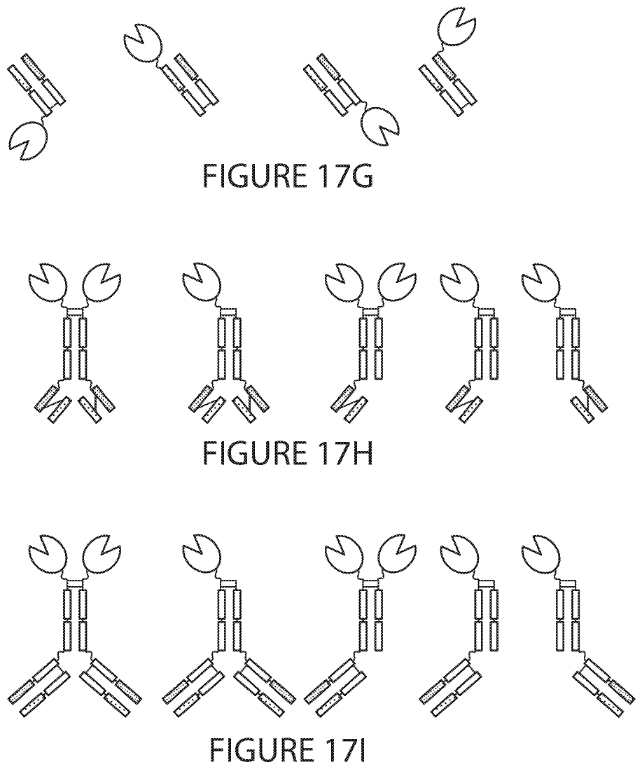

FIG. 17 depicts exemplary antibody conjugate constructs containing one or more sialidase enzymes. For example, in FIG. 17A, a first anti-CD20 antigen-binding site is depicted as 10, a second anti-CD20 antigen-binding site is depicted as 20, a sialidase is depicted as 30, and a Fab is depicted as 40. In each of the constructs depicted in FIGS. 17A-17I it is understood that the Fc may optionally be modified in some manner, e.g. using Knobs-into-Holes type technology, e.g., as depicted by 50 in FIG. 17B. Throughout FIG. 17 similar structures are depicted by similar schematic representations.

FIG. 17A depicts antibody conjugate constructs comprising a first polypeptide comprising a first immunoglobulin light chain; a second polypeptide comprising a first immunoglobulin heavy chain; a third polypeptide comprising a second immunoglobulin heavy chain; and a fourth polypeptide comprising a second immunoglobulin light chain. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first anti-CD20 antigen-binding site as depicted as 10, and the third polypeptide and the fourth polypeptide together define a second anti-CD20 antigen-binding site as depicted as 20. A sialidase enzyme as depicted as 30 can be conjugated to the N- or C-terminus of the first and second immunoglobulin light chain or the first and second immunoglobulin heavy chain.

FIG. 17B depicts antibody conjugate constructs comprising a first polypeptide comprising a first immunoglobulin light chain; a second polypeptide comprising a first immunoglobulin heavy chain; a third polypeptide comprising a second immunoglobulin heavy chain; and a fourth polypeptide comprising a second immunoglobulin light chain. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first anti-CD20 antigen-binding site, and the third polypeptide and the fourth polypeptide together define a second anti-CD20 antigen-binding site. A sialidase enzyme can be conjugated to the N- or C-terminus of the first immunoglobulin light chain or the first immunoglobulin heavy chain.

FIG. 17C depicts antibody conjugate constructs comprising a first polypeptide comprising an immunoglobulin light chain; a second polypeptide comprising an immunoglobulin heavy chain; and a third polypeptide comprising an immunoglobulin Fc domain. The first and second polypeptides can be covalently linked together and the second and third polypeptides can be covalently linked together. The covalent linkages can be disulfide bonds. In certain embodiments, the first polypeptide and the second polypeptide together define an anti-CD20 antigen-binding site. A sialidase enzyme can be conjugated to the N- or C-terminus of the first immunoglobulin light chain or the first immunoglobulin heavy chain.

FIG. 17D depicts antibody conjugate constructs comprising a first polypeptide comprising an immunoglobulin light chain; a second polypeptide comprising an immunoglobulin heavy chain; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The third polypeptide comprises the sialidase and the immunoglobulin Fc domain in an N- to C-terminal orientation. In certain embodiments, the first polypeptide and the second polypeptide together define an anti-CD20 antigen-binding site. An optional second sialidase enzyme can be conjugated to the N- or C-terminus of the first immunoglobulin light chain or the first immunoglobulin heavy chain.

FIG. 17E depicts antibody conjugate constructs comprising a first polypeptide comprising an immunoglobulin light chain; a second polypeptide comprising an immunoglobulin heavy chain; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The third polypeptide comprises the immunoglobulin Fc domain and the sialidase in an N- to C-terminal orientation. In certain embodiments, the first polypeptide and the second polypeptide together define an anti-CD20 antigen-binding site. An optional second sialidase enzyme can be conjugated to the N- or C-terminus of the first immunoglobulin light chain or the first immunoglobulin heavy chain.

FIG. 17F depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N- or C-terminus of the first immunoglobulin Fc domain or to the N- or C-terminus of the second immunoglobulin Fc domain. An optional second sialidase enzyme can be conjugated to the N- or C-terminus of the first immunoglobulin Fc domain or to the N- or C-terminus of the second immunoglobulin Fc domain.

FIG. 17G depicts antibody conjugate constructs comprising a first polypeptide comprising an immunoglobulin light chain; and a second polypeptide comprising an immunoglobulin heavy chain variable region. The first and second polypeptides can be covalently linked together. The covalent linkages can be disulfide bonds. In certain embodiments, the first polypeptide and the second polypeptide together define an anti-CD20 antigen-binding site. The sialidase enzyme can be conjugated to the N- or C-terminus of the immunoglobulin light chain or the immunoglobulin heavy chain variable region.

FIG. 17H depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N-terminus of the first immunoglobulin Fc domain or the second immunoglobulin Fc domain. An optional second sialidase enzyme can be conjugated to the N-terminus of the second immunoglobulin Fc domain or the first immunoglobulin Fc domain, respectively. A single chain variable fragment (scFv) can be conjugated to the C-terminus of the first immunoglobulin Fc domain or the second immunoglobulin Fc domain. An optional second single chain variable fragment (scFv) can be conjugated to the C-terminus of the first immunoglobulin Fc domain or the second immunoglobulin Fc domain, respectively.

FIG. 17I depicts antibody conjugate constructs similar to those depicted in FIG. 17H except that each scFv is replaced with an immunoglobulin antigen binding fragment, e.g., an Fab. For example, FIG. 17I depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N-terminus of the first immunoglobulin Fc domain or the second immunoglobulin Fc domain. An optional second sialidase enzyme can be conjugated to the N-terminus of the second immunoglobulin Fc domain or the first immunoglobulin Fc domain, respectively. An antibody fragment (Fab) can be conjugated or fused to the C-terminus of the first immunoglobulin Fc domain or the second immunoglobulin Fc domain. An optional second antibody fragment (Fab) can be conjugated or fused to the C-terminus of the second immunoglobulin Fc domain or the first immunoglobulin Fc domain, respectively. In the case of a fusion, the C terminus of the Fc domain is linked (either by a bond or an amino acid linker) to a first polypeptide chain defining an anti-CD20 immunoglobulin antigen binding fragment. In the case of antibodies that have an antigen binding site defined by a single variable region, then this may be sufficient to impart binding affinity to a target antigen, e.g., CD20. In other instances, e.g., in the case of a human antibody, the first polypeptide chain defining an immunoglobulin antigen binding fragment can be conjugated (e.g., covalently conjugated, e.g., via a disulfide bond) to a second polypeptide chain defining an immunoglobulin antigen binding fragment, there the two antigen binding fragments together define an antigen binding site for binding the target antigen, e.g., CD20.

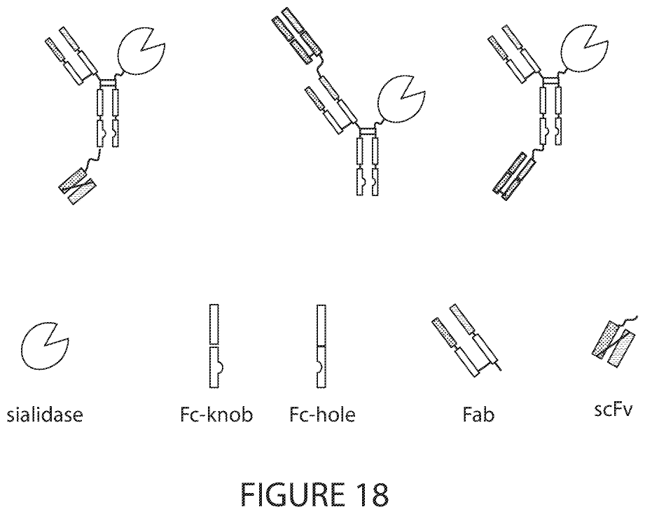

FIG. 18 depicts additional antibody conjugate constructs. For example, FIG. 18 depicts an antibody conjugate construct comprising a first polypeptide comprising an immunoglobulin light chain; a second polypeptide comprising an immunoglobulin heavy chain and an scFv; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The second polypeptide comprises the heavy chain and the scFv in an N- to C-terminal orientation. The third polypeptide comprises the sialidase and the immunoglobulin Fc domain in an N- to C-terminal orientation. In certain embodiments, the first polypeptide and the second polypeptide together define a first antigen-binding site. In certain embodiments, the scFv defines a second antigen-binding site. FIG. 18 depicts an additional antibody construct comprising a first polypeptide comprising an immunoglobulin light chain; a second polypeptide comprising an immunoglobulin heavy chain; and a third polypeptide comprising an immunoglobulin Fc domain and a first sialidase enzyme, wherein a Fab fragment is conjugated to the N-terminus of the immunoglobulin heavy chain. The first and second polypeptides can be covalently linked together and the second and third polypeptides can be covalently linked together. The covalent linkages can be disulfide bonds. The third polypeptide comprises the sialidase and the immunoglobulin Fc domain in an N- to C-terminal orientation. In certain embodiments, the first polypeptide and the second polypeptide together define a first antigen-binding site. In certain embodiments, the Fab fragment defines a second antigen-binding site. In each of the constructs depicted in FIG. 18 it is understood that an scFv, when present, may be replaced with a Fab fragment, or a Fab fragment, when present, may be replaced with an scFv. In each of the constructs depicted in FIG. 18, it is understood that the Fc may optionally be modified in some manner.

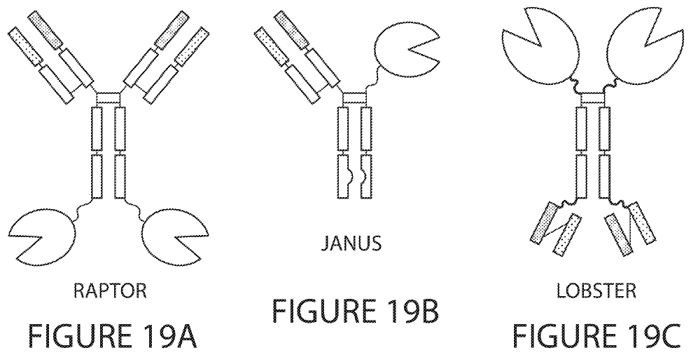

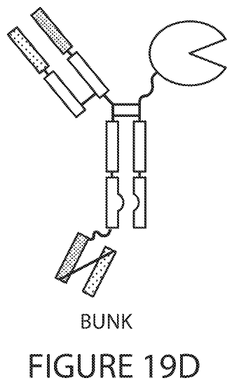

In certain embodiments, the antibody conjugate comprises a first polypeptide comprising a first immunoglobulin light chain; a second polypeptide comprising a first immunoglobulin heavy chain and a first sialidase; a third polypeptide comprising a second immunoglobulin heavy chain and a second sialidase; and a fourth polypeptide comprising a second immunoglobulin light chain. An example of this embodiment is shown in FIG. 19A. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first anti-CD20 antigen-binding site, and the third polypeptide and the fourth polypeptide together define a second anti-CD20 antigen-binding site. In certain embodiments, the second and third polypeptides comprise the first and second immunoglobulin heavy chain and the first and second sialidase, respectively, in an N- to C-terminal orientation. In certain embodiments, the second and third polypeptides comprise the first and second sialidase and the first and second immunoglobulin heavy chain, respectively, in an N- to C-terminal orientation.

In certain embodiments, the first and/or fourth polypeptide comprises the amino acid sequence of any one of SEQ ID NOs: 77, 80, or 111, or an amino acid sequence that has at least 85%, 90%, 95%, 96%, 97%, 98%, or 99% sequence identity to any one of SEQ ID NOs: 77, 80, or 111. In certain embodiments, the second and/or third polypeptide comprises the amino acid sequence of any one of SEQ ID NOs: 119 or 121, or an amino acid sequence that has at least 85%, 90%, 95%, 96%, 97%, 98%, or 99% sequence identity to any one of SEQ ID NOs: 119 or 121. In certain embodiments, the first and fourth polypeptide comprise SEQ ID NO: 111 and the second and third polypeptide comprise SEQ ID NO: 119. In certain embodiments, the first and fourth polypeptide comprise SEQ ID NO: 111 and the second and third polypeptide comprise SEQ ID NO: 121.

In certain embodiments, the antibody conjugate comprises a first polypeptide comprising an immunoglobulin light chain; a second polypeptide comprising an immunoglobulin heavy chain; and a third polypeptide comprising an immunoglobulin Fc domain and a sialidase. An example of this embodiment is shown in FIG. 19B. The first and second polypeptides can be covalently linked together and the second and third polypeptides can be covalently linked together. The covalent linkages can be disulfide bonds. In certain embodiments, the first polypeptide and the second polypeptide together define an anti-CD20 antigen-binding site. In certain embodiments, the third polypeptide comprises the sialidase and the immunoglobulin Fc domain in an N- to C-terminal orientation, or the immunoglobulin Fc domain and the sialidase in an N- to C-terminal orientation.

In certain embodiments, the first polypeptide comprises the amino acid sequence of any one of SEQ ID NOs: 64, 66, 77 80, or 111, or an amino acid sequence that has at least 85%, 90%, 95%, 96%, 97%, 98%, or 99% sequence identity to any one of SEQ ID NOs: 64, 66, 77, 80, or 111. In certain embodiments, the second polypeptide comprises the amino acid sequence of any one of SEQ ID NOs: 67, 81, 129, or 130, or an amino acid sequence that has at least 85%, 90%, 95%, 96%, 97%, 98%, or 99% sequence identity to any one of SEQ ID NOs: 67, 81, 129, or 130. In certain embodiments, the third polypeptide comprises the amino acid sequence of any one of SEQ ID NOs: 68-74, 78, 88-97, 103, 104, 106, 107, 109, 110, 113, 115, 117, 127, 128, 134, or 135, or an amino acid sequence that has at least 85%, 90%, 95%, 96%, 97%, 98%, or 99% sequence identity to any one of SEQ ID NOs: 68-74, 78, 88-97, 103, 104, 106, 107, 109, 110, 113, 115, 117, 127, 128, 134, or 135.

In certain embodiments, the third polypeptide comprises the amino acid sequence of X₁X₂SX₃X₄X₅LQX₆ESVFQSGAHAYRIPALLYLPGQQSLLAFAEQRASX₇X₈DEHAELIVX₉RRGD YDAX₁₀THQVQWX₁₁AQEVVAQAX₁₂LDGHRSMNPCPLYDX₁₃QTGTLFLFFIAIPX₁₄X₁₅VTEX₁₆Q QLQTRANVTRLX₁₇X₁₈VTSTDHGRTWSSPRDLTDAAIGPX₁₉YREWSTFAVGPGHX₂₀LQLHDRX₂₄RSLVVPAYAYRKLHPX₂₂QRPIPSAFX₂₃FLSHDHGRTWARGHFVAQDTX₂₄ECQVAEVETGEQRV VTLNARSHLRARVQAQSX₂₅NX₂₆GLDFQX₂₇SQLVKKLVEPPPX₂₈GX₂₉QGSVISFPSPRSGPGSP AQX₃₀LLYTHPTHX₃₁X₃₂QRADLGAYLNPRPPAPEAWSEPX₃₃LLAKGSX₃₄AYSDLQSMGTGPDGS PLFGX₃₅LYEANDYEEIX₃₆FX₃₇MFTLKQAFPAEYLPQGGGGSGGGGSDKTHTCPPCPAPELLGG PSVFLFPPKPKDTLMISRTPEVTCVVVDVSHEDPEVKFNWYVDGVEVHNAKTKPREEQYNSTYR VVSVLTVLHQDWLNGKEYKCKVSNKALPAPIEKTISKAKGQPREPQVYTLPPSREEMTKNQVSL TCLVKGFYPSDIAVEWESNGQPENNYKTTPPVLDSDGSFFLTSKLTVDKSRWQQGNVFSCSVMH EALHNHYTQKSLSLSPGK

(SEQ ID NO: 76), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Lys, Arg, or Glu. X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Ala, Glu or Lys, X₁₄ is Gly or Asp, X₁₅ is Gln or His, X₁₆ is Gln, Arg, or Lys, X₁₇ is Ala, Cys, Ile, Ser, Val, or Leu, X₁₈ is Gln or Leu, X₁₉ is Ala or Val, X₂₀ is Cys or Gly, X₂₁ is Ala or Gly, X₂₂ is Arg, Ile, or Lys, X₂₃ is Ala, Cys, Leu, or Val, X₂₄ is Leu, Ala, or Val, X₂₅ is Thr or Ala, X₂₆ is Asp or Gly, X₂₇ is Glu or Lys, X₂₈ is Gln, Ala, His, Phe, or Pro, X₂₉ is Cys or Val, X₃₀ is Trp or Arg, X₃₁ is Ser or Arg, X₃₂ is Trp or Lys, X₃₃ is Lys or Val, X₃₄ is Ala, Cys, Ser, or Val, X₃₅ is Cys, Leu, or Val, X₃₆ is Val or Arg, and X₃₇ is Leu, Gln, His, Ile, Lys, or Ser, and the sialidase comprises at least one mutation relative to wild-type human Neu2 (SEQ ID NO: 1).

In certain embodiments, the third polypeptide comprises the amino acid sequence of X₁ASLPX₂LQX₃ESVFQSGAHAYRIPALLYLPGQQSLLAFAEQRASKKDEHAELIVLRRGDYDAX₄THQVQWQAQEVVAQARLDGHRSMNPCPLYDX₃QTGTLFLFFIAIPGQVTEQQQLQTRANVTRLCQ VTSTDHGRTWSSPRDLTDAAIGPAYREWSTFAVGPGHCLQLHDRARSLVVPAYAYRKLHPX₆QRP IPSAFCFLSHDHGRTWARGHFVAQDTLECQVAEVETGEQRVVTLNARSHLRARVQAQSTNDGLD FQESQLVKKLVEPPPX₇GCQGSVISFPSPRSGPGSPAQWLLYTHPTHX₈X₉QRADLGAYLNPRPP APEAWSEPVLLAKGSX₁₀AYSDLQSMGTGPDGSPLFGCLYEANDYEEIX₁₁FX₁₂MFTLKQAFPAE YLPQGGGGSGGGGSDKTHTCPPCPAPELLGGPSVFLFPPKPKDTLMISRTPEVTCVVVDVSHED PEVKFNWYVDGVEVHNAKTKPREEQYNSTYRVVSVLTVLHQDWLNGKEYKCKVSNKALPAPIEK TISKAKGQPREPQVYTLPPSREEMTKNQVSLTCLVKGFYPSDIAVEWESNGQPENNYKTTPPVL DSDGSFFLTSKLTVDKSRWQQGNVFSCSVMHEALHNHYTQKSLSLSPGK

(SEQ ID NO: 75), wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Arg, Ile, or Lys, X₇ is Gln, Ala, His, Phe, or Pro, X₈ is Ser or Arg, X₉ is Trp or Lys, X₁₀ is Ala, Cys, Ser, or Val, X₁₁ is Val or Arg, and X₁₂ is Leu, Gln, His, Ile, Lys, or Ser, and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Ile or Lys, X₇ is Gln or Ala, X₈ is Ser or Arg, X₉ is Trp or Lys, X₁₀ is Ala or Cys, X₁₁ is Val or Arg, and X₁₂ is Leu or Ile.

In certain embodiments, the third polypeptide comprises the amino acid sequence of X₁X₂SX₃X₄X₅LQX₆ESVFQSGAHAYRIPALLYLPGQQSLLAFAEQRASX₇X₈DEHAELIVX₉RRGD YDAX₁₀THQVQWX₁₁AQEVVAQAX₁₂LDGHRSMNPCPLYDX₁₃QTGILFLFFIAIPX₁₄X₁₅VTEX₁₆Q QLQTRANVTRLX₁₇X₁₈VTSTDHGRTWSSPRDLTDAAIGPX₁₉YREWSTFAVGPGHX₂₀LQLHDRX₂₁RSLVVPAYAYRKLHPX₂₂QRPIPSAFX₂₃FLSHDHGRTWARGHFVAQDTX₂₄ECQVAEVETGEQRV VTLNARSHLRARVQAQSX₂₅NX₂₆GLDFQX₂₇SQLVKKLVEPPPX₂₈GX₂₉QGSVISFPSPRSGPGSP AQX₃₀LLYTHPTHX₃₁X₃₂QRADLGAYLNPRPPAPEAWSEPX₃₃LLAKGSX₃₄AYSDLQSMGTGPDGS PLFGX₃₃LYEANDYEEIX₃₆FX₃₇MFTLKQAFPAEYLPQX₃₈DKTHTCPPCPAPELLGGPSVFLFPP KPKDTLMISRTPEVTCVVVDVSHEDPEVKFNWYVDGVEVHNAKTKPREEQYNSTYRVVSVLTVL HQDWLNGKEYKCKVSNKALPAPIEKTISKAKGQPREPQVYTLPPSREEMTKNQVSLTCLVKGFY PSDIAVEWESNGQPENNYKTTPPVLDSDGSFFLTSKLTVDKSRWQQGNVFSCSVMHEALHNHYT QKSLSLSPGK

(SEQ ID NO: 96), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Lys, Arg, or Glu. X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Ala, Glu or Lys, X₁₄ is Gly or Asp, X₁₅ is Gln or His, X₁₆ is Gln, Arg, or Lys, X₁₇ is Ala, Cys, Ile, Ser, Val, or Leu, X₁₈ is Gln or Leu, X₁₉ is Ala or Val, X₂₀ is Cys or Gly, X₂₁ is Ala or Gly, X₂₂ is Arg, Ile, or Lys, X₂₃ is Ala, Cys, Leu, or Val, X₂₄ is Leu, Ala, or Val, X₂₅ is Thr or Ala, X₂₆ is Asp or Gly, X₂₇ is Glu or Lys, X₂₈ is Gln, Ala, His, Phe, or Pro, X₂₉ is Cys or Val, X₃₀ is Trp or Arg, X₃₁ is Ser or Arg, X₃₂ is Trp or Lys, X₃₃ is Lys or Val, X₃₄ is Ala, Cys, Ser, or Val, X₃₅ is Cys, Leu, or Val, X₃₆ is Val or Arg, X₃₇ is Leu, Gln, His, Ile, Lys, or Ser, and X₃₈ is GGGGSGGGGS (SEQ ID NO: 98) or EPKSS (SEQ ID NO: 99), and the sialidase comprises at least one mutation relative to wild-type human Neu2 (SEQ ID NO: 1).

In certain embodiments, the third polypeptide comprises the amino acid sequence of X₁ASLPX₂LQX₃ESVFQSGAHAYRIPALLYLPGQQSLLAFAEQRASKKDEHAELIVLRRGDYDAX₄THQVQWQAQEVVAQARLDGHRSMNPCPLYDX₃QTGTLFLFFIAIPGQVTEQQQLQTRANVTRLCQ VTSTDHGRTWSSPRDLTDAAIGPAYREWSTFAVGPGHCLQLHDRARSLVVPAYAYRKLHPX₆QRP IPSAFCFLSHDHGRTWARGHFVAQDTLECQVAEVETGEQRVVTLNARSHLRARVQAQSTNDGLD FQESQLVKKLVEPPPX₇GCQGSVISFPSPRSGPGSPAQWLLYTHPTHX₈X₉QRADLGAYLNPRPP APEAWSEPVLLAKGSX₁₀AYSDLQSMGTGPDGSPLFGCLYEANDYEEIX₁₁FX₁₂MFTLKQAFPAE YLPQX₁₃DKTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPIEKTISKAKGQ PREPQVYTLPPSREEMTKNQVSLTCLVKGFYPSDIAVEWESNGQPENNYKTTPPVLDSDGSFFL TSKLTVDKSRWQQGNVFSCSVMHEALHNHYTQKSLSLSPGK

(SEQ ID NO: 95), wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Arg, Ile, or Lys, X₇ is Gln, Ala, His, Phe, or Pro, X₈ is Ser or Arg, X₉ is Trp or Lys, X₁₀ is Ala, Cys, Ser, or Val, X₁₁ is Val or Arg, X₁₂ is Leu, Gln, His, Ile, Lys, or Ser, and X₁₃ is GGGGSGGGGS (SEQ ID NO: 98) or EPKSS (SEQ ID NO: 99), and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Ile or Lys, X₇ is Gln or Ala, X₈ is Ser or Arg, X₉ is Trp or Lys, X₁₀ is Ala or Cys, X₁₁ is Val or Arg, and X₁₂ is Leu or Ile.

In certain embodiments, the third polypeptide comprises the amino acid sequence of X₁X₂SX₃X₄X₃LQX₆ESVFQSGAHAYRIPALLYLPGQQSLLAFAEQRASX₇X₈DEHAELIVX₉RRGD YDAX₁₀THQVQWX₁₁AQEVVAQAX₁₂LX₁₃GHRSMNPCPLYDX₁₄QTGILFLFFIAIPX₁₅X₁₆VTEX₁₇QQLQTRANVTRLX₁₈X₁₉VTSTDHGRTWSSPRDLTDAAIGPX₂₀YREWSTFAVGPGHX₂₁LQLHDX₂₂X₂₃RSLVVPAYAYRKLHPX₂₄X₂₅X₂₆PPSAFX₂₇FLSHDHGRTWARGHFVX₂₈QDTX₂₉ECQVAEVX₃₀TGEQRVVTLNARSX₃₁X₃₂X₃₃X₃₄RX₃₅QAQSX₃₆NX₃₇GLDFQX₃₈X₃₉QX₄₀VKKL X₄₁EPPPX₄₂GX₄₃QGSVISFPSPRSGPGSPAQX₄₄LLYTHPTHX₄₅X₄₆QRADLGAYLNPRPPAPEA WSEPX₄₇LLAKGSX₄₈AYSDLQSMGTGPDGSPLFGX₄₉LYEANDYEEIX₅₀FX₅₁MFTLKQAFPAEYL PQX₅₂DKTHTCPPCPAPELLGGPSVFLFPPKPKDTLMISRTPEVTCVVVDVSHEDPEVKFNWYVD GVEVHNAKTKPREEQYNSTYRVVSVLTVLHQDWLNGKEYKCKVSNKALPAPIEKTISKAKGQPR EPQVYTLPPSREEMTKNQVSLTCLVKGFYPSDIAVEWESNGQPENNYKTTPPVLDSDGSFFLTS KLTVDKSRWQQGNVFSCSVMHEALHNHYTQKSLSLSPGK

(SEQ ID NO: 132), wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Lys, Arg, or Glu, X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Asp or Pro, X₁₄ is Ala, Glu or Lys, X₁₅ is Gly or Asp, X₁₆ is Gln or His, X₁₇ is Gln, Arg, or Lys, X₁₈ is Ala, Cys, Ile, Ser, Val, or Leu, X₁₉ is Gln, Leu, Glu, Phe, His, Ile, Leu, or Tyr, X₂₀ is Ala or Val, X₂₁ is Cys or Gly, X₂₂ is Arg or Pro, X₂₃ is Ala or Gly, X₂₄ is Arg, Ile, or Lys, X₂₅ is Gln or Pro, X₂₆ is Arg or Pro, X₂₇ is Ala, Cys, Leu, or Val, X₂₈ is Ala, Cys, Asn, Ser, or Thr, X₂₉ is Leu, Ala, or Val, X₃₀ is Glu or Pro, X₃₁ is His or Pro, X₃₂ is Leu, Asp, Asn, or Tyr, X₃₃ is Arg, Ala, Asp, Leu, Gln, or Tyr, X₃₄ is Ala, Cys, Phe, Gly, His, Ile, Lys, Leu, Met, Asn, Gln, Arg, Ser, Val, Trp, or Tyr, X₃₅ is Val, Ile, or Lys, X₃₆ is Thr or Ala, X₃₇ is Asp or Gly, X₃₈ is Glu, Lys, or Pro, X₃₉ is Ser or Cys, X₄₀ is Leu, Asp, Phe, Gln, or Thr, X₄₁ is Val or Phe, X₄₂ is Gln, Ala, His, Phe, Pro, Ser, or Thr, X₄₃ is Cys or Val, X₄₄ is Trp or Arg, X₄₅ is Ser, Arg, Ala, Asp, Glu, Phe, Gly, His, Ile, Lys, Leu, Met, Asn, Pro, Gln, Thr, Val, Trp, or Tyr, X₄₆ is Trp, Lys, Ala, Asp, Glu, Phe, Gly, His, Ile, Lys, Leu, Met, Asn, Pro, Gln, Arg, Ser, Thr, Val, or Tyr, X₄₇ is Lys or Val, X₄₈ is Ala, Cys, Ser, or Val, X₄₉ is Cys, Leu, or Val, X₅₀ is Val or Arg, X₅₁ is Leu, Gln, His, Ile, Lys, or Ser, X₅₂ is GGGGS (SEQ ID NO: 126), GGGGSGGGGS (SEQ ID NO: 98), or EPKSS (SEQ ID NO: 99), and the sialidase comprises at least one mutation relative to wild-type human Neu2 (SEQ ID NO: 1).

In certain embodiments, the third polypeptide comprises the amino acid sequence of X₁ASLPX₂LQX₃ESVFQSGAHAYRIPALLYLPGQQSLLAFAEQRASKKDEHAELIVLRRGDYDAX₄THQVQWQAQEVVAQARLDGHRSMNPCPLYDX₅QTGTLFLFFIAIPGQVTEQQQLQTRANVTRLCX₆VISTDHGRTWSSPRDLTDAAIGPAYREWSTFAVGPGHCLQLHDRARSLVVPAYAYRKLHPX₇QR PIPSAFCFLSHDHGRTWARGHFVAQDTLECQVAEVETGEQRVVTLNARSHLRX₈RVQAQSTNDGL DFQESQLVKKLVEPPPX₉GCQGSVISFPSPRSGPGSPAQWLLYTHPTHX₁₀X₁₁QRADLGAYLNPR PPAPEAWSEPVLLAKGSX₁₂AYSDLQSMGTGPDGSPLFGCLYEANDYEEIX₁₃FX₁₄MFTLKQAFP AEYLPQX₁₃DKTHTCPPCPAPELLGGPSVFLFPPKPKDTLMISRTPEVTCVVVDVSHEDPEVKFN WYVDGVEVHNAKTKPREEQYNSTYRVVSVLTVLHQDWLNGKEYKCKVSNKALPAPIEKTISKAK GQPREPQVYTLPPSREEMTKNQVSLTCLVKGFYPSDIAVEWESNGQPENNYKTTPPVLDSDGSF FLTSKLTVDKSRWQQGNVFSCSVMHEALHNHYTQKSLSLSPGK

(SEQ ID NO: 131), wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Gln, Leu, Glu, Phe, His, Ile, Leu, or Tyr, X₇ is Arg, Ile, or Lys, X₈ is Ala, Cys, Phe, Gly, His, Ile, Lys, Leu, Met, Asn, Gln, Arg, Ser, Val, Trp, or Tyr, X₉ is Gln, Ala, His, Phe, Pro, Ser, or Thr, X₁₀ is Ser, Arg, Ala, Asp, Glu, Phe, Gly, His, Ile, Lys, Leu, Met, Asn, Pro, Gln, Thr, Val, Trp, or Tyr, X₁₁ is Trp, Lys, Ala, Asp, Glu, Phe, Gly, His, Ile, Lys, Leu, Met, Asn, Pro, Gln, Arg, Ser, Thr, Val, or Tyr, X₁₂ is Ala, Cys, Ser, or Val, X₁₃ is Val or Arg, X₁₄ is Leu, Gln, His, Ile, Lys, or Ser, X₁₅ is GGGGS (SEQ ID NO: 126), GGGGSGGGGS (SEQ ID NO: 98), or EPKSS (SEQ ID NO: 99), and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Gln or Tyr, X₇ is Ile or Lys, X₈ is Ala or Thr, X₉ is Gln, Ala, or Thr, X₁₀ is Ser, Arg, or Ala, X₁₁ is Trp, Lys, or Arg, X₁₂ is Ala or Cys, X₁₃ is Val or Arg, and X₁₄ is Leu or Ile.

In certain embodiments, the first polypeptide comprises SEQ ID NO: 66, the second polypeptide comprises SEQ ID NO: 67, and the third polypeptide comprises SEQ ID NO: 68. In certain embodiments, the first polypeptide comprises SEQ ID NO: 66, the second polypeptide comprises SEQ ID NO: 67, and the third polypeptide comprises SEQ ID NO: 69. In certain embodiments, the first polypeptide comprises SEQ ID NO: 66, the second polypeptide comprises SEQ ID NO: 67, and the third polypeptide comprises SEQ ID NO: 70. In certain embodiments, the first polypeptide comprises SEQ ID NO: 66, the second polypeptide comprises SEQ ID NO: 67, and the third polypeptide comprises SEQ ID NO: 71. In certain embodiments, the first polypeptide comprises SEQ ID NO: 66, the second polypeptide comprises SEQ ID NO: 67, and the third polypeptide comprises SEQ ID NO: 72. In certain embodiments, the first polypeptide comprises SEQ ID NO: 66, the second polypeptide comprises SEQ ID NO: 67, and the third polypeptide comprises SEQ ID NO: 73. In certain embodiments, the first polypeptide comprises SEQ ID NO: 66, the second polypeptide comprises SEQ ID NO: 67, and the third polypeptide comprises SEQ ID NO: 74. In certain embodiments, the first polypeptide comprises SEQ ID NO: 66, the second polypeptide comprises SEQ ID NO: 67, and the third polypeptide comprises SEQ ID NO: 78. In certain embodiments, the first polypeptide comprises SEQ ID NO: 66, the second polypeptide comprises SEQ ID NO: 67, and the third polypeptide comprises SEQ ID NO: 88. In certain embodiments, the first polypeptide comprises SEQ ID NO: 66, the second polypeptide comprises SEQ ID NO: 67, and the third polypeptide comprises SEQ ID NO: 89. In certain embodiments, the first polypeptide comprises SEQ ID NO: 66, the second polypeptide comprises SEQ ID NO: 67, and the third polypeptide comprises SEQ ID NO: 90. In certain embodiments, the first polypeptide comprises SEQ ID NO: 66, the second polypeptide comprises SEQ ID NO: 67, and the third polypeptide comprises SEQ ID NO: 91. In certain embodiments, the first polypeptide comprises SEQ ID NO: 66, the second polypeptide comprises SEQ ID NO: 67, and the third polypeptide comprises SEQ ID NO: 92. In certain embodiments, the first polypeptide comprises SEQ ID NO: 66, the second polypeptide comprises SEQ ID NO: 67, and the third polypeptide comprises SEQ ID NO: 93. In certain embodiments, the first polypeptide comprises SEQ ID NO: 66, the second polypeptide comprises SEQ ID NO: 67, and the third polypeptide comprises SEQ ID NO: 94. In certain embodiments, the first polypeptide comprises SEQ ID NO: 66, the second polypeptide comprises SEQ ID NO: 67, and the third polypeptide comprises SEQ ID NO: 97. In certain embodiments, the first polypeptide comprises SEQ ID NO: 66, the second polypeptide comprises SEQ ID NO: 67, and the third polypeptide comprises SEQ ID NO: 103. In certain embodiments, the first polypeptide comprises SEQ ID NO: 66, the second polypeptide comprises SEQ ID NO: 67, and the third polypeptide comprises SEQ ID NO: 104. In certain embodiments, the first polypeptide comprises SEQ ID NO: 66, the second polypeptide comprises SEQ ID NO: 67, and the third polypeptide comprises SEQ ID NO: 106. In certain embodiments, the first polypeptide comprises SEQ ID NO: 66, the second polypeptide comprises SEQ ID NO: 67, and the third polypeptide comprises SEQ ID NO: 107. In certain embodiments, the first polypeptide comprises SEQ ID NO: 66, the second polypeptide comprises SEQ ID NO: 67, and the third polypeptide comprises SEQ ID NO: 109. In certain embodiments, the first polypeptide comprises SEQ ID NO: 66, the second polypeptide comprises SEQ ID NO: 67, and the third polypeptide comprises SEQ ID NO: 110. In certain embodiments, the first polypeptide comprises SEQ ID NO: 66, the second polypeptide comprises SEQ ID NO: 67, and the third polypeptide comprises SEQ ID NO: 134. In certain embodiments, the first polypeptide comprises SEQ ID NO: 66, the second polypeptide comprises SEQ ID NO: 67, and the third polypeptide comprises SEQ ID NO: 135. In certain embodiments, the first polypeptide comprises SEQ ID NO: 66, the second polypeptide comprises SEQ ID NO: 129, and the third polypeptide comprises SEQ ID NO: 127. In certain embodiments, the first polypeptide comprises SEQ ID NO: 66, the second polypeptide comprises SEQ ID NO: 129, and the third polypeptide comprises SEQ ID NO: 128.

In certain embodiments, the first polypeptide comprises SEQ ID NO: 80, the second polypeptide comprises SEQ ID NO: 81, and the third polypeptide comprises SEQ ID NO: 68. In certain embodiments, the first polypeptide comprises SEQ ID NO: 80, the second polypeptide comprises SEQ ID NO: 81, and the third polypeptide comprises SEQ ID NO: 69. In certain embodiments, the first polypeptide comprises SEQ ID NO: 80, the second polypeptide comprises SEQ ID NO: 81, and the third polypeptide comprises SEQ ID NO: 70. In certain embodiments, the first polypeptide comprises SEQ ID NO: 80, the second polypeptide comprises SEQ ID NO: 81, and the third polypeptide comprises SEQ ID NO: 71. In certain embodiments, the first polypeptide comprises SEQ ID NO: 80, the second polypeptide comprises SEQ ID NO: 81, and the third polypeptide comprises SEQ ID NO: 72. In certain embodiments, the first polypeptide comprises SEQ ID NO: 80, the second polypeptide comprises SEQ ID NO: 81, and the third polypeptide comprises SEQ ID NO: 73. In certain embodiments, the first polypeptide comprises SEQ ID NO: 80, the second polypeptide comprises SEQ ID NO: 81, and the third polypeptide comprises SEQ ID NO: 74. In certain embodiments, the first polypeptide comprises SEQ ID NO: 80, the second polypeptide comprises SEQ ID NO: 81, and the third polypeptide comprises SEQ ID NO: 78. In certain embodiments, the first polypeptide comprises SEQ ID NO: 80, the second polypeptide comprises SEQ ID NO: 81, and the third polypeptide comprises SEQ ID NO: 88. In certain embodiments, the first polypeptide comprises SEQ ID NO: 80, the second polypeptide comprises SEQ ID NO: 81, and the third polypeptide comprises SEQ ID NO: 89. In certain embodiments, the first polypeptide comprises SEQ ID NO: 80, the second polypeptide comprises SEQ ID NO: 81, and the third polypeptide comprises SEQ ID NO: 90. In certain embodiments, the first polypeptide comprises SEQ ID NO: 80, the second polypeptide comprises SEQ ID NO: 81, and the third polypeptide comprises SEQ ID NO: 91. In certain embodiments, the first polypeptide comprises SEQ ID NO: 80, the second polypeptide comprises SEQ ID NO: 81, and the third polypeptide comprises SEQ ID NO: 92. In certain embodiments, the first polypeptide comprises SEQ ID NO: 80, the second polypeptide comprises SEQ ID NO: 81, and the third polypeptide comprises SEQ ID NO: 93. In certain embodiments, the first polypeptide comprises SEQ ID NO: 80, the second polypeptide comprises SEQ ID NO: 81, and the third polypeptide comprises SEQ ID NO: 94. In certain embodiments, the first polypeptide comprises SEQ ID NO: 80, the second polypeptide comprises SEQ ID NO: 81, and the third polypeptide comprises SEQ ID NO: 97. In certain embodiments, the first polypeptide comprises SEQ ID NO: 80, the second polypeptide comprises SEQ ID NO: 81, and the third polypeptide comprises SEQ ID NO: 103. In certain embodiments, the first polypeptide comprises SEQ ID NO: 80, the second polypeptide comprises SEQ ID NO: 81, and the third polypeptide comprises SEQ ID NO: 104. In certain embodiments, the first polypeptide comprises SEQ ID NO: 80, the second polypeptide comprises SEQ ID NO: 81, and the third polypeptide comprises SEQ ID NO: 106. In certain embodiments, the first polypeptide comprises SEQ ID NO: 80, the second polypeptide comprises SEQ ID NO: 81, and the third polypeptide comprises SEQ ID NO: 107. In certain embodiments, the first polypeptide comprises SEQ ID NO: 80, the second polypeptide comprises SEQ ID NO: 81, and the third polypeptide comprises SEQ ID NO: 109. In certain embodiments, the first polypeptide comprises SEQ ID NO: 80, the second polypeptide comprises SEQ ID NO: 81, and the third polypeptide comprises SEQ ID NO: 110. In certain embodiments, the first polypeptide comprises SEQ ID NO: 80, the second polypeptide comprises SEQ ID NO: 81, and the third polypeptide comprises SEQ ID NO: 134. In certain embodiments, the first polypeptide comprises SEQ ID NO: 80, the second polypeptide comprises SEQ ID NO: 81, and the third polypeptide comprises SEQ ID NO: 135. In certain embodiments, the first polypeptide comprises SEQ ID NO: 80, the second polypeptide comprises SEQ ID NO: 130, and the third polypeptide comprises SEQ ID NO: 127. In certain embodiments, the first polypeptide comprises SEQ ID NO: 80, the second polypeptide comprises SEQ ID NO: 130, and the third polypeptide comprises SEQ ID NO: 128.

In certain embodiments, the first polypeptide comprises SEQ ID NO: 111, the second polypeptide comprises SEQ ID NO: 81, and the third polypeptide comprises SEQ ID NO: 68. In certain embodiments, the first polypeptide comprises SEQ ID NO: 111, the second polypeptide comprises SEQ ID NO: 81, and the third polypeptide comprises SEQ ID NO: 69. In certain embodiments, the first polypeptide comprises SEQ ID NO: 111, the second polypeptide comprises SEQ ID NO: 81, and the third polypeptide comprises SEQ ID NO: 70. In certain embodiments, the first polypeptide comprises SEQ ID NO: 111, the second polypeptide comprises SEQ ID NO: 81, and the third polypeptide comprises SEQ ID NO: 71. In certain embodiments, the first polypeptide comprises SEQ ID NO: 111, the second polypeptide comprises SEQ ID NO: 81, and the third polypeptide comprises SEQ ID NO: 72. In certain embodiments, the first polypeptide comprises SEQ ID NO: 111, the second polypeptide comprises SEQ ID NO: 81, and the third polypeptide comprises SEQ ID NO: 73. In certain embodiments, the first polypeptide comprises SEQ ID NO: 111, the second polypeptide comprises SEQ ID NO: 81, and the third polypeptide comprises SEQ ID NO: 74. In certain embodiments, the first polypeptide comprises SEQ ID NO: 111, the second polypeptide comprises SEQ ID NO: 81, and the third polypeptide comprises SEQ ID NO: 78. In certain embodiments, the first polypeptide comprises SEQ ID NO: 111, the second polypeptide comprises SEQ ID NO: 81, and the third polypeptide comprises SEQ ID NO: 88. In certain embodiments, the first polypeptide comprises SEQ ID NO: 111, the second polypeptide comprises SEQ ID NO: 81, and the third polypeptide comprises SEQ ID NO: 89. In certain embodiments, the first polypeptide comprises SEQ ID NO: 111, the second polypeptide comprises SEQ ID NO: 81, and the third polypeptide comprises SEQ ID NO: 90. In certain embodiments, the first polypeptide comprises SEQ ID NO: 111, the second polypeptide comprises SEQ ID NO: 81, and the third polypeptide comprises SEQ ID NO: 91. In certain embodiments, the first polypeptide comprises SEQ ID NO: 111, the second polypeptide comprises SEQ ID NO: 81, and the third polypeptide comprises SEQ ID NO: 92. In certain embodiments, the first polypeptide comprises SEQ ID NO: 111, the second polypeptide comprises SEQ ID NO: 81, and the third polypeptide comprises SEQ ID NO: 93. In certain embodiments, the first polypeptide comprises SEQ ID NO: 111, the second polypeptide comprises SEQ ID NO: 81, and the third polypeptide comprises SEQ ID NO: 94. In certain embodiments, the first polypeptide comprises SEQ ID NO: 111, the second polypeptide comprises SEQ ID NO: 81, and the third polypeptide comprises SEQ ID NO: 97. In certain embodiments, the first polypeptide comprises SEQ ID NO: 111, the second polypeptide comprises SEQ ID NO: 81, and the third polypeptide comprises SEQ ID NO: 103. In certain embodiments, the first polypeptide comprises SEQ ID NO: 111, the second polypeptide comprises SEQ ID NO: 81, and the third polypeptide comprises SEQ ID NO: 104. In certain embodiments, the first polypeptide comprises SEQ ID NO: 111, the second polypeptide comprises SEQ ID NO: 81, and the third polypeptide comprises SEQ ID NO: 106. In certain embodiments, the first polypeptide comprises SEQ ID NO: 111, the second polypeptide comprises SEQ ID NO: 81, and the third polypeptide comprises SEQ ID NO: 107. In certain embodiments, the first polypeptide comprises SEQ ID NO: 111, the second polypeptide comprises SEQ ID NO: 81, and the third polypeptide comprises SEQ ID NO: 109. In certain embodiments, the first polypeptide comprises SEQ ID NO: 111, the second polypeptide comprises SEQ ID NO: 81, and the third polypeptide comprises SEQ ID NO: 110. In certain embodiments, the first polypeptide comprises SEQ ID NO: 111, the second polypeptide comprises SEQ ID NO: 81, and the third polypeptide comprises SEQ ID NO: 113. In certain embodiments, the first polypeptide comprises SEQ ID NO: 111, the second polypeptide comprises SEQ ID NO: 81, and the third polypeptide comprises SEQ ID NO: 115. In certain embodiments, the first polypeptide comprises SEQ ID NO: 111, the second polypeptide comprises SEQ ID NO: 81, and the third polypeptide comprises SEQ ID NO: 134. In certain embodiments, the first polypeptide comprises SEQ ID NO: 111, the second polypeptide comprises SEQ ID NO: 81, and the third polypeptide comprises SEQ ID NO: 145. In certain embodiments, the first polypeptide comprises SEQ ID NO: 111, the second polypeptide comprises SEQ ID NO: 130, and the third polypeptide comprises SEQ ID NO: 127. In certain embodiments, the first polypeptide comprises SEQ ID NO: 111, the second polypeptide comprises SEQ ID NO: 130, and the third polypeptide comprises SEQ ID NO: 128.

In certain embodiments, the antibody conjugate comprises a first polypeptide comprising a first sialidase, a first immunoglobulin Fc domain, and a first single chain variable fragment (scFv) (it is also understood that the scFv may be replaced by a first polypeptide chain of an immunoglobulin antigen binding fragment, e.g., Fab fragment); and a second polypeptide comprising a second sialidase, a second immunoglobulin Fc domain, and a second single chain variable fragment (scFv) (it is also understood that the scFv may be replaced by a second polypeptide chain of an immunoglobulin antigen binding fragment, e.g., Fab fragment). An example of this embodiment is shown in FIG. 19C. The first and second polypeptides can be covalently linked together. The covalent linkages can be disulfide bonds. In certain embodiments, the first scFv defines a first anti-CD20 antigen-binding site, and the second scFv defines a second anti-CD20 antigen-binding site. In certain embodiments, the first polypeptide comprises the first sialidase, the first immunoglobulin Fc domain, and the first scFv in an N- to C-terminal orientation. In certain embodiments, the first polypeptide comprises the first scFv, the first immunoglobulin Fc domain, and the first sialidase in an N- to C-terminal orientation. In certain embodiments, the second polypeptide comprises the second sialidase, the second immunoglobulin Fc domain, and the second scFv in an N- to C-terminal orientation. In certain embodiments, the second polypeptide comprises the second scFv, the second immunoglobulin Fc domain, and the second sialidase in an N- to C-terminal orientation.

In certain embodiments, the antibody conjugate comprises: a first polypeptide comprising an immunoglobulin light chain; a second polypeptide comprising an immunoglobulin heavy chain and a single chain variable fragment (scFv) (it is also understood that the scFv may be replaced by a first polypeptide chain of an immunoglobulin antigen binding fragment, e.g., Fab fragment); and a third polypeptide comprising an immunoglobulin Fc domain and a sialidase. An example of this embodiment is shown in FIG. 19D. The first and second polypeptides can be covalently linked together and the second and third polypeptides can be covalently linked together. The covalent linkages can be disulfide bonds. In certain embodiments, the first polypeptide and the second polypeptide together define a first anti-CD20 antigen-binding site (i.e., the immunoglobulin light chain and immunoglobulin heavy chain together define a first anti-CD20 antigen-binding site). In certain embodiments, the scFv defines a second anti-CD20 antigen-binding site. In certain embodiments, the second polypeptide comprises the immunoglobulin heavy chain and the scFv in an N- to C-terminal orientation, or the scFv and the immunoglobulin heavy chain in an N- to C-terminal orientation. In certain embodiments, the third polypeptide comprises the sialidase and the immunoglobulin Fc domain in an N- to C-terminal orientation, or the sialidase and the immunoglobulin Fc domain in an N- to C-terminal orientation.

In certain embodiments, the antibody conjugate has a molecular weight from about 135 kDa to about 165 kDa, e.g., about 140 kDa. In other embodiments, the antibody conjugate has a molecular weight from about 215 kDa to about 245 kDa, e.g., about 230 kDa.

In certain embodiments, the antibody conjugate comprises two polypeptides that each comprise an immunoglobulin Fc domain, and the first polypeptide has either a “knob” mutation, e.g., T366Y, or a “hole” mutation, e.g., Y407T, for heterodimerization with the second polypeptide, and the second polypeptide has either a respective “knob” mutation, e.g., T366Y, or a “hole” mutation, e.g., Y407T, for heterodimerization with the first polypeptide (residue numbers according to EU numbering, Kabat, E. A., et al. (1991) supra). For example, in certain embodiments, the antibody comprises two polypeptides that each comprise an immunoglobulin Fc domain derived from human IgG1 Fc domain, and the first polypeptide comprises a Y407T mutation (e.g., the first polypeptide comprises SEQ ID NO: 32 or 100), and the second polypeptide comprises a T366Y mutation (e.g., the second polypeptide comprises SEQ ID NO: 33 or 101).

As used herein, the term “multispecific antibody” is understood to mean an antibody that specifically binds to at least two different antigens, i.e., an antibody that comprises at least two antigen-binding sites that bind to at least two different antigens. As used herein, the term “bispecific antibody” is understood to mean an antibody that specifically binds to two different antigens, i.e., an antibody that comprises two antigen-binding sites each of which bind to separate and distinct antigens. In other words, a first binding site binds a first antigen and a second binding site binds a second, different antigen. A multispecific or bispecific antibody may, for example, be a human or humanized antibody, and/or be a full length antibody or an antibody fragment (e.g., a F(ab′)₂ bispecific antibody).

The present invention encompasses antibody conjugates comprising antibody fragments, which may be generated by traditional means, such as enzymatic digestion, or by recombinant techniques. For a review of certain antibody fragments, see Hudson et al. (2003) supra.

In certain embodiments, the antibody conjugate or fusion protein can be covalently or non-covalently associated with a biological modifier, wherein the biological modifier can be used to enhance the solubility of the antibody, increase binding specificity, decrease immunogenicity or toxicity or modify the pharmacokinetic profile of the antibody. For example, the biological modifier can be used to increase the molecular weight of the antibody to increase its circulating half-life.

It is contemplated that the antibody conjugate or fusion protein may be covalently bound to one or more (for example, 2, 3, 4, 5, 6, 8, 9, 10 or more) biological modifiers that may comprise linear or branched polymers. Exemplary biological modifiers may include, for example, a variety of polymers, such as those described in U.S. Pat. No. 7,842,789. Particularly useful are polyalkylene ethers such as polyethylene glycol (PEG) and derivatives thereof (for example, alkoxy polyethylene glycol, for example, methoxypolyethylene glycol, ethoxypolyethylene glycol and the like); block copolymers of polyoxyethylene and polyoxypropylene (Pluronics); polymethacrylates; carbomers; and branched or unbranched polysaccharides which comprise the saccharide monomers such as D-mannose, D- and L-galactose, fucose, fructose, D-xylose, L-arabinose, and D-glucuronic acid.

In other embodiments, the biological modifier can be a hydrophilic polyvinyl polymer such as polyvinyl alcohol and polyvinylpyrrolidone (PVP)-type polymers. The biological modifier can be a functionalized polyvinylpyrrolidone, for example, carboxy or amine functionalized on one (or both) ends of the polymer (as available from PolymerSource). Alternatively, the biological modifier can include Poly N-(2-hydroxypropyl)methacrylamide (HPMA), or functionalized HPMA (amine, carboxy, etc.), Poly(N-isopropylacrylamide) or functionalized poly(N-isopropylacrylamide). Alternatively, the biological modifier can include Poly N-(2-hydroxypropyl)methacrylamide (HPMA), or functionalized HPMA (amine, carboxy, etc.), Poly(N-isopropylacrylamide) or functionalized poly(N-isopropylacrylamide). The modifier prior to conjugation need not be, but preferably is, water soluble, but the final conjugate should be water soluble.

In general, the biological modifier may have a molecular weight from about 2 kDa to about 5 kDa, from about 2 kDa to about 10 kDa, from about 2 kDa to about 20 kDa, from about 2 kDa to about 30 kDa, from about 2 kDa to about 40 kDa, from about 2 kDa to about 50 kDa, from about 2 kDa to about 60 kDa, from about 2 kDa to about 70 kDa, from about 2 kDa to about 80 kDa, from about 2 kDa to about 90 kDa, from about 2 kDa to about 100 kDa, from about 2 kDa to about 150 kDa, from about 5 kDa to about 10 kDa, from about 5 kDa to about 20 kDa, from about 5 kDa to about 30 kDa, from about 5 kDa to about 40 kDa, from about 5 kDa to about 50 kDa, from about 5 kDa to about 60 kDa, from about 5 kDa to about 70 kDa, from about 5 kDa to about 80 kDa, from about 5 kDa to about 90 kDa, from about 5 kDa to about 100 kDa, from about 5 kDa to about 150 kDa, from about 10 kDa to about 20 kDa, from about 10 kDa to about 30 kDa, from about 10 kDa to about 40 kDa, from about 10 kDa to about 50 kDa, from about 10 kDa to about 60 kDa, from about 10 kDa to about 70 kDa, from about 10 kDa to about 80 kDa, from about 10 kDa to about 90 kDa, from about 10 kDa to about 100 kDa, from about 10 kDa to about 150 kDa, from about 20 kDa to about 30 kDa, from about 20 kDa to about 40 kDa, from about 20 kDa to about 50 kDa, from about 20 kDa to about 60 kDa, from about 20 kDa to about 70 kDa, from about 20 kDa to about 80 kDa, from about 20 kDa to about 90 kDa, from about 20 kDa to about 100 kDa, from about 20 kDa to about 150 kDa, from about 30 kDa to about 40 kDa, from about 30 kDa to about 50 kDa, from about 30 kDa to about 60 kDa, from about 30 kDa to about 70 kDa, from about 30 kDa to about 80 kDa, from about 30 kDa to about 90 kDa, from about 30 kDa to about 100 kDa, from about 30 kDa to about 150 kDa, from about 40 kDa to about 50 kDa, from about 40 kDa to about 60 kDa, from about 40 kDa to about 70 kDa, from about 40 kDa to about 80 kDa, from about 40 kDa to about 90 kDa, from about 40 kDa to about 100 kDa, from about 40 kDa to about 150 kDa, from about 50 kDa to about 60 kDa, from about 50 kDa to about 70 kDa, from about 50 kDa to about 80 kDa, from about 50 kDa to about 90 kDa, from about 50 kDa to about 100 kDa, from about 50 kDa to about 150 kDa, from about 60 kDa to about 70 kDa, from about 60 kDa to about 80 kDa, from about 60 kDa to about 90 kDa, from about 60 kDa to about 100 kDa, from about 60 kDa to about 150 kDa, from about 70 kDa to about 80 kDa, from about 70 kDa to about 90 kDa, from about 70 kDa to about 100 kDa, from about 70 kDa to about 150 kDa, from about 80 kDa to about 90 kDa, from about 80 kDa to about 100 kDa, from about 80 kDa to about 150 kDa, from about 90 kDa to about 100 kDa, from about 90 kDa to about 150 kDa, or from about 100 kDa to about 150 kDa.

It is contemplated that the antibody conjugate or fusion protein is attached to about 10 or fewer polymer molecules (e.g., 9, 8, 7, 6, 5, 4, 3, 2, or 1), each polymer molecule having a molecular weight of at least about 20,000 D, or at least about 30,000 D, or at least about 40,000 D.

Although a variety of polymers can be used as biological modifiers, it is contemplated that the antibody conjugates or fusion proteins described herein may be attached to polyethylene glycol (PEG) polymers. In one embodiment, the antibody conjugate or fusion protein described herein is covalently attached to at least one PEG having an actual MW of at least about 20,000 D. In another embodiment, the antibody conjugate or fusion protein described herein is covalently attached to at least one PEG having an actual MW of at least about 30,000 D. In another embodiment, the antibody conjugate or fusion protein described herein is covalently attached to at least one PEG having an actual MW of at least about 40,000 D. In certain embodiments, the PEG is methoxyPEG(5000)-succinimidylpropionate (mPEG-SPA), methoxyPEG(5000)-succinimidylsuccinate (mPEG-SS). Such PEGS are commercially available from Nektar Therapeutics or SunBiowest.

Attachment sites on an antibody conjugate or fusion protein for a biological modifier include the N-terminal amino group and epsilon amino groups found on lysine residues, as well as other amino, imino, carboxyl, sulfhydryl, hydroxyl or other hydrophilic groups. The polymer may be covalently bonded directly to the antibody conjugate or fusion protein with or without the known use of a multifunctional (ordinarily bifunctional) crosslinking agent using chemistries and used in the art. For example, sulfhydryl groups can be derivatized by coupling to maleimido-substituted PEG (e.g. alkoxy-PEG amine plus sulfosuccinimidyl 4-(N-maleimidomethyl)cyclohexane-1-carboxylate), or PEG-maleimide commercially available from Shearwater Polymers, Inc., Huntsville, Ala.).

### II. Methods of Making a Fusion Protein or Antibody Conjugate

Methods for producing fusion proteins, e.g., those disclosed herein, antibodies, or antibody conjugates, e.g., those disclosed herein, are known in the art. For example, DNA molecules encoding light chain variable regions and/or heavy chain variable regions can be synthesized chemically or by recombinant DNA methodologies. For example, the sequences of the antibodies can be cloned from hybridomas by conventional hybridization techniques or polymerase chain reaction (PCR) techniques, using the appropriate synthetic nucleic acid primers. The resulting DNA molecules encoding the variable regions of interest can be ligated to other appropriate nucleotide sequences, including, for example, constant region coding sequences, and expression control sequences, to produce conventional gene expression constructs (i.e., expression vectors) encoding the desired antibodies. Production of defined gene constructs is within routine skill in the art.

Nucleic acids encoding desired fusion proteins, and/or antibody conjugates can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In embodiments involving fusion proteins comprising an antibody or portion thereof, the expression vector optionally contains sequences encoding all or part of a constant region, enabling an entire, or a part of, a heavy or light chain to be expressed. The gene construct can be introduced into eukaryotic host cells using conventional techniques.

The host cells express a fusion protein and/or antibody conjugate comprising a sialidase and V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments involving fusion proteins and/or antibody conjugates, a host cell is transfected with a single vector expressing a polypeptide expressing a sialidase and an entire, or part of, a heavy chain (e.g., a heavy chain variable region) or a sialidase and a light chain (e.g., a light chain variable region), or a polypeptide expressing an entire, or part of, a heavy chain (e.g., a heavy chain variable region) or a light chain (e.g., a light chain variable region). In some embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain, wherein in (a) or in (b), the polypeptide may also comprise a sialidase. In some embodiments, a host cell is co-transfected with more than one expression vector (e.g., one expression vector expressing a polypeptide comprising an entire, or part of, a heavy chain or heavy chain variable region, optionally comprising a sialidase fused thereto, and another expression vector expressing a polypeptide comprising an entire, or part of, a light chain or light chain variable region, optionally comprising a sialidase fused thereto).

A polypeptide comprising a fusion protein, e.g., a fusion protein comprising an immunoglobulin heavy chain variable region or light chain variable region, can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags.

In embodiments in which a fusion protein and/or antibody conjugate is produced, a sialidase fused to a monoclonal antibody, Fc domain, or an antigen-binding domain of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The sialidase will be fused to one or more of the chains. The intact fusion protein and/or antibody conjugate can be harvested and purified or isolated using techniques known in the art, e.g., Protein A, Protein G, affinity tags such as glutathione-S-transferase (GST) or histidine tags. It is within ordinary skill in the art to express the heavy chain and the light chain from a single expression vector or from two separate expression vectors.

In certain embodiments, in order to express a protein, e.g., a fusion protein, as a secreted protein, a native N-terminal signal sequence of the protein is replaced, e.g., with MDMRVPAQLLGLLLLWLPGARC (SEQ ID NO: 28). In certain embodiments, to express a protein, e.g., a fusion protein, as a secreted protein, an N-terminal signal sequence, e.g., MDMRVPAQLLGLLLLWLPGARC (SEQ ID NO: 28), is added. Additional exemplary N-terminal signal sequences include signal sequences from interleukin-2, CD-5, IgG kappa light chain, trypsinogen, serum albumin, and prolactin. In certain embodiments, in order to express a protein, e.g., a fusion protein, as a secreted protein, a C terminal lysosomal signal motif, e.g., YGTL (SEQ ID NO: 29) is removed.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an antibody are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL. 169:1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, N.Y.), which involves a vaccinia virus-based vector to express antibodies in mammalian cells. High levels of combinatorial diversity of IgG heavy and light chains can be produced. See, e.g., U.S. Pat. No. 6,706,477 (Zauderer); U.S. Pat. No. 6,800,442 (Zauderer); and U.S. Pat. No. 6,872,518 (Zauderer). Another approach for converting a mouse antibody into a form suitable for use in humans is technology practiced commercially by KaloBios Pharmaceuticals, Inc. (Palo Alto, Calif.). This technology involves the use of a proprietary human “acceptor” library to produce an “epitope focused” library for antibody selection. Another approach for modifying a mouse antibody into a form suitable for medical use in humans is HUMAN ENGINEERING™ technology, which is practiced commercially by XOMA (US) LLC. See, e.g., International (PCT) Publication No. WO 93/11794 and U.S. Pat. No. 5,766,886 (Studnicka); U.S. Pat. No. 5,770,196 (Studnicka); U.S. Pat. No. 5,821,123 (Studnicka); and U.S. Pat. No. 5,869,619 (Studnicka).

Any suitable approach, including any of the above approaches, can be used to reduce or eliminate human immunogenicity of an antibody.

In addition, it is possible to create fully human antibodies in mice. Fully human mAbs lacking any non-human sequences can be prepared from human immunoglobulin transgenic mice by techniques referenced in, e.g., Lonberg et al., NATURE 368:856-859, 1994; Fishwild et al., NATURE BIOTECHNOLOGY 14:845-851, 1996; and Mendez et al., NATURE GENETICS 15:146-156, 1997. Fully human monoclonal antibodies can also be prepared and optimized from phage display libraries by techniques referenced in, e.g., Knappik et al., J. MOL. BIOL. 296:57-86, 2000; and Krebs et al., J. IMMUNOL. METH. 254:67-84 2001).

The present invention encompasses fusion proteins comprising antibody fragments, which may be generated by traditional means, such as enzymatic digestion, or by recombinant techniques. For a review of certain antibody fragments, see Hudson et al. (2003) NAT. MED. 9:129-134.

Various techniques have been developed for the production of antibody fragments. Traditionally, these fragments were derived via proteolytic digestion of intact antibodies (see, e.g., Morimoto et al. (1992) JOURNAL OF BIOCHEMICAL AND BIOPHYSICAL METHODS 24:107-117; and Brennan et al. (1985) SCIENCE 229:81). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab′)₂ fragments (Carter et al. (1992) BIO/TECHNOLOGY 10:163-167). According to another approach, F(ab′)₂ fragments can be isolated directly from recombinant host cell culture. Fab and F(ab′)₂ fragments with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See U.S. Pat. Nos. 5,571,894 and 5,587,458.

Methods for making bispecific antibodies are known in the art. See Milstein and Cuello (1983) NATURE 305:537, International (PCT) Publication No. WO93/08829, and Traunecker et al. (1991) EMBO J., 10:3655. For further details of generating bispecific antibodies see, for example, Suresh et al. (1986) METHODS ENZYMOL. 121:210. Bispecific antibodies include cross-linked or “heteroconjugate” or “heterodimer” antibodies. For example, one of the antibodies in the heterodimer can be coupled to avidin, the other to biotin. Heterodimer antibodies may be made using any convenient cross-linking method. Suitable cross-linking agents are well known in the art, and are disclosed in U.S. Pat. No. 4,676,980, along with a number of cross-linking techniques.

Examples of heterodimeric or asymmetric IgG-like molecules include but are not limited to those obtained with the following technologies or using the following formats: Triomab/Quadroma, Knobs-into-Holes, CrossMabs, electrostatically-matched antibodies, LUZ-Y, Strand Exchange Engineered Domain body, Biclonic and DuoBody.

Advantages of using antibody fragments (e.g., F(ab) and F(ab′)₂ fragments) include the elimination of non-specific binding between Fc portions of antibodies and Fc receptors on cells (such as macrophages, dendritic cells, neutrophils, NK cells and B cells). In addition, they may be able to penetrate tissues more efficiently due to their smaller size.

Heterodimeric antibodies, or asymmetric antibodies, allow for greater flexibility and new formats for attaching a variety of drugs to the antibody arms. One of the general formats for creating a heterodimeric antibody is the “knobs-into-holes” format. This format is specific to the heavy chain part of the constant region in antibodies. The “knobs” part is engineered by replacing a small amino acid with a larger one, which fits into a “hole”, which is engineered by replacing a large amino acid with a smaller one. What connects the “knobs” to the “holes” are the disulfide bonds between each chain. The “knobs-into-holes” shape facilitates antibody dependent cell mediated cytotoxicity. Single chain variable fragments (scFv) are connected to the variable domain of the heavy and light chain via a short linker peptide. The linker is rich in glycine, which gives it more flexibility, and serine/threonine, which gives it specificity. Two different scFv fragments can be connected together, via a hinge region, to the constant domain of the heavy chain or the constant domain of the light chain. This gives the antibody bispecificity, allowing for the binding specificities of two different antigens. The “knobs-into-holes” format enhances heterodimer formation but doesn't suppress homodimer formation.

Several approaches to support heterodimerization have been described, for example in International (PCT) Publication Nos. WO96/27011, WO98/050431, WO2007/110205, WO2007/147901, WO2009/089004, WO2010/129304, WO2011/90754, WO2011/143545, WO2012/058768, WO2013/157954, and WO2013/096291, and European Patent Publication No. EP1870459. Typically, in the approaches known in the art, the CH3 domain of the first heavy chain and the CH₃ domain of the second heavy chain are both engineered in a complementary manner so that the heavy chain comprising one engineered CH₃ domain can no longer homodimerize with another heavy chain of the same structure (e.g. a CH₃-engineered first heavy chain can no longer homodimerize with another CH₃-engineered first heavy chain; and a CH₃-engineered second heavy chain can no longer homodimerize with another CH₃-engineered second heavy chain). Thereby the heavy chain comprising one engineered CH₃ domain is forced to heterodimerize with another heavy chain comprising the CH₃ domain, which is engineered in a complementary manner. As a result, the CH₃ domain of the first heavy chain and the CH₃ domain of the second heavy chain are engineered in a complementary manner by amino acid substitutions, such that the first heavy chain and the second heavy chain are forced to heterodimerize, whereas the first heavy chain and the second heavy chain can no longer homodimerize (e.g., for steric reasons).

### III. Pharmaceutical Compositions

For therapeutic use, a fusion protein and/or antibody conjugate preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(—)-3-hydroxybutyric acid. Sustained release compositions may also include liposomes that can be prepared by any of several methods known in the art.

Pharmaceutical compositions containing a sialidase fusion protein or an antibody conjugate disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, a sialidase fusion protein or an antibody conjugate disclosed herein is administered by IV infusion. In certain embodiments, a sialidase fusion protein or an antibody conjugate disclosed herein is administered by intratumoral injection. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL TM (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

In certain embodiments, a pharmaceutical composition may contain a stabilizing agent. In certain embodiments, the stabilizing agent is a cation, such as a divalent cation. In certain embodiments, the cation is calcium or magnesium. The cation can be in the form of a salt, such as calcium chloride (CaCl₂) or magnesium chloride (MgCl₂).

In certain embodiments, the stabilizing agent is present in an amount from about 0.05 mM to about 5 mM. For example, the stabilizing agent may be present in an amount of from about 0.05 mM to about 4 mM, from about 0.05 mM to about 3 mM, from about 0.05 mM to about 2 mM, from about 0.05 mM to about 1 mM, from about 0.05 mM to about 0.5 mM, from about 0.5 mM to about 4 mM, from about 0.5 mM to about 3 mM, from about 0.5 mM to about 2 mM, from about 0.5 mM to about 1 mM, from about 1 mM to about 4 mM, from about 1 mM to about 3 mM, of from about 1 mM to about 2 mM.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

The compositions described herein may be administered locally or systemically. Administration will generally be parenteral administration. In a preferred embodiment, the pharmaceutical composition is administered subcutaneously and in an even more preferred embodiment intravenously. Preparations for parenteral administration include sterile aqueous or non-aqueous solutions, suspensions, and emulsions.

Generally, a therapeutically effective amount of active component, for example, a fusion protein and/or antibody conjugate, is in the range of 0.1 mg/kg to 100 mg/kg, e.g., 1 mg/kg to 100 mg/kg, 1 mg/kg to 10 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the fusion protein and/or antibody conjugate, and the disease being treated. Exemplary dosing frequencies are once per day, once per week and once every two weeks. A preferred route of administration is parenteral, e.g., intravenous infusion. In certain embodiments, a fusion protein and/or antibody conjugate is lyophilized, and then reconstituted in buffered saline, at the time of administration.

### IV. Therapeutic Uses

The compositions and methods disclosed herein can be used to treat various forms of cancer in a subject or inhibit cancer growth in a subject. The invention provides a method of treating a cancer in a subject. The method comprises administering to the subject an effective amount of a sialidase anti-CD20 fusion protein and/or antibody conjugate, e.g., a fusion protein or antibody conjugate disclosed herein, either alone or in a combination with another therapeutic agent to treat the cancer in the subject. The term “effective amount” as used herein refers to the amount of an active agent (e.g., fusion protein according to the present invention) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma).

In certain embodiments the cancer is an epithelial cancer, e.g., an epithelial cancer that upregulates the expression of sialylated glycans. Exemplary epithelial cancers include, but are not limited to, endometrial cancer, colon cancer, ovarian cancer, cervical cancer, vulvar cancer, uterine cancer or fallopian tube cancer, breast cancer, prostate cancer, lung cancer, pancreatic cancer, urinary cancer, bladder cancer, head and neck cancer, oral cancer and liver cancer. Epithelial cancers also include carcinomas, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, baso squamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

In certain embodiments, the cancer is selected from a lymphoma (e.g., a B-cell lymphoma, Hodgkin lymphoma, lymphoblastic lymphoma), a leukemia (e.g., hairy cell leukemia, B-cell chronic lymphocytic leukemia, acute myeloid leukemia (AML), acute lymphoblastic leukemia), and a melanoma.

In certain embodiments, the cancer is an adenocarcinoma. In certain embodiments, the cancer is a metastatic cancer. In certain embodiments, the cancer is a refractory cancer.

In certain embodiments, the cancer is resistant to or non-responsive to treatment with an antibody, e.g., an antibody with ADCC activity, e.g., rituximab or ofatumumab.

In certain embodiments, the cancer is a CD20 expressing cancer, e.g., the cancer comprises cells that express CD20. CD20 may be expressed from late pro-B cells through memory cells. It can be found on B-cell lymphomas, hairy cell leukemia, B-cell chronic lymphocytic leukemia, and melanoma cancer stem cells. Immunohistochemistry can be used to determine the presence of CD20 on cells in histological tissue sections. Because CD20 remains present on the cells of most B-cell neoplasms, and is absent on otherwise similar appearing T-cell neoplasms, it can be very useful in diagnosing conditions such as B-cell lymphomas and leukemias. CD20 is expressed in a vast majority of mature B-cell neoplasms and some cases of B-lymphoblastic leukemia/lymphomas, plasma-cell myelomas, Hodgkin lymphomas, T-cell neoplasms, and acute myeloid leukemias (AMLs). For example, 90% of B cell lymphomas stain positive for CD20 (Chu et al. (2006) AM J CLIN PATHOL 126:534), 40% of pre-B acute lymphoblastic leukemias (ALLs) and lymphoblastic lymphoma (LBLs) stain positive for CD20, 80% of nodular lymphocyte predominant Hodgkin lymphomas stain positive for CD20, and 20% of classic Hodgkin lymphomas stain positive for CD20. For methods of immunostaining CD20 and evaluating its presence in tumor diagnosis and treatment, see for example Naeim et al. (2018) Principles of Immunophenotyping in Atlas of Hematopathology (Second Edition).

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein, is administered in combination with one or more additional therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g. a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodialator, a statin, an anti-inflammatory agent (e.g. methotrexate), or an NSAID. In certain embodiments, the additional therapy may include a combination of therapeutics of different classes.

In certain embodiments, a method or composition described herein is administered in combination with a checkpoint inhibitor. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), durvalumab (AstraZeneca), MEDI4736, avelumab, and BMS 936559 (Bristol Myers Squibb Co.).

In certain embodiments, a method or composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

In certain embodiments, a method or composition described herein is administered in combination with (i) a PD-1 or PD-L1 inhibitor, e.g., a PD-1 or PD-L1 inhibitor disclosed herein, and (ii) CTLA-4 inhibitor, e.g., a CTLA-4 inhibitor disclosed herein.

In certain embodiments, a method or composition described herein is administered in combination with an IDO inhibitor. Exemplary IDO inhibitors include 1-methyl-D-tryptophan (known as indoximod), epacadostat (INCB24360), navoximod (GDC-0919), and BMS-986205.

Exemplary cytotoxic agents that can be administered in combination with a method or composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a method or composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, l-dehydrotestosterone, glucocorticoids, procaine, tetracaine, lidocaine, propranolol, puromycin, ricin, or maytansinoids.

The invention also provides a method of increasing the expression of HLA-DR, CD86, CD83, IFNγ, IL-1b, IL-6, TNFα, IL-17A, IL-2, or IL-6 in a cell, tissue, or subject. The method comprises contacting the cell, tissue, or subject with an effective amount of a fusion protein and/or antibody conjugate, e.g., a fusion protein or antibody conjugate disclosed herein. In certain embodiments, the cell is selected from a dendritic cell and a peripheral blood mononuclear cell (PBMC).

In certain embodiments, expression of HLA-DR, CD86, CD83, IFNγ, IL-1b, IL-6, TNFα, IL-17A, IL-2, or IL-6 in the cell, tissue, or subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell or tissue that has not been contacted with the fusion protein or antibody conjugate. Gene expression may be measured by any suitable method known in the art, for example, by ELISA, or by Luminex multiplex assays.

The invention also provides a method of promoting infiltration of immune cells into a tumor in a subject in need thereof. The method comprises administering to the subject an effective amount of a fusion protein and/or antibody conjugate, e.g., a fusion protein or antibody conjugate disclosed herein. In certain embodiments, the immune cells are T-cells, e.g., CD4+ and/or CD8+ T-cells, e.g., CD69⁺CD8⁺ and/or GzmB⁺CD8⁺ T-cells. In certain embodiments, the immune cells are natural killer (NK) cells.

In certain embodiments, the infiltration of immune cells into the tumor in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical tumor and/or subject that has not been administered the fusion protein or antibody conjugate. Infiltration of immune cells into a tumor may be measured by any suitable method known in the art, for example, antibody staining.

The invention also provides a method of increasing the number of circulating natural killer (NK) cells in a subject in need thereof. The method comprises administering to the subject an effective amount of a fusion protein and/or antibody conjugate, e.g., a fusion protein or antibody conjugate disclosed herein, so as to increase the number of circulating NK cells relative to prior to administration of the fusion protein, antibody conjugate or pharmaceutical composition.

In certain embodiments, the number of circulating NK cells in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical subject that has not been administered the fusion protein or antibody conjugate. Circulating NK cells in a subject may be measured by any suitable method known in the art, for example, antibody staining.

The invention also provides a method of increasing the number of T-cells in the draining lymph node in a subject in need thereof. The method comprises administering to the subject an effective amount of a fusion protein and/or antibody conjugate, e.g., a fusion protein or antibody conjugate disclosed herein, so as to increase the number of T-cells in the draining lymph node relative to prior to administration of the fusion protein, antibody conjugate or pharmaceutical composition. In certain embodiments, the immune cells are T-cells, e.g., CD4+ and/or CD8+ T-cells.

In certain embodiments, the number of T-cells in the draining lymph node in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical subject that has not been administered the fusion protein or antibody conjugate. T-cells in the draining lymph node in a subject may be measured by any suitable method known in the art, for example, antibody staining.

The invention also provides a method of increasing expression of Cd3, Cd4, Cd8, Cd274, Ctla4, Icos, Pdcd1, Lag3, Il6, Il1b, Il2, Ifng, Ifna1, Mx1, Gzmb, Cxcl9, Cxcl12, and/or Ccl5 in a cell, tissue, or subject. The method comprises contacting the cell, tissue, or subject with an effective amount of a fusion protein and/or antibody conjugate, e.g., a fusion protein or antibody conjugate disclosed herein, so as to increase the expression of Cd3, Cd4, Cd8, Cd274, Ctla4, Icos, Pdcd1, Lag3, Il6, Il1b, Il2, Ifng, Ifna1, Mx1, Gzmb, Cxcl9, Cxcl12, and/or Ccl5 relative to the cell, tissue or subject prior to contact with the fusion protein, antibody conjugate or pharmaceutical composition.

In certain embodiments, expression of Cd3, Cd4, Cd8, Cd274, Ctla4, Icos, Pdcd1, Lag3, Il6, Il1b, Il2, Ifng, Ifna1, Mx1, Gzmb, Cxcl9, Cxcl12, and/or Ccl5 in the cell, tissue, or subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell, tissue, or subject that has not been contacted with the fusion protein or antibody conjugate. Gene expression may be measured by any suitable method known in the art, for example, by ELISA, Luminex multiplex assays, or Nanostring technology.

The invention also provides a method of removing sialic acid from a cell or tissue. The method comprises contacting the cell or tissue with an effective amount of a fusion protein and/or antibody conjugate, e.g., a fusion protein or antibody conjugate disclosed herein. The invention also provides a method of removing sialic acid from a cell in a subject, the method comprising administering to the subject an effective amount of a pharmaceutical composition comprising a fusion protein and/or antibody conjugate, e.g., a fusion protein or antibody conjugate disclosed herein, thereby to remove sialic acid from the cell.

In certain embodiments, the cell is tumor cell, dendritic cell (DC) or monocyte. In certain embodiments, the cell is a monocyte, and the method results in increased expression of an MHC-II molecule (e.g., HLA-DR) on the monocyte. In certain embodiments, expression of an MHC-II molecule in the cell or tissue is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell or tissue that has not been contacted with the fusion protein and/or antibody conjugate. Gene expression may be measured by any suitable method known in the art, for example, by ELISA, by Luminex multiplex assays, or by flow cytometry.

The invention also provides a method of enhancing phagocytosis of a tumor cell. The method comprises contacting the tumor cell with a fusion protein and/or antibody conjugate, e.g., a fusion protein or antibody conjugate disclosed herein, in an amount effective to remove sialic acid from the tumor cell, thereby enhancing phagocytosis of the tumor cell. In certain embodiments, the disclosure relates to a method of increasing phagocytosis of a tumor cell in a subject, the method comprising administering to the subject an effective amount of a pharmaceutical composition a fusion protein and/or antibody conjugate, e.g., a fusion protein or antibody conjugate disclosed herein, in an amount effective to remove sialic acid from the tumor cell, thereby to increase phagocytosis of the tumor cell.

In certain embodiments, phagocytosis is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical tumor cell or population of tumor cells that has not or have not been contacted with the fusion protein and/or antibody conjugate. Phagocytosis may be measured by any suitable method known in the art.

The invention also provides a method of activating a dendritic cell (DC). The method comprises contacting the DC with a tumor cell that has been treated with a fusion protein and/or antibody conjugate, e.g., a fusion protein or antibody conjugate disclosed herein. In certain embodiments, the disclosure relates to a method of activating a dendritic cell (DC) or a population of DCs in a subject, the method comprising administering to the subject an amount of a pharmaceutical composition comprising a fusion protein and/or antibody conjugate, e.g., a fusion protein or antibody conjugate disclosed herein, effective to remove sialic acid from a tumor cell in the subject, thereby to activate the DC or the population of DCs in the subject.

In certain embodiments, activation of the DC or a population of DCs is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical DC or population of DCs that has not or have not been contacted with a tumor cell that has been treated with the fusion protein and/or antibody conjugate. Activation may be measured by any suitable method known in the art.

The invention also provides a method of reducing Siglec-15 binding activity, thereby to increase anti-tumor activity in a tumor microenvironment, the method comprising contacting a T cell with a fusion protein and/or antibody conjugate, e.g., a fusion protein or antibody conjugate disclosed herein. In certain embodiments, the disclosure relates to a method of reducing Siglec-15 binding activity, thereby to increase anti-tumor activity in a tumor microenvironment of a patient, the method comprising administering to the subject an effective amount of a pharmaceutical composition comprising a fusion protein and/or antibody conjugate, e.g., a fusion protein or antibody conjugate disclosed herein, thereby to increase anti-tumor activity (e.g., T cell activity) in the subject.

In certain embodiments, Siglec-15 binding activity is reduced by at least about 10%, at least about 20%, at least about 50%, at least about 75%, or about 100%, relative to Siglec-15 that has not or have not been contacted with the fusion protein and/or antibody conjugate. Binding may be measured by any suitable method known in the art.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This example describes the construction of recombinant human sialidases (Neu1, Neu2, and Neu3).

The human sialidases Neu1, Neu2, Neu3 (isoform 1), and Neu4 (isoform 1) were expressed as secreted proteins with a 10×His tag. To express Neu1 as a secreted protein, the native N terminal signal peptide (MTGERPSTALPDRRWGPRILGFWGGCRVWVFAAIFLLLSLAASWSKA; SEQ ID NO: 27) was replaced by MDMRVPAQLLGLLLLWLPGARC (SEQ ID NO: 28), and the C terminal lysosomal signal motif (YGTL; SEQ ID NO: 29) was removed. To express Neu2, Neu3, and Neu4 as secreted proteins, the N terminal signal peptide MDMRVPAQLLGLLLLWLPGARC (SEQ ID NO: 28) was added to each.

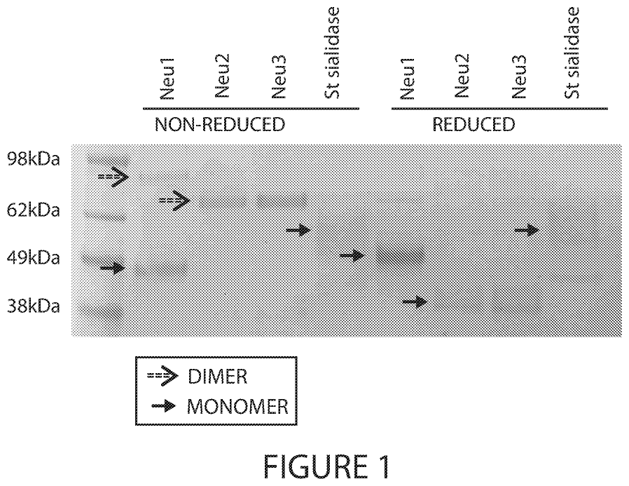

Sialidases were expressed in a 200 mL transfection of HEK293F human cells in 24-well plates using the pCEP4 mammalian expression vector with an N-terminal 6×His tag. Sialidases were purified using Ni-NTA columns, quantified with a UV-Vis spectrophotometer (NanoDrop), and examined by SDS-PAGE as shown in FIG. 1. Neu1 expressed well, with a yield of ˜3 μg/ml, and was present primarily in a monomeric form. Neu2 and Neu3 expression each gave yields of ˜0.15 μg/mL and each were present primarily in a dimeric form. Neu4 had no detectable expression yield as measured by NanoDrop. Bacterial sialidase from Salmonella typhimurium (St-sialidase; SEQ ID NO: 30), which was used as a positive control for expression, gave a comparable yield to Neu1, and was present primarily in a monomeric form.

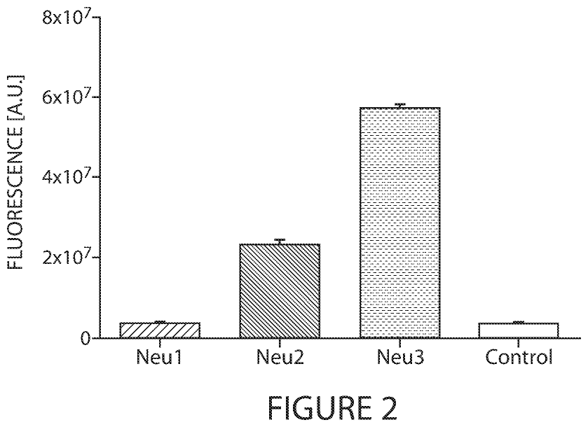

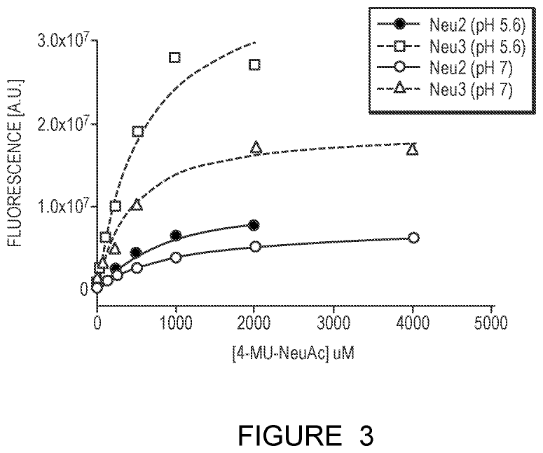

The activity of the recombinantly expressed sialidases was assayed by measuring the release of sialic acid from the fluorogenic substrate 4-methylumbelliferyl-N-acetylneuraminic acid (4MU-NeuAc). As shown in FIG. 2, Neu1 has no detectable activity above a no-enzyme control, which is consistent with previous reports indicating that Neu1 is inactive unless it is in complex with beta-galactosidase and protective protein/cathepsin A (PPCA). Neu2 and Neu3 were active. An enzyme kinetics assay was performed with Neu2 and Neu3. A fixed concentration of enzyme at 1 nM was incubated with fluorogenic substrate 4MU-NeuAc at concentrations ranging from 4000 μM to 7.8 μM. Assays were conducted at both acidic (pH 5.6) and neutral (pH 7) conditions. As shown in FIG. 3, both Neu2 and Neu3 were active at acidic and neutral conditions and showed enzyme kinetics that were comparable to those previously reported.

Most of the recombinantly expressed sialidases ran as aggregates or dimers on a non-reducing SDS-PAGE gel. Subsequent treatment with the reducing agent dithiothreitol (DTT) resulted in a monomeric form of the enzyme that ran at 42 kDa on a reducing SDS-PAGE gel (FIG. 1).

### Example 2

This example describes the construction of recombinant human sialidases with mutations that increase expression and/or activity of the sialidase.

### A. Rational Design

Structural and sequence analysis identified residues A93 and P62 of Neu2 as candidates for substitutions to increase solubility and/or expression. In particular, a comparison of homologous sialidase sequences showed a preference for D or E amino acid residues at positions corresponding to position 93 of Neu2, and a preference for G amino acid residues at positions corresponding to position 62 of Neu2.

The beta-propeller family of proteins are usually stabilized by extensive hydrogen bonding interactions at the N- and C-termini of the protein. A structural analysis revealed that Neu2, which is a member of the beta-propeller family, appears to lack these stabilizing interactions. In contrast, sialidases from Salmonella typhimurium and Micromonospora viridifaciens (the bacterial sialidase most homologous to human Neu2) have extensive hydrogen bonding interactions at their N- and C-termini. Accordingly, residues K9, V363, and L365 of Neu2 were mutated to promote hydrogen bonding between the N- and C-termini of Neu2.

### B. Phage Display

Neu2 was expressed in a phage display system allowing for screening of Neu2 variants for both expression level and resistance to heat denaturation. Neu2 with V6Y and I187K substitutions was used as a template for library preparation. Designed phage display libraries 1, 2, and 3 are depicted in TABLEs 10-12, respectively. Each library included all of the possible combinations of the mutations depicted. A fourth library included random mutations generated by error prone PCR.

TABLE 10
Phage Library 1

| Wild-Type | | | |
|---|---|---|---|
| Neu2 Amino | | Substituting | Codon |
| Acid(s) | Design | Amino Acids | Usage |
| 184-188 | Adjust length of 5 wild-type | S, N, R, K, T, G, | RVM |
| LHPIQ | residues from 2 to 5 and | D, E, A | |
| | substitute each residue with | | |
| | one of 9 polar amino acids | | |
| P190, I191 | Substitute with one of 5 non- | F, I, L, M, V | NTK |
| | polar amino acids | | |
| C219 | Substitute with one of 12 polar | R, N, D, C, G, H, | NDT |
| | or nonpolar amino acids | I, L, F, S, Y, V | |
| L217 | Substitute with one of 12 polar | R, N, D, C, G, H, | NDT |
| | or nonpolar amino acids | I, L, F, S, Y, V | |
| T216 and | Insert one of 12 polar or | S, T, Y, L, F, A, | NHT |
| L217 | nonpolar amino acids between | P, V, I, N, D, H | |
| | the wild-type amino acids | | |
| G271 | Substitute with one of 9 polar | S, N, R, K, T, G, | RVM |
| | amino acids | D, E, A | |
| C272 | Substitute with one of 9 polar | S, N, R, K, T, G, | RVM |
| | amino acids | D, E, A | |

TABLE 11
Phage Library 2

| Wild-Type | | | |
|---|---|---|---|
| Neu2 Amino | | Substituting | Codon |
| Acid(s) | Design | Amino Acids | Usage |
| 156-165 | Substitute with one of | R, N, D, C, G, H, | NDT |
| TFAVGPGHCL | 12 hydrophobic or | I, L, F, S, Y, V | |
| | polar amino acids | | |
| V176 | Substitute with one of | R, N, D, C, G, H, | NDT |
| | 12 hydrophobic or | I, L, F, S, Y, V | |
| | polar amino acids | | |
| P177 | Substitute with one of | S, T, Y, L, F, A, | NHT |
| | 12 hydrophobic or | P, V, I, N, D, H | |
| | polar amino acids | | |
| A178 | Substitute with one of | S, T, Y, L, F, A, | NHT |
| | 12 hydrophobic or | P, V, I, N, D, H | |
| | polar amino acids | | |
| A194 | Substitute with one of | S, T, Y, L, F, A, | NHT |
| | 12 hydrophobic or | P, V, I, N, D, H | |
| | polar amino acids | | |

TABLE 12
Phage Library 3

| Wild-Type | | | |
|---|---|---|---|
| Neu2 Amino | | Substituting | Codon |
| Acid(s) | Design | Amino Acids | Usage |
| F13 | Substitute with one of 9 polar | S, N, R, K, T, G, | RMV |
| | amino acids chain AAs | D, E, A | |
| L4 | Substitute with one of 12 | S, T, Y, L, F, A, | NHT |
| | hydrophobic or polar amino | P, V, I, N, D, H | |
| | acids | | |
| L7, V12 | Substitute with one of 6 polar | F, Y, S, I, T, N | WHT |
| | or aromatic amino acids | | |
| I22, A24 | Substitute with one of 9 polar | S, N, R, K, T, G, | RMV |
| | amino acids | D, E, A | |
| V325, L326, | Substitute with one of 6 polar | F, Y, S, I, T, N | WHT |
| and L327 | or aromatic amino acids | | |
| L365 | Substitute with one of 6 polar | F, Y, S, I, T, N | WHT |
| | or aromatic amino acids | | |
| P89 | Substitute with one of 5 non- | F, I, L, M, V | NTK |
| | polar amino acids | | |
| L34, A36, | Substitute with one of 12 | S, T, Y, L, F, A, | NHT |
| and V363 | hydrophobic or polar amino | P, V, I, N, D, H | |
| | acids | | |

The codon usage columns in TABLES 10-12 represent degenerate codon codes used in the design of the library, where the first, second, and third positions of a given codon encoding an amino acid are as shown in TABLE 13 and as described in Mena et al. (2005) PROTEIN ENG DES SEL. 18(12):559-61.

TABLE 13

| Degenerate | |
|---|---|
| Codon Code | Mixed bases |
| R | A, G |
| Y | C, T/U |
| M | A, C |
| K | G, T/U |
| S | G, C |
| W | A, T |
| H | A, C, T/U |
| B | G, C, T (or U) |
| V | A, C, G |
| D | A, G, T/U |
| N | A, C, G, T/U |

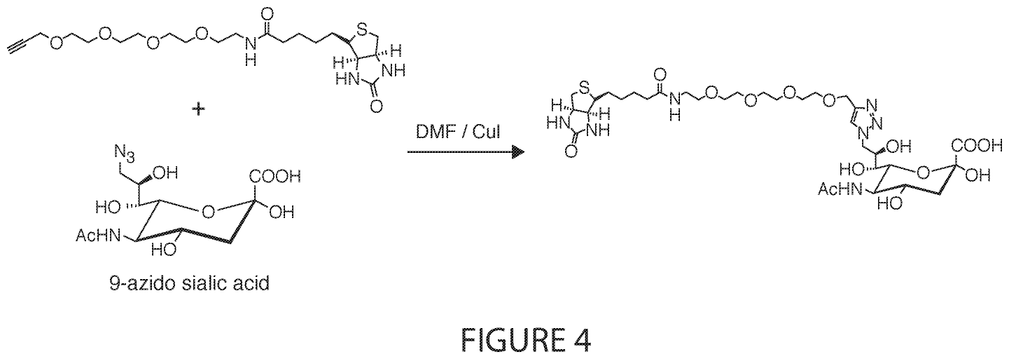

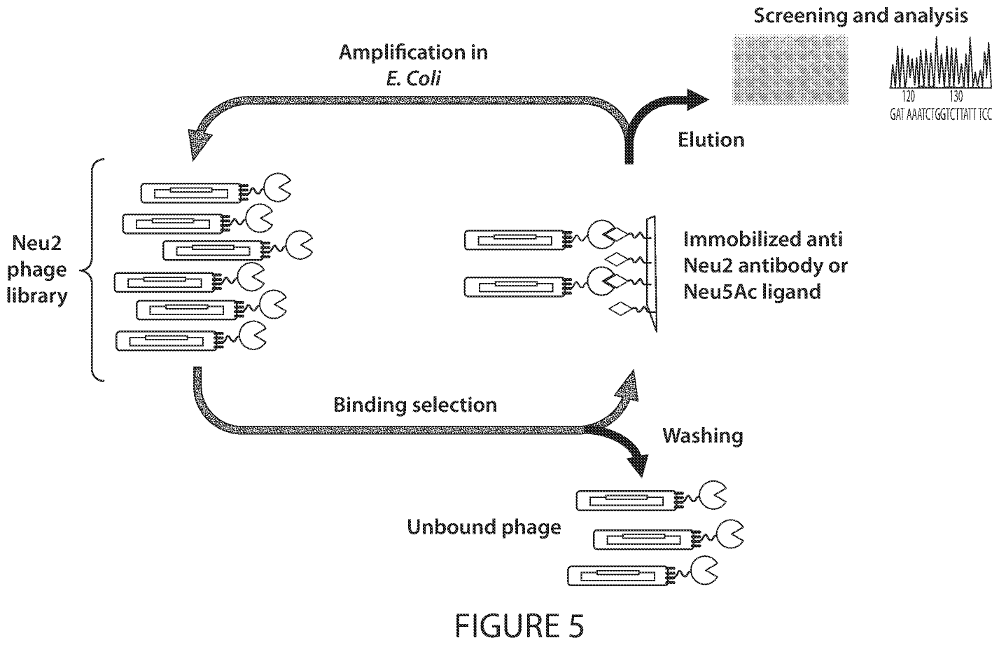

The phage display libraries were screened for binding to a conformation-specific antibody and/or a sialic acid biotinylated probe after heating to enrich for thermal stability and expression. The sialic acid biotinylated probe and its synthesis is depicted in FIG. 4. An exemplary phage display screening procedure is depicted in FIG. 5. Briefly, phage libraries expressing the desired Neu2 variants were generated. Phage were screened for binding to immobilized anti-Neu2 antibody and/or sialic acid biotinylated probe. Following washing to remove unbound phage, bound phage were eluted from the antibody or probe and analyzed as appropriate.

### C. Yeast Display

Neu2 was also expressed in a yeast display system allowing for screening of Neu2 variants for both expression level and resistance to heat denaturation. Neu2 with V6Y and I187K substitutions was used as a template for library preparation. Designed yeast display libraries 1a, 1b, 1c, 1d, 2a, 2b, 2c, 3a, 3b, and 3c are depicted in TABLEs 14-23, respectively. Each library included all of the possible combinations of the mutations depicted. Five additional sublibraries were generated by error prone PCR, at an approximate average rate of 1, 2, 3, 4, and 5 substitutions per enzyme.

TABLE 14
Yeast Library 1a

| Wild-Type Neu2 | | Codon |
|---|---|---|
| Amino Acid(s) | Substituting Amino Acids | Usage |
| L184 | All | NNK |
| H185 | All | NNK |
| P186 | None (Wild-type) | |
| K187 | None (Wild-type) | |
| Q188 | None (Wild-type) | |
| P190 | A, D, G, H, I, L, N, P, R, S, T, V | VNT |
| I191 | A, D, F, H, I, L, N, P, S, T, V, Y | NHT |
| C219 | None (Wild-type) | |
| G271 | All | NNK |
| C272 | All | NNK |

Total Diversity: 2.30E+07

TABLE 15
Yeast Library 1b

| Wild-Type Neu2 | | Codon |
|---|---|---|
| Amino Acid(s) | Substituting Amino Acids | Usage |
| L184 | A, D, F, H, I, L, N, P, S, T, V, Y | NHT |
| H185 | All | NNK |
| P186 | A, D, F, H, I, L, N, P, S, T, V, Y | NHT |
| K187 | None (Wild-type) | |
| Q188 | None (Wild-type) | |
| P190 | A, D, G, H, I, L, N, P, R, S, T, V | VNT |
| I191 | A, D, F, H, I, L, N, P, S, T, V, Y | NHT |
| C219 | None (Wild-type) | |
| G271 | A, D, E, G, K, N, R, S, T | RVM |
| C272 | A, C, D, G, H, N, P, R, S, T | NVT |

Total Diversity: 4.11E+07

TABLE 16
Yeast Library 1c

| Wild-Type Neu2 | | Codon |
|---|---|---|
| Amino Acid(s) | Substituting Amino Acids | Usage |
| L184 | A, D, F, H, I, L, N, P, S, T, V, Y | NHT |
| H185 | A, D, E, G, K, N, R, S, T | RVM |
| P186 | A, D, F, H, I, L, N, P, S, T, V, Y | NHT |
| K187 | A, D, E, G, K, N, R, S, T | RVM |
| Q188 | None (Wild-type) | |
| P190 | F, L, I, V | NTT |
| I191 | A, D, F, H, I, L, N, P, S, T, V, Y | NHT |
| C219 | None (Wild-type) | |
| G271 | A, D, E, G, K, N, R, S, T | RVM |
| C272 | A, D, F, H, L, P, S, V, Y | BHT |

Total Diversity: 4.53E+07

TABLE 17
Yeast Library 1d

| Wild-Type Neu2 | | Codon |
|---|---|---|
| Amino Acid(s) | Substituting Amino Acids | Usage |
| L184 | A, D, F, H, L, P, S, V, Y | BHT |
| H185 | A, D, E, G, K, N, R, S, T | RVM |
| P186 | A, D, F, H, L, P, S, V, Y | BHT |
| K187 | A, D, E, G, K, N, R, S, T | RVM |
| Q188 | A, D, F, H, L, P, S, V, Y | BHT |
| P190 | None (Wild-type) | |
| I191 | A, D, E, G, K, N, R, S, T | RVM |
| C219 | None (Wild-type) | |
| G271 | A, D, E, G, K, N, R, S, T | RVM |
| C272 | A, D, F, H, L, P, S, V, Y | BHT |

Total Diversity: 4.30E+07

TABLE 18
Yeast Library 2a

| Wild-Type Neu2 | | Codon |
|---|---|---|
| Amino Acid(s) | Substituting Amino Acids | Usage |
| T156 | R, N, D, C, G, H, I, L, F, S, Y, V | NDT |
| F157 | None (Wild-type) | |
| A158 | R, N, D, C, G, H, I, L, F, S, Y, V | NDT |
| V159 | None (Wild-type) | |
| G160 | R, N, D, C, G, H, I, L, F, S, Y, V | NDT |
| P161 | None (Wild-type) | |
| G162 | R, N, D, C, G, H, I, L, F, S, Y, V | NDT |
| H163 | None (Wild-type) | |
| C164 | R, N, D, C, G, H, I, L, F, S, Y, V | NDT |
| L165 | None (Wild-type) | |
| V176 | L, V, P, A, H, D | SHT |
| P177 | L, V, P, A, H, D | SHT |
| A178 | L, V, P, A, H, D | SHT |
| A194 | L, V, P, A, H, D | SHT |

Total Diversity: 3.22E+08

TABLE 19
Yeast Library 2b

| Wild-Type Neu2 | | Codon |
|---|---|---|
| Amino Acid(s) | Substituting Amino Acids | Usage |
| T156 | None (Wild-type) | |
| F157 | R, N, D, C, G, H, I, L, F, S, Y, V | NDT |
| A158 | None (Wild-type) | |
| V159 | R, N, D, C, G, H, I, L, F, S, Y, V | NDT |
| G160 | None (Wild-type) | |
| P161 | R, N, D, C, G, H, I, L, F, S, Y, V | NDT |
| G162 | None (Wild-type) | |
| H163 | R, N, D, C, G, H, I, L, F, S, Y, V | NDT |
| C164 | None (Wild-type) | |
| L165 | R, N, D, C, G, H, I, L, F, S, Y, V | NDT |
| V176 | L, V, P, A, H, D | SHT |
| P177 | L, V, P, A, H, D | SHT |
| A178 | L, V, P, A, H, D | SHT |
| A194 | L, V, P, A, H, D | SHT |

Total Diversity: 3.22E+08

TABLE 20
Yeast Library 2c

| Wild-Type Neu2 | | Codon |
|---|---|---|
| Amino Acid(s) | Substituting Amino Acids | Usage |
| T156 | A, D, G, H, N, P, R, S, T | WC |
| F157 | A, D, F, H, L, P, S, V, Y | BHT |
| A158 | A, D, G, H, N, P, R, S, T | WC |
| V159 | A, D, F, H, L, P, S, V, Y | BHT |
| G160 | A, D, G, H, N, P, R, S, T | WC |
| P161 | A, D, F, H, L, P, S, V, Y | BHT |
| G162 | A, D, G, H, N, P, R, S, T | WC |
| H163 | A, D, F, H, L, P, S, V, Y | BHT |
| C164 | A, D, G, H, N, P, R, S, T | WC |
| L165 | A, D, F, H, L, P, S, V, Y | BHT |
| V176 | None (Wild-type) | |
| P177 | None (Wild-type) | |
| A178 | None (Wild-type) | |
| A194 | None (Wild-type) | |

Total Diversity: 3.49E+09

TABLE 21
Yeast Library 3a

| Wild-Type Neu2 | | Codon |
|---|---|---|
| Amino Acid(s) | Substituting Amino Acids | Usage |
| L4 | S, T, Y, L, F, A, P, V, I, N, D, H | NHT |
| L7 | F, Y, S, I, T, N | WHT |
| V12 | None (Wild-type) | |
| F13 | None (Wild-type) | |
| I22 | S, N, R, K, T, G, D, E, A | RMV |
| A24 | S, N, R, K, T, G, D, E, A | RMV |
| L34 | None (Wild-type) | |
| A36 | None (Wild-type) | |
| H64 | F, Y, S, I, T, N | WHT |
| P89 | F, I, L, V | NTT |
| C164 | None (Wild-type) | |
| V325 | F, Y, S, I, T, N | WHT |
| L326 | F, Y, S, I, T, N | WHT |
| L327 | F, Y, S, I, T, N | WHT |
| C332 | None (Wild-type) | |
| V363 | S, T, Y, L, F, A, P, V, I, N, D, H | NHT |

Total Diversity: 3.63E+08

TABLE 22
Yeast Library 3b

| Wild-Type Neu2 | | Codon |
|---|---|---|
| Amino Acid(s) | Substituting Amino Acids | Usage |
| L4 | None (Wild-type) | |
| L7 | F, Y, S, I, T, N | WHT |
| V12 | S, T, Y, L, F, A, P, V, I, N, D, H | NHT |
| F13 | S, N, R, K, T, G, D, E, A | RMV |
| I22 | S, N, R, K, T, G, D, E, A | RMV |
| A24 | S, N, R, K, T, G, D, E, A | RMV |
| L34 | S, T, Y, L, F, A, P, V, I, N, D, H | NHT |
| A36 | S, T, Y, L, F, A, P, V, I, N, D, H | NHT |
| H64 | None (Wild-type) | |
| P89 | None (Wild-type) | |
| C164 | A, G, S, T | RST |
| V325 | None (Wild-type) | |
| L326 | None (Wild-type) | |
| L327 | None (Wild-type) | |
| C332 | A, D, G, H, N, P, R, S, T | VVC |
| V363 | None (Wild-type) | |

Total Diversity: 2.72E+08

TABLE 23
Yeast Library 3c

| Wild-Type Neu2 | | Codon |
|---|---|---|
| Amino Acid(s) | Substituting Amino Acids | Usage |
| L4 | None (Wild-type) | |
| L7 | None (Wild-type) | |
| V12 | None (Wild-type) | |
| F13 | None (Wild-type) | |
| I22 | S, T, Y, L, F, A, P, V, I, N, D, H | NHT |
| A24 | S, T, Y, L, F, A, P, V, I, N, D, H | NHT |
| L34 | None (Wild-type) | |
| A36 | None (Wild-type) | |
| H64 | F, Y, S, I, T, N | WHT |
| P89 | S, T, Y, L, F, A, P, V, I, N, D, H | NHT |
| C164 | A, G, S, T | RST |
| V325 | A, D, F, H, L, P, S, V, Y | BHT |
| L326 | A, D, F, H, L, P, S, V, Y | BHT |
| L327 | A, D, F, H, L, P, S, V, Y | BHT |
| C332 | A, D, G, H, N, P, R, S, T | WC |
| V363 | None (Wild-type) | |

Total Diversity: 2.72E+08

The codon usage columns in TABLES 14-23 represent degenerate codon codes used in the design of the library, where the first, second, and third positions of a given codon encoding an amino acid are as shown herein above in TABLE 13 and as described in Mena et al. (2005) PROTEIN ENG DES SEL. 18(12):559-61.

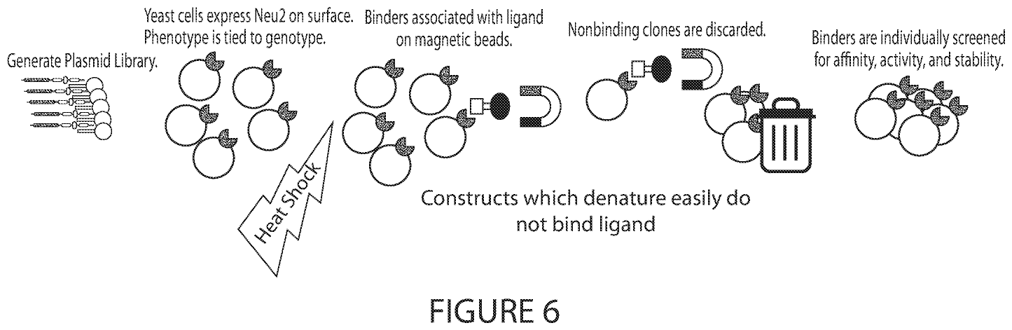

The yeast display libraries were screened for binding to a conformation-specific antibody and/or a sialic acid biotinylated probe after heating to enrich for thermal stability and expression. An exemplary yeast display screening procedure is depicted in FIG. 6. Briefly, a plasmid library encoding for the desired Neu2 variants, and yeast cells expressing the desired Neu2 variants on the surface, were generated. Yeast cells were heat shocked and then screened for binding to anti-Neu2 antibody and/or sialic acid biotinylated probe on magnetic beads. The magnetic beads were isolated to remove unbound cells, and bound cells were further analyzed for Neu2 affinity, activity, and stability as appropriate.

### D. Results

Mutant sialidases including mutations identified using the rational design, phage display, and yeast display approaches described in this Example were expressed as secreted proteins with a C-terminal human Fc tag in Expi293F cells using the pCEP4 mammalian expression vector. Expression was assayed using a ForteBio Octet with anti-human Fc sensors and Western blot and enzymatic activity was assayed using the fluorogenic substrate 4MU-NeuAc as described above.

Expression and activity levels for the mutant sialidases are shown in TABLE 24. In TABLE 24, enzymatic activity is indicated as “+++,” which denotes activity >2 fold higher than wild-type Neu2, “++,” which denotes activity comparable to wild-type Neu2, “+,” which denotes activity lower than wild-type Neu2, or “−,” which denotes no detectable activity, and expression is indicated as “++++,” which denotes expression >15 fold higher than wildtype-Neu2, “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression.

TABLE 24

| Identifier | Mutation(s) | Activity | Expression |
|---|---|---|---|
| Neu2-M104 | M1D, V6Y, P62G, I187K, C332A | + | ++++ |
| Neu2-M105 | M1D, V6Y, K9D, I187K, C332A, | + | +++ |
| | V363R, L365I | | |
| Neu2-M106 | M1D, V6Y, P62G, A93E, I187K, | ++ | ++++ |
| | C332A | | |
| Neu2-M107 | M1D, V6Y, K9D, I187K, C332A, | + | ++++ |
| | V363R, L365K | | |
| Neu2-M108 | M1D, V6Y, K9D, I187K, C332A, | + | ++++ |
| | V363R, L365S | | |
| Neu2-M109 | M1D, V6Y, K9D, I187K, C332A, | + | ++++ |
| | V363R, L365Q | | |
| Neu2-M110 | M1D, V6Y, K9D, I187K, C332A, | + | +++ |
| | V363R, L365H | | |
| Neu2-Ml 11 | M1D, V6Y, A93K, I187K, C332A | ++ | ++ |
| Neu2-M112 | M1D, V6Y, A93E, I187K, C332A | ++ | ++ |
| Neu2-M113 | V6Y, I22N, C125Y, I187K, S301C, | − | +++ |
| | E319D | | |
| Neu2-M114 | V6Y, P62T, C125F, I187K, A222D | − | +++ |
| Neu2-M115 | V6Y, I187K, W292R | + | +++ |
| Neu2-M116 | V6Y, G107D, I187K | + | +++ |
| Neu2-M117 | C125L | + | + |
| Neu2-M118 | V6Y, C125L | + | ++ |
| Neu2-M119 | C125L, I187K | ++ | ++ |
| Neu2-M120 | V6Y, C125L, I187K | + | +++ |
| Neu2-M121 | M1D, V6Y, K45A, I187K, C332A | ++ | ++ |
| Neu2-M122 | M1D, V6Y, Q270A, I187K, C332A | ++ | +++ |
| Neu2-M123 | M1D, V6Y, K44R, K45R, I187K, | + | ++ |
| | C332A | | |
| Neu2-M124 | M1D, V6Y, Q112R, I187K, C332A | + | ++ |
| Neu2-M125 | M1D, V6Y, Q270F, I187K, C332A | + | ++ |
| Neu2-M126 | M1D, V6Y, I187K, S301R, W302K, | ++ | +++ |
| | C332A | | |
| Neu2-M127 | M1D, V6Y, K44E, K45E, I187K, | ++ | + |
| | C332A | | |
| Neu2-M128 | M1D, V6Y, I187K, L217V, C332A | + | ++ |
| Neu2-M129 | M1D, V6Y, I187K, L217A, C332A | + | ++ |
| Neu2-M130 | M1D, V6Y, I187K, C332A, Y359A | − | +++ |
| Neu2-M131 | M1D, V6Y, I187K, C332A, Y359S | − | +++ |
| Neu2-M132 | M1D, V6Y, K44E, K45E, I187K, | ++ | ++ |
| | S301R, W302K, C332A | | |
| Neu2-M133 | M1D, V6Y, Q112R, I187K, S301R, | ++ | +++ |
| | W302K, C332A | | |
| Neu2-M134 | M1D, V6Y, I187K, Q270A, S301R, | ++ | +++ |
| | W302K, C332A | | |
| Neu2-M135 | M1D, V6Y, K44E, K45E, Q112R, | + | + |
| | I187K, C332A | | |
| Neu2-M136 | M1D, V6Y, K44E, K45E, I187K, | ++ | ++ |
| | Q270A, C332A | | |
| Neu2-M137 | M1D, V6Y, K45A, I187K, Q270A, | ++ | +++ |
| | C332A | | |
| Neu2-M138 | M1D, V6Y, I187K, Q270H, C332A | ++ | +++ |
| Neu2-M139 | M1D, V6Y, I187K, Q270P, C332A | + | +++ |
| Neu2-M140 | M1D, V6Y, Q112K, I187K, C332A | ++ | ++ |
| Neu2-M141 | M1D, V6Y, P62S, I187K, Q270A, | + | +++ |
| | S301R, W302K, C332A | | |
| Neu2-M142 | M1D, V6Y, P62T, I187K, Q270A, | + | +++ |
| | S301R, W302K, C332A | | |
| Neu2-M143 | M1D, V6Y, P62N, I187K, Q270A, | + | +++ |
| | S301R, W302K, C332A | | |
| Neu2-M144 | V6Y, P62H, I187K | + | +++ |
| Neu2-M145 | V6Y, Q108H, I187K | + | ++ |
| Neu2-M146 | M1D, V6Y, P62H, I187K, C332A | ++ | +++ |
| Neu2-M147 | M1D, V6Y, P62G, I187K, C332A | ++ | ++ |
| Neu2-M148 | V6Y, P62G, I187K | + | ++ |
| Neu2-M149 | M1D, V6Y, P62H, H87K | ++ | ++ |
| Neu2-M150 | M1D, V6Y, Q108H, I187K | ++ | ++ |
| Neu2-M151 | M1D, V6Y, P62F, I187K, C332A | + | − |
| Neu2-M152 | M1D, V6Y, P62I, I187K, C332A | + | − |
| Neu2-M153 | M1D, V6Y, P62N, I187K, C332A | ++ | +++ |
| Neu2-M154 | M1D, V6Y, P62D, I187K, C332A | ++ | + |
| Neu2-M155 | M1D, V6Y, P62E, I187K, C332A | ++ | +++ |
| Neu2-M156 | V6Y, C164G, I187K, T249A | + | + |
| Neu2-M157 | V6Y, C164G, I187K | + | + |
| Neu2-M158 | V6Y, Q126L, I187K D251G | + | ++ |
| Neu2-M159 | V6Y, L54M, Q69H, R78K, A171G, | + | + |
| | I187K | | |
| Neu2-M160 | V6Y, P62T, I187K | + | ++ |
| Neu2-M161 | V6Y, A150V, I187K | + | ++ |
| Neu2-M162 | P5H, V6Y, P62S, I187K | + | ++ |
| Neu2-M163 | V6Y, C164G, I187K | + | + |

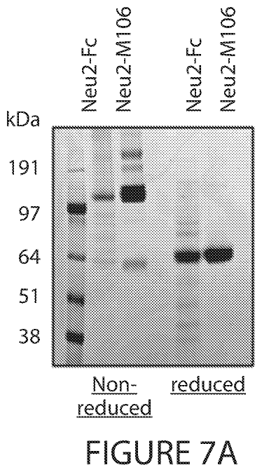

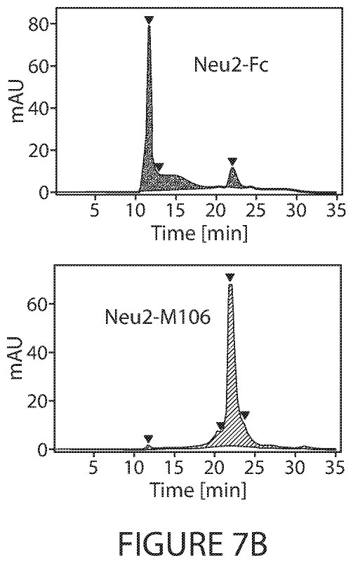

To confirm these results, Neu2-M106 (with amino acid sequence SEQ ID NO: 48, encoded by nucleotide sequence SEQ ID NO: 85) was expressed and purified with a protein A column. FIG. 7A is an image of an SDS-PAGE gel showing recombinant wildtype human Neu2 and Neu2 variant M106 (each with a C-terminal human Fc tag) under non-reducing and reducing conditions. FIG. 7B is an SEC-HPLC trace for recombinant wildtype human Neu2 and Neu2 variant M106 (each with a C-terminal human Fc tag). While Neu2-Fc had a yield of 0.3 mg/liter following protein-A purification, and monomer content of 7% as determined by SEC, Neu2-M106 had a yield of 20 mg/liter, and a monomer content of 85%.

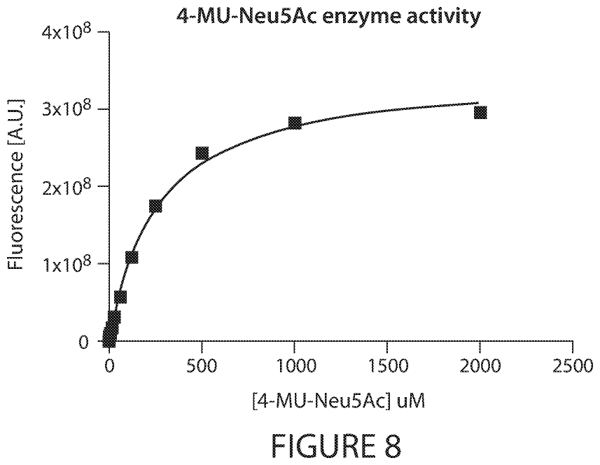

The enzyme kinetics of Neu2-M106 were assayed by measuring the release of sialic acid from the fluorogenic substrate 4-methylumbelliferyl-N-acetylneuraminic acid (4MU-NeuAc) as described above. A fixed concentration of enzyme at 2 μg/well was incubated with fluorogenic substrate 4MU-NeuAc at concentrations ranging from 4 mM to 0.03 μM. FIG. 8 depicts the enzyme activity of Neu2 variant M106. Enzymatic activity of Neu2-M106 was comparable to wildtype Neu2, with a KM determined to be 230 μM.

Together, these results show that the mutations identified by rational design, phage display, and/or yeast display approaches described herein can increase stability and/or expression of a sialidase.

### Example 3

This example describes the construction of recombinant human sialidases with mutations that increase expression and/or activity of the sialidase.

Unless indicated otherwise, mutant sialidases in this Example were expressed as secreted proteins with a C-terminal human Fc tag in Expi293F cells using the pCEP4 mammalian expression vector. Expression was assayed using a ForteBio Octet with anti-human Fc sensors and Western blot and enzymatic activity was assayed using the fluorogenic substrate 4MU-NeuAc as described above.

Mutant Neu2 sialidases were constructed including rationally designed substitutions at position Q126. Inspection of the Neu2 crystal structure revealed that mutation of Q126 may increase interactions with neighboring amino acid residues.

Additional mutant Neu2 sialidases were constructed including rationally designed substitution at position Q270. Inspection of the Neu2 crystal structure revealed that mutation of Q270 to certain amino acids may stabilize interactions with R237 and stabilize binding in the substrate pocket.

Additional mutant Neu2 sialidases were constructed including a substitution of an amino acid residue in a beta turn with a proline (for example D80P, R189P, and/or H239P substitutions). Substitution with a proline at these positions may, for example, stabilize the protein by influencing local protein folding.

Expression and activity levels for the resulting mutant sialidases are shown in TABLE 25. In TABLE 25, enzymatic activity is indicated as “++,” which denotes activity comparable to wild-type Neu2, “+,” which denotes activity lower than wild-type Neu2, or “−,” which denotes no detectable activity, and expression is indicated as “+++++”, which denotes expression >40 fold higher than wildtype-Neu2, “++++”, which denotes expression >15 fold higher than wildtype-Neu2, “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression.

TABLE 25

| Identifier | Mutation(s) | Activity | Expression |
|---|---|---|---|
| Neu2-M164 | M1D, V6Y, P62G, A93E, | ++ | ++++ |
| | Q126E, I187K, C332A | | |
| Neu2-M165 | M1D, V6Y, P62G, A93E, | ++ | ++++ |
| | Q126I, I187K, C332A | | |
| Neu2-M166 | M1D, V6Y, P62G, A93E, | + | +++++ |
| | Q126L, I187K, C332A | | |
| Neu2-M167 | M1D, V6Y, P62G, A93E, | + | +++++ |
| | Q126Y, I187K, C332A | | |
| Neu2-M168 | M1D, V6Y, P62G, A93E, | + | +++++ |
| | Q126F, I187K, C332A | | |
| Neu2-M169 | M1D, V6Y, P62G, A93E, | ++ | ++++ |
| | Q126H, I187K, C332A | | |
| Neu2-M170 | M1D, V6Y, P62G, A93E, | ++ | ++++ |
| | I187K, Q270S, C332A | | |
| Neu2-M171 | M1D, V6Y, P62G, A93E, | ++ | ++++ |
| | I187K, Q270T, C332A | | |
| Neu2-M172 | M1D, V6Y, P62G, A93E, | ++ | +++++ |
| | Q126Y, I187K, Q270T, | | |
| | C332A | | |
| Neu2-M173 | M1D, V6Y, P62G, A93E, | ++ | +++++ |
| | Q126Y, I187K, A242F, | | |
| | Q270T, C332A | | |
| Neu2-M174 | M1D, V6Y, P62G, D80P, | ++ | ++++ |
| | A93E, I187K, C332A | | |
| Neu2-M175 | M1D, V6Y, P62G, A93E, | ++ | +++ |
| | R170P, I187K, C332A | | |
| Neu2-M176 | M1D, V6Y, P62G, A93E, | ++ | ++++ |
| | I187K, Q188P, C332A | | |
| Neu2-M177 | M1D, V6Y, P62G, A93E, | ++ | ++++ |
| | I187K, R189P, C332A | | |
| Neu2-M178 | M1D, V6Y, P62G, A93E, | + | ++++ |
| | I187K, E225P, C332A | | |
| Neu2-M179 | M1D, V6Y, P62G, A93E, | ++ | ++++ |
| | I187K, H239P, C332A | | |
| Neu2-M180 | M1D, V6Y, P62G, A93E, | ++ | +++ |
| | I187K, E257P, C332A | | |

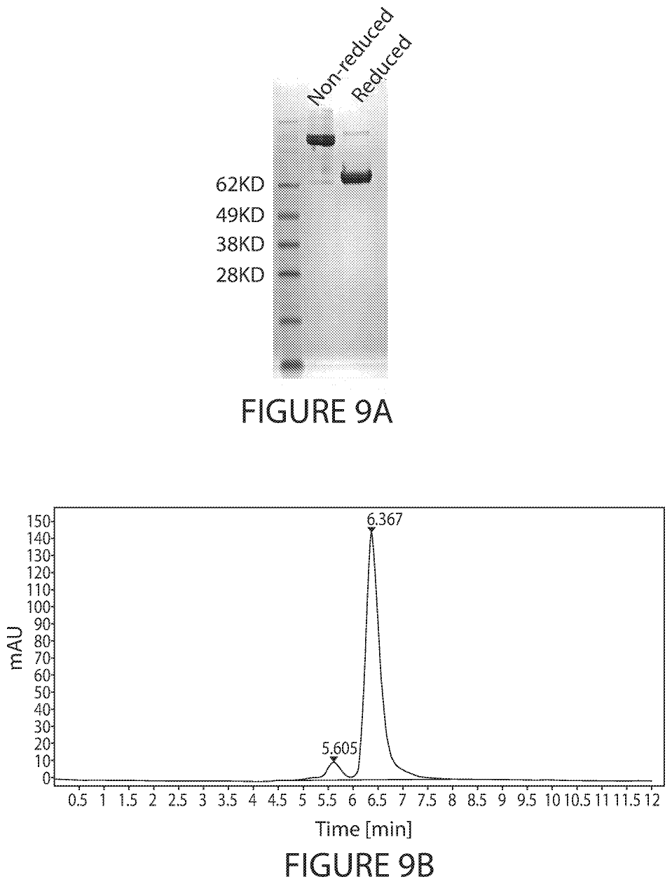

To confirm these results, Neu2-M173 (with amino acid sequence SEQ ID NO: 108, encoded by nucleotide sequence SEQ ID NO: 123) was expressed with a C-terminal human Fc tag and purified with a protein A and a ceramic hydroxyapatite (CHT) column. FIG. 9A is an image of an SDS-PAGE gel showing Neu2-M173-Fc (with a C-terminal human Fc tag) under non-reducing and reducing conditions. FIG. 9B is an SEC-HPLC trace for Neu2-M173-Fc (with a C-terminal human Fc tag). Neu2-M173-Fc had a yield of 120 mg/liter, and a monomer content of 90%.

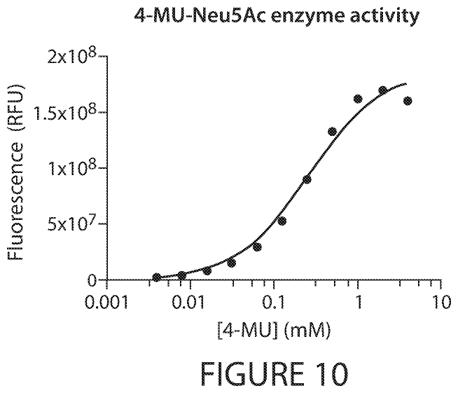

The enzyme kinetics of Neu2-M173-Fc were assayed by measuring the release of sialic acid from the fluorogenic substrate 4-methylumbelliferyl-N-acetylneuraminic acid (4MU-NeuAc) as described above. A fixed concentration of enzyme at 2 μg/well was incubated with fluorogenic substrate 4MU-NeuAc at concentrations ranging from 4 mM to 0.03 μM. FIG. 10 depicts the enzyme activity of Neu2-M173-Fc. Enzymatic activity of Neu2-M173-Fc was comparable to wildtype Neu2, with a KM determined to be 230 μM.

Additional mutant Neu2 sialidases were constructed including rationally designed substitutions at positions S301 and/or W302. Mutations of S301 and/or W302 may influence interactions with neighboring amino acid residues and/or substrate.

Expression and activity levels for the mutant sialidases are shown in TABLE 26. In TABLE 26, enzymatic activity is indicated as “++,” which denotes activity comparable to wild-type Neu2, “+,” which denotes activity lower than wild-type Neu2, or “−,” which denotes no detectable activity, and expression is indicated as “+++++”, which denotes expression >40 fold higher than wildtype-Neu2, “++++”, which denotes expression >15 fold higher than wildtype-Neu2, “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression.

TABLE 26

| Identifier | Mutation(s) | Activity | Expression |
|---|---|---|---|
| Neu2-M182 | M1D, V6Y, P62G, A93E, I187K, | ++ | ++++ |
| | S301A, C332A | | |
| Neu2-M183 | M1D, V6Y, P62G, A93E, I187K, | ++ | ++++ |
| | S301D, C332A | | |
| Neu2-M184 | M1D, V6Y, P62G, A93E, I187K, | ++ | +++ |
| | S301E, C332A | | |
| Neu2-M185 | M1D, V6Y, P62G, A93E, I187K, | + | ++++ |
| | S301F, C332A | | |
| Neu2-M186 | M1D, V6Y, P62G, A93E, I187K, | + | ++++ |
| | S301H, C332A | | |
| Neu2-M187 | M1D, V6Y, P62G, A93E, I187K, | ++ | +++++ |
| | S301K, C332A | | |
| Neu2-M188 | M1D, V6Y, P62G, A93E, I187K, | ++ | ++++ |
| | S301L, C332A | | |
| Neu2-M189 | M1D, V6Y, P62G, A93E, I187K, | ++ | ++++ |
| | S301M, C332A | | |
| Neu2-M190 | M1D, V6Y, P62G, A93E, I187K, | ++ | ++++ |
| | S301N, C332A | | |
| Neu2-M191 | M1D, V6Y, P62G, A93E, I187K, | ++ | ++++ |
| | S301P, C332A | | |
| Neu2-M192 | M1D, V6Y, P62G, A93E, I187K, | ++ | +++ |
| | S301Q, C332A | | |
| Neu2-M193 | M1D, V6Y, P62G, A93E, I187K, | ++ | +++++ |
| | S301R, C332A | | |
| Neu2-M194 | M1D, V6Y, P62G, A93E, I187K, | ++ | ++++ |
| | S301T, C332A | | |
| Neu2-M195 | M1D, V6Y, P62G, A93E, I187K, | ++ | ++++ |
| | S301V, C332A | | |
| Neu2-M196 | M1D, V6Y, P62G, A93E, I187K, | + | ++++ |
| | S301W, C332A | | |
| Neu2-M197 | M1D, V6Y, P62G, A93E, I187K, | ++ | ++++ |
| | S301Y, C332A | | |
| Neu2-M198 | M1D, V6Y, P62G, A93E, I187K, | + | ++++ |
| | W302A, C332A | | |
| Neu2-M199 | M1D, V6Y, P62G, A93E, I187K, | +++ | +++ |
| | W302D, C332A | | |
| Neu2-M200 | M1D, V6Y, P62G, A93E, I187K, | + | ++++ |
| | W302F, C332A | | |
| Neu2-M201 | M1D, V6Y, P62G, A93E, I187K, | ++ | ++++ |
| | W302G, C332A | | |
| Neu2-M202 | M1D, V6Y, P62G, A93E, I187K, | + | ++++ |
| | W302H, C332A | | |
| Neu2-M203 | M1D, V6Y, P62G, A93E, I187K, | + | ++++ |
| | W302I, C332A | | |
| Neu2-M204 | M1D, V6Y, P62G, A93E, I187K, | ++ | ++++ |
| | W302L, C332A | | |
| Neu2-M205 | M1D, V6Y, P62G, A93E, I187K, | + | ++ |
| | W302M, C332A | | |
| Neu2-M206 | M1D, V6Y, P62G, A93E, I187K, | + | ++++ |
| | W302N, C332A | | |
| Neu2-M207 | M1D, V6Y, P62G, A93E, I187K, | − | ++++ |
| | W302P, C332A | | |
| Neu2-M208 | M1D, V6Y, P62G, A93E, I187K, | ++ | ++++ |
| | W302Q, C332A | | |
| Neu2-M209 | M1D, V6Y, P62G, A93E, I187K, | ++ | +++++ |
| | W302R, C332A | | |
| Neu2-M210 | M1D, V6Y, P62G, A93E, I187K, | ++ | ++++ |
| | W302S, C332A | | |
| Neu2-M211 | M1D, V6Y, P62G, A93E, I187K, | ++ | ++++ |
| | W302T, C332A | | |
| Neu2-M212 | M1D, V6Y, P62G, A93E, I187K, | + | ++++ |
| | W302V, C332A | | |
| Neu2-M213 | M1D, V6Y, P62G, A93E, I187K, | + | ++++ |
| | W302Y, C332A | | |
| Neu2-M214 | M1D, V6Y, P62G, A93E, I187K, | ++ | ++++ |
| | S301A, W302A, C332A | | |
| Neu2-M215 | M1D, V6Y, P62G, A93E, I187K, | + | ++++ |
| | S301A, W302R, C332A | | |
| Neu2-M216 | M1D, V6Y, P62G, A93E, I187K, | ++ | ++++ |
| | S301A, W302S, C332A | | |
| Neu2-M217 | M1D, V6Y, P62G, A93E, I187K, | ++ | ++++ |
| | S301A, W302T, C332A | | |
| Neu2-M218 | M1D, V6Y, P62G, A93E, I187K, | + | ++++ |
| | S301K, W302S, C332A | | |
| Neu2-M219 | M1D, V6Y, P62G, A93E, I187K, | ++ | +++++ |
| | S301K, W302R, C332A | | |
| Neu2-M219 | M1D, V6Y, P62G, A93E, I187K, | ++ | ++++ |
| | S301K, W302T, C332A | | |
| Neu2-M220 | M1D, V6Y, P62G, A93E, I187K, | + | ++++ |
| | S301N, W302S, C332A | | |
| Neu2-M221 | M1D, V6Y, P62G, A93E, I187K, | ++ | ++++ |
| | S301N, W302T, C332A | | |
| Neu2-M222 | M1D, V6Y, P62G, A93E, I187K, | + | ++++ |
| | S301T, W302R, C332A | | |

### Example 4

This example describes the construction of recombinant human sialidases with mutations that reduce proteolytic cleavage.

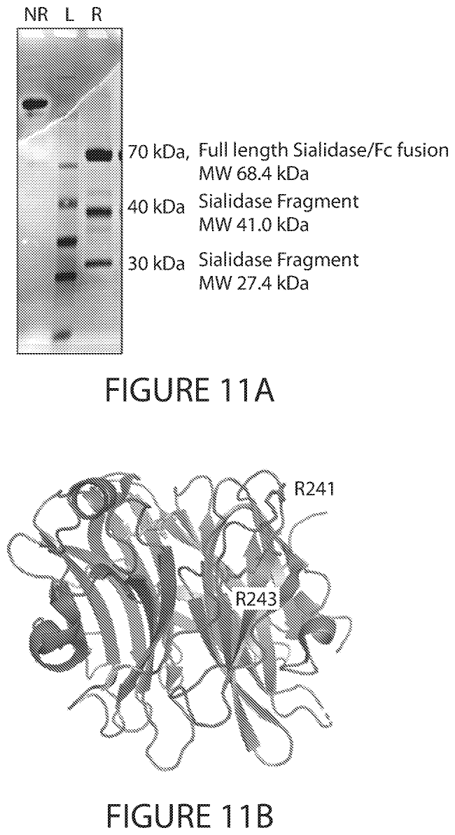

Neu2-M106 (as described in Example 2, and with amino acid sequence SEQ ID NO: 48) was expressed as an Fc-fused single chain protein using a CHO cell expression system in a large scale (10 L) high cell density production and purified with a protein A column. The resulting protein was analyzed by SDS-PAGE. Results are shown in FIG. 11. Under reducing conditions, the protein included a mixture of full length (70 kDa, approx. 50%) and cleaved (40 kDa and 30 kDa, approx. 50%) fractions. However, in non-reducing conditions, there was no cleavage and the protein remained as a single chain (FIG. 11). Additionally, when Neu2-M106 was expressed on a smaller scale (with a shorter duration of cell culture and lower cell density) there was no cleavage and the protein remained as a single chain. A preliminary mass spectrometry analysis showed that the 40 kDa and 30 kDa molecular weight fractions observed under reducing conditions following large scale production are a result of cleavage between amino acid residues R243 and V244 of the sialidase. The enzymatic activity of cleaved Neu2-M106 was similar to that of uncleaved Neu2-M106.

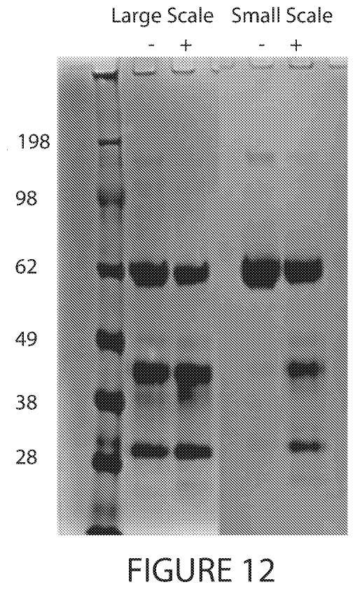

It was hypothesized that the cleavage of Neu2-M106 could be due to the activity of intracellular proteases released as a result of cell lysis during protein production, harvesting, and/or purification. To test this hypothesis, cleaved Neu2-M106 (prepared using the large scale-production described above that results in cleavage) and uncleaved Neu2-M106 (prepared using the smaller scale production described above that does not result in cleavage) were both incubated with trypsin and analyzed by SDS-PAGE under reducing conditions (FIG. 12). Briefly, trypsin digestion reactions were performed by incubation of trypsin (5 μL, 0.005% solution in PBS) with Neu2-M106 (25 μL, 0.25 mg/mL in PBS pH 8.0) for 5 minutes on ice. Reactions were stopped by addition of LDS gel loading buffer (5 μL) and run on a reducing SDS-PAGE gel to observe trypsin mediated cleavage. The SDS-PAGE analysis showed that incubation of the uncleaved Neu2-M106 with trypsin resulted in the same cleavage pattern as that of the cleaved Neu2-M106. Additionally, incubation of the cleaved Neu2-M106 with trypsin resulted in increased intensity of the bands corresponding to the cleavage products.

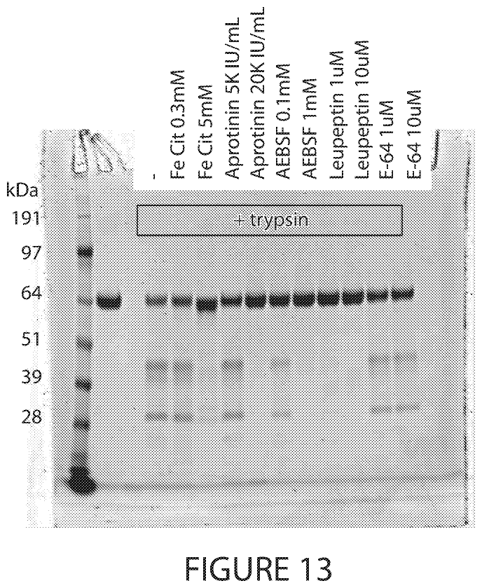

Neu2-M106 was also incubated with trypsin in the presence of various protease inhibitors. Briefly, trypsin digestion reactions were performed by incubation of trypsin (0.005%) with Neu2-M106 (0.5 mg/mL) and protease inhibitor for 5 minutes on ice. Reactions were stopped by addition of LDS gel loading buffer and run on a reducing SDS-PAGE gel to observe trypsin mediated cleavage. Inhibitors used included iron citrate (at 0.3 and 5 mM), aprotinin (at 5,000 and 20,000 U/mL), AEBSF (at 0.1 and 1 mM), leupeptin (at 1 and 10 μM) or E-64 (at 1 and 10 μM). As seen in FIG. 13, protease inhibitors reduced the extent of trypsin cleavage.

Together, these results confirm that cleavage of Neu2-M106 following large-scale production is due to trypsin or a member of a similar class of proteases.

We next attempted to rationally design recombinant human sialidases with mutations that increase resistance to trypsin cleavage.

Unless indicated otherwise, in the remainder of this Example mutant sialidases were expressed as secreted proteins with a C-terminal human Fc tag in Expi293F cells (on a 50 mL scale) using the pCEP4 mammalian expression vector. The resulting protein was purified using a Protein A column. Expression was assayed using a ForteBio Octet with anti-human Fc sensors and Western blot and enzymatic activity was assayed using the fluorogenic substrate 4MU-NeuAc as described above. Protease cleavage was assayed by SDS-PAGE as described above.

First, R243 was mutated to different polar/charged amino acids such as K, E, H, N and Q. However, these mutations of R243 resulted in complete loss of activity and reduction in expression yields (R243 is also a conserved amino acid among similar sialidases).

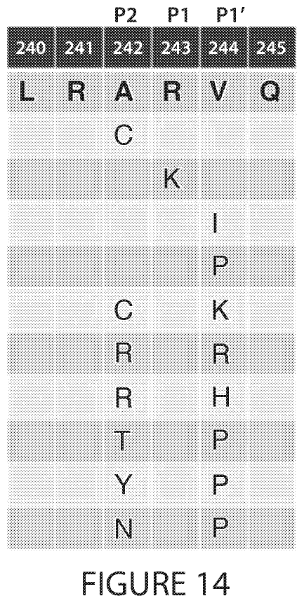

Next, various amino residues surrounding the cleavage site were mutated and tested for expression, activity and trypsin cleavage resistance. Substitutions and combinations of substitutions that were tested are shown in FIG. 14. All mutations were tested in a Neu2-M106 background (i.e., including M1D, V6Y, P62G, A93E, I187K, and C332A substitutions).

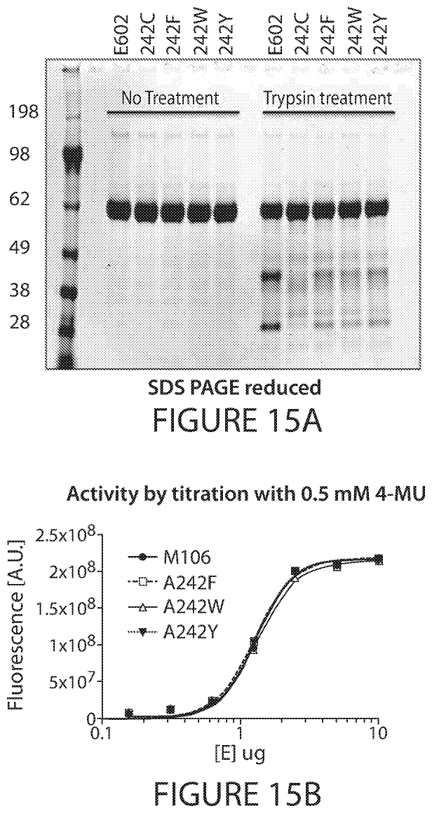

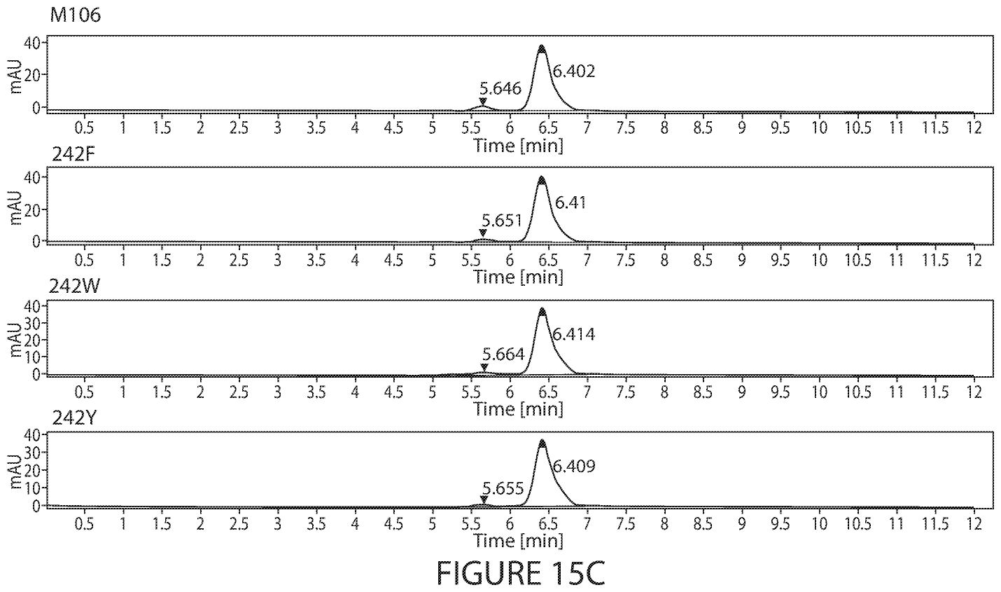

Most of the mutant sialidases depicted in FIG. 14 expressed well, however only two of the mutant sialidases (including V244I or A242C mutations) were active. The A242C mutation resulted in greater than 10 fold improved trypsin resistance and slightly lower activity (both relative to Neu2-M106). However, having an unpaired cysteine could be a potential liability, so, A242 was mutated to all 19 other amino acids and assayed for activity and trypsin resistance. As shown in FIG. 15, mutation of A242 to aromatic amino acids such as F, W and Y resulted in a dramatic improvement in trypsin cleavage resistance compared to Neu2-M106 (FIG. 15A) and similar enzymatic activity to Neu2-M106 (FIG. 15B). SEC analysis showed that proteins containing each of these mutations had a similar pattern to that of Neu2-M106 and more than 95% monomer content (FIG. 15C).

Structural analysis showed that replacing A242 with an aromatic amino acid could provide additional hydrophobic or stacking interactions to L260 and V265 (nonpolar amino acids located in the vicinity of the A242). Therefore, L260 and V265 were also mutated to phenylalanine. Along with these mutations several other rationally designed mutants, which could provide extra stability, for example by increasing stacking interactions, were also tested for expression, activity, and protease resistance.

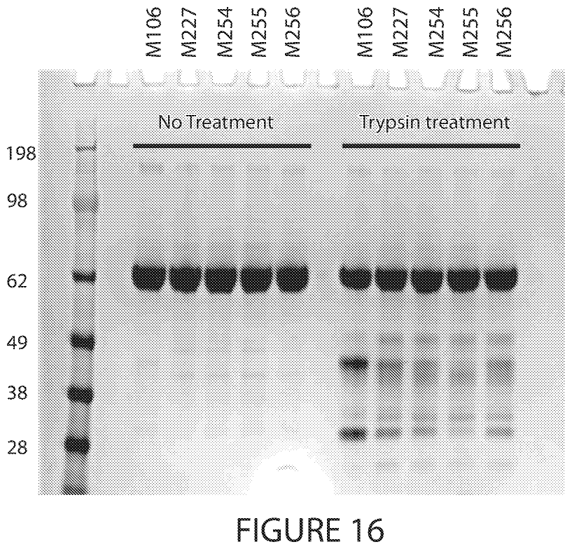

Select results are shown in FIG. 16. As shown in FIG. 16, the combination of R241Y and A242F mutations (Neu2-M255) resulted in the most resistance to trypsin cleavage (a greater than 10 fold improved trypsin resistance relative to Neu2-M106).

Expression, activity, and protease resistance levels for the mutant sialidases are shown in TABLE 27. In TABLE 27, enzymatic activity is indicated as “++,” which denotes activity comparable to wild-type Neu2, “+,” which denotes activity lower than wild-type Neu2, or “−,” which denotes no detectable activity, and expression is indicated as “+++++”, which denotes expression >40 fold higher than wildtype-Neu2, “++++”, which denotes expression >15 fold higher than wildtype-Neu2, “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression. Protease/trypsin resistance is indicated as “+++,” which denotes resistance >10 fold higher than Neu2-M106; “++,” which denotes resistance >5 fold higher than Neu2-M106, “+,” denotes resistance comparable to Neu2-M106, or “−,” which denotes resistance lower than Neu2-M106. NT=not tested.

TABLE 27

| | | | | Protease |
|---|---|---|---|---|
| Identifier | Mutation(s) | Activity | Expression | resistance |
| Neu2-M223 | M1D, V6Y, P62G, A93E, | ++ | +++++ | + |
| | I187K, L240Y, C332A | | | |
| Neu2-M224 | M1D, V6Y, P62G, A93E, | + | +++ | +++ |
| | I187K, A242C, C332A | | | |
| Neu2-M225 | M1D, V6Y, P62G, A93E, | − | ++++ | − |
| | I187K, A242D, C332A | | | |
| Neu2-M226 | M1D, V6Y, P62G, A93E, | − | +++ | − |
| | I187K, A242E, C332A | | | |
| Neu2-M227 | M1D, V6Y, P62G, A93E, | ++ | ++++ | ++ |
| | I187K, A242F, C332A | | | |
| Neu2-M228 | M1D, V6Y, P62G, A93E, | + | +++ | + |
| | I187K, A242G, C332A | | | |
| Neu2-M229 | M1D, V6Y, P62G, A93E, | + | +++ | + |
| | I187K, A242H, C332A | | | |
| Neu2-M230 | M1D, V6Y, P62G, A93E, | + | +++ | + |
| | I187K, A242I, C332A | | | |
| Neu2-M231 | M1D, V6Y, P62G, A93E, | + | ++++ | − |
| | I187K, A242K, C332A | | | |
| Neu2-M232 | M1D, V6Y, P62G, A93E, | + | +++ | + |
| | I187K, A242L, C332A | | | |
| Neu2-M233 | M1D, V6Y, P62G, A93E, | + | +++ | + |
| | I187K, A242M, C332A | | | |
| Neu2-M234 | M1D, V6Y, P62G, A93E, | + | ++++ | ++ |
| | I187K, A242N, C332A | | | |
| Neu2-M235 | M1D, V6Y, P62G, A93E, | + | +++ | ++ |
| | I187K, A242Q, C332A | | | |
| Neu2-M236 | M1D, V6Y, P62G, A93E, | + | ++++ | − |
| | I187K, A242R, C332A | | | |
| Neu2-M237 | M1D, V6Y, P62G, A93E, | + | +++ | − |
| | I187K, A242S, C332A | | | |
| Neu2-M238 | M1D, V6Y, P62G, A93E, | − | +++ | + |
| | I187K, A242T, C332A | | | |
| Neu2-M239 | M1D, V6Y, P62G, A93E, | + | +++ | + |
| | I187K, A242V, C332A | | | |
| Neu2-M240 | M1D, V6Y, P62G, A93E, | ++ | ++++ | ++ |
| | I187K, A242W, C332A | | | |
| Neu2-M241 | M1D, V6Y, P62G, A93E, | ++ | ++++ | ++ |
| | I187K, A242Y, C332A | | | |
| Neu2-M242 | M1D, V6Y, P62G, A93E, | ++ | ++++ | ++ |
| | I187K, A242F, L260F, | | | |
| | C332A | | | |
| Neu2-M243 | M1D, V6Y, P62G, A93E, | + | ++++ | ++ |
| | I187K, A242F, V265F, | | | |
| | C332A | | | |
| Neu2-M244 | M1D, V6Y, P62G, A93E, | + | ++++ | ++ |
| | I187K, A242F, A213C, | | | |
| | C332A | | | |
| Neu2-M245 | M1D, V6Y, P62G, A93E, | + | ++++ | ++ |
| | I187K, A242F, A213S, | | | |
| | C332A | | | |
| Neu2-M246 | M1D, V6Y, P62G, A93E, | ++ | ++++ | ++ |
| | I187K, A242F, A213T, | | | |
| | C332A | | | |
| Neu2-M247 | M1D, V6Y, P62G, A93E, | + | ++ | NT |
| | I187K, A242F, A213N, | | | |
| | C332A | | | |
| Neu2-M248 | M1D, V6Y, P62G, A93E, | ++ | ++++ | ++ |
| | I187K, A242F, A213C, | | | |
| | S258C, C332A | | | |
| Neu2-M249 | M1D, V6Y, P62G, A93E, | ++ | +++++ | + |
| | I187K, L240Y, L260F, | | | |
| | C332A | | | |
| Neu2-M250 | M1D, V6Y, P62G, A93E, | + | + | NT |
| | I187K, L240D, L260T, | | | |
| | C332A | | | |
| Neu2-M251 | M1D, V6Y, P62G, A93E, | + | ++ | NT |
| | I187K, L240N, L260D, | | | |
| | C332A | | | |
| Neu2-M252 | M1D, V6Y, P62G, A93E, | ++ | ++++ | + |
| | I187K, L240N, L260T, | | | |
| | C332A | | | |
| Neu2-M253 | M1D, V6Y, P62G, A93E, | ++ | ++++ | + |
| | I187K, L240N, L260Q, | | | |
| | C332A | | | |
| Neu2-M254 | M1D, V6Y, P62G, A93E, | ++ | +++ | ++ |
| | I187K, R241A, A242F, | | | |
| | C332A | | | |
| Neu2-M255 | M1D, V6Y, P62G, A93E, | ++ | +++ | +++ |
| | I187K, R241Y, A242F, | | | |
| | C332A | | | |
| Neu2-M256 | M1D, V6Y, P62G, A93E, | ++ | +++++ | ++ |
| | Q126Y, I187K, L240Y, | | | |
| | A242F, Q270T, C332A | | | |
| Neu2-M257 | M1D, V6Y, P62G, A93E, | ++ | ++++ | + |
| | I187K, V244I, C332A | | | |
| Neu2-M258 | M1D, V6Y, P62G, A93E, | + | +++ | ++ |
| | I187K, A242C, V244K, | | | |
| | C332A | | | |

### Example 5

This Example describes the construction and expression of a Rituximab sialidase genetic fusion protein, and a Rituximab sialidase conjugate containing the fusion protein, with a mutated human sialidase.

The architecture for four exemplary types of antibody sialidase conjugates (ASCs) is depicted in FIG. 19. The first type of ASC, referred to as “Raptor,” includes an antibody (with two heavy chains and two light chains) with a sialidase fused at the C-terminus of each heavy chain of the antibody (FIG. 19A). The second type of ASC, referred to as “Janus,” contains one antibody arm (with one heavy chain and one light chain), and one sialidase-Fc fusion with a sialidase fused at the N-terminus of one arm of the Fc. Each Fc domain polypeptide in the Janus ASC contains either the “knob” (T366Y) or “hole” (Y407T) mutation for heterodimerization (residue numbers according to EU numbering, Kabat, E. A., et al. (1991) supra) (FIG. 19B). The third type of ASC, referred to as “Lobster,” contains two Fc domain polypeptides each with a sialidase fused at the N-terminus of the Fc and a scFv fused at the C-terminus of the Fc (FIG. 19C). The fourth type of ASC, referred to as “Bunk,” contains one antibody arm (with one heavy chain and one light chain) with an scFv fused at the C-terminus of one arm of the Fc and one sialidase-Fc fusion with a sialidase fused at the N-terminus of the other arm of the Fc. Each Fc domain polypeptide in the Bunk ASC contains either the “knob” (T366Y) or “hole” (Y407T) mutation for heterodimerization (residue numbers according to EU numbering, Kabat, E. A., et al. (1991) supra) (FIG. 19D).

A Janus Antibody Sialidase Conjugate (ASC) was made using Neu2 with M1D, V6Y, I187K, and C332A mutations and the variable region of Rituximab. This Janus Rituximab (including a first polypeptide chain with amino acid sequence SEQ ID NO: 66, encoded by nucleotide sequence SEQ ID NO: 86, a second polypeptide chain with amino acid sequence SEQ ID NO: 67, encoded by nucleotide sequence SEQ ID NO: 87, and a third polypeptide chain with amino acid sequence SEQ ID NO: 78, encoded by nucleotide sequence SEQ ID NO: 79) was expressed and characterized for purity using SDS-PAGE and enzymatic activity using 4MU-NeuAc as described below.

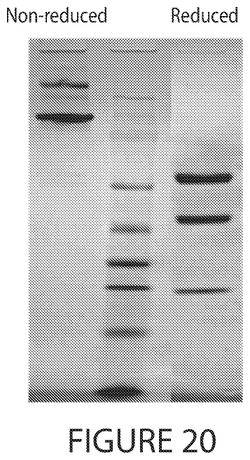

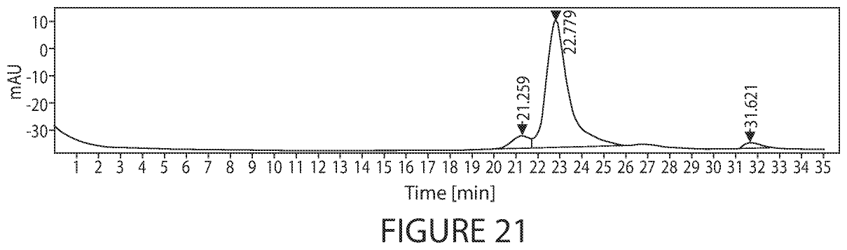

Janus Rituximab was expressed in a 200 mL transfection of HEK293F human cells in 24-well plates using the pCEP4 mammalian expression vector. Janus Rituximab was purified using Ni-NTA columns, quantified with a UV-Vis spectrophotometer (NanoDrop), and examined by SDS-PAGE as shown in FIG. 20. The Rituximab based Janus ASC expressed with a yield of ˜15 μg/mL and showed good monomer purity after purification (for example, see FIG. 21 which shows an SEC-HPLC trace having a large peak at ˜23 min).

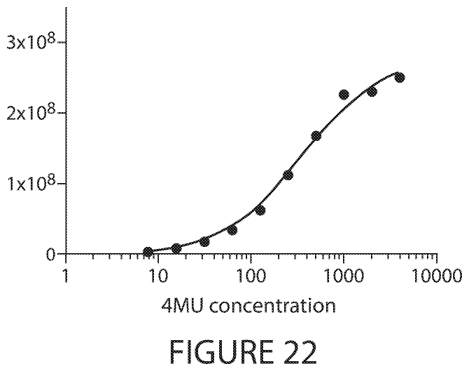

The activity of the recombinantly expressed Janus Rituximab was assayed by measuring the release of sialic acid from the fluorogenic substrate 4-methylumbelliferyl-N-acetylneuraminic acid (4MU-NeuAc). Specifically, an enzyme kinetics assay was performed by incubating a fixed concentration of enzyme at 1 nM with fluorogenic substrate 4MU-NeuAc at concentrations ranging from 4000 μM to 7.8 μM. As shown in FIG. 22, Janus Rituximab was active, causing the release of sialic acid which generated fluorescence. Assays were conducted at neutral (pH 7) conditions.

### Example 6

This Example describes the construction and expression of an Ofatumumab sialidase genetic fusion protein, and an Ofatumumab sialidase conjugate containing the fusion protein, with a mutated human sialidase.

A Janus Antibody Sialidase Conjugate (ASC) was made using Neu2 with M1D, V6Y, I187K, and C332A mutations and the variable region of Ofatumumab. This Janus Ofatumumab (including a first polypeptide chain with amino acid sequence SEQ ID NO: 111, encoded by nucleotide sequence SEQ ID NO: 112, a second polypeptide chain with amino acid sequence SEQ ID NO: 81, encoded by nucleotide sequence SEQ ID NO: 83, and a third polypeptide chain with amino acid sequence SEQ ID NO: 78, encoded by nucleotide sequence SEQ ID NO: 79) was expressed and characterized for purity using SDS-PAGE and enzymatic activity using 4MU-NeuAc as described below.

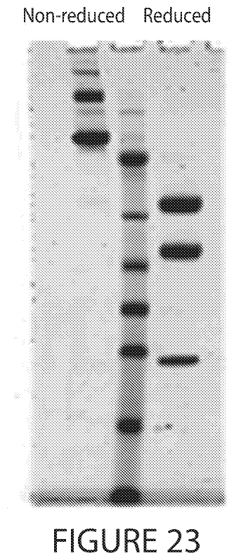

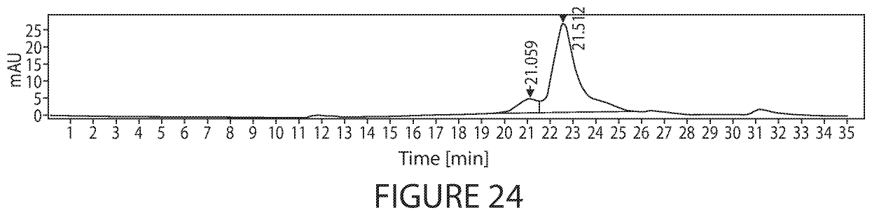

Janus Ofatumumab was expressed in a 200 mL transfection of HEK293F human cells in 24-well plates using the pCEP4 mammalian expression vector. Janus Ofatumumab was purified using Ni-NTA columns, quantified with a UV-Vis spectrophotometer (NanoDrop), and examined by SDS-PAGE as shown in FIG. 23. The Ofatumumab-based Janus ASC expressed with a yield of ˜20 μg/mL and showed good monomer purity after purification (for example, see FIG. 24 which shows an SEC-HPLC trace having a large peak at ˜22.5 min).

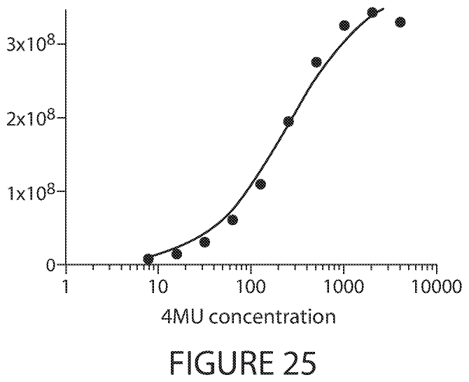

The activity of the recombinantly expressed Janus Ofatumumab was assayed by measuring the release of sialic acid from the fluorogenic substrate 4-methylumbelliferyl-N-acetylneuraminic acid (4MU-NeuAc). Specifically, an enzyme kinetics assay was performed by incubating a fixed concentration of enzyme at 1 nM with fluorogenic substrate 4MU-NeuAc at concentrations ranging from 4000 μM to 7.8 μM. As shown in FIG. 25, Janus Ofatumumab was active, causing the release of sialic acid which generated fluorescence. Assays were conducted at neutral (pH 7) conditions.

### Example 7

This Example describes the desialylation of transgenic cell lines by anti-CD20 antibody sialidase conjugates (ASCs) containing human sialidases.

Two mouse cell lines, mouse lymphoma A20 and T cell leukemia EL4 were genetically transduced to overexpress human CD20 to establish transgenic cell lines A20-CD20 and EL4-CD20. Desialylation status of the cell lines was determined following treatment with Janus Rituximab and Janus Ofatumumab molecules (as described in Examples 5 and 6).

100 K cells were seeded in 10% RPMI media in 96-well plate and treated with ASCs at a final concentration of 10 μg/ml. The cells were incubated at 37° C. overnight and the next day stained for desialylation using Peanut Agglutinin (PNA; Vector Labs). Increased PNA staining is correlated with the removal of terminal sialic acids and exposure of underlying galactose.

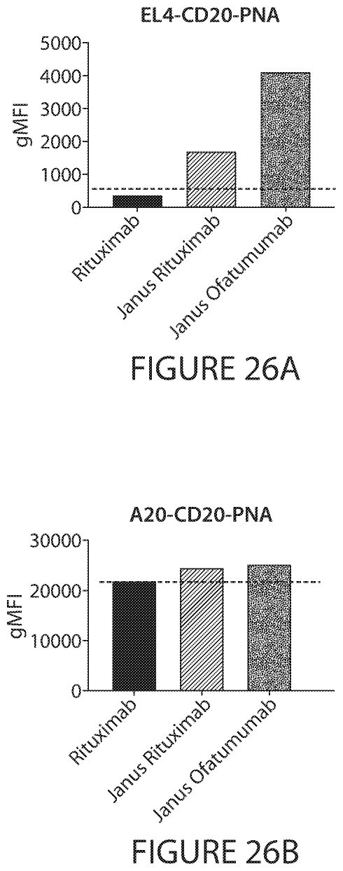

Treatment with both Janus Rituximab and Janus Ofatumumab increased PNA staining. As shown in FIG. 26A, EL4-CD20 cells, which have a low basal level of PNA staining, showed increased PNA staining when treated with Janus Rituximab or Janus Ofatumumab, indicating that the ASCs have sialidase activity. As shown in FIG. 26B, A20-CD20 cells, which have a high basal level of PNA staining, showed a slight increase in PNA staining over the negative control (rituximab), indicating that the ASCs have sialidase activity.

### Example 8

In this Example, anti-CD20 antibody sialidase conjugates (ASCs) containing human sialidases were tested for their ability to induce apoptosis in a human B-cell lymphoma cell line.

100 K cells of each of the human B-cell lymphoma cell lines Raji and Ramos were seeded in 96 well plates and treated with Janus Rituximab or Janus Ofatumumab (as described in Examples 5 and 6) at a 10 μg/ml final concentration. Cells were incubated for 24 hours and assayed for Annexin-V staining, a measure of apoptosis, using flow cytometry.

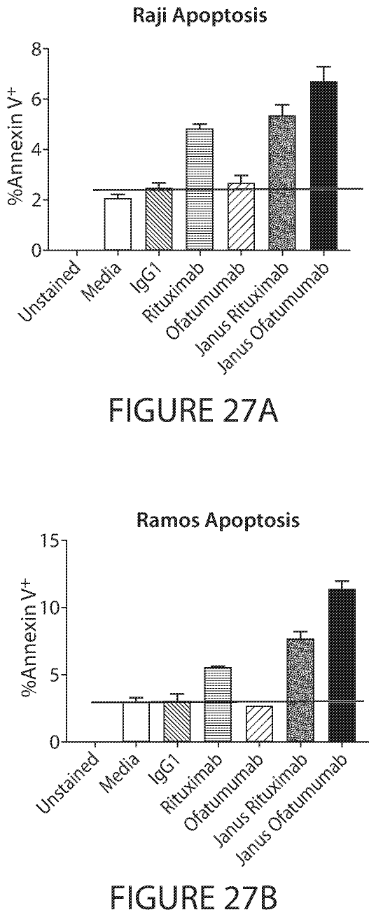

Treatment with Janus Rituximab and Janus Ofatumumab resulted in increased Annexin-V staining in both cell lines (see FIGS. 27A and 27B). Annexin-V staining for the ASCs was comparable to or slightly higher than for Rituximab or Ofatumumab. These results indicate that Janus Rituximab and Janus Ofatumumab induce apoptosis in a human lymphoma cell line.

### Example 9

This Example describes the construction and expression of additional ofatumumab sialidase genetic fusion proteins including a mutated human sialidase based on the Janus format.

A Janus Antibody Sialidase Conjugate (ASC) was made using Neu2 with M1D, V6Y, P62G, A93E, I187K, and C332A mutations and the variable region of ofatumumab. This ASC, referred to as Janus Ofatumumab #2, included a first polypeptide chain with amino acid sequence SEQ ID NO: 111, encoded by nucleotide sequence SEQ ID NO: 112, a second polypeptide chain with amino acid sequence SEQ ID NO: 81, encoded by nucleotide sequence SEQ ID NO: 83, and a third polypeptide chain with amino acid sequence SEQ ID NO: 113, encoded by nucleotide sequence SEQ ID NO: 114.

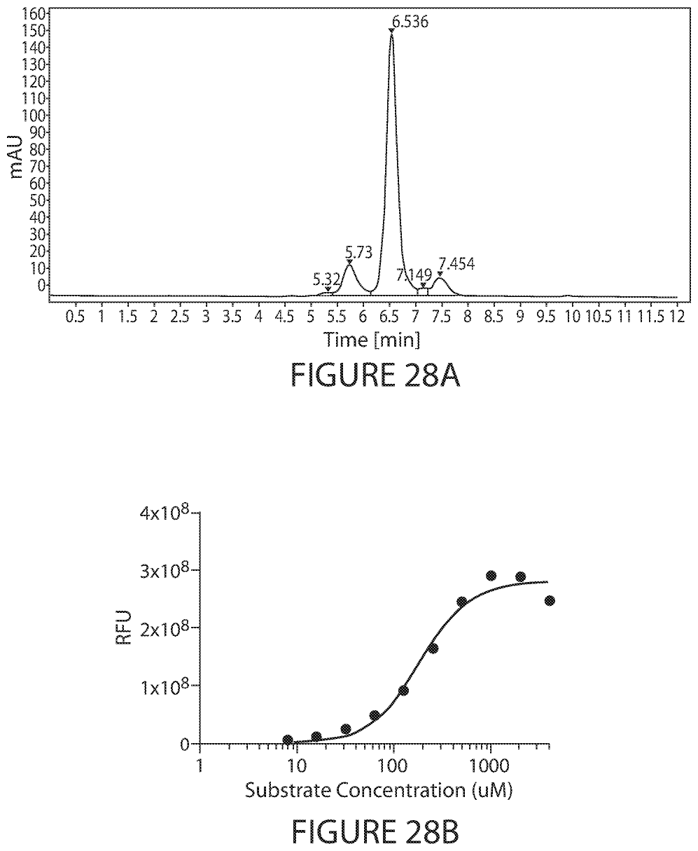

Janus Ofatumumab #2 was expressed and characterized as described in Example 6, with the exception that an AdvanceBio SEC 130 Å, 7.8×300 mm, 2.7 μm, LC column was used in place of a Cytiva Superdex 30 Increase 10/300 GL, 10×300 mm column. Janus Ofatumumab #2 showed good monomer purity after purification, as shown in FIG. 28A. The activity of the recombinantly expressed Janus Ofatumumab #2 was assayed using 4MU-NeuAc. As shown in FIG. 28B, Janus Ofatumumab #2 was active, causing a concentration dependent release of sialic acid which generated fluorescence. Assays were conducted at neutral (pH 7) conditions using 2 μg protein.

An additional Janus ASC was made using Neu2 with M1D, V6Y, P62G, A93E, I187K, and C332A mutations and the variable region of ofatumumab. This ASC, referred to as Janus Ofatumumab #3, included a first polypeptide chain with amino acid sequence SEQ ID NO: 111, encoded by nucleotide sequence SEQ ID NO: 112, a second polypeptide chain with amino acid sequence SEQ ID NO: 81, encoded by nucleotide sequence SEQ ID NO: 83, and a third polypeptide chain with amino acid sequence SEQ ID NO: 115, encoded by nucleotide sequence SEQ ID NO: 116. In the third polypeptide chain of Janus Ofatumumab #3, the sialidase and the Fc domain were separated by an IgG-based hinge region.

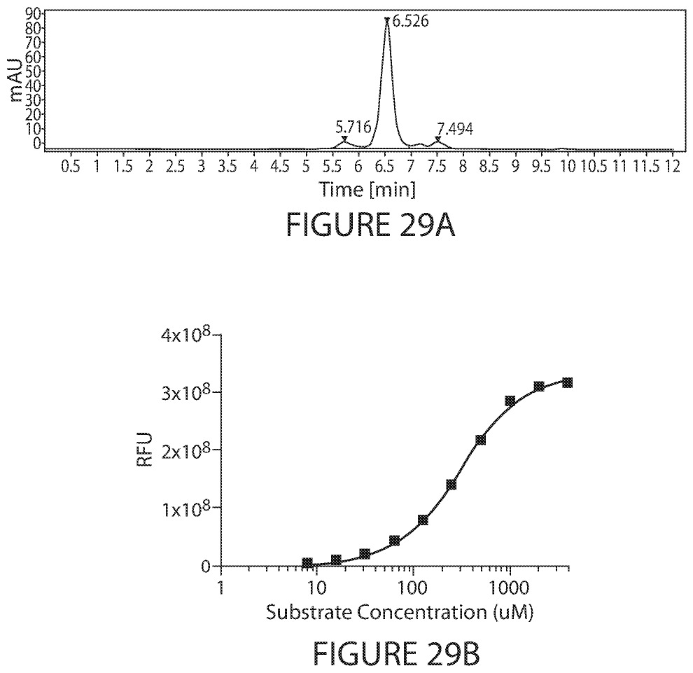

Janus Ofatumumab #3 was expressed and characterized as described in Example 6, with the exception that an AdvanceBio SEC 130 Å, 7.8×300 mm, 2.7 μm, LC column was used in place of a Cytiva Superdex 30 Increase 10/300 GL, 10×300 mm column. Janus Ofatumumab #3 showed good monomer purity after purification, as shown in FIG. 29A. The activity of the recombinantly expressed Janus Ofatumumab #3 was assayed using 4MU-NeuAc. As shown in FIG. 29B, Janus Ofatumumab #3 was active, causing a concentration dependent release of sialic acid which generated fluorescence. Assays were conducted at neutral (pH 7) conditions using 2 μg protein.

A control Janus ASC was made using enzymatically inactive Neu2 with M1D, V6Y, K9D, I187K, C332A, A93E, V363R, L365R, E218A, and C219N mutations and the variable region of ofatumumab. This ASC, referred to as Janus Ofatumumab LOF, included a first polypeptide chain with amino acid sequence SEQ ID NO: 111, encoded by nucleotide sequence SEQ ID NO: 112, a second polypeptide chain with amino acid sequence SEQ ID NO: 81, encoded by nucleotide sequence SEQ ID NO: 83, and a third polypeptide chain with amino acid sequence SEQ ID NO: 117, encoded by nucleotide sequence SEQ ID NO: 118.

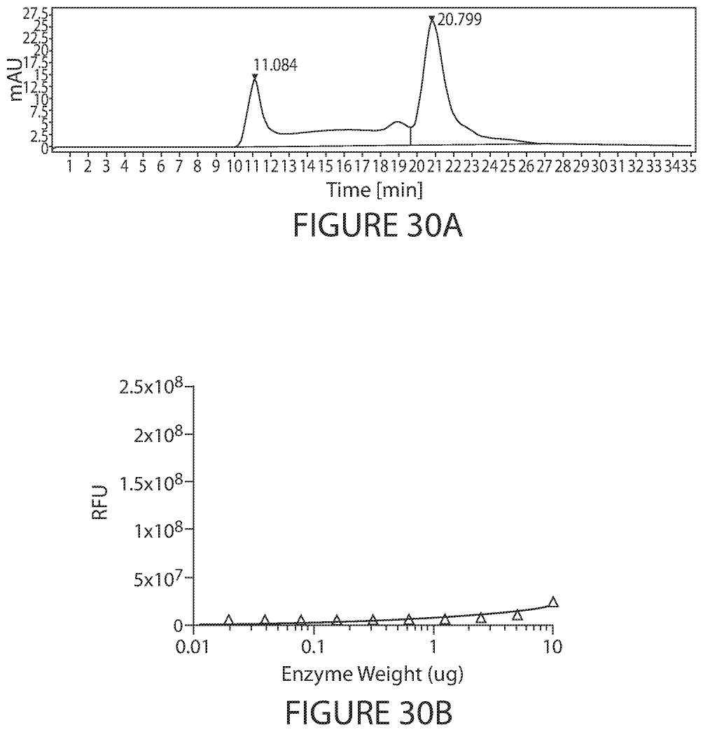

Janus Ofatumumab LOF was expressed and characterized as described in Example 6 and showed acceptable monomer purity after purification, as seen in FIG. 30A. The activity of the recombinantly expressed Janus Ofatumumab LOF was assayed using 4MU-NeuAc. As shown in FIG. 30B, Janus Ofatumumab LOF was inactive. Assays were conducted at neutral (pH 7) conditions using 0.5 mM substrate and titrating down from 10 μg protein.

### Example 10

This Example describes the construction and expression of ofatumumab sialidase genetic fusion proteins including a mutated human sialidase based on the Raptor format.

A Raptor Antibody Sialidase Conjugate (ASC) was made using Neu2 with M1D, V6Y, P62G, A93E, I187K, and C332A mutations fused to the heavy chain of ofatumumab. This ASC, referred to as Raptor Ofatumumab #1, included and first and fourth polypeptide chain with amino acid sequence SEQ ID NO: 111, encoded by nucleotide sequence SEQ ID NO: 112, and a second and third polypeptide chain with amino acid sequence SEQ ID NO: 119, encoded by nucleotide sequence SEQ ID NO: 120.

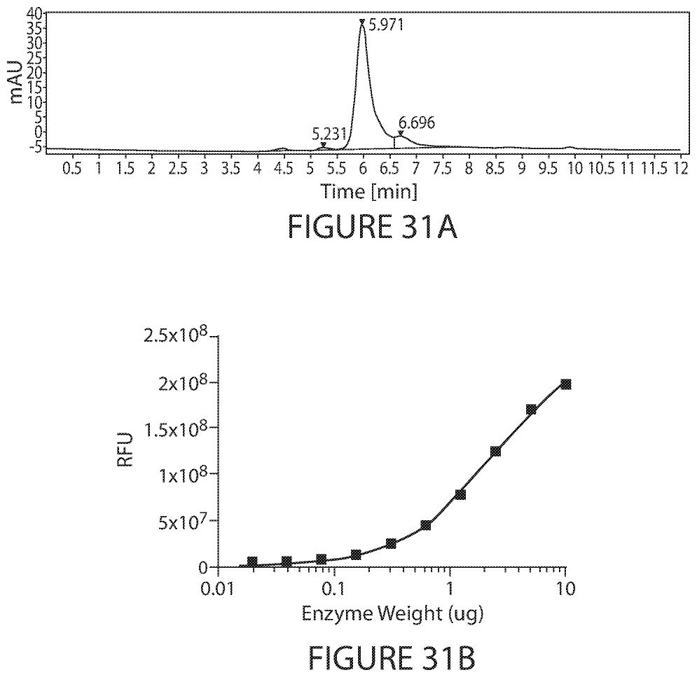

Raptor Ofatumumab #1 was expressed and characterized as described in Example 6, with the exception that an AdvanceBio SEC 130 Å, 7.8×300 mm, 2.7 μm, LC column was used in place of a Cytiva Superdex 30 Increase 10/300 GL, 10×300 mm column. Raptor Ofatumumab #1 showed good monomer purity after purification, as seen in FIG. 31A. The activity of the recombinantly expressed Raptor Ofatumumab #1 was assayed using 4MU-NeuAc. As shown in FIG. 31B, Raptor Ofatumumab #1 was active, causing the concentration dependent release of sialic acid which generated fluorescence. Assays were conducted at neutral (pH 7) conditions using 0.5 mM substrate and titrating down from 10 μg protein.

A second Raptor ASC was made using Neu2 with M1D, V6Y, P62G, A93E, Q126Y, I187K, Q270T, C332A mutations fused to the heavy chain of ofatumumab. This ASC, referred to as Raptor Ofatumumab #2, included and first and fourth polypeptide chain with amino acid sequence SEQ ID NO: 111, encoded by nucleotide sequence SEQ ID NO: 112, and a second and third polypeptide chain with amino acid sequence SEQ ID NO: 121, encoded by nucleotide sequence SEQ ID NO: 122.

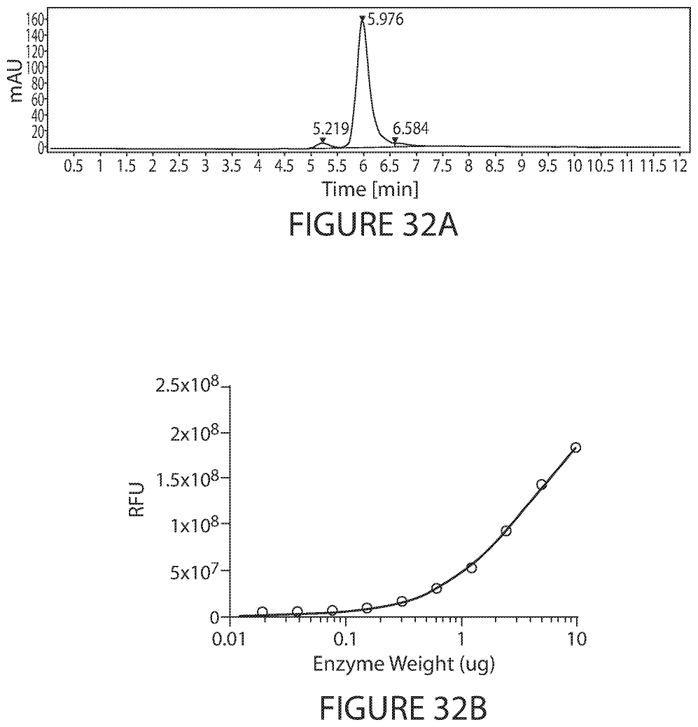

Raptor Ofatumumab #2 was expressed and characterized as described in Example 6, with the exception that an AdvanceBio SEC 130 Å, 7.8×300 mm, 2.7 μm, LC column was used in place of a Cytiva Superdex 30 Increase 10/300 GL, 10×300 mm column. Raptor Ofatumumab #2 showed good monomer purity after purification, as seen in FIG. 32A. The activity of the recombinantly expressed Raptor Ofatumumab #2 was assayed using 4MU-NeuAc. As shown in FIG. 32B, Raptor Ofatumumab #2 was active, causing the concentration dependent release of sialic acid which generated fluorescence. Assays were conducted at neutral (pH 7) conditions using 0.5 mM substrate and titrating down from 10 μg protein.

### Example 11

This Example describes the biochemical characterization of various anti-CD20 antibody sialidase conjugates (ASCs) containing mutated human sialidases as generated in Examples 9 and 10.

Human Raji and Ramos cells express CD20 and were used to test the ability of ASCs to remove cell surface sialic acid. 50,000 cells/well were seeded and incubated with test articles at 37° C. for 4 hours. Cells were then stained with a hexameric version of the extracellular domain of human Siglec 9, termed Hydra9 (as described in International (PCT) Application Publication No. WO2019/237070). Hydra9 preferentially binds to terminal sialic acid containing structures. A decrease in Hydra9 staining is indicative of the removal of terminal sialic acids by the sialidase portion of an ASC. Alternatively, cells were stained with PNA, a lectin that binds to terminal galactose residues. An increase in PNA staining is indicative of the removal of terminal sialic acids by the sialidase portion of an ASC and exposure of the underlying galactose. Hydra9 staining is done by incubating with 50 nM Hydra9 for 10 minutes at room temperature, followed by a secondary goat anti mouse IgG2 antibody (1:500) in FACS staining buffer. PNA staining is done at 1 μg/ml.

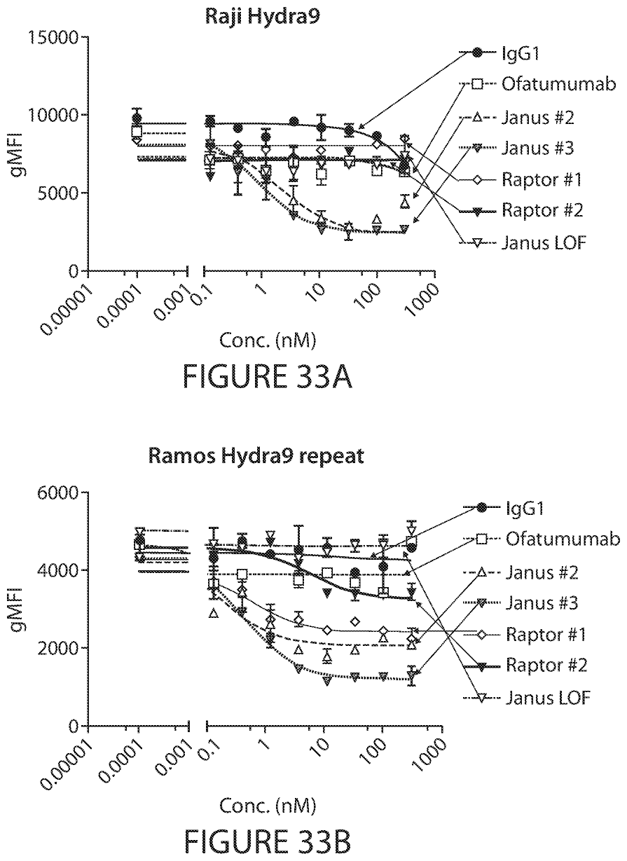

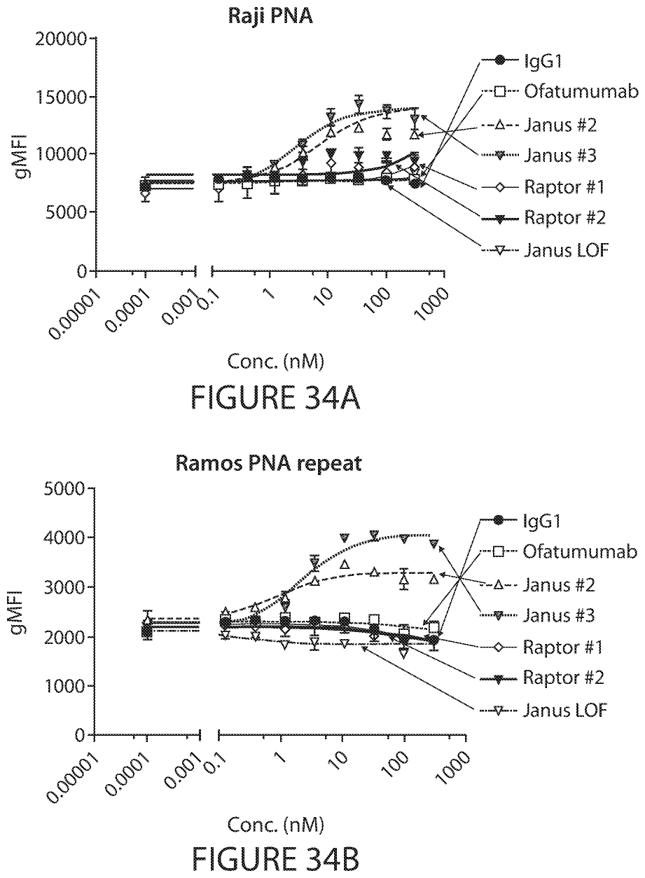

As can be seen in FIG. 33, treatment of Raji cells (FIG. 33A) as well as Ramos cells (FIG. 33B) with certain ASCs resulted in a reduction of Hydra9 staining, indicative of sialic acid removal by the ASCs. For example, Janus Ofatumumab #2 (as described in Example 9), Janus Ofatumumab #3 (as described in Example 9), Raptor Ofatumumab #1 (as described in Example 10) and Raptor Ofatumumab #2 (as described in Example 10) all showed activity against either Raji or Ramos cells as a substrate. Janus based constructs were all active with EC50s in the low nanomolar range on both Raji and Ramos cells. As can be seen FIG. 34, treatment of Raji cells (FIG. 34A) as well as Ramos cells (FIG. 34B) with certain ASCs resulted in an increase of PNA staining, indicative of sialic acid removal by the ASCs. For example, Janus Ofatumumab #2 and Janus Ofatumumab #3 showed activity against either Raji or Ramos cells as a substrate with EC50s in the low nanomolar range.

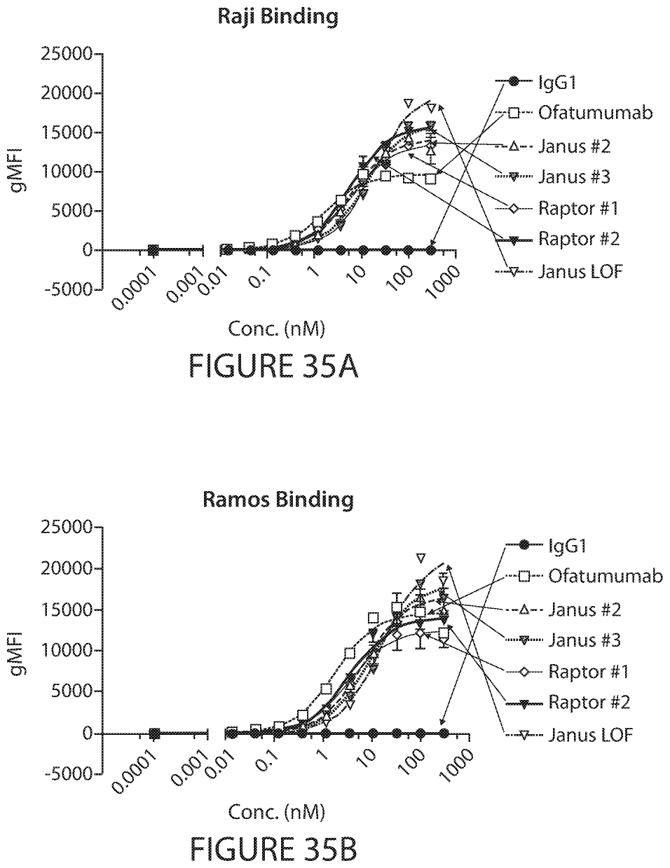

Binding of the various ofatumumab based ASCs was tested using Raji and Ramos cells, both of which express endogenous CD20 on their cell surfaces. 50,000 cells/well were seeded and treated with isotype control, ofatumumab, Janus Ofatumumab #2 (as described in Example 9), Janus Ofatumumab #3 (as described in Example 9), Raptor Ofatumumab #1 (as described in Example 10), Raptor Ofatumumab #2 (as described in Example 10), and Janus Ofatumumab LOF (as described in Example 9). The plate was incubated on ice for 30 minutes. Unbound material was removed by washing two times after 30 minutes. Cells were stained with anti-human Fc gamma AF-647 antibody, incubated on ice for 15 minutes, washed and run on FACS. As can be seen in FIG. 35, all of the ofatumumab ASCs bound with comparable efficiency to either Raji cells (FIG. 35A) or Ramos cells (FIG. 35B).

### Example 12

This Example describes the in vivo administration of anti-CD20 antibody sialidase conjugates (ASCs) containing human sialidases.

ASCs are tested in a mouse syngeneic tumor model injected with a murine breast cancer cell line expressing human CD20 (e.g., EL4 CD20 cells). Female C57/BL6 mice, 6-8 weeks of age, are inoculated subcutaneously in the right lower flank region with EL4-CD20 tumor cells (5×10⁵) in 0.1 ml of PBS for tumor development. Mice are randomly allocated to 4 groups of 5 animals each when tumors reached 50-100 mm³, mean ˜75-100 mm³.

Mice are treated via intraperitoneal injection of 10 mg/kg of either an anti-CD20 ASC (e.g., Janus Rituximab or Janus Ofatumumab), Rituximab, Ofatumumab, or vehicle control, and tumor volume (mm³) is recorded. Mean tumor volumes for the individual mice for the indicated treatments are determined. Complete Responses (CR, defined as regression below the limit of palpitation at any point during the study) are also determined.

It is expected that an anti-CD20 ASC (e.g., Janus Rituximab or Janus Ofatumumab) will reduce tumor volume and increase the number of CRs as compared to vehicle control. It is also expected that an anti-CD20 ASC (e.g., Janus Rituximab or Janus Ofatumumab) will reduce tumor volume comparably or better than Rituximab or Ofatumumab, respectively.

### Example 13

This Example describes the in vivo administration of anti-CD20 antibody sialidase conjugates (ASCs) containing human sialidases.

ASCs were tested in a mouse syngeneic intravenous dissemination model using a murine breast cancer cell line expressing human CD20 (EL4 CD20 cells). Female C57/BL6 mice, 6-8 weeks of age, were IV injected with 500,000 cells per mouse. Mice were subsequently dosed as described in TABLE 28 with isotype control, ofatumumab, Janus Ofatumumab #2 (as described in Example 9), or Janus Ofatumumab LOF (as described in Example 9). Body weights and clinical observations were recorded daily.

TABLE 28

| | # of | | Dose | Tumor | | Dose volume | |
|---|---|---|---|---|---|---|---|
| Group | mice | Treatment | (mg/kg) | route | Route | mL/kg | Schedule |
| 1 | 10 | Isotype control | 10 | IV | IP | 5 | Twice weekly |
| | | | | | | | for 5 doses |
| 2 | 20 | Ofatumumab | 10 | IV | IP | 10 | Twice weekly |
| | | | | | | | for 5 doses |
| 3 | 20 | Janus | 10 | IV | IP | 5 | Twice weekly |
| | | Ofatumumab #2 | | | | | for 5 doses |
| 4 | 20 | Janus | 10 | IV | IP | 5 | Twice weekly |
| | | Ofatumumab | | | | | for 5 doses |
| | | LOF | | | | | |

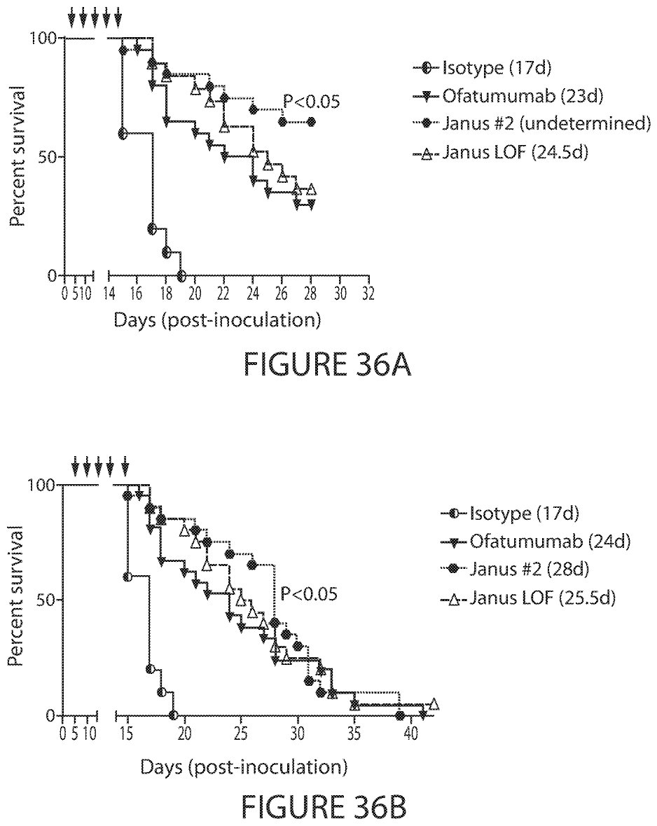

FIG. 36 depicts survival curves for mice in each group. FIG. 36A depicts the survival as of day 28 and FIG. 36B depicts the overall survival. As shown in FIG. 36A, as of day 28 Janus Ofatumumab #2 increased survival relative to ofatumumab and Janus Ofatumumab LOF. As shown in FIG. 36B, at the conclusion of the experiment, when ofatumumab was compared to isotype control, ofatumumab demonstrated a shift in survival, with the 50% survival point shifting from 17 days to 24 days. Janus Ofatumumab #2 demonstrated a larger survival shift to 28 days. Janus Ofatumumab LOF demonstrated a similar survival to ofatumumab of 25.5 days.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCE LISTING
SEQ ID NO: 1:
MASLPVLQKESVFQSGAHAYRIPALLYLPGQQSLLAFAEQ
RASKKDEHAELIVLRRGDYDAPTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPIQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSCAYSDLQSMGTGPDGSPLFGCLYEANDYE
EIVFLMFTLKQAFPAEYLPQ
SEQ ID NO: 2:
MEDLRPMATCPVLQKETLFRTGVHAYRIPALLYLKKQKTL
LAFAEKRASKTDEHAELIVLRRGSYNEATNRVKWQPEEVV
TQAQLEGHRSMNPCPLYDKQTKTLFLFFIAVPGRVSEHHQ
LHTKVNVTRLCCVSSTDHGRTWSPIQDLTETTIGSTHQEV
VATFAVGPGHCLQLRNPAGSLLVPAYAYRKLHPAQKPTPF
AFCFISLDHGHTWKLGNFVAENSLECQVAEVGTGAQRMVY
LNARSFLGARVQAQSPNDGLDFQDNRVVSKLVEPPHGCHG
SVVAFHNPISKPHALDTWLLYTHPTDSRNRTNLGVYLNQM
PLDPTAWSEPTLLAMGICAYSDLQNMGQGPDGSPQFGCLY
ESGNYEEIIFLIFTLKQAFPTVFDAQ
SEQ ID NO: 3:
EDLRP
SEQ ID NO: 4:
MEDLRP
SEQ ID NO: 5:
DKTHTCPPCPAPELLGGPSVFLFPPKPKDTLMISRTPEVT
CVVVDVSHEDPEVKFNWYVDGVEVHNAKTKPREEQYNSTY
RVVSVLTVLHQDWLNGKEYKCKVSNKALPAPIEKTISKAK
GQPREPQVYTLPPSREEMTKNQVSLTCLVKGFYPSDIAVE
WESNGQPENNYKTTPPVLDSDGSFFLYSKLTVDKSRWQQG
NVFSCSVMHEALHNHYTQKSLSLSPGK
SEQ ID NO: 6:
ACAGTGGAAAAGTCCGTGGTGTTCAAGGCCGAGGGCGAGC
ACTTCACCGACCAGAAAGGCAATACCATCGTCGGCTCTGG
CAGCGGCGGCACCACCAAGTACTTTAGAATCCCCGCCATG
TGCACCACCAGCAAGGGCACCATTGTGGTGTTCGCCGACG
CCAGACACAACACCGCCAGCGATCAGAGCTTCATCGATAC
CGCTGCCGCCAGATCTACCGATGGCGGCAAGACCTGGAAC
AAGAAGATCGCCATCTACAACGACCGCGTGAACAGCAAGC
TGAGCAGAGTGATGGACCCTACCTGCATCGTGGCCAACAT
CCAGGGCAGAGAAACCATCCTGGTCATGGTCGGAAAGTGG
AACAACAACGATAAGACCTGGGGCGCCTACAGAGACAAGG
CCCCTGATACCGATTGGGACCTCGTGCTGTACAAGAGCAC
CGATGACGGCGTGACCTTCAGCAAGGTGGAAACAAACATC
CACGACATCGTGACCAAGAACGGCACCATCTCTGCCATGC
TCGGCGGCGTTGGATCTGGCCTGCAACTGAATGATGGCAA
GCTGGTGTTCCCCGTGCAGATGGTCCGAACAAAGAATATC
ACCACCGTGCTGAATACCAGCTTCATCTACAGCACCGACG
GCATCACATGGTCCCTGCCTAGCGGCTACTGTGAAGGCTT
TGGCAGCGAGAACAACATCATCGAGTTCAACGCCAGCCTG
GTCAACAACATCCGGAACAGCGGCCTGCGGAGAAGCTTCG
AGACAAAGGACTTCGGAAAGACGTGGACCGAGTTTCCTCC
AATGGACAAGAAGGTGGACAACCGGAACCACGGCGTGCAG
GGCAGCACAATCACAATCCCTAGCGGCAACAAACTGGTGG
CCGCTCACTCTAGCGCCCAGAACAAGAACAACGACTACAC
CAGAAGCGACATCAGCCTGTACGCCCACAACCTGTACAGC
GGCGAAGTGAAGCTGATCGACGACTTCTACCCCAAAGTGG
GCAATGCCAGCGGAGCCGGCTACAGCTGTCTGAGCTACCG
GAAAAATGTGGACAAAGAAACCCTGTACGTGGTGTACGAG
GCCAACGGGAGCATCGAGTTTCAGGACCTGAGCAGACATC
TGCCCGTGATCAAGAGCTACAAC
SEQ ID NO: 7:
ENDFGLVQPLVTMEQLLWVSGRQIGSVDTFRIPLITATPR
GTLLAFAEARKMSSSDEGAKFIALRRSMDQGSTWSPTAFI
VNDGDVPDGLNLGAVVSDVETGVVFLFYSLCAHKAGCQVA
STMLVWSKDDGVSWSTPRNLSLDIGTEVFAPGPGSGIQKQ
REPRKGRLIVCGHGTLERDGVFCLLSDDHGASWRYGSGVS
GIPYGQPKQENDFNPDECQPYELPDGSVVINARNQNNYHC
HCRIVLRSYDACDTLRPRDVTFDPELVDPWAAGAVVTSSG
IVFFSNPAHPEFRVNLTLRVVSFSNGTSWRKETVQLWPGP
SGYSSLATLEGSMDGEEQAPQLYVLYEKGRNHYTESISVA
KISV
SEQ ID NO: 8:
MEEVTTCSFNSPLFRQEDDRGITYRIPALLYIPPTHTFLA
FAEKRSTRRDEDALHLVLRRGLRIGQLVQWGPLKPLMEAT
LPGHRTMNPCPVWEQKSGCVFLFFICVRGHVTERQQIVSG
RNAARLCFIYSQDAGCSWSEVRDLTEEVIGSELKHWATFA
VGPGHGIQLQSGRLVIPAYTYYIPSWFFCFQLPCKTRPHS
LMIYSDDLGVTWHHGRLIRPMVTVECEVAEVTGRAGHPVL
YCSARTPNRCRAEALSTDHGEGFQRLALSRQLCEPPHGCQ
GSVVSFRPLEIPHRCQDSSSKDAPTIQQSSPGSSLRLEEE
AGTPSESWLLYSHPTSRKQRVDLGIYLNQTPLEAACWSRP
WILHCGPCGYSDLAALEEEGLFGCLFECGTKQECEQIAFR
LFTHREILSHLQGDCTSPGRNPSQFKSN
SEQ ID NO: 9:
MRPADLPPRPMEESPASSSAPTETEEPGSSAEVMEEVTTC
SFNSPLFRQEDDRGITYRIPALLYIPPTHTFLAFAEKRST
RRDEDALHLVLRRGLRIGQLVQWGPLKPLMEATLPGHRTM
NPCPVWEQKSGCVFLFFICVRGHVTERQQIVSGRNAARLC
FIYSQDAG
CSWSEVRDLTEEVIGSELKHWATFAVGPGHGIQLQSGRLV
IPAYTYYIPSWFFCFQLPCKTRPHSLMIYSDDLGVTWHHG
RLIRPMVTVECEVAEVTGRAGHPVLYCSARTPNRCRAEAL
STDHGEGFQRLALSRQLCEPPHGCQGSVVSFRPLEIPHRC
QDSSSKDAPTIQQSSPGSSLRLEEEAGTPSESWLLYSHPT
SRKQRVDLGIYLNQTPLEAACWSRPWILHCGPCGYSDLAA
LEEEGLFGCLFECGTKQECEQIAFRLFTHREILSHLQGDC
TSPGRNPSQFKSN
SEQ ID NO: 10:
MGVPRTPSRTVLFERERTGLTYRVPSLLPVPPGPTLLAFV
EQRLSPDDSHAHRLVLRRGTLAGGSVRWGALHVLGTAALA
EHRSMNPCPVHDAGTGTVFLFFIAVLGHTPEAVQIATGRN
AARLCCVASRDAGLSWGSARDLTEEAIGGAVQDWATFAVG
PGHGVQLPSGRLLVPAYTYRVDRRECFGKICRTSPHSFAF
YSDDHGRTWRCGGLVPNLRSGECQLAAVDGGQAGSFLYCN
ARSPLGSRVQALSTDEGTSFLPAERVASLPETAWGCQGSI
VGFPAPAPNRPRDDSWSVGPGSPLQPPLLGPGVHEPPEEA
AVDPRGGQVPGGPFSRLQPRGDGPRQPGPRPGVSGDVGSW
TLALPMPFAAPPQSPTWLLYSHPVGRRARLHMGIRLSQSP
LDPRSWTEPWVIYEGPSGYSDLASIGPAPEGGLVFACLYE
SGARTSYDEISFCTFSLREVLENVPASPKPPNLGDKPRGC
CWPS
SEQ ID NO: 11:
MMSSAAFPRWLSMGVPRTPSRTVLFERERTGLTYRVPSLL
PVPPGPTLLAFVEQRLSPDDSHAHRLVLRRGTLAGGSVRW
GALHVLGTAALAEHRSMNPCPVHDAGTGTVFLFFIAVLGH
TPEAVQIATGRNAARLCCVASRDAGLSWGSARDLTEEAIG
GAVQDWATFAVGPGHGVQLPSGRLLVPAYTYRVDRRECFG
KICRTSPHSFAFYSDDHGRTWRCGGLVPNLRSGECQLAAV
DGGQAGSFLYCNARSPLGSRVQALSTDEGTSFLPAERVAS
LPETAWGCQGSIVGFPAPAPNRPRDDSWSVGPGSPLQPPL
LGPGVHEPPEEAAVDPRGGQVPGGPFSRLQPRGDGPRQPG
PRPGVSGDVGSWTLALPMPFAAPPQSPTWLLYSHPVGRRA
RLHMGIRLSQSPLDPRSWTEPWVIYEGPSGYSDLASIGPA
PEGGLVFACLYESGARTSYDEISFCTFSLREVLENVPASP
KPPNLGDKPRGCCWPS
SEQ ID NO: 12:
MASLP
SEQ ID NO: 13:
ASLP
SEQ ID NO: 14:
TVEKSWF
SEQ ID NO: 15:
GDYDAPTHQVQW
SEQ ID NO: 16:
SMDQGSTW
SEQ ID NO: 17:
STDGGKTW
SEQ ID NO: 18:
PRPPAPEA
SEQ ID NO: 19:
QTPLEAAC
SEQ ID NO: 20:
NPRPPAPEA
SEQ ID NO: 21:
SQNDGES
SEQ ID NO: 22:
LSHSLST
SEQ ID NO: 23:
GAGAACGACTTTGGACTGGTGCAGCCTCTGGTCACCATGG
AACAGCTGCTGTGGGTTTCCGGCAGACAGATCGGCAGCGT
GGACACCTTCAGAATCCCTCTGATCACCGCCACACCTAGA
GGCACCCTGCTGGCCTTTGCCGAGGCCAGAAAGATGAGCA
GCTCTGACGAGGGCGCCAAGTTTATTGCCCTGAGGCGGTC
TATGGACCAGGGCTCTACATGGTCCCCTACCGCCTTCATC
GTGAACGATGGCGACGTGCCCGATGGCCTGAATCTGGGAG
CTGTGGTGTCCGATGTGGAAACCGGCGTGGTGTTCCTGTT
CTACAGCCTGTGTGCCCACAAGGCCGGTTGTCAGGTGGCC
AGCACAATGCTCGTGTGGTCCAAGGACGACGGCGTGTCCT
GGTCTACCCCTAGAAACCTGAGCCTGGACATCGGCACCGA
AGTGTTTGCTCCAGGACCTGGCTCTGGCATCCAGAAGCAG
AGAGAGCCCAGAAAGGGCAGACTGATCGTGTGTGGCCACG
GCACCCTTGAGAGAGATGGCGTTTTCTGCCTGCTGAGCGA
CGATCATGGCGCCTCTTGGAGATACGGCAGCGGAGTGTCT
GGAATCCCTTACGGCCAGCCTAAGCAAGAGAACGATTTCA
ACCCCGACGAGTGCCAGCCTTACGAGCTGCCTGATGGCAG
CGTCGTGATCAACGCCCGGAACCAGAACAACTACCACTGC
CACTGCCGGATCGTGCTGAGAAGCTACGACGCCTGCGATA
CCCTGCGGCCTAGAGATGTGACCTTCGATCCTGAGCTGGT
GGACCCTGTTGTTGCCGCTGGTGCCGTCGTGACATCTAGC
GGCATCGTGTTCTTCAGCAACCCTGCTCACCCCGAGTTCA
GAGTGAATCTGACCCTGCGGTGGTCCTTCAGCAATGGCAC
AAGCTGGCGGAAAGAAACCGTGCAGCTTTGGCCTGGACCT
AGCGGCTACTCTTCTCTGGCTACACTGGAAGGCAGCATGG
ACGGCGAAGAACAGGCCCCTCAGCTGTACGTGCTGTACGA
GAAGGGCAGAAACCACTACACCGAGAGCATCAGCGTGGCC
AAGATCAGCGTT
SEQ ID NO: 24:
ATGGCCAGCCTGCCTGTGCTGCAGAAAGAAAGCGTGTTCC
AGTCTGGCGCCCACGCCTACAGAATTCCCGCTCTGCTGTA
TCTGCCAGGCCAGCAGTCTCTGCTGGCTTTCGCTGAACAG
CGGGCCAGCAAGAAGGATGAGCACGCCGAACTGATCGTGC
TGCGGAGAGGCGATTACGACGCCCCTACACATCAGGTGCA
GTGGCAGGCTCAAGAGGTGGTGGCTCAGGCTAGACTGGAC
GGCCACAGATCTATGAACCCCTGTCCTCTGTACGATGCCC
AGACCGGCACACTGTTTCTGTTCTTTATCGCTATCCCCGG
CCAAGTGACCGAGCAGCAGCAGCTGCAGACAAGAGCCAAC
GTGACCAGACTGTGTCAAGTGACCTCCACCGACCACGGCA
GAACCTGGTCTAGCCCTAGAGATCTGACCGACGCCGCCAT
CGGACCTGCCTATAGAGAGTGGTCCACCTTCGCCGTTGGA
CCTGGACACTGTCTCCAGCTGCACGACAGGGCTAGATCTC
TGGTGGTGCCTGCCTACGCCTATAGAAAGCTGCACCCCAT
CCAGCGGCCTATTCCTAGCGCCTTCTGCTTTCTGAGCCAC
GATCACGGCAGGACATGGGCCAGAGGACATTTCGTGGCCC
AGGACACACTGGAATGCCAGGTGGCCGAAGTGGAAACCGG
CGAGCAGAGAGTCGTGACCCTGAACGCCAGATCTCACCTG
AGAGCCAGAGTGCAGGCCCAGAGCACAAACGACGGCCTGG
ATTTCCAAGAGAGCCAGCTGGTCAAGAAACTGGTGGAACC
TCCTCCACAGGGCTGTCAGGGAAGCGTGATCAGCTTTCCA
TCTCCTAGAAGCGGCCCTGGCTCTCCTGCTCAGTGGCTGC
TGTATACACACCCCACACACAGCTGGCAGAGAGCCGATCT
GGGCGCCTACCTGAATCCTAGACCTCCTGCTCCTGAGGCT
TGGAGCGAACCTGTTCTGCTGGCCAAGGGCAGCTGTGCCT
ACAGCGATCTGCAGTCTATGGGCACAGGCCCTGATGGCAG
CCCTCTGTTTGGCTGTCTGTACGAGGCCAACGACTACGAA
GAGATCGTGTTCCTGATGTTCACCCTGAAGCAGGCCTTTC
CAGCCGAGTACCTGCCTCAA
SEQ ID NO: 25:
ATGGAGGAAGTGACCACCTGTAGCTTCAACAGCCCTCTGT
TCCGGCAAGAGGACGACCGGGGCATCACCTACAGAATCCC
TGCTCTGCTGTACATCCCTCCTACACACACCTTTCTGGCC
TTCGCCGAGAAGCGGAGCACCAGACGAGATGAAGATGCCC
TGCACCTGGTGCTGAGAAGAGGCCTGAGAATCGGACAGCT
GGTGCAGTGGGGACCTCTGAAGCCTCTGATGGAAGCCACA
CTGCCCGGCCACAGAACCATGAATCCTTGTCCTGTGTGGG
AGCAGAAAAGCGGCTGCGTGTTCCTGTTCTTCATCTGCGT
GCGGGGCCACGTGACCGAGAGACAGCAAATCGTGTCCGGC
AGAAACGCCGCCAGACTGTGCTTCATCTACAGCCAGGATG
CCGGCTGCTCTTGGAGCGAAGTTCGGGATCTGACCGAAGA
AGTGATCGGCAGCGAGCTGAAGCACTGGGCCACATTTGCT
GTTGGCCCTGGCCACGGAATCCAGCTGCAATCTGGCAGAC
TGGTCATCCCCGCCTACACCTACTATATCCCCAGCTGGTT
CTTCTGCTTCCAACTGCCTTGCAAGACCCGGCCTCACAGC
CTGATGATCTACAGCGACGATCTGGGCGTGACATGGCACC
ACGGCAGACTGATCAGACCCATGGTCACCGTGGAATGCGA
GGTGGCCGAAGTGACAGGCAGAGCTGGACACCCTGTGCTG
TACTGCTCTGCCAGAACACCCAACCGGTGTAGAGCCGAGG
CTCTGTCTACAGATCACGGCGAGGGCTTTCAGAGACTGGC
CCTCTCTAGACAGCTGTGCGAACCTCCTCATGGCTGTCAG
GGCAGCGTGGTGTCCTTCAGACCTCTGGAAATCCCTCACC
GGTGCCAGGACAGCAGCTCTAAGGATGCCCCTACCATCCA
GCAGTCTAGCCCTGGCAGCAGCCTGAGACTGGAAGAGGAA
GCCGGAACACCTAGCGAGAGCTGGCTGCTGTACTCTCACC
CCACCAGCAGAAAGCAGAGAGTGGACCTGGGCATCTACCT
GAATCAGACCCCTCTGGAAGCCGCCTGTTGGAGCAGACCT
TGGATTCTGCACTGTGGCCCTTGCGGCTACTCTGATCTGG
CCGCTCTGGAAGAAGAGGGCCTGTTCGGCTGCCTGTTTGA
GTGCGGCACAAAGCAAGAGTGCGAGCAGATCGCCTTCCGG
CTGTTCACCCACAGAGAGATCCTGAGCCATCTGCAGGGCG
ACTGCACAAGCCCAGGCAGAAATCCCAGCCAGTTCAAGAG
CAAC
SEQ ID NO: 26:
ATGGGCGTGCCCAGAACACCCAGCAGAACCGTGCTGTTCG
AGAGAGAGAGGACCGGCCTGACCTACAGAGTGCCTTCTCT
GCTGCCTGTGCCTCCTGGACCTACACTGCTGGCCTTCGTG
GAACAGAGACTGAGCCCCGATGATTCTCACGCCCACAGAC
TGGTGCTGAGAAGAGGAACACTGGCTGGCGGCTCTGTTAG
ATGGGGAGCACTGCATGTGCTGGGCACAGCTGCTCTTGCC
GAGCACAGATCCATGAATCCCTGTCCTGTGCACGACGCCG
GAACCGGCACAGTGTTTCTGTTCTTTATCGCCGTGCTGGG
CCACACACCTGAGGCCGTTCAAATTGCCACCGGCAGAAAT
GCCGCCAGACTGTGTTGTGTGGCCTCCAGAGATGCCGGCC
TGTCTTGGGGATCTGCCAGAGATCTGACCGAGGAAGCCAT
TGGCGGAGCCGTTCAGGATTGGGCCACATTTGCTGTTGGA
CCTGGACACGGCGTGCAGCTGCCAAGTGGTAGACTGCTGG
TGCCTGCCTACACATACAGAGTGGATCGGAGAGAGTGCTT
CGGAAAGATCTGCCGGACAAGCCCTCACAGCTTCGCCTTC
TACTCCGACGATCACGGCCGGACTTGGAGATGTGGTGGCC
TGGTGCCTAATCTGAGAAGCGGCGAATGTCAACTGGCCGC
CGTTGATGGTGGACAGGCTGGCAGCTTCCTGTACTGCAAC
GCCAGATCTCCTCTGGGCTCTAGAGTGCAGGCCCTGTCTA
CCGATGAGGGCACCAGTTTTCTGCCCGCCGAAAGAGTTGC
CTCTCTGCCTGAAACAGCCTGGGGCTGTCAGGGCTCTATC
GTGGGATTTCCTGCTCCTGCTCCAAACAGACCCCGGGACG
ATTCTTGGAGTGTCGGCCCTGGATCTCCACTGCAGCCTCC
ATTGCTTGGACCAGGCGTTCACGAGCCACCTGAAGAGGCT
GCCGTTGATCCTAGAGGCGGACAAGTTCCTGGCGGCCCTT
TTAGCAGACTGCAGCCAAGAGGCGACGGCCCTAGACAACC
TGGACCAAGACCTGGCGTCAGCGGAGATGTTGGCTCTTGG
ACACTGGCCCTGCCTATGCCTTTTGCCGCTCCTCCTCAGT
CTCCTACCTGGCTGCTGTACTCTCACCCTGTTGGCAGACG
GGCCAGACTGCACATGGGCATCAGACTGTCTCAGAGCCCT
CTGGACCCCAGAAGCTGGACAGAGCCTTGGGTCATCTATG
AGGGCCCTAGCGGCTACAGCGATCTGGCCTCTATTGGCCC
AGCTCCTGAAGGCGGACTGGTGTTCGCTTGTCTGTATGAG
AGCGGCGCCAGAACCAGCTACGACGAGATCAGCTTCTGCA
CCTTCAGCCTGCGCGAGGTGCTGGAAAATGTGCCCGCCTC
TCCTAAGCCTCCTAACCTGGGCGATAAGCCTAGAGGCTGT
TGCTGGCCATCT
SEQ ID NO: 27:
MTGERPSTALPDRRWGPRILGFWGGCRVWVFAAIFLLLSL
AASWSKA
SEQ ID NO: 28:
MDMRVPAQLLGLLLLWLPGARC
SEQ ID NO: 29:
YGTL
SEQ ID NO: 30:
MTVEKSVVFKAEGEHFTDQKGNTIVGSGSGGTTKYFRIPA
MCTTSKGTIVVFADARHNTASDOSFIDTAAARSTDGGKTW
NKKIAIYNDRVNSKLSRVMDPTCIVANIQGRETILVMVGK
WNNNDKTWGAYRDKAPDTDWDLVLYKSTDDGVTFSKVETN
IHDIVTKNGTISAMLGGVGSGLQLNDGKLVFPVQMVRTKN
ITTVLNTSFIYSTDGITWSLPSGYCEGFGSENNIIEFNAS
LVNNIRNSGLRRSFETKDFGKTWTEFPPMDKKVDNRNHGV
QGSTITIPSGNKLVAAHSSAQNKNNDYTRSDISLYAHNLY
SGEVKLIDDFYPKVGNASGAGYSCLSYRKNVDKETLYVVY
EANGSIEFQDLSRHLPVIKSYN
SEQ ID NO: 31:
EPKSCDKTHTCPPCPAPELLGGPSVFLFPPKPKDTLMISR
TPEVTCVVVDVSHEDPEVKFNWYVDGVEVHNAKTKPREEQ
YNSTYRVVSVLTVLHQDWLNGKEYKCKVSNKALPAPIEKT
ISKAKGQPREPQVYTLPPSREEMTKNQVSLTCLVKGFYPS
DIAVEWESNGQPENNYKTTPPVLDSDGSFFLYSKLTVDKS
RWQQGNVFSCSVMHEALHNHYTQKSLSLSPGK
SEQ ID NO: 32:
DKTHTCPPCPAPELLGGPSVFLFPPKPKDTLMISRTPEVT
CVVVDVSHEDPEVKFNWYVDGVEVHNAKTKPREEQYNSTY
RVVSVLTVLHQDWLNGKEYKCKVSNKALPAPIEKTISKAK
GQPREPQVYTLPPSREEMTKNQVSLTCLVKGFYPSDIAVE
WESNGQPENNYKTTPPVLDSDGSFFLTSKLTVDKSRWQQG
NVFSCSVMHEALHNHYTQKSLSLSPGK
SEQ ID NO: 33:
EPKSCDKTHTCPPCPAPELLGGPSVFLFPPKPKDTLMISR
TPEVTCVVVDVSHEDPEVKFNWYVDGVEVHNAKTKPREEQ
YNSTYRVVSVLTVLHQDWLNGKEYKCKVSNKALPAPIEKT
ISKAKGQPREPQVYTLPPSREEMTKNQVSLYCLVKGFYPS
DIAVEWESNGQPENNYKTTPPVLDSDGSFFLYSKLTVDKS
RWQQGNVFSCSVMHEALHNHYTQKSLSLSPGK
SEQ ID NO: 34:
ATGAGACCTGCGGACCTGCCCCCGCGCCCCATGGAAGAAT
CCCCGGCGTCCAGCTCTGCCCCGACAGAGACGGAGGAGCC
GGGGTCCAGTGCAGAGGTCATGGAAGAAGTGACAACATGC
TCCTTCAACAGCCCTCTGTTCCGGCAGGAAGATGACAGAG
GGATTACCTACCGGATCCCAGCCCTGCTCTACATACCCCC
CACCCACACCTTCCTGGCCTTTGCAGAGAAGCGTTCTACG
AGGAGAGATGAGGATGCTCTCCACCTGGTGCTGAGGCGAG
GGTTGAGGATTGGGCAGTTGGTACAGTGGGGGCCCCTGAA
GCCACTGATGGAAGCCACACTACCGGGGCATCGGACCATG
AACCCCTGTCCTGTATGGGAGCAGAAGAGTGGTTGTGTGT
TCCTGTTCTTCATCTGTGTGCGGGGCCATGTCACAGAGCG
TCAACAGATTGTGTCAGGCAGGAATGCTGCCCGCCTTTGC
TTCATCTACAGTCAGGATGCTGGATGTTCATGGAGTGAGG
TGAGGGACTTGACTGAGGAGGTCATTGGCTCAGAGCTGAA
GCACTGGGCCACATTTGCTGTGGGCCCAGGTCATGGCATC
CAGCTGCAGTCAGGGAGACTGGTCATCCCTGCGTATACCT
ACTACATCCCTTCCTGGTTCTTTTGCTTCCAGCTACCATG
TAAAACCAGGCCTCATTCTCTGATGATCTACAGTGATGAC
CTAGGGGTCACATGGCACCATGGTAGACTCATTAGGCCCA
TGGTTACAGTAGAATGTGAAGTGGCAGAGGTGACTGGGAG
GGCTGGCCACCCTGTGCTATATTGCAGTGCCCGGACACCA
AACAGGTGCCGGGCAGAGGCGCTCAGCACTGACCATGGTG
AAGGCTTTCAGAGACTGGCCCTGAGTCGACAGCTCTGTGA
GCCCCCACATGGTTGCCAAGGGAGTGTGGTAAGTTTCCGG
CCCCTGGAGATCCCACATAGGTGCCAGGACTCTAGCAGCA
AAGATGCACCCACCATTCAGCAGAGCTCTCCAGGCAGTTC
ACTGAGGCTGGAGGAGGAAGCTGGAACACCGTCAGAATCA
TGGCTCTTGTACTCACACCCAACCAGTAGGAAACAGAGGG
TTGACCTAGGTATCTATCTCAACCAGACCCCCTTGGAGGC
TGCCTGCTGGTCCCGCCCCTGGATCTTGCACTGTGGGCCC
TGTGGCTACTCTGATCTGGCTGCTCTGGAGGAGGAGGGCT
TGTTTGGGTGTTTGTTTGAATGTGGGACCAAGCAAGAGTG
TGAGCAGATTGCCTTCCGCCTGTTTACACACCGGGAGATC
CTGAGTCACCTGCAGGGGGACTGCACCAGCCCTGGTAGGA
ACCCAAGCCAATTCAAAAGCAAT
SEQ ID NO: 35:
ATGATGAGCTCTGCAGCCTTCCCAAGGTGGCTGAGCATGG
GGGTCCCTCGTACCCCTTCACGGACAGTGCTCTTCGAGCG
GGAGAGGACGGGCCTGACCTACCGCGTGCCCTCGCTGCTC
CCCGTGCCCCCCGGGCCCACCCTGCTGGCCTTTGTGGAGC
AGCGGCTCAGCCCTGACGACTCCCACGCCCACCGCCTGGT
GCTGAGGAGGGGCACGCTGGCCGGGGGCTCCGTGCGGTGG
GGTGCCCTGCACGTGCTGGGGACAGCAGCCCTGGCGGAGC
ACCGGTCCATGAACCCCTGCCCTGTGCACGATGCTGGCAC
GGGCACCGTCTTCCTCTTCTTCATCGCGGTGCTGGGCCAC
ACGCCTGAGGCCGTGCAGATCGCCACGGGAAGGAACGCCG
CGCGCCTCTGCTGTGTGGCCAGCCGTGACGCCGGCCTCTC
GTGGGGCAGCGCCCGGGACCTCACCGAGGAGGCCATCGGT
GGTGCCGTGCAGGACTGGGCCACATTCGCTGTGGGTCCCG
GCCACGGTGTGCAGCTGCCCTCAGGCCGCCTGCTGGTACC
CGCCTACACCTACCGCGTGGACCGCCGAGAGTGTTTTGGC
AAGATCTGCCGGACCAGCCCTCACTCCTTCGCCTTCTACA
GCGATGACCACGGCCGCACCTGGCGCTGTGGAGGCCTCGT
GCCCAACCTGCGCTCAGGCGAGTGCCAGCTGGCAGCGGTG
GACGGTGGGCAGGCCGGCAGCTTCCTCTACTGCAATGCCC
GGAGCCCACTGGGCAGCCGTGTGCAGGCGCTCAGCACTGA
CGAGGGCACCTCCTTCCTGCCCGCAGAGCGCGTGGCTTCC
CTGCCCGAGACTGCCTGGGGCTGCCAGGGCAGCATCGTGG
GCTTCCCAGCCCCCGCCCCCAACAGGCCACGGGATGACAG
TTGGTCAGTGGGCCCCGGGAGTCCCCTCCAGCCTCCACTC
CTCGGTCCTGGAGTCCACGAACCCCCAGAGGAGGCTGCTG
TAGACCCCCGTGGAGGCCAGGTGCCTGGTGGGCCCTTCAG
CCGTCTGCAGCCTCGGGGGGATGGCCCCAGGCAGCCTGGC
CCCAGGCCTGGGGTCAGTGGGGATGTGGGGTCCTGGACCC
TGGCACTCCCCATGCCCTTTGCTGCCCCGCCCCAGAGCCC
CACGTGGCTGCTGTACTCCCACCCAGTGGGGCGCAGGGCT
CGGCTACACATGGGTATCCGCCTGAGCCAGTCCCCGCTGG
ACCCGCGCAGCTGGACAGAGCCCTGGGTGATCTACGAGGG
CCCCAGCGGCTACTCCGACCTGGCGTCCATCGGGCCGGCC
CCTGAGGGGGGCCTGGTTTTTGCCTGCCTGTACGAGAGCG
GGGCCAGGACCTCCTATGATGAGATTTCCTTTTGTACATT
CTCCCTGCGTGAGGTCCTGGAGAACGTGCCCGCCAGCCCC
AAACCGCCCAACCTTGGGGACAAGCCTCGGGGGTGCTGCT
GGCCCTCC
SEQ ID NO: 36:
MRFKNVKKTALMLAMFGMATSSNAALFDYNATGDTEFDSP
AKQGWMQDNTNNGSGVLTNADGMPAWLVQGIGGRAQWTYS
LSTNQHAQASSFGWRMTTEMKVLSGGMITNYYANGTQRVL
PIISLDSSGNLVVEFEGQTGRTVLATGTAATEYHKFELVF
LPGSNPSASFYFDGKLIRDNIQPTASKQNMIVWGNGSSNT
DGVAAYRDIKFEIQGDVIFRGPDRIPSIVASSVTPGVVTA
FAEKRVGGGDPGALSNTNDIITRTSRDGGITWDTELNLTE
QINVSDEFDFSDPRPIYDPSSNTVLVSYARWPTDAAQNGD
RIKPWMPNGIFYSVYDVASGNWQAPIDVTDQVKERSFQIA
GWGGSELYRRNTSLNSQQDWQSNAKIRIVDGAANQIQVAD
GSRKYVVTLSIDESGGLVANLNGVSAPIILQSEHAKVHSF
HDYELQYSALNHTTTLFVDGQQITTWAGEVSQENNIQFGN
ADAQIDGRLHVQKIVLTQQGHNLVEFDAFYLAQQTPEVEK
DLEKLGWTKIKTGNTMSLYGNASVNPGPGHGITLTRQQNI
SGSQNGRLIYPAIVLDRFFLNVMSIYSDDGGSNWQTGSTL
PIPFRWKSSSILETLEPSEADMVELQNGDLLLTARLDFNQ
IVNGVNYSPRQQFLSKDGGITWSLLEANNANVFSNISTGT
VDASITRFEQSDGSHFLLFTNPQGNPAGTNGRQNLGLWFS
FDEGVTWKGPIQLVNGASAYSDIYQLDSENAIVIVETDNS
NMRILRMPITLLKQKLTLSQN
SEQ ID NO: 37:
TTGTCAATCAAGATGAGTTCACAACGAAGAAGAGCATCGA
TTCACAAGGAAACAGATTCTAATATAAAGGGAGTAGATAT
GCGTTTCAAAAACGTAAAGAAAACCGCTTTAATGCTTGCA
ATGTTCGGTATGGCGACAAGCTCAAACGCCGCACTTTTTG
ACTATAACGCAACGGGTGACACTGAGTTTGACAGTCCAGC
CAAACAGGGATGGATGCAAGACAACACGAATAATGGCAGC
GGCGTTTTAACCAATGCAGATGGAATGCCCGCTTGGTTGG
TGCAAGGTATTGGAGGGAGAGCTCAATGGACATATTCTCT
CTCTACTAATCAACATGCCCAAGCATCAAGTTTCGGTTGG
CGAATGACGACAGAAATGAAAGTGCTCAGTGGTGGAATGA
TCACAAACTACTACGCCAACGGCACTCAGCGTGTCTTACC
CATCATTTCATTAGATAGCAGTGGTAACTTAGTTGTTGAG
TTTGAAGGGCAAACTGGACGCACCGTTTTGGCAACCGGCA
CAGCAGCAACGGAATATCATAAATTTGAATTGGTATTCCT
TCCTGGAAGTAACCCATCCGCTAGCTTTTACTTCGATGGC
AAACTCATTCGTGACAACATCCAGCCGACTGCATCAAAAC
AAAATATGATCGTATGGGGGAATGGCTCATCAAATACGGA
TGGTGTCGCCGCTTATCGTGATATTAAGTTTGAAATTCAA
GGCGACGTCATCTTCAGAGGCCCAGACCGTATACCGTCCA
TTGTAGCAAGTAGCGTAACACCAGGGGTGGTAACCGCATT
TGCAGAGAAACGTGTGGGGGGAGGAGATCCCGGTGCTCTG
AGTAATACCAATGACATAATCACTCGTACCTCACGAGATG
GCGGTATAACTTGGGATACCGAGCTCAACCTCACTGAGCA
AATCAATGTCAGTGATGAGTTTGATTTCTCCGATCCTCGG
CCTATCTATGATCCTTCCTCCAATACGGTTCTTGTCTCTT
ATGCTCGATGGCCGACCGATGCCGCTCAAAACGGAGATCG
AATAAAACCATGGATGCCAAACGGTATTTTTTACAGCGTC
TATGATGTTGCATCAGGGAACTGGCAAGCGCCTATCGATG
TTACCGATCAGGTGAAAGAACGCAGTTTCCAAATCGCTGG
TTGGGGTGGTTCAGAGCTGTATCGCCGAAATACCAGCCTA
AATAGCCAGCAAGACTGGCAATCAAACGCTAAGATCCGAA
TTGTTGATGGTGCAGCGAACCAGATACAAGTTGCCGATGG
TAGCCGAAAATATGTTGTCACACTGAGTATTGATGAATCA
GGTGGTCTAGTCGCTAATCTAAACGGTGTTAGTGCTCCGA
TTATCCTGCAATCTGAACACGCAAAGGTACACTCTTTCCA
TGACTACGAACTTCAATATTCGGCGTTAAACCACACCACA
ACGTTATTCGTGGATGGTCAGCAAATCACAACTTGGGCTG
GCGAAGTATCGCAGGAGAACAACATTCAGTTTGGTAATGC
GGATGCCCAAATTGACGGCAGACTGCATGTGCAAAAAATT
GTTCTCACACAGCAAGGCCATAACCTCGTGGAGTTTGATG
CTTTCTATTTAGCACAGCAAACCCCTGAAGTAGAGAAAGA
CCTTGAAAAGCTTGGTTGGACAAAAATTAAAACGGGCAAC
ACCATGAGTTTGTATGGAAATGCCAGTGTCAACCCAGGAC
CGGGTCATGGCATCACCCTTACTCGACAACAAAATATCAG
TGGCAGCCAAAACGGCCGCTTGATCTACCCAGCGATTGTG
CTTGATCGTTTCTTCTTGAACGTCATGTCTATTTACAGTG
ATGATGGCGGTTCAAACTGGCAAACCGGTTCAACACTCCC
TATCCCCTTTCGCTGGAAGAGTTCGAGTATCCTAGAAACT
CTCGAACCTAGTGAAGCTGATATGGTTGAACTCCAAAACG
GTGATCTACTCCTTACTGCACGCCTTGATTTTAACCAAAT
CGTTAATGGTGTGAACTATAGCCCACGCCAGCAATTTTTG
AGTAAAGATGGTGGAATCACGTGGAGCCTACTTGAGGCTA
ACAACGCTAACGTCTTTAGCAATATCAGTACTGGTACCGT
TGATGCTTCTATTACTCGGTTCGAGCAAAGTGACGGTAGC
CATTTCTTACTCTTTACTAACCCACAAGGAAACCCTGCGG
GGACAAATGGCAGGCAAAATCTAGGCTTATGGTTTAGCTT
CGATGAAGGGGTGACATGGAAAGGACCAATTCAACTTGTT
AATGGTGCATCGGCATATTCTGATATTTATCAATTGGATT
CGGAAAATGCGATTGTCATTGTTGAAACGGATAATTCAAA
TATGCGAATTCTTCGTATGCCTATCACATTGCTAAAACAG
AAGCTGACCTTATCGCAAAACTAA
SEQ ID NO: 38:
MVGADPTRPRGPLSYWAGRRGQGLAAIFLLLVSAAESEAR
AEDDFSLVQPLVTMEQLLWVSGKQIGSVDTFRIPLITATP
RGTLLAFAEARKKSASDEGAKFIAMRRSTDQGSTWSSTAF
IVDDGEASDGLNLGAVVNDVDTGIVFLIYTLCAHKVNCQV
ASTMLVWSKDDGISWSPPRNLSVDIGTEMFAPGPGSGIQK
QREPGKGRLIVCGHGTLERDGVFCLLSDDHGASWHYGTGV
SGIPFGQPKHDHDFNPDECQPYELPDGSVIINARNQNNYH
CRCRIVLRSYDACDTLRPRDVTFDPELVDPVVAAGALATS
SGIVFFSNPAHPEFRVNLTLRWSFSNGTSWLKERVQVWPG
PSGYSSLTALENSTDGKKQPPQLFVLYEKGLNRYTESISM
VKISVYGTL
SEQ ID NO: 39:
MTVQPSPWFSDLRPMATCPVLQKETLFRTGVHAYRIPALL
YLKKQKTLLAFAEKRASKTDEHAELIVLRRGSYNEATNRV
KWQPEEVVTQAQLEGHRSMNPCPLYDKQTKTLFLFFIAVP
GRVSEHHQLHTKVNVTRLCCVSSTDHGRTWSPIQDLTETT
IGSTHQEVVATFAVGPGHCLQLRNPAGSLLVPAYAYRKLH
PAQKPTPFAFCEISLDHGHTWKLGNEVAENSLECQVAEVG
TGAQRMVYLNARSFLGARVQAQSPNDGLDFQDNRVVSKLV
EPPHGCHGSVVAFHNPISKPHALDTWLLYTHPTDSRNRTN
LGVYLNQMPLDPTAWSEPTLLAMGICAYSDLQNMGQGPDG
SPQFGCLYESGNYEEIIFLIFTLKQAFPTVEDAQ
SEQ ID NO: 40:
MEEVPPYSLSSTLFQQEEQSGVTYRIPALLYLPPTHTFLA
FAEKRTSVRDEDAACLVLRRGLMKGRSVQWGPQRLLMEAT
LPGHRTMNPCPVWEKNTGRVYLFFICVRGHVTERCQIVWG
KNAARLCFLCSEDAGCSWGEVKDLTEEVIGSEVKRWATFA
VGPGHGIQLHSGRLIIPAYAYYVSRWFLCFACSVKPHSLM
IYSDDFGVTWHHGKFIEPQVTGECQVAEVAGTAGNPVLYC
SARTPSRFRAEAFSTDSGGCFQKPTLNPQLHEPRTGCQGS
VVSFRPLKMPNTYQDSIGKGAPATQKCPLLDSPLEVEKGA
ETPSATWLLYSHPTSKRKRINLGIYYNRNPLEVNCWSRPW
ILNRGPSGYSDLAVVEEQDLVACLFECGEKNEYERIDFCL
FSDHEVLSCEDCTSPSSD
SEQ ID NO: 41:
METAGAPFCFHVDSLVPCSYWKVMGPTRVPRRTVLFQRER
TGLTYRVPALLCVPPRPTLLAFAEQRLSPDDSHAHRLVLR
RGTLTRGSVRWGTLSVLETAVLEEHRSMNPCPVLDEHSGT
IFLFFIAVLGHTPEAVQIATGKNAARLCCVTSCDAGLTWG
SVRDLTEEAIGAALQDWATFAVGPGHGVQLRSGRLLVPAY
TYHVDRRECFGKICWTSPHSLAFYSDDHGISWHCGGLVPN
LRSGECQLAAVDGDFLYCNARSPLGNRVQALSADEGTSFL
PGELVPTLAETARGCQGSIVGFLAPPSIEPQDDRWTGSPR
NTPHSPCFNLRVQESSGEGARGLLERWMPRLPLCYPQSRS
PENHGLEPGSDGDKTSWTPECPMSSDSMLQSPTWLLYSHP
AGRRARLHMGIYLSRSPLDPHSWTEPWVIYEGPSGYSDLA
FLGPMPGASLVFACLFESGTRTSYEDISFCLFSLADVLEN
VPTGLEMLSLRDKAQGHCWPS
SEQ ID NO: 42:
GGGTCACATGCTGATGGACTAATTGGAGTCGCGGCAGCGC
GGGCTGCGGCCCCCAAGGGGAGGGGTCGGAGTGACGTGCG
CGCTTTTAAAGGGCCGAGGTCAGCTGACGGCTTGCCACCG
GTGACCAGTTCCTGGACAGGGATCGCCGGGAGCTATGGTG
GGGGCAGACCCGACCAGACCCCGGGGACCGCTGAGCTATT
GGGCGGGCCGTCGGGGTCAGGGGCTCGCAGCGATCTTCCT
GCTCCTGGTGTCCGCGGCGGAATCCGAGGCCAGGGCAGAG
GATGACTTCAGCCTGGTGCAGCCGCTGGTGACCATGGAGC
AGCTGCTGTGGGTGAGCGGGAAGCAGATCGGCTCTGTAGA
CACTTTCCGCATCCCGCTCATCACAGCCACCCCTCGGGGC
ACGCTCCTGGCCTTCGCTGAGGCCAGGAAAAAATCTGCAT
CCGATGAGGGGGCCAAGTTCATCGCCATGAGGAGGTCCAC
GGACCAGGGTAGCACGTGGTCCTCTACAGCCTTCATCGTA
GACGATGGGGAGGCCTCCGATGGCCTGAACCTGGGCGCTG
TGGTGAACGATGTAGACACAGGGATAGTGTTCCTTATCTA
TACCCTCTGTGCTCACAAGGTCAACTGCCAGGTGGCCTCT
ACCATGTTGGTTTGGAGTAAGGACGACGGCATTTCCTGGA
GCCCACCCCGGAATCTCTCTGTGGATATTGGCACAGAGAT
GTTTGCCCCTGGACCTGGCTCAGGCATTCAGAAACAGCGG
GAGCCTGGGAAGGGCCGGCTCATTGTGTGTGGACACGGGA
CGCTGGAGCGAGATGGGGTCTTCTGTCTCCTCAGTGATGA
CCACGGTGCCTCCTGGCACTACGGCACTGGAGTGAGCGGC
ATTCCCTTTGGCCAGCCCAAACACGATCACGATTTCAACC
CCGACGAGTGCCAGCCCTACGAGCTTCCAGATGGCTCGGT
CATCATCAACGCCCGGAACCAGAATAACTACCATTGCCGC
TGCAGGATCGTCCTCCGCAGCTATGACGCCTGTGACACCC
TCAGGCCCCGGGATGTGACCTTCGACCCTGAGCTCGTGGA
CCCTGTGGTAGCTGCAGGAGCACTAGCCACCAGCTCCGGC
ATTGTCTTCTTCTCCAATCCAGCCCACCCTGAGTTCCGAG
TGAACCTGACCCTGCGCTGGAGTTTCAGCAATGGTACATC
CTGGCAGAAGGAGAGGGTCCAGGTGTGGCCGGGACCCAGC
GGCTACTCGTCCCTGACAGCCCTGGAAAACAGCACGGATG
GAAAGAAGCAGCCCCCGCAGCTGTTCGTTCTGTACGAGAA
AGGCCTGAACCGGTACACCGAGAGCATCTCCATGGTCAAA
ATCAGCGTCTACGGCACGCTCTGAGCCCCGTGCCCAAAGG
ACACCAAGTCCTGGTCGCTGACTTCACAGCTCTCTGGACC
ATCTGCAGAGGGTGCCTGAAACACAGCTCTTCCTCTGAAC
TCTGACCTTTTGCAACTTCTCATCAACAGGGAAGTCTCTT
CGTTATGACTTAACACCCAGCTTCCTCTCGGGGCAGGAAG
TCCCTCCGTCACCAAGAGCACTTTTTTCCAGTATGCTGGG
GATGGCCCCTGTCCATTCTCTTCCAGGACAACGGAGCTGT
GCCTTTCTGGGACAGGATGGGGGAGGGGCTCCCCCTGGAG
AGATGAACAGATACGAACTCAGGGAACTGAGAAGGCCCGG
TGTCCTAGGGTACAAAGGCAGGTACTAGATGTGATTGCTG
AAAGTCCCCAGGGCAGAGTGTCCTTTCAGAGCAAGGATAA
GCACACCTAGGTGTGCACCTTTGATTATTTATGAATCGAA
ATATTTGTAACTTAAAATTTTTGATGCAGAAAAAGCGTTT
GTGGAGTCTGTGGTTCTGTCTGCTCACGCCTTCCCAATTG
CCTCCTGGAGAGACAGGAAGGCAGCTGGAAGAGGAGCCGA
TGTACTTACTGGGAAGCAGAAACCCCTAGATTCCATCCTG
GCTGCTGCTGTTTGCAAGTGTCAAAGATGGGGGGGCGTGT
TTATATTTTATATTTCTAAGATGGGGTGGCATAGGAAATA
GGGAACAGATGTGTAAAACCAGATGGGAAGGACAGTCTGT
GAGAAAGGAGCAAGCAGTTGCTGCAGGTGTGGGAGAGCAA
AGCCCTTCTCCACGTGGAAAGAGCCCAGATGGACGCTAAG
CATGTTGGGCACCTGTAACCCCGCACTCGCTGGACTGACG
GTGTAGCTCAGTGGTGGAGCTAGTACTTGGAACGCCTAAG
ACTCTGGGTTCAGTCCTTGGGGGGGGGGGTATGTGTTTAT
TGAGAGGAAGGTGTACGTACTGTAGGTCAGAGGACAGCTT
ACTGGAGTTGTCTCTCTCCTTCACGCTGTGAGTCCTGTGG
AATGACCTCAGGTGTCAGAGTTGGGGGCAGGTGCCTTTGC
CAGCTGAGCCATCTTGCTGTCTCTGCTTTAATTTAAAAAA
AAAAAAAAAAAAGAATATTAAGGTCTGAGGGATTCGGGCT
GCGTTCATTTCAATTAGAGGGTCATATTTCTTTTGACATT
TCTTCTCTAAGAAATGTTAAGATCATTTGTTCTGTGTGAT
AGAGGTATAGCTCCATTGTATGTCAGCAGTGAGGGATCCT
GTGCATTTTATCCAGAGTTTGTACGGTGTTCTAGGGGCTG
CTAGTGCAGCCCAGTGCTAAACACTTCAGCATGCACAAGG
CCTCAATCAGTGCATGCATGTGCACACACACACAGACACA
CACGTAGACACTGACACAGGTACACAAATACACACTGGCC
CACATGTACACATCGACTCACAGGTACACAGACCCACTTT
GACACACATATACACAGACACAAACGCACTGGCACACACA
TATACACAGGCACACATGGATAGATGGACACACGTGTACA
CATACACACACACACAGAAATACAAATGTTCAGGTTTTCT
AAAAAAAAAAAAATTAGAGACGTGTTGACTTCATTTTTAG
CAAAAATCCTGTCATGTATCTTAAAGTGGATTGAACCCAC
TATGTAGCCCAGGCTGGCCTCCAAATGGGCATCCTTCTGC
CTCAGTCTCCCGAGGGCTAGGATAACAGGAGTATGCCATC
ACACCTGGCTAATAGAAATTTTCAAAATTGTTTGTTTGAA
GGTGACTCTTACTATATTGCCTAACTGATCTCCAGTTCGT
GAAATCCTCCTGCCTCAGAACCAGGACTGTCAATATAACC
CACCAAGACAGGCCAACATTCACAATTGATTGTTAGTTTG
TGGTCTGAATCAAGGTCTTATACTGTAGCCCAGGCTAGCC
CGGAATACACGATATCTCCAGTGCTTCAGATCCTCAGTTC
TAACTAAGCATGGCCACATCCATGTTTAACTGCAAATTTG
ATGTTACCATGGTTTGGTTTGGTTTGGTTTGGTTTGGTTT
GGTTTGGTTTGGTTTTTTGGCCATTTTTTTTTTCTCATGC
TGAGGCCTTGTGCTCTCAAGTTGGGGAGACAGCATGGAGG
GTAGCTGCAACTGTAACCCCAGTTCCAGGGGACCTGACAC
CCTCTGGCCTCCACAAGTATTAGGCACATCTGTGGTGCAC
AGACATACAATCAGGCAAAATATTCATACACATAAAATAA
AATAATTTAAAACAAAAGCAAAAATCAGGACCTAAGAAAA
AAATCTATTCCTGATTCTTTTATGTTTTGTTTGTATTTTA
TCAAGACAGGGTTGTTTCTCTGTATAGCCCTGGCTGTCTT
GGAATTCACTCTGTAGACCAGGCTGGCCTCAAACTCAGAA
ATCCTCCTGCCTTTGCCTTCCAAGTGCTGGAATTAAAGGC
ATGCGCCACC
SEQ ID NO: 43:
GACATGACCCAAACGGCCCCTGGCTGCAAGGTAATATCGG
AAGTTGACTAAGAATGGACGCCCCACCACTGACTGACCCG
CCCCCTGAGTCTGAGATTGGACTTGTCTCTGGATACAGTC
ATACTTTGAGGTACTAGAAGTTAGAAACTGTTAGGTTACT
CAGTTCAGTCCATGACAGTCCAACCTTCTCCATGGTTTTC
CGATCTCAGGCCCATGGCGACCTGCCCTGTCCTGCAGAAG
GAGACACTGTTCCGCACAGGCGTCCATGCTTACAGAATCC
CTGCTCTGCTCTACCTGAAGAAGCAGAAGACCCTGCTGGC
CTTTGCGGAAAAGCGAGCCAGCAAGACGGATGAGCACGCA
GAGTTGATTGTCCTGAGAAGAGGAAGCTACAACGAAGCCA
CCAACCGTGTCAAGTGGCAGCCTGAGGAAGTGGTGACCCA
AGCCCAGCTGGAAGGCCACCGCTCCATGAATCCATGTCCC
TTGTATGACAAGCAAACAAAGACCCTCTTCCTTTTCTTCA
TCGCTGTCCCTGGGCGTGTATCAGAACATCATCAGCTCCA
CACTAAGGTTAATGTCACACGGCTGTGCTGTGTCAGCAGC
ACTGACCATGGGAGGACCTGGAGCCCCATCCAGGACCTCA
CAGAGACCACCATTGGCAGCACTCATCAGGAATGGGCCAC
ATTTGCTGTGGGTCCTGGGCATTGTCTGCAGCTGCGGAAC
CCAGCTGGGAGCCTGCTGGTACCTGCTTATGCCTACCGGA
AACTGCACCCTGCTCAGAAGCCTACCCCCTTTGCCTTCTG
CTTCATCAGCCTTGACCATGGGCACACATGGAAACTAGGC
AACTTTGTGGCTGAAAACTCACTGGAGTGCCAGGTGGCTG
AGGTTGGCACTGGAGCTCAGAGGATGGTATATCTCAATGC
TAGGAGCTTCCTGGGAGCCAGGGTCCAGGCACAAAGTCCT
AATGATGGTCTGGATTTCCAGGACAACCGGGTAGTGAGTA
AGCTTGTAGAGCCCCCCCACGGGTGTCATGGAAGTGTGGT
TGCCTTCCACAACCCCATCTCTAAGCCACATGCCTTAGAC
ACATGGCTTCTTTATACACACCCTACAGACTCCAGGAATA
GAACCAACCTGGGTGTGTACCTAAACCAGATGCCACTAGA
TCCCACAGCCTGGTCAGAGCCCACCCTGCTGGCCATGGGC
ATCTGTGCCTACTCAGACTTACAGAACATGGGGCAAGGCC
CTGATGGCTCCCCACAGTTTGGGTGTCTGTATGAATCAGG
TAACTATGAAGAGATCATTTTCCTCATATTCACCCTGAAG
CAAGCTTTCCCCACTGTATTTGATGCCCAGTGATCTCAGT
GCACGTGGCCCAAAGGGCTTCCTTGTGCTTCAAAACACCC
ATCTCTCTTTGCTTCCAGCATCCTCTGGACTCTTGAGTCC
AGCTCTTGGGTAACTTCCTCAGGAGGATGCAGAGAATTTG
GTCTCTTGACTCTCTGCAGGCCTTATTGTTTCAGCCTCTG
GTTCTCTTTTCAGCCCAGAAATCAAAGGAGCCTGGCTTTC
CTCAGCCTGTTGGCAGGGCAGGTGGGGACAGTATATATAG
AGGCTGCCATTCTGCATGTCGGTTGTCACTATGCTAGTTT
AACCTGCCTGTTTCCCCATGCCTAGTGTTTGAATGAGTAT
TAATAAAATATCCAACCCAGCCCATTTCTTCCTGGAAAAA
AA
SEQ ID NO: 44:
ACTGCGCGGTGAAGGGGCGTGGCCTGGCCGGGGAGGTTGA
CACCCAGACGCTGCTCTCAGTCCTCTGGCGCCTGCTCCCC
AGCGCATTCCTTCTGCTCCTGGGATATTTGTCTCATTACT
GCCAGTTCTTGCGCAGCGGTCACTGGGTTCGTTTCAGCGT
CTGTGGTTTCTGTCGCTGTTATCCAGTCTCCATCGCCCCA
GCTCAGCTTCAGGCCTTCTTCCGAGACTCCACGGGAGAGC
CCAGAGAGCCTCCGGAGCCGAAGCCATGGAGGAAGTCCCA
CCCTACTCCCTCAGCAGCACCCTGTTCCAGCAGGAAGAAC
AGAGTGGGGTGACCTACCGGATCCCAGCCCTGCTGTACCT
TCCTCCCACCCACACCTTCCTGGCCTTTGCAGAGAAGCGG
ACCTCAGTCAGAGATGAGGATGCTGCCTGCCTGGTGCTCA
GACGAGGGCTGATGAAGGGGCGCTCTGTACAGTGGGGCCC
CCAACGGCTACTGATGGAGGCCACATTACCTGGGCATCGC
ACCATGAACCCCTGCCCTGTGTGGGAGAAAAATACTGGCC
GTGTGTACCTGTTTTTCATCTGTGTGCGGGGCCATGTTAC
TGAGAGGTGCCAGATTGTGTGGGGCAAAAATGCCGCCCGT
CTCTGCTTCCTTTGCAGTGAAGATGCCGGCTGCTCTTGGG
GTGAAGTGAAAGACTTGACCGAGGAGGTCATTGGCTCAGA
GGTGAAGCGCTGGGCCACATTTGCTGTGGGCCCAGGTCAT
GGCATCCAGCTACACTCGGGAAGGCTGATCATCCCCGCCT
ATGCCTACTATGTCTCACGTTGGTTTCTCTGCTTTGCGTG
TTCAGTCAAGCCCCATTCCCTGATGATCTACAGTGATGAC
TTTGGAGTCACATGGCACCATGGCAAGTTCATTGAGCCCC
AGGTGACAGGGGAGTGCCAAGTGGCCGAAGTGGCTGGGAC
GGCTGGTAACCCTGTGCTCACTGCAGTGCCCGAACACCAA
GCCGATTTCGAGCAGAGGCTTTTAGTACTGATAGTGGTGG
CTGCTTTCAGAAGCCAACCCTGAACCCACAACTCCATGAG
CCTCGAACCGGCTGCCAAGGTAGTGTAGTGAGCTTCCGGC
CTTTGAAGATGCCAAATACCTATCAAGACTCAATTGGCAA
AGGTGCTCCCGCTACTCAGAAGTGCCCTCTGCTGGACAGT
CCTCTGGAGGTGGAGAAAGGAGCTGAAACACCATCAGCAA
CATGGCTCTTGTACTCACATCCAACTAGCAAGAGGAAGAG
GATTAACCTAGGCATCTACTACAACCGGAACCCCTTGGAG
GTGAACTGCTGGTCCCGCCCGTGGATCTTGAACCGTGGGC
CCAGTGGCTACTCTGATCTGGCTGTTGTGGAAGAACAGGA
CTTGGTGGCGTGTTTGTTTGAGTGTGGGGAGAAGAATGAG
TATGAGCGGATTGACTTCTGTCTGTTTTCAGACCATGAGG
TCCTGAGCTGTGAAGACTGTACCAGCCCTAGTAGCGACTA
AAGCCAAATCAAGACGGATGAGTGAGGCCCAGCTTCCCAC
AGAAAGGAATGGCAGCTACAGCCAGGGTAACAGAGGTCTC
TGATGTCTAGAGAAAACTCTAAAAACTAATAATCTGCTCC
TTGAATTTTTTCACTTTTCCCTTCAATGAGCATGGTGAAA
ATTGTGCCATATCTTACATAACGAGGCTCTTGAACTGGGA
GTTTGAATCTCTTCTCTTCCCATTAAAAGGAGAGGCCATG
TGCTCGCTTCGCGTTCGACAAAGCCTGGATTCTGATCTTG
AGTGGAAGCCACAGGCTTGTCTTTTCCAATGGTTCACTGC
TCACCTGAGTATTAGGTGATGTGTAGGTGCCTTGGCCAGA
AGAAAGATCTGTGTTGTTGTATTTTTTTAAATTTATTTAT
TTACTATATGTAAGTACACTGCAGCTGTCTTCAGACACAC
CAGAAGAGGGCGTCAGATCTCATTAGAGATGGTTGTGAGC
CACCATGTGGTTGCTGGGATTTGAACTCAGGACCTTCAGA
AGAGCAGTCAGTGCTCTTAACTACTGAGCCATCTCTCAAG
CCCCGCATTGCTGTATTTTTAATAAGAAAAATGCCCTTAT
CCTTCCAATAATGCCTGGAGCTGTACAAATTCTCTGTCTT
AGAAGACTTGAGAAAGCAGAACTGTAAGGTCAGATGCTTT
CTCCAGCCTTGATGCTGTGTTCCACCTTCCCTTCCTCATC
CAGAAAACAGTTACTAGGGAGAAAATGAGAAACCCATGCC
AGCTGCCCTTGATGATGGTTGATAACGGTGCTTATTGCTT
TTGATGTCATTACCTCTGTTAGAGATGAATCAGAGTCAGA
GGTCCTTAGCTGCATCCACCCATTTCCAGGGGGACATTCT
AACACTGCTGAACAGTCAGCTAAAATGAGAGCTGTGTGTC
CTAGCCTGATTCCAGGTTAGTCATGATGCTTCCTGGAGCT
GGGCTTTTATCTAATCCCAGGAGCCATCTAGGGGAGGCTC
AGAGCTAGCAGGTGATCTTCCTGAGATGGTTTCACCGTGA
CAGGTGAACCATGAGCCCTTCCAAGCAAGGCCAAAGGACA
ACATTATAGGAAAGATTTCTAGTATTAATATGCCTTTTCT
CTGTGTGTGTACTGTCTTGTAGTGATGCTATATAGACAAA
TAGATGATTTCTTATTTTTTGTTTGTTTGTTTGTTTTTTT
GTTTTTCTGTAGCCCTAGCTGTCCTGGAACTCACTTTGTA
AACCAGGCTGGCCTCGATCTCAGAAATCCGCCTGCCTCTG
CCTCCCGAGTGCTGGGATTAAAGGTGTGCACGAGCACACC
TTAATGATGATCCTATAAGTATTCCTAAAATTATACTAGT
AATTATTAACTCCTTTATAATAGGACTGCTATTAAAGCCC
TCGCTGATATGAAAACTACAGTGAGAACTCTGCCAGTCTT
CACATGTCATAATTAGTTCTGAGATAGAAAGCAGGCATTT
ACAACTTAGAACACATTTCTTAGAGCTGTAAAACAATTAA
CTAGAGGTCATAAAAGGGAATGAAAGATTTATTGTAGGTG
CTAGGACAGAACATAAAATATTGACTGGGCTTATCTATAT
GAAACTTCATTGTTAACTTTTACACAAGAATTATGGTTTT
TAACTTTCAGTGAACCTGCGGAGCTAGTGACAGAAGAGAA
ATGTCTAGTTAGATAACTACTCTTAATGGAAATTCACATA
AACATCTGTTGCCATCTTCTTTTTGAATTTATGTTTAAAC
TTGTGAATGTTTGAATTAGACACTACGCGAGCACATAGAA
AATAAAGAACTAAGCGTGAA
SEQ ID NO: 45:
GGACAGTGTGCATCACGGAGCTTGTGGCCCAGACTGTGCC
TGGCAGACCCAGAGGACCTAAGGCTTGGCTCTAGTGGTGG
TCAGCACAGCCCTCGGTGGTCTGCGGAGCCTGATATTGCT
TTACGTAAGGGCTGTTCTGCTGTGCATCTCCTGTGTCTGA
AGCTATTCGCCATGGAGACTGCTGGAGCTCCCTTCTGCTT
CCATGTGGACTCCCTGGTACCTTGCTCCTACTGGAAGGTT
ATGGGGCCCACGCGTGTTCCCAGGAGAACGGTGCTCTTCC
AGAGGGAAAGGACGGGCCTGACCTACCGTGTGCCTGCGTT
ACTCTGTGTGCCTCCCAGGCCTACTCTGCTGGCCTTCGCG
GAACAGCGACTTAGCCCTGATGACTCCCATGCCCACCGCC
TGGTGCTACGGAGGGGCACGCTGACCAGGGGCTCAGTGCG
GTGGGGCACTCTGAGTGTACTGGAGACTGCAGTACTGGAG
GAGCACAGGTCTATGAACCCTTGCCCGGTGCTGGATGAGC
ACTCTGGTACCATCTTCCTCTTCTTCATTGCCGTGCTGGG
CCACACACCGGAGGCCGTGCAAATCGCCACTGGCAAGAAC
GCTGCTCGCCTCTGCTGTGTGACCAGCTGTGACGCTGGCC
TCACCTGGGGCAGTGTTCGAGATCTCACTGAGGAAGCCAT
TGGTGCTGCATTGCAGGACTGGGCCACCTTTGCTGTGGGT
CCGGGCCATGGAGTTCAGCTGCGCTCGGGTCGCCTGCTTG
TTCCTGCTTACACCTATCATGTGGACCGACGGGAATGTTT
TGGCAAGATCTGCTGGACCAGTCCCCACTCCTTGGCATTC
TACAGTGATGATCATGGGATCTCCTGGCATTGTGGAGGCC
TTGTGCCCAACCTACGCTCTGGAGAGTGCCAACTGGCTGC
GGTAGATGGAGACTTTCTCTACTGTAATGCTCGAAGCCCT
CTGGGTAACCGTGTGCAGGCACTGAGTGCTGATGAAGGCA
CGTCCTTCCTACCAGGGGAGCTGGTGCCTACATTGGCAGA
GACGGCTCGTGGTTGCCAGGGTAGCATTGTGGGCTTCCTA
GCTCCACCCTCAATCGAGCCTCAGGATGACCGGTGGACAG
GGAGTCCTAGGAACACCCCACATTCCCCATGCTTCAATCT
CAGAGTACAGGAGTCTTCGGGGGAAGGTGCCAGAGGTCTT
CTTGAACGTTGGATGCCCAGGTTGCCTCTCTGCTACCCAC
AGTCCCGGAGCCCAGAGAATCATGGCCTAGAGCCTGGGTC
AGATGGAGATAAGACATCCTGGACTCCGGAATGTCCTATG
TCCTCTGATTCCATGCTTCAGAGCCCCACATGGCTACTAT
ATTCCCACCCAGCAGGGCGTAGAGCTCGGCTCCACATGGG
AATCTACCTGAGCCGATCCCCCTTGGATCCCCACAGCTGG
ACAGAGCCCTGGGTGATCTATGAGGGCCCCAGTGGCTACT
CTGACCTTGCCTTTCTTGGGCCTATGCCTGGGGCATCCCT
GGTTTTTGCCTGTCTGTTTGAGAGCGGGACCAGGACTTCC
TATGAAGACATTTCTTTTTGCTTGTTCTCACTGGCGGATG
TCCTGGAGAATGTGCCCACTGGCTTAGAGATGCTAAGTCT
CAGGGATAAGGCTCAGGGGCATTGCTGGCCCTCTTGATGG
CCTCACCCTCTCGTAGCCGCCTGGAGAGGAAGGGTAGACT
ATATAGAGGAGGTTAGGGGTAGGTCAGCATGATGCTAGGA
TGGAGAGAGCTCTGTCCCCTCGTGGATGGTGGTGGTGACT
CACCCGGGGGGCCAGCTGCTTTCTGAGTGCAAATGAGAAA
AATAAAGAGCTGCGCTGTGACTTTTCTTTCCACATCAAAG
CTTGGGTGTCAGTGCTTTAGCTTGATGCTCTGATCACCAT
GCAAATCTTCCACCGGCGCCTTGCTCAGCTTTCATATCCC
AAGGGTGCCTGGGAGGAAGGCAACAGGGACAGTGGACATC
ACTGCACCACTTTCCACGACCCTGTGTGCCAACCTCAGCC
ACTTTGAAACATGCTGATGACTGAGGTCTGTTCACTTTCT
TAATTTCAAGCAGGAGAAGCAGGTTGGGGAGCCAGCCTCC
CCAGCTAGAGGGGACAGAACTTGACTTGAGCAGGGGGGTA
CCTCCTAGGACCTGCTCCATGTGCCTACTTCTTTACCCTT
CTCTAGAGAGGGCTCTTGTCCTGTCAGAGCTGTTTTCTCC
CTTCTCTTGTTTTTTCTTTTTCAAGACTGTTTCTCTGTGT
TAGCCCTGGCTGTCCTGGATCTCACTCTGTAGATCAGGCT
GACCTTGAGTTCAAAGCTCCATCTGCCTCTACTTCTCACA
TTACTGTGATTAAAGGCATATACTACCACTGCCTGGTGCC
CTTTTGTATTTCTTATTAAAGTCCTAATGTCTGATTATAA
AAACAGTCTGTGTGGGCTGGAGTGATGGCTTACTCAGTAA
AGCACTTGCCATGGAATCTGGGCAATCTGAGTTTCATTTT
TAGCATCCTGTAAAAATCCCAATTTGATGGTGTACTTGTA
ATGTCAGCATGGAGAGGCAGAGATAGGTAAGTTCCCCAAG
ACTCTTTGAACCGACAGCTTGGCCTCACTGGCACATTCCA
GGTCTCAGTGAGAGACCCTGCCTCAAAATACAAAGAAAGA
GCTGCTGAAGAGTGGGTCAGAGTTGACCTCTGATCTCCGG
AAGTATATGATACACACCCGTGCATGCACTCTTGCTTACA
AAATAAAAAGCAAAACAAAACCCCAACAGGTATATGGCCA
TTTTAGAAAAATTAGAAGATTTAGAAAGCTATACATAAAA
AAAAATGACCTAAAGAAAAATCTTTAGTGTTCTGGGCACT
ATCCCTATCAAACCACTGTGTTCTTTGGCCAAGCCTTGGG
GTGGACACTGTTTTGAGGTGGGTCCTGTTATCTCCACTAG
GTAGTGGAGTTTTGTGTCAGACTAACTGGGTCTTAAAGCT
GTCTTTAAGGCCATCAGGAGCTACTGACTTGCCTGCCTCA
GCAGAGCATATCCTGAAGGTCGGGGTTAAGTCTCCTTCCC
GAGCGAGTTGCCTTCCAGTGGGCCCCTGGACTCCTAGGTC
CTCAGCGCTCATCAGCTGCCAAGGACTCTGAGGGAATGTC
CTCTGACTGTGGCCCCGAAAGGTAGGGGAGGGGGATGTGC
TTAGGCTTAGGACAGGGTCCTGTTTCAGTCTGCCTTCACT
GTTAGTAGCACTGTGCCACATGGCACAGACTGGGCGAGCT
TTAAAGGAAGGAGGTTGATATTGGTTCCCACTTCTGGGGA
TCATGGTTGAGCAGCCTTGTCTGATGATGGTTGTCTTGAT
GGTAGATCGTGAGGTAGTTGATGAAGGTATGACATGGTGA
GAAACTCTGTGTGTGTGTGTTATTTTCTCTGTGTTCTACC
TATACATCTATCTATGTATATATGTATCTATCTATCTACC
TGGAGGCTGGAGAGATAGCTTAGTGGTTAAGAACATTTGT
TGTTCTTGCATAGTCCTGGATTTAAATTTTCAGCACCCAC
ATGGCAGCTCACAACAACCCATAAATCCAGTTTCAGAGGA
TCCAACCTCTGATATACCATGTCAGCCAGAGCAGACACGG
CTGAAGGTGGTTTGATCCCCGTATGGAGAGGTGACAATTG
GGAAGAGAGAAAGATCAACTTAACCATGCAAGGAACAGGA
AGTTAAATACTGAACAGGGAAGGTAAAGGCAGGAAGTAGA
TGTAGAGGGCAAATCAATGAAACCCAAACATACCCAAATT
ACGCTAAACACACACTGACATGCCAATTAAAAGGACAAAT
TGGCTCCACTGGCAAAACCAAAACAGACACTGAAGATCCA
AACAGTCACATGCCAACTACCGCGGAGGGAGACAGACACA
GAGAAGACCGTGACAGACACTTGGACACTCTTGAGAGTGG
ATGTGCAGGAAGAGAGCTCTGCCAGTGGAGAAGAAAGCAC
TCAGAAGAAAGTGACAGCAGCTGTAAATTTGTATTCTGCT
AATGTTATGTTCCAAAGTTGAAAGCAAAATTGTACCAATT
CATAAGAACAAACAGGCTGACTCTCAGTTGTGACTGAACG
TCTCTCAGTAACTGACGGGGCGAGCAGGCCAAAGGAGAGT
CGGCTCAGAAGGGTGCATAGCCACGCCAAATCAAATAAGC
AAGTACAACCGGCAGGCTCTATTTCTAGCACAAAGGGGTC
TGTGCCTCATTCTGTGCTTGGGTCAGAGCTTGGGTCTCTC
ATTTGGATGTAAGTGGTGTAGTGGAGAAGCAGGAAATAAT
CCGGAGCGCATATTTTGATTTTAACATAAGTGCTGATTTG
GGAGGGAGTTTTGTCAAATTGTGTTTTTACAATGTTTTTT
TTTTTTTAAATGATGCTTTTTTGTAAAGTGTAGAAATGTG
ATATAAGATTGGTTCTGCTACATTCAGTTTCTATAAAAGT
GGTTCTAAAATATTGTACTGTCAATCATCTCATGATTATT
CTACTGTACACATTACTGACTTTGTATGTAATAATTAATA
TTAGAAGAAAATATAATTTATTTGAATATAAAAAAAAAAA
AAAAAAAA
SEQ ID NO: 46:
X₁ASLPX₂LQX₃ESVFQSGAHAYRIPALLYLPGQQSLLAF
AEQRASKKDEHAELIVLRRGDYDAX₄THQVQWQAQEVVAQ
ARLDGHRSMNPCPLYDX₅QTGTLFLFFIAIPGQVTEQQQL
QTRANVTRLCQVTSTDHGRTWSSPRDLTDAAIGPAYREWS
TFAVGPGHCLQLHDRARSLVVPAYAYRKLHPX₆QRPIPSA
FCFLSHDHGRTWARGHFVAQDTLECQVAEVETGEQRVVTL
NARSHLRARVQAQSTNDGLDFQESQLVKKLVEPPPX₇GCQ
GSVISFPSPRSGPGSPAQWLLYTHPTHX₈X₉QRADLGAYL
NPRPPAPEAWSEPVLLAKGSX₁₀AYSDLQSMGTGPDGSPL
FGCLYEANDYEEIX₁₁FX₁₂MFTLKQAFPAEYLPQ
SEQ ID NO: 47:
X₁X₂SX₃X₄X₅LQX₆ESVFQSGAHAYRIPALLYLPGQQSL
LAFAEQRASX₇X₈DEHAELIVX₉RRGDYDAX₁₀THQVQWX₁₁
AQEVVAQAX₁₂LDGHRSMNPCPLYDX₁₃QTGTLFLFFI
AIPX₁₄X₁₅VTEX₁₆QQLQTRANVTRLX₁₇X₁₈VTSTDHG
RTWSSPRDLTDAAIGPX₁₉YREWSTFAVGPGHX₂₀LQLHD
RX₂₁RSLVVPAYAYRKLHPX₂₂QRPIPSAFX₂₃FLSHDHG
RTWARGHFVAQDTX₂₄ECQVAEVETGEQRVVTLNARSHL
RARVQAQSX₂₅NX₂₆GLDFQX₂₇SQLVKKLVEPPPX₂₈GX₂₉
QGSVISFPSPRSGPGSPAQX₃₀LLYTHPTHX₃₁X₃₂QRA
DLGAYLNPRPPAPEAWSEPX₃₃LLAKGSX₃₄AYSDLQSMG
TGPDGSPLFGX₃₅LYEANDYEEIX₃₆FX₃₇MFTLKQAFPA
EYLPQ
SEQ ID NO: 48:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQ
SEQ ID NO: 49:
DASLPYLQDESVFQSGAHAYRIPALLYLPGQQSLLAFAEQ
RASKKDEHAELIVLRRGDYDAPTHQVQWQAQEVVAQARLD
GHRSMNPCPLYDEQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIRFIMFTLKQAFPAEYLPQ
SEQ ID NO: 50:
DASLPYLQKESVFQSGAHAYRIPALLYLPGQQSLLAFAEQ
RASKKDEHAELIVLRRGDYDANTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQ
SEQ ID NO: 51:
DASLPYLQKESVFQSGAHAYRIPALLYLPGQQSLLAFAEQ
RASKKDEHAELIVLRRGDYDAPTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPAGCQGSVISFP
SPRSGPGSPAQWLLYTHPTHRKQRADLGAYLNPRPPAPEA
WSEPVLLAKGSAAYSDLQSMGTGPDGSPLFGCLYEANDYE
EIVFLMFTLKQAFPAEYLPQ
SEQ ID NO: 52:
DASLPYLQKESVFQSGAHAYRIPALLYLPGQQSLLAFAEQ
RASKKDEHAELIVLRRGDYDASTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPAGCQGSVISFP
SPRSGPGSPAQWLLYTHPTHRKQRADLGAYLNPRPPAPEA
WSEPVLLAKGSAAYSDLQSMGTGPDGSPLFGCLYEANDYE
EIVFLMFTLKQAFPAEYLPQ
SEQ ID NO: 53:
DASLPYLQKESVFQSGAHAYRIPALLYLPGQQSLLAFAEQ
RASKKDEHAELIVLRRGDYDATTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPAGCQGSVISFP
SPRSGPGSPAQWLLYTHPTHRKQRADLGAYLNPRPPAPEA
WSEPVLLAKGSAAYSDLQSMGTGPDGSPLFGCLYEANDYE
EIVFLMFTLKQAFPAEYLPQ
SEQ ID NO: 54:
DASLPYLQKESVFQSGAHAYRIPALLYLPGQQSLLAFAEQ
RASKKDEHAELIVLRRGDYDANTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPAGCQGSVISFP
SPRSGPGSPAQWLLYTHPTHRKQRADLGAYLNPRPPAPEA
WSEPVLLAKGSAAYSDLQSMGTGPDGSPLFGCLYEANDYE
EIVFLMFTLKQAFPAEYLPQ
SEQ ID NO: 55:
ASLPYLQKESVFQSGAHAYRIPALLYLPGQQSLLAFAEQR
ASKKDEHAELIVLRRGDYDAPTHQVQWQAQEVVAQARLDG
HRSMNPCPLYDAQTGTLFLFFIAIPGQVTEQQQLQTRANV
TRLCQVTSTDHGRTWSSPRDLTDAAIGPAYREWSTFAVGP
GHCLQLHDRARSLVVPAYAYRKLHPKQRPIPSAFCFLSHD
HGRTWARGHFVAQDTLECQVAEVETGEQRVVTLNARSHLR
ARVQAQSTNDGLDFQESQLVKKLVEPPPQGCQGSVISFPS
PRSGPGSPAQWLLYTHPTHSWQRADLGAYLNPRPPAPEAW
SEPVLLAKGSCAYSDLQSMGTGPDGSPLFGCLYEANDYEE
IVFLMFTLKQAFPAEYLPQ
SEQ ID NO: 56:
MASLPYLQKESVFQSGAHAYRIPALLYLPGQQSLLAFAEQ
RASKKDEHAELIVLRRGDYDAPTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSCAYSDLQSMGTGPDGSPLFGCLYEANDYE
EIVFLMFTLKQAFPAEYLPQ
SEQ ID NO: 57:
ASLPYLQKESVFQSGAHAYRIPALLYLPGQQSLLAFAEQR
ASKKDEHAELIVLRRGDYDAPTHQVQWQAQEVVAQARLDG
HRSMNPCPLYDAQTGTLFLFFIAIPGQVTEQQQLQTRANV
TRLCQVTSTDHGRTWSSPRDLTDAAIGPAYREWSTFAVGP
GHCLQLHDRARSLVVPAYAYRKLHPKQRPIPSAFCFLSHD
HGRTWARGHFVAQDTLECQVAEVETGEQRVVTLNARSHLR
ARVQAQSTNDGLDFQESQLVKKLVEPPPQGCQGSVISFPS
PRSGPGSPAQWLLYTHPTHSWQRADLGAYLNPRPPAPEAW
SEPVLLAKGSAAYSDLQSMGTGPDGSPLFGCLYEANDYEE
IVFLMFTLKQAFPAEYLPQ
SEQ ID NO: 58:
DASLPYLQKESVFQSGAHAYRIPALLYLPGQQSLLAFAEQ
RASKKDEHAELIVLRRGDYDAPTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQ
SEQ ID NO: 59:
DASLPYLQKESVFQSGAHAYRIPALLYLPGQQSLLAFAEQ
RASKKDEHAELIVLRRGDYDAPTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSCAYSDLQSMGTGPDGSPLFGCLYEANDYE
EIVFLMFTLKQAFPAEYLPQ
SEQ ID NO: 60:
AASLPYLQKESVFQSGAHAYRIPALLYLPGQQSLLAFAEQ
RASKKDEHAELIVLRRGDYDAPTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQ
SEQ ID NO: 61:
MASLPYLQKESVFQSGAHAYRIPALLYLPGQQSLLAFAEQ
RASKKDEHAELIVLRRGDYDAPTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQ
SEQ ID NO: 62:
AASLPYLQKESVFQSGAHAYRIPALLYLPGQQSLLAFAEQ
RASKKDEHAELIVLRRGDYDAPTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSCAYSDLQSMGTGPDGSPLFGCLYEANDYE
EIVFLMFTLKQAFPAEYLPQ
SEQ ID NO: 63:
QVQLQQPGAELVKPGASVKMSCKASGYTFTSYNMHWVKQT
PGRGLEWIGAIYPGNGDTSYNQKFKGKATLTADKSSSTAY
MQLSSLTSEDSAVYYCARSTYYGGDWYFNVWGAGTTVTVS
AASTKGPSVFPLAPSSKSTSGGTAALGCLVKDYFPEPVTV
SWNSGALTSGVHTFPAVLQSSGLYSLSSVVTVPSSSLGTQ
TYICNVNHKPSNTKVDKKAEPKSCDKTHTCPPCPAPELLG
GPSVFLFPPKPKDTLMISRTPEVTCVVVDVSHEDPEVKFN
WYVDGVEVHNAKTKPREEQYNSTYRVVSVLTVLHQDWLNG
KEYKCKVSNKALPAPIEKTISKAKGQPREPQVYTLPPSRD
ELTKNQVSLTCLVKGEYPSDIAVEWESNGQPENNYKTTPP
VLDSDGSFFLYSKLTVDKSRWQQGNVFSCSVMHEALHNHY
TQKSLSLSPGK
SEQ ID NO: 64:
QIVLSQSPAILSASPGEKVTMTCRASSSVSYIHWFQQKPG
SSPKPWIYATSNLASGVPVRFSGSGSGTSYSLTISRVEAE
DAATYYCQQWTSNPPTFGGGTKLEIKRTVAAPSVFIFPPS
DEQLKSGTASVVCLLNNFYPREAKVQWKVDNALQSGNSQE
SVTEQDSKDSTYSLSSTLTLSKADYEKHKVYACEVTHQGL
SSPVTKSFNRGEC
SEQ ID NO: 65:
EVQLVESGGGLVQPGRSLRLSCAASGFTFNDYAMHWVRQA
PGKGLEWVSTISWNSGSIGYADSVKGRFTISRDNAKKSLY
LQMNSLRAEDTALYYCAKDIQYGNYYYGMDVWGQGTTVTV
SSASTKGPSVFPLAPGSSKSTSGTAALGCLVKDYFPEPVT
VSWNSGALTSGVHTFPAVLQSSGLYSLSSVVTVPSSSLGT
QTYICNVNHKPSNTKVDKKVEP
SEQ ID NO: 66:
QIVLSQSPAILSASPGEKVTMTCRASSSVSYIHWFQQKPG
SSPKPWIYATSNLASGVPVRFSGSGSGTSYSLTISRVEAE
DAATYYCQQWTSNPPTFGGGTKLEIKRTVAAPSVFIFPPS
DEQLKSGTASVVCLLNNFYPREAKVQWKVDNALQSGNSQE
SVTEQDSKDSTYSLSSTLTLSKADYEKHKVYACEVTHQGL
SSPVTKSFNRGEC
SEQ ID NO: 67:
QVQLQQPGAELVKPGASVKMSCKASGYTFTSYNMHWVKQT
PGRGLEWIGAIYPGNGDTSYNQKFKGKATLTADKSSSTAY
MQLSSLTSEDSAVYYCARSTYYGGDWYFNVWGAGTTVTVS
AASTKGPSVFPLAPSSKSTSGGTAALGCLVKDYFPEPVTV
SWNSGALTSGVHTFPAVLQSSGLYSLSSVVTVPSSSLGTQ
TYICNVNHKPSNTKVDKKVEPKSCDKTHTCPPCPAPELLG
GPSVFLFPPKPKDTLMISRTPEVTCVVVDVSHEDPEVKFN
WYVDGVEVHNAKTKPREEQYNSTYRVVSVLTVLHQDWLNG
KEYKCKVSNKALPAPIEKTISKAKGQPREPQVYTLPPSRE
EMTKNQVSLYCLVKGFYPSDIAVEWESNGQPENNYKTTPP
VLDSDGSFFLYSKLTVDKSRWQQGNVFSCSVMHEALHNHY
TQKSLSLSPGK
SEQ ID NO: 68:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCQVTSTDHGRTWSSPRDLTDAATGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK
SEQ ID NO: 69:
DASLPYLQDESVFQSGAHAYRIPALLYLPGQQSLLAFAEQ
RASKKDEHAELIVLRRGDYDAPTHQVQWQAQEVVAQARLD
GHRSMNPCPLYDEQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIRFIMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK
SEQ ID NO: 70:
DASLPYLQKESVFQSGAHAYRIPALLYLPGQQSLLAFAEQ
RASKKDEHAELIVLRRGDYDANTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK
SEQ ID NO: 71:
DASLPYLQKESVFQSGAHAYRIPALLYLPGQQSLLAFAEQ
RASKKDEHAELIVLRRGDYDAPTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPAGCQGSVISFP
SPRSGPGSPAQWLLYTHPTHRKQRADLGAYLNPRPPAPEA
WSEPVLLAKGSAAYSDLQSMGTGPDGSPLFGCLYEANDYE
EIVFLMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK
SEQ ID NO: 72:
DASLPYLQKESVFQSGAHAYRIPALLYLPGQQSLLAFAEQ
RASKKDEHAELIVLRRGDYDASTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPAGCQGSVISFP
SPRSGPGSPAQWLLYTHPTHRKQRADLGAYLNPRPPAPEA
WSEPVLLAKGSAAYSDLQSMGTGPDGSPLFGCLYEANDYE
EIVFLMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK
SEQ ID NO: 73:
DASLPYLQKESVFQSGAHAYRIPALLYLPGQQSLLAFAEQ
RASKKDEHAELIVLRRGDYDATTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPAGCQGSVISFP
SPRSGPGSPAQWLLYTHPTHRKQRADLGAYLNPRPPAPEA
WSEPVLLAKGSAAYSDLQSMGTGPDGSPLFGCLYEANDYE
EIVFLMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK
SEQ ID NO: 74:
DASLPYLQKESVFQSGAHAYRIPALLYLPGQQSLLAFAEQ
RASKKDEHAELIVLRRGDYDANTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPAGCQGSVISFP
SPRSGPGSPAQWLLYTHPTHRKQRADLGAYLNPRPPAPEA
WSEPVLLAKGSAAYSDLQSMGTGPDGSPLFGCLYEANDYE
EIVFLMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK
SEQ ID NO: 75:
X₁ASLPX₂LQX₃ESVFQSGAHAYRIPALLYLPGQQSLLAF
AEQRASKKDEHAELIVLRRGDYDAX₄THQVQWQAQEVVAQ
ARLDGHRSMNPCPLYDX₅QTGTLFLFFIAIPGQVTEQQQL
QTRANVTRLCQVTSTDHGRTWSSPRDLTDAAIGPAYREWS
TFAVGPGHCLQLHDRARSLVVPAYAYRKLHPX₆QRPIPSA
FCFLSHDHGRTWARGHFVAQDTLECQVAEVETGEQRVVTL
NARSHLRARVQAQSTNDGLDFQESQLVKKLVEPPPX₇GCQ
GSVISFPSPRSGPGSPAQWLLYTHPTHX₈X₉QRADLGAYL
NPRPPAPEAWSEPVLLAKGSX₁₀AYSDLQSMGTGPDGSPL
FGCLYEANDYEEIX₁₁FX₁₂MFTLKQAFPAEYLPQGGGGS
GGGGSDKTHTCPPCPAPELLGGPSVFLFPPKPKDTLMISR
TPEVTCVVVDVSHEDPEVKFNWYVDGVEVHNAKTKPREEQ
YNSTYRVVSVLTVLHQDWLNGKEYKCKVSNKALPAPIEKT
ISKAKGQPREPQVYTLPPSREEMTKNQVSLTCLVKGFYPS
DIAVEWESNGQPENNYKTTPPVLDSDGSFFLTSKLTVDKS
RWQQGNVFSCSVMHEALHNHYTQKSLSLSPGK
SEQ ID NO: 76:
X₁X₂SX₃X₄X₅LQX₆ESVFQSGAHAYRIPALLYLPGQQSL
LAFAEQRASX₇X₈DEHAELIVX₉RRGDYDAX₁₀THQVQWX
₁₁AQEVVAQAX₁₂LDGHRSMNPCPLYDX₁₃QTGTLFLFFI
AIPX₁₄X₁₅VTEX₁₆QQLQTRANVTRLX₁₇X₁₈VTSTDHG
RTWSSPRDLTDAAIGPX₁₉YREWSTFAVGPGHX₂₀LQLHD
RX₂₁RSLVVPAYAYRKLHPX₂₂QRPIPSAFX₂₃FLSHDHG
RTWARGHFVAQDTX₂₄ECQVAEVETGEQRVVTLNARSHLR
ARVQAQSX₂₅NX₂₆GLDFQX₂₇SQLVKKLVEPPPX₂₈GX₂₉
QGSVISFPSPRSGPGSPAQX₃₀LLYTHPTHX₃₁X₃₂QRA
DLGAYLNPRPPAPEAWSEPX₃₃LLAKGSX₃₄AYSDLQSMG
TGPDGSPLFGX₃₅LYEANDYEEIX₃₆FX₃₇MFTLKQAFPA
EYLPQGGGGSGGGGSDKTHTCPPCPAPELLGGPSVFLFPP
KPKDTLMISRTPEVTCVVVDVSHEDPEVKFNWYVDGVEVH
NAKTKPREEQYNSTYRVVSVLTVLHQDWLNGKEYKCKVSN
KALPAPIEKTISKAKGQPREPQVYTLPPSREEMTKNQVSL
TCLVKGFYPSDIAVEWESNGQPENNYKTTPPVLDSDGSFF
LTSKLTVDKSRWQQGNVFSCSVMH
SEQ ID NO: 77:
EIVLTQSPATLSLSPGERATLSCRASQSVSSYLAWYQQKP
GQAPRLLIYDASNRATGIPARFSGSGSGTDFTLTISSLEP
EDFAVYYCQQRSNWPITFGQGTRLEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNR
SEQ ID NO: 78:
DASLPYLQKESVFQSGAHAYRIPALLYLPGQQSLLAFAEQ
RASKKDEHAELIVLRRGDYDAPTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK
SEQ ID NO: 79:
GATGCATCTCTGCCTTACCTGCAGAAAGAAAGCGTGTTCC
AGTCTGGCGCCCACGCCTACAGAATTCCCGCTCTGCTGTA
TCTGCCAGGCCAGCAGTCTCTGCTGGCTTTCGCTGAACAG
CGGGCCAGCAAGAAGGATGAGCACGCCGAACTGATCGTGC
TGCGGAGAGGCGATTACGACGCCCCTACACATCAGGTGCA
GTGGCAGGCTCAAGAGGTGGTGGCTCAGGCTAGACTGGAC
GGCCACAGATCTATGAACCCCTGTCCTCTGTACGATGCCC
AGACCGGCACACTGTTTCTGTTCTTTATCGCTATCCCCGG
CCAAGTGACCGAGCAGCAGCAGCTGCAGACAAGAGCCAAC
GTGACCAGACTGTGTCAAGTGACCTCCACCGACCACGGCA
GAACCTGGTCTAGCCCTAGAGATCTGACCGACGCCGCCAT
CGGACCTGCCTATAGAGAGTGGTCCACCTTCGCCGTTGGA
CCTGGACACTGTCTCCAGCTGCACGACAGGGCTAGATCTC
TGGTGGTGCCTGCCTACGCCTATAGAAAGCTGCACCCCAA
ACAGCGGCCTATTCCTAGCGCCTTCTGCTTTCTGAGCCAC
GATCACGGCAGGACATGGGCCAGAGGACATTTCGTGGCCC
AGGACACACTGGAATGCCAGGTGGCCGAAGTGGAAACCGG
CGAGCAGAGAGTCGTGACCCTGAACGCCAGATCTCACCTG
AGAGCCAGAGTGCAGGCCCAGAGCACAAACGACGGCCTGG
ATTTCCAAGAGAGCCAGCTGGTCAAGAAACTGGTGGAACC
TCCTCCACAGGGCTGTCAGGGAAGCGTGATCAGCTTTCCA
TCTCCTAGAAGCGGCCCTGGCTCTCCTGCTCAGTGGCTGC
TGTATACACACCCCACACACAGCTGGCAGAGAGCCGATCT
GGGCGCCTACCTGAATCCTAGACCTCCTGCTCCTGAGGCT
TGGAGCGAACCTGTTCTGCTGGCCAAGGGCAGCGCCGCCT
ACAGCGATCTGCAGTCTATGGGCACAGGCCCTGATGGCAG
CCCTCTGTTTGGCTGTCTGTACGAGGCCAACGACTACGAA
GAGATCGTGTTCCTGATGTTCACCCTGAAGCAGGCCTTTC
CAGCCGAGTACCTGCCTCAAGGCGGAGGTGGAAGTGGCGG
AGGCGGATCCGACAAAACTCACACATGCCCACCGTGCCCA
GCACCTGAACTCCTGGGGGGACCGTCAGTCTTCCTCTTCC
CCCCAAAACCCAAGGACACCCTCATGATCTCCCGGACCCC
TGAGGTCACATGCGTGGTGGTGGACGTGAGCCACGAAGAC
CCTGAGGTCAAGTTCAACTGGTACGTGGACGGCGTGGAGG
TGCATAATGCCAAGACAAAGCCGCGGGAGGAGCAGTACAA
CAGCACGTACCGTGTGGTCAGCGTCCTCACCGTCCTGCAC
CAGGACTGGCTGAATGGCAAGGAGTACAAGTGCAAGGTCT
CCAACAAAGCCCTCCCAGCCCCCATCGAGAAAACCATCTC
CAAAGCCAAAGGGCAGCCCCGAGAACCACAGGTCTACACC
CTGCCCCCATCCCGGGAGGAGATGACCAAGAACCAGGTCA
GCCTGACCTGCCTGGTCAAAGGCTTCTATCCCAGCGACAT
CGCCGTGGAGTGGGAGAGCAATGGGCAGCCGGAGAACAAC
TACAAGACCACGCCTCCCGTGCTGGACTCCGACGGCTCCT
TCTTCCTCACTAGCAAGCTCACCGTGGACAAGAGCAGGTG
GCAGCAGGGGAACGTCTTCTCATGCTCCGTGATGCATGAG
GCTCTGCACAACCACTACACGCAGAAGAGCCTCTCCCTGT
CTCCGGGTAAA
SEQ ID NO: 80:
EIVLTQSPATLSLSPGERATLSCRASQSVSSYLAWYQQKP
GQAPRLLIYDASNRATGIPARFSGSGSGTDFTLTISSLEP
EDFAVYYCQQRSNWPITFGQGTRLEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC
SEQ ID NO: 81:
EVQLVESGGGLVQPGRSLRLSCAASGFTFNDYAMHWVRQA
PGKGLEWVSTISWNSGSIGYADSVKGRFTISRDNAKKSLY
LQMNSLRAEDTALYYCAKDIQYGNYYYGMDVWGQGTTVTV
SSASTKGPSVFPLAPSSKSTSGGTAALGCLVKDYFPEPVT
VSWNSGALTSGVHTFPAVLQSSGLYSLSSVVTVPSSSLGT
QTYICNVNHKPSNTKVDKKVEPKSCDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLYCLVKGFYPSDIAVEWESNGQPENNYKTTP
PVLDSDGSFFLYSKLTVDKSRWQQGNVFSCSVMHEALHNH
YTQKSLSLSPGK
SEQ ID NO: 82:
GAGATCGTGCTGACACAGTCTCCCGCCACACTGTCACTGT
CTCCAGGCGAAAGAGCCACACTGAGCTGTAGAGCCAGCCA
GAGCGTGTCCTCTTACCTGGCCTGGTATCAGCAGAAGCCT
GGACAGGCTCCCCGGCTGCTGATCTACGATGCCAGCAATA
GAGCCACAGGCATCCCCGCCAGATTTTCTGGCTCTGGCAG
CGGCACCGATTTCACCCTGACCATAAGCAGCCTGGAACCT
GAGGACTTCGCCGTGTACTACTGCCAGCAGAGAAGCAACT
GGCCCATCACCTTTGGCCAGGGCACCAGACTGGAAATCAA
GCGTACGGTGGCTGCACCATCTGTCTTCATCTTCCCGCCA
TCTGATGAGCAGTTGAAATCTGGAACTGCCTCTGTTGTGT
GCCTGCTGAATAACTTCTATCCCAGAGAGGCCAAAGTACA
GTGGAAGGTGGATAACGCCCTCCAATCGGGTAACTCCCAG
GAGAGTGTCACAGAGCAGGACAGCAAGGACAGCACCTACA
GCCTCAGCAGCACCCTGACGCTGAGCAAAGCAGACTACGA
GAAACACAAAGTCTACGCCTGCGAAGTCACCCATCAGGGC
CTGAGCTCGCCCGTCACAAAGAGCTTCAACAGGGGAGAGT
GT
SEQ ID NO: 83:
GCAGCCTGGCAGATCTCTGAGACTGTCTTGTGCCGCCAGC
GGCTTCACCTTCAACGACTATGCCATGCACTGGGTCCGAC
AGGCCCCTGGCAAAGGACTTGAATGGGTGTCCACCATCAG
CTGGAACAGCGGCTCTATCGGCTACGCCGATTCCGTGAAG
GGCAGATTCACCATCTCCAGAGACAACGCCAAGAAGTCCC
TGTACCTGCAGATGAACAGCCTGAGAGCCGAGGACACAGC
CCTGTACTACTGCGCCAAGGACATCCAGTACGGCAACTAC
TACTACGGCATGGACGTGTGGGGCCAGGGCACAACAGTGA
CAGTCTCTTCTGCTAGCACCAAGGGCCCATCCGTCTTCCC
CCTGGCACCCTCCTCCAAGAGCACCTCTGGGGGCACAGCG
GCCCTGGGCTGCCTGGTCAAGGACTACTTCCCCGAACCGG
TGACGGTGTCCTGGAACTCAGGCGCTCTGACCAGCGGCGT
GCACACCTTCCCGGCTGTCCTACAGTCCTCAGGACTCTAC
TCCCTCAGCAGCGTGGTGACCGTGCCCTCCAGCAGCTTGG
GCACCCAGACCTACATCTGCAACGTGAATCACAAGCCCAG
CAACACCAAGGTGGACAAGAAAGTTGAGCCCAAATCTTGT
GACAAAACTCACACATGCCCACCGTGCCCAGCACCTGAAC
TCCTGGGGGGACCGTCAGTCTTCCTCTTCCCCCCAAAACC
CAAGGACACCCTCATGATCTCCCGGACCCCTGAGGTCACA
TGCGTGGTGGTGGACGTGAGCCACGAAGACCCTGAGGTCA
AGTTCAACTGGTACGTGGACGGCGTGGAGGTGCATAATGC
CAAGACAAAGCCGCGGGAGGAGCAGTACAACAGCACGTAC
CGTGTGGTCAGCGTCCTCACCGTCCTGCACCAGGACTGGC
TGAATGGCAAGGAGTACAAGTGCAAGGTCTCCAACAAAGC
CCTCCCAGCCCCCATCGAGAAAACCATCTCCAAAGCCAAA
GGGCAGCCCCGAGAACCACAGGTCTACACCCTGCCCCCAT
CCCGGGAGGAGATGACCAAGAACCAGGTCAGCCTGTACTG
CCTGGTCAAAGGCTTCTATCCCAGCGACATCGCCGTGGAG
TGGGAGAGCAATGGGCAGCCGGAGAACAACTACAAGACCA
CGCCTCCCGTGCTGGACTCCGACGGCTCCTTCTTCCTCTA
TAGCAAGCTCACCGTGGACAAGAGCAGGTGGCAGCAGGGG
AACGTCTTCTCATGCTCCGTGATGCATGAGGCTCTGCACA
ACCACTACACGCAGAAGAGCCTCTCCCTGTCTCCGGGTAA
A
SEQ ID NO: 84:
GATGCATCTCTGCCTTACCTGCAGAAAGAAAGCGTGTTCC
AGTCTGGCGCCCACGCCTACAGAATTCCCGCTCTGCTGTA
TCTGCCAGGCCAGCAGTCTCTGCTGGCTTTCGCTGAACAG
CGGGCCAGCAAGAAGGATGAGCACGCCGAACTGATCGTGC
TGCGGAGAGGCGATTACGACGCCGGCACACATCAGGTGCA
GTGGCAGGCTCAAGAGGTGGTGGCTCAGGCTAGACTGGAC
GGCCACAGATCTATGAACCCCTGTCCTCTGTACGATGAAC
AGACCGGCACACTGTTTCTGTTCTTTATCGCTATCCCCGG
CCAAGTGACCGAGCAGCAGCAGCTGCAGACAAGAGCCAAC
GTGACCAGACTGTGTCAAGTGACCTCCACCGACCACGGCA
GAACCTGGTCTAGCCCTAGAGATCTGACCGACGCCGCCAT
CGGACCTGCCTATAGAGAGTGGTCCACCTTCGCCGTTGGA
CCTGGACACTGTCTCCAGCTGCACGACAGGGCTAGATCTC
TGGTGGTGCCTGCCTACGCCTATAGAAAGCTGCACCCCAA
ACAGCGGCCTATTCCTAGCGCCTTCTGCTTTCTGAGCCAC
GATCACGGCAGGACATGGGCCAGAGGACATTTCGTGGCCC
AGGACACACTGGAATGCCAGGTGGCCGAAGTGGAAACCGG
CGAGCAGAGAGTCGTGACCCTGAACGCCAGATCTCACCTG
AGAGCCAGAGTGCAGGCCCAGAGCACAAACGACGGCCTGG
ATTTCCAAGAGAGCCAGCTGGTCAAGAAACTGGTGGAACC
TCCTCCACAGGGCTGTCAGGGAAGCGTGATCAGCTTTCCA
TCTCCTAGAAGCGGCCCTGGCTCTCCTGCTCAGTGGCTGC
TGTATACACACCCCACACACAGCTGGCAGAGAGCCGATCT
GGGCGCCTACCTGAATCCTAGACCTCCTGCTCCTGAGGCT
TGGAGCGAACCTGTTCTGCTGGCCAAGGGCAGCGCTGCCT
ACAGCGATCTGCAGTCTATGGGCACAGGCCCTGATGGCAG
CCCTCTGTTTGGCTGTCTGTACGAGGCCAACGACTACGAA
GAGATCGTGTTCCTGATGTTCACCCTGAAGCAGGCCTTTC
CAGCCGAGTACCTGCCTCAAGGCGGAGGTGGAAGTGGCGG
AGGCGGATCCGACAAAACTCACACATGCCCACCGTGCCCA
GCACCTGAACTCCTGGGGGGACCGTCAGTCTTCCTCTTCC
CCCCAAAACCCAAGGACACCCTCATGATCTCCCGGACCCC
TGAGGTCACATGCGTGGTGGTGGACGTGAGCCACGAAGAC
CCTGAGGTCAAGTTCAACTGGTACGTGGACGGCGTGGAGG
TGCATAATGCCAAGACAAAGCCGCGGGAGGAGCAGTACAA
CAGCACGTACCGTGTGGTCAGCGTCCTCACCGTCCTGCAC
CAGGACTGGCTGAATGGCAAGGAGTACAAGTGCAAGGTCT
CCAACAAAGCCCTCCCAGCCCCCATCGAGAAAACCATCTC
CAAAGCCAAAGGGCAGCCCCGAGAACCACAGGTCTACACC
CTGCCCCCATCCCGGGAGGAGATGACCAAGAACCAGGTCA
GCCTGACCTGCCTGGTCAAAGGCTTCTATCCCAGCGACAT
CGCCGTGGAGTGGGAGAGCAATGGGCAGCCGGAGAACAAC
TACAAGACCACGCCTCCCGTGCTGGACTCCGACGGCTCCT
TCTTCCTCACTAGCAAGCTCACCGTGGACAAGAGCAGGTG
GCAGCAGGGGAACGTCTTCTCATGCTCCGTGATGCATGAG
GCTCTGCACAACCACTACACGCAGAAGAGCCTCTCCCTGT
CTCCGGGTAAA
SEQ ID NO: 85:
GATGCATCTCTGCCTTACCTGCAGAAAGAAAGCGTGTTCC
AGTCTGGCGCCCACGCCTACAGAATTCCCGCTCTGCTGTA
TCTGCCAGGCCAGCAGTCTCTGCTGGCTTTCGCTGAACAG
CGGGCCAGCAAGAAGGATGAGCACGCCGAACTGATCGTGC
TGCGGAGAGGCGATTACGACGCCGGCACACATCAGGTGCA
GTGGCAGGCTCAAGAGGTGGTGGCTCAGGCTAGACTGGAC
GGCCACAGATCTATGAACCCCTGTCCTCTGTACGATGAAC
AGACCGGCACACTGTTTCTGTTCTTTATCGCTATCCCCGG
CCAAGTGACCGAGCAGCAGCAGCTGCAGACAAGAGCCAAC
GTGACCAGACTGTGTCAAGTGACCTCCACCGACCACGGCA
GAACCTGGTCTAGCCCTAGAGATCTGACCGACGCCGCCAT
CGGACCTGCCTATAGAGAGTGGTCCACCTTCGCCGTTGGA
CCTGGACACTGTCTCCAGCTGCACGACAGGGCTAGATCTC
TGGTGGTGCCTGCCTACGCCTATAGAAAGCTGCACCCCAA
ACAGCGGCCTATTCCTAGCGCCTTCTGCTTTCTGAGCCAC
GATCACGGCAGGACATGGGCCAGAGGACATTTCGTGGCCC
AGGACACACTGGAATGCCAGGTGGCCGAAGTGGAAACCGG
CGAGCAGAGAGTCGTGACCCTGAACGCCAGATCTCACCTG
AGAGCCAGAGTGCAGGCCCAGAGCACAAACGACGGCCTGG
ATTTCCAAGAGAGCCAGCTGGTCAAGAAACTGGTGGAACC
TCCTCCACAGGGCTGTCAGGGAAGCGTGATCAGCTTTCCA
TCTCCTAGAAGCGGCCCTGGCTCTCCTGCTCAGTGGCTGC
TGTATACACACCCCACACACAGCTGGCAGAGAGCCGATCT
GGGCGCCTACCTGAATCCTAGACCTCCTGCTCCTGAGGCT
TGGAGCGAACCTGTTCTGCTGGCCAAGGGCAGCGCTGCCT
ACAGCGATCTGCAGTCTATGGGCACAGGCCCTGATGGCAG
CCCTCTGTTTGGCTGTCTGTACGAGGCCAACGACTACGAA
GAGATCGTGTTCCTGATGTTCACCCTGAAGCAGGCCTTTC
CAGCCGAGTACCTGCCTCAA
SEQ ID NO: 86:
CAGATCGTGCTGTCTCAGTCTCCTGCCATCCTGTCTGCTA
GCCCTGGCGAGAAAGTGACCATGACCTGTAGAGCCAGCAG
CAGCGTGTCCTACATCCACTGGTTCCAGCAGAAGCCTGGC
AGCAGCCCTAAGCCTTGGATCTACGCCACAAGCAATCTGG
CCAGCGGCGTGCCAGTCAGATTTTCTGGCTCTGGCAGCGG
CACCAGCTACAGCCTGACAATCTCTAGAGTGGAAGCCGAG
GACGCCGCCACCTACTATTGTCAGCAGTGGACCAGCAATC
CTCCTACCTTTGGCGGAGGCACCAAGCTGGAAATCAAGGC
CGTACGGTGGCTGCACCATCTGTCTTCATCTTCCCGCCAT
CTGATGAGCAGTTGAAATCTGGAACTGCCTCTGTTGTGTG
CCTGCTGAATAACTTCTATCCCAGAGAGGCCAAAGTACAG
TGGAAGGTGGATAACGCCCTCCAATCGGGTAACTCCCAGG
AGAGTGTCACAGAGCAGGACAGCAAGGACAGCACCTACAG
CCTCAGCAGCACCCTGACGCTGAGCAAAGCAGACTACGAG
AAACACAAAGTCTACGCCTGCGAAGTCACCCATCAGGGCC
TGAGCTCGCCCGTCACAAAGAGCTTCAACAGGGGAGAGTG
T
SEQ ID NO: 87:
CAGGTTCAGCTGCAACAGCCTGGCGCCGAACTTGTGAAAC
CTGGCGCCTCTGTGAAGATGAGCTGCAAGGCCAGCGGCTA
CACCTTCACCAGCTACAACATGCACTGGGTCAAGCAGACC
CCTGGCAGAGGCCTGGAATGGATCGGAGCCATCTATCCCG
GCAACGGCGACACCTCCTACAACCAGAAGTTCAAGGGCAA
AGCCACACTGACCGCCGACAAGAGCAGCAGCACAGCCTAC
ATGCAGCTGAGCAGCCTGACCAGCGAAGATAGCGCCGTGT
ACTACTGCGCCAGAAGCACCTATTACGGCGGCGACTGGTA
CTTCAACGTGTGGGGAGCTGGCACCACCGTGACAGTTTCT
GCTGCTAGCACCAAGGGCCCATCCGTCTTCCCCCTGGCAC
CCTCCTCCAAGAGCACCTCTGGGGGCACAGCGGCCCTGGG
CTGCCTGGTCAAGGACTACTTCCCCGAACCGGTGACGGTG
TCCTGGAACTCAGGCGCTCTGACCAGCGGCGTGCACACCT
TCCCGGCTGTCCTACAGTCCTCAGGACTCTACTCCCTCAG
CAGCGTGGTGACCGTGCCCTCCAGCAGCTTGGGCACCCAG
ACCTACATCTGCAACGTGAATCACAAGCCCAGCAACACCA
AGGTGGACAAGAAAGTTGAGCCCAAATCTTGTGACAAAAC
TCACACATGCCCACCGTGCCCAGCACCTGAACTCCTGGGG
GGACCGTCAGTCTTCCTCTTCCCCCCAAAACCCAAGGACA
CCCTCATGATCTCCCGGACCCCTGAGGTCACATGCGTGGT
GGTGGACGTGAGCCACGAAGACCCTGAGGTCAAGTTCAAC
TGGTACGTGGACGGCGTGGAGGTGCATAATGCCAAGACAA
AGCCGCGGGAGGAGCAGTACAACAGCACGTACCGTGTGGT
CAGCGTCCTCACCGTCCTGCACCAGGACTGGCTGAATGGC
AAGGAGTACAAGTGCAAGGTCTCCAACAAAGCCCTCCCAG
CCCCCATCGAGAAAACCATCTCCAAAGCCAAAGGGCAGCC
CCGAGAACCACAGGTCTACACCCTGCCCCCATCCCGGGAG
GAGATGACCAAGAACCAGGTCAGCCTGTACTGCCTGGTCA
AAGGCTTCTATCCCAGCGACATCGCCGTGGAGTGGGAGAG
CAATGGGCAGCCGGAGAACAACTACAAGACCACGCCTCCC
GTGCTGGACTCCGACGGCTCCTTCTTCCTCTATAGCAAGC
TCACCGTGGACAAGAGCAGGTGGCAGCAGGGGAACGTCTT
CTCATGCTCCGTGATGCATGAGGCTCTGCACAACCACTAC
ACGCAGAAGAGCCTCTCCCTGTCTCCGGGTAAA
SEQ ID NO: 88:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLTSKLTVDKSRWQQGNVFSCSVMHEALHNH
YTQKSLSLSPGK
SEQ ID NO: 89:
DASLPYLQDESVFQSGAHAYRIPALLYLPGQQSLLAFAEQ
RASKKDEHAELIVLRRGDYDAPTHQVQWQAQEVVAQARLD
GHRSMNPCPLYDEQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIRFIMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLTSKLTVDKSRWQQGNVFSCSVMHEALHNH
YTQKSLSLSPGK
SEQ ID NO: 90:
DASLPYLQKESVFQSGAHAYRIPALLYLPGQQSLLAFAEQ
RASKKDEHAELIVLRRGDYDANTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLTSKLTVDKSRWQQGNVFSCSVMHEALHNH
YTQKSLSLSPGK
SEQ ID NO: 91:
DASLPYLQKESVFQSGAHAYRIPALLYLPGQQSLLAFAEQ
RASKKDEHAELIVLRRGDYDAPTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPAGCQGSVISFP
SPRSGPGSPAQWLLYTHPTHRKQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLTSKLTVDKSRWQQGNVFSCSVMHEALHNH
YTQKSLSLSPGK
SEQ ID NO: 92:
DASLPYLQKESVFQSGAHAYRIPALLYLPGQQSLLAFAEQ
RASKKDEHAELIVLRRGDYDASTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPAGCQGSVISFP
SPRSGPGSPAQWLLYTHPTHRKQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLTSKLTVDKSRWQQGNVFSCSVMHEALHNH
YTQKSLSLSPGK
SEQ ID NO: 93:
DASLPYLQKESVFQSGAHAYRIPALLYLPGQQSLLAFAEQ
RASKKDEHAELIVLRRGDYDATTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPAGCQGSVISFP
SPRSGPGSPAQWLLYTHPTHRKQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLTSKLTVDKSRWQQGNVFSCSVMHEALHNH
YTQKSLSLSPGK
SEQ ID NO: 94:
DASLPYLQKESVFQSGAHAYRIPALLYLPGQQSLLAFAEQ
RASKKDEHAELIVLRRGDYDANTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPAGCQGSVISFP
SPRSGPGSPAQWLLYTHPTHRKQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLTSKLTVDKSRWQQGNVFSCSVMHEALHNH
YTQKSLSLSPGK
SEQ ID NO: 95:
X₁ASLPX₂LQX₃ESVFQSGAHAYRIPALLYLPGQQSLLAF
AEQRASKKDEHAELIVLRRGDYDAX₄THQVQWQAQEVVAQ
ARLDGHRSMNPCPLYDX₅QTGTLFLFFIAIPGQVTEQQQL
QTRANVTRLCQVTSTDHGRTWSSPRDLTDAAIGPAYREWS
TFAVGPGHCLQLHDRARSLVVPAYAYRKLHPX₆QRPIPSA
FCFLSHDHGRTWARGHFVAQDTLECQVAEVETGEQRVVTL
NARSHLRARVQAQSTNDGLDFQESQLVKKLVEPPPX₇GCQ
GSVISFPSPRSGPGSPAQWLLYTHPTHX₈X₉QRADLGAYL
NPRPPAPEAWSEPVLLAKGSX₁₀AYSDLQSMGTGPDGSPL
FGCLYEANDYEEIX₁₁FX₁₂MFTLKQAFPAEYLPQX₁₃DK
THTCPPCPAPELLGGPSVFLFPPKPKDTLMISRTPEVTCV
VVDVSHEDPEVKFNWYVDGVEVHNAKTKPREEQYNSTYRV
VSVLTVLHQDWLNGKEYKCKVSNKALPAPIEKTISKAKGQ
PREPQVYTLPPSREEMTKNQVSLTCLVKGFYPSDIAVEWE
SNGQPENNYKTTPPVLDSDGSFFLTSKLTVDKSRWQQGNV
FSCSVMHEALHNHYTQKSLSLSPGK
SEQ ID NO: 96:
X₁X₂SX₃X₄X₅LQX₆ESVFQSGAHAYRIPALLYLPGQQSL
LAFAEQRASX₇X₈DEHAELIVX₉RRGDYDAX₁₀THQVQWX₁₁
AQEVVAQAX₁₂LDGHRSMNPCPLYDX₁₃QTGTLFLFFI
AIPX₁₄X₁₅VTEX₁₆QQLQTRANVTRLX₁₇X₁₈VTSTDHG
RTWSSPRDLTDAAIGPX₁₉YREWSTFAVGPGHX₂₀LQLHD
RX₂₁RSLVVPAYAYRKLHPX₂₂QRPIPSAFX₂₃FLSHDHGR
TWARGHFVAQDTX₂₄ECQVAEVETGEQRVVTLNARSHLR
ARVQAQSX₂₅NX₂₆GLDFQX₂₇SQLVKKLVEPPPX₂₈GX₂₉
QGSVISFPSPRSGPGSPAQX₃₀LLYTHPTHX₃₁X₃₂QRA
DLGAYLNPRPPAPEAWSEPX₃₃LLAKGSX₃₄AYSDLQSMG
TGPDGSPLFGX₃₅LYEANDYEEIX₃₆FX₃₇MFTLKQAFPA
EYLPQX₃₈DKTHTCPPCPAPELLGGPSVFLFPPKPKDTLM
ISRTPEVTCVVVDVSHEDPEVKFNWYVDGVEVHNAKTKPR
EEQYNSTYRVVSVLTVLHQDWLNGKEYKCKVSNKALPAPI
EKTISKAKGQPREPQVYTLPPSREEMTKNQVSLTCLVKGF
YPSDIAVEWESNGQPENNYKTTPPVLDSDGSFFLTSKLTV
DKSRWQQGNVFSCSVMHEALHNHYTQKSLSLSPGK
SEQ ID NO: 97:
DASLPYLQKESVFQSGAHAYRIPALLYLPGQQSLLAFAEQ
RASKKDEHAELIVLRRGDYDAPTHQVQWQAQEVVAQARLD
GHRSMNPCPLYDAQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLTSKLTVDKSRWQQGNVFSCSVMHEALHNH
YTQKSLSLSPGK
SEQ ID NO: 98:
GGGGSGGGGS
SEQ ID NO: 99:
EPKSS
SEQ ID NO: 100:
EPKSCDKTHTCPPCPAPELLGGPSVFLFPPKPKDTLMISR
TPEVTCVVVDVSHEDPEVKFNWYVDGVEVHNAKTKPREEQ
YNSTYRVVSVLTVLHQDWLNGKEYKCKVSNKALPAPIEKT
ISKAKGQPREPQVYTLPPSREEMTKNQVSLTCLVKGFYPS
DIAVEWESNGQPENNYKTTPPVLDSDGSFFLTSKLTVDKS
RWQQGNVFSCSVMHEALHNHYTQKSLSLSPGK
SEQ ID NO: 101:
DKTHTCPPCPAPELLGGPSVFLFPPKPKDTLMISRTPEVT
CVVVDVSHEDPEVKFNWYVDGVEVHNAKTKPREEQYNSTY
RVVSVLTVLHQDWLNGKEYKCKVSNKALPAPIEKTISKAK
GQPREPQVYTLPPSREEMTKNQVSLYCLVKGFYPSDIAVE
WESNGQPENNYKTTPPVLDSDGSFFLYSKLTVDKSRWQQG
NVFSCSVMHEALHNHYTQKSLSLSPGK
SEQ ID NO: 102:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHARQRADLGAYLNPRPPAPEA
WSEPVLLAKGSAAYSDLQSMGTGPDGSPLFGCLYEANDYE
EIVFLMFTLKQAFPAEYLPQ
SEQ ID NO: 103:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHARQRADLGAYLNPRPPAPEA
WSEPVLLAKGSAAYSDLQSMGTGPDGSPLFGCLYEANDYE
EIVFLMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK
SEQ ID NO: 104:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHARQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLTSKLTVDKSRWQQGNVFSCSVMHEALHNH
YTQKSLSLSPGK
SEQ ID NO: 105:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCYVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPTGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQ
SEQ ID NO: 106:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCYVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPTGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK
SEQ ID NO: 107:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCYVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPTGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLTSKLTVDKSRWQQGNVFSCSVMHEALHNH
YTQKSLSLSPGK
SEQ ID NO: 108:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCYVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RFRVQAQSTNDGLDFQESQLVKKLVEPPPTGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQ
SEQ ID NO: 109:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCYVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RFRVQAQSTNDGLDFQESQLVKKLVEPPPTGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK
SEQ ID NO: 110:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCYVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RFRVQAQSTNDGLDFQESQLVKKLVEPPPTGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLTSKLTVDKSRWQQGNVFSCSVMHEALHNH
YTQKSLSLSPGK
SEQ ID NO: 111:
EIVLTQSPATLSLSPGERATLSCRASQSVSSYLAWYQQKP
GQAPRLLIYDASNRATGIPARFSGSGSGTDFTLTISSLEP
EDFAVYYCQQRSNWPITFGQGTRLEIKRTVAAPSVFIFPP
SDEQLKSGTASVVCLLNNEYPREAKVQWKVDNALQSGNSQ
ESVTEQDSKDSTYSLSSTLTLSKADYEKHKVYACEVTHQG
LSSPVTKSFNR
SEQ ID NO: 112:
GAGATCGTGCTGACACAGTCTCCCGCCACACTGTCACTGT
CTCCAGGCGAAAGAGCCACACTGAGCTGTAGAGCCAGCCA
GAGCGTGTCCTCTTACCTGGCCTGGTATCAGCAGAAGCCT
GGACAGGCTCCCCGGCTGCTGATCTACGATGCCAGCAATA
GAGCCACAGGCATCCCCGCCAGATTTTCTGGCTCTGGCAG
CGGCACCGATTTCACCCTGACCATAAGCAGCCTGGAACCT
GAGGACTTCGCCGTGTACTACTGCCAGCAGAGAAGCAACT
GGCCCATCACCTTTGGCCAGGGCACCAGACTGGAAATCAA
GCGTACGGTGGCTGCACCATCTGTCTTCATCTTCCCGCCA
TCTGATGAGCAGTTGAAATCTGGAACTGCCTCTGTTGTGT
GCCTGCTGAATAACTTCTATCCCAGAGAGGCCAAAGTACA
GTGGAAGGTGGATAACGCCCTCCAATCGGGTAACTCCCAG
GAGAGTGTCACAGAGCAGGACAGCAAGGACAGCACCTACA
GCCTCAGCAGCACCCTGACGCTGAGCAAAGCAGACTACGA
GAAACACAAAGTCTACGCCTGCGAAGTCACCCATCAGGGC
CTGAGCTCGCCCGTCACAAAGAGCTTCAACAGG
SEQ ID NO: 113
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK
SEQ ID NO: 114
GCATCTCTGCCTTACCTGCAGAAAGAAAGCGTGTTCCAGT
CTGGCGCCCACGCCTACAGAATTCCCGCTCTGCTGTATCT
GCCAGGCCAGCAGTCTCTGCTGGCTTTCGCTGAACAGCGG
GCCAGCAAGAAGGATGAGCACGCCGAACTGATCGTGCTGC
GGAGAGGCGATTACGACGCCGGCACACATCAGGTGCAGTG
GCAGGCTCAAGAGGTGGTGGCTCAGGCTAGACTGGACGGC
CACAGATCTATGAACCCCTGTCCTCTGTACGATGAACAGA
CCGGCACACTGTTTCTGTTCTTTATCGCTATCCCCGGCCA
AGTGACCGAGCAGCAGCAGCTGCAGACAAGAGCCAACGTG
ACCAGACTGTGTCAAGTGACCTCCACCGACCACGGCAGAA
CCTGGTCTAGCCCTAGAGATCTGACCGACGCCGCCATCGG
ACCTGCCTATAGAGAGTGGTCCACCTTCGCCGTTGGACCT
GGACACTGTCTCCAGCTGCACGACAGGGCTAGATCTCTGG
TGGTGCCTGCCTACGCCTATAGAAAGCTGCACCCCAAACA
GCGGCCTATTCCTAGCGCCTTCTGCTTTCTGAGCCACGAT
CACGGCAGGACATGGGCCAGAGGACATTTCGTGGCCCAGG
ACACACTGGAATGCCAGGTGGCCGAAGTGGAAACCGGCGA
GCAGAGAGTCGTGACCCTGAACGCCAGATCTCACCTGAGA
GCCAGAGTGCAGGCCCAGAGCACAAACGACGGCCTGGATT
TCCAAGAGAGCCAGCTGGTCAAGAAACTGGTGGAACCTCC
TCCACAGGGCTGTCAGGGAAGCGTGATCAGCTTTCCATCT
CCTAGAAGCGGCCCTGGCTCTCCTGCTCAGTGGCTGCTGT
ATACACACCCCACACACAGCTGGCAGAGAGCCGATCTGGG
CGCCTACCTGAATCCTAGACCTCCTGCTCCTGAGGCTTGG
AGCGAACCTGTTCTGCTGGCCAAGGGCAGCGCTGCCTACA
GCGATCTGCAGTCTATGGGCACAGGCCCTGATGGCAGCCC
TCTGTTTGGCTGTCTGTACGAGGCCAACGACTACGAAGAG
ATCGTGTTCCTGATGTTCACCCTGAAGCAGGCCTTTCCAG
CCGAGTACCTGCCTCAAGGCGGAGGTGGAAGTGGCGGAGG
CGGATCCGACAAAACTCACACATGCCCACCGTGCCCAGCA
CCTGAACTCCTGGGGGGACCGTCAGTCTTCCTCTTCCCCC
CAAAACCCAAGGACACCCTCATGATCTCCCGGACCCCTGA
GGTCACATGCGTGGTGGTGGACGTGAGCCACGAAGACCCT
GAGGTCAAGTTCAACTGGTACGTGGACGGCGTGGAGGTGC
ATAATGCCAAGACAAAGCCGCGGGAGGAGCAGTACAACAG
CACGTACCGTGTGGTCAGCGTCCTCACCGTCCTGCACCAG
GACTGGCTGAATGGCAAGGAGTACAAGTGCAAGGTCTCCA
ACAAAGCCCTCCCAGCCCCCATCGAGAAAACCATCTCCAA
AGCCAAAGGGCAGCCCCGAGAACCACAGGTCTACACCCTG
CCCCCATCCCGGGAGGAGATGACCAAGAACCAGGTCAGCC
TGACCTGCCTGGTCAAAGGCTTCTATCCCAGCGACATCGC
CGTGGAGTGGGAGAGCAATGGGCAGCCGGAGAACAACTAC
AAGACCACGCCTCCCGTGCTGGACTCCGACGGCTCCTTCT
TCCTCACTAGCAAGCTCACCGTGGACAAGAGCAGGTGGCA
GCAGGGGAACGTCTTCTCATGCTCCGTGATGCATGAGGCT
CTGCACAACCACTACACGCAGAAGAGCCTCTCCCTGTCTC
CGGGTAAA
SEQ ID NO: 115
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLTSKLTVDKSRWQQGNVFSCSVMHEALHNH
YTQKSLSLSPGK
SEQ ID NO: 116
GCATCTCTGCCTTACCTGCAGAAAGAAAGCGTGTTCCAGT
CTGGCGCCCACGCCTACAGAATTCCCGCTCTGCTGTATCT
GCCAGGCCAGCAGTCTCTGCTGGCTTTCGCTGAACAGCGG
GCCAGCAAGAAGGATGAGCACGCCGAACTGATCGTGCTGC
GGAGAGGCGATTACGACGCCGGCACACATCAGGTGCAGTG
GCAGGCTCAAGAGGTGGTGGCTCAGGCTAGACTGGACGGC
CACAGATCTATGAACCCCTGTCCTCTGTACGATGAACAGA
CCGGCACACTGTTTCTGTTCTTTATCGCTATCCCCGGCCA
AGTGACCGAGCAGCAGCAGCTGCAGACAAGAGCCAACGTG
ACCAGACTGTGTCAAGTGACCTCCACCGACCACGGCAGAA
CCTGGTCTAGCCCTAGAGATCTGACCGACGCCGCCATCGG
ACCTGCCTATAGAGAGTGGTCCACCTTCGCCGTTGGACCT
GGACACTGTCTCCAGCTGCACGACAGGGCTAGATCTCTGG
TGGTGCCTGCCTACGCCTATAGAAAGCTGCACCCCAAACA
GCGGCCTATTCCTAGCGCCTTCTGCTTTCTGAGCCACGAT
CACGGCAGGACATGGGCCAGAGGACATTTCGTGGCCCAGG
ACACACTGGAATGCCAGGTGGCCGAAGTGGAAACCGGCGA
GCAGAGAGTCGTGACCCTGAACGCCAGATCTCACCTGAGA
GCCAGAGTGCAGGCCCAGAGCACAAACGACGGCCTGGATT
TCCAAGAGAGCCAGCTGGTCAAGAAACTGGTGGAACCTCC
TCCACAGGGCTGTCAGGGAAGCGTGATCAGCTTTCCATCT
CCTAGAAGCGGCCCTGGCTCTCCTGCTCAGTGGCTGCTGT
ATACACACCCCACACACAGCTGGCAGAGAGCCGATCTGGG
CGCCTACCTGAATCCTAGACCTCCTGCTCCTGAGGCTTGG
AGCGAACCTGTTCTGCTGGCCAAGGGCAGCGCTGCCTACA
GCGATCTGCAGTCTATGGGCACAGGCCCTGATGGCAGCCC
TCTGTTTGGCTGTCTGTACGAGGCCAACGACTACGAAGAG
ATCGTGTTCCTGATGTTCACCCTGAAGCAGGCCTTTCCAG
CCGAGTACCTGCCTCAAGAGCCCAAATCTTCTGACAAAAC
TCACACATGCCCACCGTGCCCAGCACCTGAACTCCTGGGG
GGACCGTCAGTCTTCCTCTTCCCCCCAAAACCCAAGGACA
CCCTCATGATCTCCCGGACCCCTGAGGTCACATGCGTGGT
GGTGGACGTGAGCCACGAAGACCCTGAGGTCAAGTTCAAC
TGGTACGTGGACGGCGTGGAGGTGCATAATGCCAAGACAA
AGCCGCGGGAGGAGCAGTACAACAGCACGTACCGTGTGGT
CAGCGTCCTCACCGTCCTGCACCAGGACTGGCTGAATGGC
AAGGAGTACAAGTGCAAGGTCTCCAACAAAGCCCTCCCAG
CCCCCATCGAGAAAACCATCTCCAAAGCCAAAGGGCAGCC
CCGAGAACCACAGGTCTACACCCTGCCCCCATCCCGGGAG
GAGATGACCAAGAACCAGGTCAGCCTGACCTGCCTGGTCA
AAGGCTTCTATCCCAGCGACATCGCCGTGGAGTGGGAGAG
CAATGGGCAGCCGGAGAACAACTACAAGACCACGCCTCCC
GTGCTGGACTCCGACGGCTCCTTCTTCCTCACTAGCAAGC
TCACCGTGGACAAGAGCAGGTGGCAGCAGGGGAACGTCTT
CTCATGCTCCGTGATGCATGAGGCTCTGCACAACCACTAC
ACGCAGAAGAGCCTCTCCCTGTCTCCGGGTAAA
SEQ ID NO: 117
DASLPYLQDESVFQSGAHAYRIPALLYLPGQQSLLAFAEQ
RASKKDEHAELIVLRRGDYDAPTHQVQWQAQEVVAQARLD
GHRSMNPCPLYDEQTGTLFLFFIAIPGQVTEQQQLQTRAN
VTRLCQVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLANQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIRFRMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK
SEQ ID NO: 118
GATGCATCTCTGCCTTACCTGCAGGATGAAAGCGTGTTCC
AGTCTGGCGCCCACGCCTACAGAATTCCCGCTCTGCTGTA
TCTGCCAGGCCAGCAGTCTCTGCTGGCTTTCGCTGAACAG
CGGGCCAGCAAGAAGGATGAGCACGCCGAACTGATCGTGC
TGCGGAGAGGCGATTACGACGCCcctACACATCAGGTGCA
GTGGCAGGCTCAAGAGGTGGTGGCTCAGGCTAGACTGGAC
GGCCACAGATCTATGAACCCCTGTCCTCTGTACGATGAAC
AGACCGGCACACTGTTTCTGTTCTTTATCGCTATCCCCGG
CCAAGTGACCGAGCAGCAGCAGCTGCAGACAAGAGCCAAC
GTGACCAGACTGTGTCAAGTGACCTCCACCGACCACGGCA
GAACCTGGTCTAGCCCTAGAGATCTGACCGACGCCGCCAT
CGGACCTGCCTATAGAGAGTGGTCCACCTTCGCCGTTGGA
CCTGGACACTGTCTCCAGCTGCACGACAGGGCTAGATCTC
TGGTGGTGCCTGCCTACGCCTATAGAAAGCTGCACCCCAA
ACAGCGGCCTATTCCTAGCGCCTTCTGCTTTCTGAGCCAC
GATCACGGCAGGACATGGGCCAGAGGACATTTCGTGGCCC
AGGACACACTGGCGAATCAGGTGGCCGAAGTGGAAACCGG
CGAGCAGAGAGTCGTGACCCTGAACGCCAGATCTCACCTG
AGAGCCAGAGTGCAGGCCCAGAGCACAAACGACGGCCTGG
ATTTCCAAGAGAGCCAGCTGGTCAAGAAACTGGTGGAACC
TCCTCCACAGGGCTGTCAGGGAAGCGTGATCAGCTTTCCA
TCTCCTAGAAGCGGCCCTGGCTCTCCTGCTCAGTGGCTGC
TGTATACACACCCCACACACAGCTGGCAGAGAGCCGATCT
GGGCGCCTACCTGAATCCTAGACCTCCTGCTCCTGAGGCT
TGGAGCGAACCTGTTCTGCTGGCCAAGGGCAGCGCTGCCT
ACAGCGATCTGCAGTCTATGGGCACAGGCCCTGATGGCAG
CCCTCTGTTTGGCTGTCTGTACGAGGCCAACGACTACGAA
GAGATCCGTTTCCGTATGTTCACCCTGAAGCAGGCCTTTC
CAGCCGAGTACCTGCCTCAAGGCGGAGGTGGAAGTGGCGG
AGGCGGATCcGACAAAACTCACACATGCCCACCGTGCCCA
GCACCTGAACTCCTGGGGGGACCGTCAGTCTTCCTCTTCC
CCCCAAAACCCAAGGACACCCTCATGATCTCCCGGACCCC
TGAGGTCACATGCGTGGTGGTGGACGTGAGCCACGAAGAC
CCTGAGGTCAAGTTCAACTGGTACGTGGACGGCGTGGAGG
TGCATAATGCCAAGACAAAGCCGCGGGAGGAGCAGTACAA
CAGCACGTACCGTGTGGTCAGCGTCCTCACCGTCCTGCAC
CAGGACTGGCTGAATGGCAAGGAGTACAAGTGCAAGGTCT
CCAACAAAGCCCTCCCAGCCCCCATCGAGAAAACCATCTC
CAAAGCCAAAGGGCAGCCCCGAGAACCACAGGTcTACACC
CTGCCCCCATCCCGGGAGGAGATGACCAAGAACCAGGTCA
GCCTGACCTGCCTGGTCAAAGGCTTCTATCCCAGCGACAT
CGCCGTGGAGTGGGAGAGCAATGGGCAGCCGGAGAACAAC
TACAAGACCACGCCTCCCGTGCTGGACTCCGACGGCTCCT
TCTTCCTCacTAGCAAGCTCACCGTGGACAAGAGCAGGTG
GCAGCAGGGGAACGTCTTCTCATGCTCCGTGATGCATGAG
GCTCTGCACAACCACTACACGCAGAAGAGCCTCTCCCTGT
CTCCGGGTAAA
SEQ ID NO: 119
EVQLVESGGGLVQPGRSLRLSCAASGFTFNDYAMHWVRQA
PGKGLEWVSTISWNSGSIGYADSVKGRFTISRDNAKKSLY
LQMNSLRAEDTALYYCAKDIQYGNYYYGMDVWGQGTTVTV
SSASTKGPSVFPLAPSSKSTSGGTAALGCLVKDYFPEPVT
VSWNSGALTSGVHTFPAVLQSSGLYSLSSVVTVPSSSLGT
QTYICNVNHKPSNTKVDKKVEPKSCDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLYSKLTVDKSRWQQGNVFSCSVMHEALHNH
YTQKSLSLSPGKGGGGSGGGGSDASLPYLQKESVFQSGAH
AYRIPALLYLPGQQSLLAFAEQRASKKDEHAELIVLRRGD
YDAGTHQVQWQAQEVVAQARLDGHRSMNPCPLYDEQTGTL
FLFFIAIPGQVTEQQQLQTRANVTRLCQVTSTDHGRTWSS
PRDLTDAAIGPAYREWSTFAVGPGHCLQLHDRARSLVVPA
YAYRKLHPKQRPIPSAFCFLSHDHGRTWARGHFVAQDTLE
CQVAEVETGEQRVVTLNARSHLRARVQAQSTNDGLDFQES
QLVKKLVEPPPQGCQGSVISFPSPRSGPGSPAQWLLYTHP
THSWQRADLGAYLNPRPPAPEAWSEPVLLAKGSAAYSDLQ
SMGTGPDGSPLFGCLYEANDYEEIVFLMFTLKQAFPAEYL
PQ
SEQ ID NO: 120
GAGGTGCAGCTGGTTGAATCTGGCGGAGGACTGGTGCAGC
CTGGCAGATCTCTGAGACTGTCTTGTGCCGCCAGCGGCTT
CACCTTCAACGACTATGCCATGCACTGGGTCCGACAGGCC
CCTGGCAAAGGACTTGAATGGGTGTCCACCATCAGCTGGA
ACAGCGGCTCTATCGGCTACGCCGATTCCGTGAAGGGCAG
ATTCACCATCTCCAGAGACAACGCCAAGAAGTCCCTGTAC
CTGCAGATGAACAGCCTGAGAGCCGAGGACACAGCCCTGT
ACTACTGCGCCAAGGACATCCAGTACGGCAACTACTACTA
CGGCATGGACGTGTGGGGCCAGGGCACAACAGTGACAGTC
TCTTCTGCTAGCACCAAGGGCCCATCCGTCTTCCCCCTGG
CACCCTCCTCCAAGAGCACCTCTGGGGGCACAGCGGCCCT
GGGCTGCCTGGTCAAGGACTACTTCCCCGAACCGGTGACG
GTGTCCTGGAACTCAGGCGCTCTGACCAGCGGCGTGCACA
CCTTCCCGGCTGTCCTACAGTCCTCAGGACTCTACTCCCT
CAGCAGCGTGGTGACCGTGCCCTCCAGCAGCTTGGGCACC
CAGACCTACATCTGCAACGTGAATCACAAGCCCAGCAACA
CCAAGGTGGACAAGAAAGTTGAGCCCAAATCTTGTGACAA
AACTCACACATGCCCACCGTGCCCAGCACCTGAACTCCTG
GGGGGACCGTCAGTCTTCCTCTTCCCCCCAAAACCCAAGG
ACACCCTCATGATCTCCCGGACCCCTGAGGTCACATGCGT
GGTGGTGGACGTGAGCCACGAAGACCCTGAGGTCAAGTTC
AACTGGTACGTGGACGGCGTGGAGGTGCATAATGCCAAGA
CAAAGCCGCGGGAGGAGCAGTACAACAGCACGTACCGTGT
GGTCAGCGTCCTCACCGTCCTGCACCAGGACTGGCTGAAT
GGCAAGGAGTACAAGTGCAAGGTCTCCAACAAAGCCCTCC
CAGCCCCCATCGAGAAAACCATCTCCAAAGCCAAAGGGCA
GCCCCGAGAACCACAGGTCTACACCCTGCCCCCATCCCGG
GAGGAGATGACCAAGAACCAGGTCAGCCTGACCTGCCTGG
TCAAAGGCTTCTATCCCAGCGACATCGCCGTGGAGTGGGA
GAGCAATGGGCAGCCGGAGAACAACTACAAGACCACGCCT
CCCGTGCTGGACTCCGACGGCTCCTTCTTCCTCTATAGCA
AGCTCACCGTGGACAAGAGCAGGTGGCAGCAGGGGAACGT
CTTCTCATGCTCCGTGATGCATGAGGCTCTGCACAACCAC
TACACGCAGAAGAGCCTCTCCCTGTCTCCGGGTAAAGGCG
GAGGTGGAAGTGGCGGAGGCGGATCCGATGCATCTCTGCC
TTACCTGCAGAAAGAAAGCGTGTTCCAGTCTGGCGCCCAC
GCCTACAGAATTCCCGCTCTGCTGTATCTGCCAGGCCAGC
AGTCTCTGCTGGCTTTCGCTGAACAGCGGGCCAGCAAGAA
GGATGAGCACGCCGAACTGATCGTGCTGCGGAGAGGCGAT
TACGACGCCGGCACACATCAGGTGCAGTGGCAGGCTCAAG
AGGTGGTGGCTCAGGCTAGACTGGACGGCCACAGATCTAT
GAACCCCTGTCCTCTGTACGATGAACAGACCGGCACACTG
TTTCTGTTCTTTATCGCTATCCCCGGCCAAGTGACCGAGC
AGCAGCAGCTGCAGACAAGAGCCAACGTGAGCAGACTGTG
TCAAGTGAGCTCCACCGAGCACGGCAGAACCTGGTCTAGC
CCTAGAGATCTGACCGACGCCGCCATCGGACCTGCCTATA
GAGAGTGGTCCACCTTCGCCGTTGGACCTGGACACTGTCT
CCAGCTGCACGACAGGGCTAGATCTCTGGTGGTGCCTGCC
TACGCCTATAGAAAGCTGCACCCCAAACAGCGGCCTATTC
CTAGCGCCTTCTGCTTTCTGAGCCACGATCACGGCAGGAC
ATGGGCCAGAGGACATTTCGTGGCCCAGGACACACTGGAA
TGCCAGGTGGCCGAAGTGGAAACCGGCGAGCAGAGAGTCG
TGACCCTGAACGCCAGATCTCACCTGAGAGCCAGAGTGCA
GGCCCAGAGCACAAACGACGGCCTGGATTTCCAAGAGAGC
CAGCTGGTCAAGAAACTGGTGGAACCTCCTCCACAGGGCT
GTCAGGGAAGCGTGATCAGCTTTCCATCTCCTAGAAGCGG
CCCTGGCTCTCCTGCTCAGTGGCTGCTGTATACACACCCC
ACACACAGCTGGCAGAGAGCCGATCTGGGCGCCTACCTGA
ATCCTAGACCTCCTGCTCCTGAGGCTTGGAGCGAACCTGT
TCTGCTGGCCAAGGGCAGCGCTGCCTACAGCGATCTGCAG
TCTATGGGCACAGGCCCTGATGGCAGCCCTCTGTTTGGCT
GTCTGTACGAGGCCAACGACTACGAAGAGATCGTGTTCCT
GATGTTCACCCTGAAGCAGGCCTTTCCAGCCGAGTACCTG
CCTCAA
SEQ ID NO: 121
EVQLVESGGGLVQPGRSLRLSCAASGFTFNDYAMHWVRQA
PGKGLEWVSTISWNSGSIGYADSVKGRFTISRDNAKKSLY
LQMNSLRAEDTALYYCAKDIQYGNYYYGMDVWGQGTTVTV
SSASTKGPSVFPLAPSSKSTSGGTAALGCLVKDYFPEPVT
VSWNSGALTSGVHTFPAVLQSSGLYSLSSVVTVPSSSLGT
QTYICNVNHKPSNTKVDKKVEPKSCDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLYSKLTVDKSRWQQGNVFSCSVMHEALHNH
YTQKSLSLSPGKGGGGSGGGGSDASLPYLQKESVFQSGAH
AYRIPALLYLPGQQSLLAFAEQRASKKDEHAELIVLRRGD
YDAGTHQVQWQAQEVVAQARLDGHRSMNPCPLYDEQTGTL
FLFFIAIPGQVTEQQQLQTRANVTRLCYVTSTDHGRTWSS
PRDLTDAAIGPAYREWSTFAVGPGHCLQLHDRARSLVVPA
YAYRKLHPKQRPIPSAFCFLSHDHGRTWARGHFVAQDTLE
CQVAEVETGEQRVVTLNARSHLRARVQAQSTNDGLDFQES
QLVKKLVEPPPTGCQGSVISFPSPRSGPGSPAQWLLYTHP
THSWQRADLGAYLNPRPPAPEAWSEPVLLAKGSAAYSDLQ
SMGTGPDGSPLFGCLYEANDYEEIVFLMFTLKQAFPAEYL
PQ
SEQ ID NO: 122
GAGGTGCAGCTGGTTGAATCTGGCGGAGGACTGGTGCAGC
CTGGCAGATCTCTGAGACTGTCTTGTGCCGCCAGCGGCTT
CACCTTCAACGACTATGCCATGCACTGGGTCCGACAGGCC
CCTGGCAAAGGACTTGAATGGGTGTCCACCATCAGCTGGA
ACAGCGGCTCTATCGGCTACGCCGATTCCGTGAAGGGCAG
ATTCACCATCTCCAGAGACAACGCCAAGAAGTCCCTGTAC
CTGCAGATGAACAGCCTGAGAGCCGAGGACACAGCCCTGT
ACTACTGCGCCAAGGACATCCAGTACGGCAACTACTACTA
CGGCATGGACGTGTGGGGCCAGGGCACAACAGTGACAGTC
TCTTCTGCTAGCACCAAGGGCCCATCCGTCTTCCCCCTGG
CACCCTCCTCCAAGAGCACCTCTGGGGGCACAGCGGCCCT
GGGCTGCCTGGTCAAGGACTACTTCCCCGAACCGGTGACG
GTGTCCTGGAACTCAGGCGCTCTGACCAGCGGCGTGCACA
CCTTCCCGGCTGTCCTACAGTCCTCAGGACTCTACTCCCT
CAGCAGCGTGGTGACCGTGCCCTCCAGCAGCTTGGGCACC
CAGACCTACATCTGCAACGTGAATCACAAGCCCAGCAACA
CCAAGGTGGACAAGAAAGTTGAGCCCAAATCTTGTGACAA
AACTCACACATGCCCACCGTGCCCAGCACCTGAACTCCTG
GGGGGACCGTCAGTCTTCCTCTTCCCCCCAAAACCCAAGG
ACACCCTCATGATCTCCCGGACCCCTGAGGTCACATGCGT
GGTGGTGGACGTGAGCCACGAAGACCCTGAGGTCAAGTTC
AACTGGTACGTGGACGGCGTGGAGGTGCATAATGCCAAGA
CAAAGCCGCGGGAGGAGCAGTACAACAGCACGTACCGTGT
GGTCAGCGTCCTCACCGTCCTGCACCAGGACTGGCTGAAT
GGCAAGGAGTACAAGTGCAAGGTCTCCAACAAAGCCCTCC
CAGCCCCCATCGAGAAAACCATCTCCAAAGCCAAAGGGCA
GCCCCGAGAACCACAGGTCTACACCCTGCCCCCATCCCGG
GAGGAGATGACCAAGAACCAGGTCAGCCTGACCTGCCTGG
TCAAAGGCTTCTATCCCAGCGACATCGCCGTGGAGTGGGA
GAGCAATGGGCAGCCGGAGAACAACTACAAGACCACGCCT
CCCGTGCTGGACTCCGACGGCTCCTTCTTCCTCTATAGCA
AGCTCACCGTGGACAAGAGCAGGTGGCAGCAGGGGAACGT
CTTCTCATGCTCCGTGATGCATGAGGCTCTGCACAACCAC
TACACGCAGAAGAGCCTCTCCCTGTCTCCGGGTAAAGGCG
GAGGTGGAAGTGGCGGAGGCGGATCCGATGCATCTCTGCC
TTACCTGCAGAAAGAAAGCGTGTTCCAGTCTGGCGCCCAC
GCCTACAGAATTCCCGCTCTGCTGTATCTGCCAGGCCAGC
AGTCTCTGCTGGCTTTCGCTGAACAGCGGGCCAGCAAGAA
GGATGAGCACGCCGAACTGATCGTGCTGCGGAGAGGCGAT
TACGACGCCGGCACACATCAGGTGCAGTGGCAGGCTCAAG
AGGTGGTGGCTCAGGCTAGACTGGACGGCCACAGATCTAT
GAACCCCTGTCCTCTGTACGATGAACAGACCGGCACACTG
TTTCTGTTCTTTATCGCTATCCCCGGCCAAGTGACCGAGC
AGCAGCAGCTGCAGACAAGAGCCAACGTGAGCAGACTGTG
TTACGTGAGCTCGAGCGAGGAGGGCAGAACCTGGTCTAGC
CCTAGAGATCTGACCGACGCCGCCATCGGACCTGCCTATA
GAGAGTGGTCCACCTTCGCCGTTGGACCTGGACACTGTCT
CCAGCTGCACGACAGGGCTAGATCTCTGGTGGTGCCTGCC
TACGCCTATAGAAAGCTGCACCCCAAACAGCGGCCTATTC
CTAGCGCCTTCTGCTTTCTGAGCCACGATCACGGCAGGAC
ATGGGCCAGAGGACATTTCGTGGCCCAGGACACACTGGAA
TGCCAGGTGGCCGAAGTGGAAACCGGCGAGCAGAGAGTCG
TGACCCTGAACGCCAGATCTCACCTGAGAGCCAGAGTGCA
GGCCCAGAGCACAAACGACGGCCTGGATTTCCAAGAGAGC
CAGCTGGTCAAGAAACTGGTGGAACCTCCTCCAACCGGCT
GTCAGGGAAGCGTGATCAGCTTTCCATCTCCTAGAAGCGG
CCCTGGCTCTCCTGCTCAGTGGCTGCTGTATACACACCCC
ACACACAGCTGGCAGAGAGCCGATCTGGGCGCCTACCTGA
ATCCTAGACCTCCTGCTCCTGAGGCTTGGAGCGAACCTGT
TCTGCTGGCCAAGGGCAGCGCTGCCTACAGCGATCTGCAG
TCTATGGGCACAGGCCCTGATGGCAGCCCTCTGTTTGGCT
GTCTGTACGAGGCCAACGACTACGAAGAGATCGTGTTCCT
GATGTTCACCCTGAAGCAGGCCTTTCCAGCCGAGTACCTG
CCTCAA
SEQ ID NO: 123:
gatGCATCTCTGCCTTACCTGCAGAAAGAAAGCGTGTTCC
AGTCTGGCGCCCACGCCTACAGAATTCCCGCTCTGCTGT
ATCTGCCAGGCCAGCAGTCTCTGCTGGCTTTCGCTGAAC
AGCGGGCCAGCAAGAAGGATGAGCACGCCGAACTGATCG
TGCTGCGGAGAGGCGATTACGACGCCggcACACATCAGG
TGCAGTGGCAGGCTCAAGAGGTGGTGGCTCAGGCTAGAC
TGGACGGCCACAGATCTATGAACCCCTGTCCTCTGTACG
ATgaaCAGACCGGCACACTGTTTCTGTTCTTTATCGCTAT
CCCCGGCCAAGTGACCGAGCAGCAGCAGCTGCAGACAAG
AGCCAACGTGACCAGACTGTGTtacGTGACCTCCACCGA
CCACGGCAGAACCTGGTCTAGCCCTAGAGATCTGACCGA
CGCCGCCATCGGACCTGCCTATAGAGAGTGGTCCACCTT
CGCCGTTGGACCTGGACACTGTCTCCAGCTGCACGACAG
GGCTAGATCTCTGGTGGTGCCTGCCTACGCCTATAGAAA
GCTGCACCCCAAACAGCGGCCTATTCCTAGCGCCTTCTG
CTTTCTGAGCCACGATCACGGCAGGACATGGGCCAGAGG
ACATTTCGTGGCCCAGGACACACTGGAATGCCAGGTGGC
CGAAGTGGAAACCGGCGAGCAGAGAGTCGTGACCCTGAA
CGCCAGATCTCACCTGAGATTCAGAGTGCAGGCCCAGAG
CACAAACGACGGCCTGGATTTCCAAGAGAGCCAGCTGGT
CAAGAAACTGGTGGAACCTCCTCCAaccGGCTGTCAGGGA
AGCGTGATCAGCTTTCCATCTCCTAGAAGCGGCCCTGGC
TCTCCTGCTCAGTGGCTGCTGTATACACACCCCACACAC
AGCTGGCAGAGAGCCGATCTGGGCGCCTACCTGAATCCT
AGACCTCCTGCTCCTGAGGCTTGGAGCGAACCTGTTCTG
CTGGCCAAGGGCAGCgctGCCTACAGCGATCTGCAGTCTA
TGGGCACAGGCCCTGATGGCAGCCCTCTGTTTGGCTGTC
TGTACGAGGCCAACGACTACGAAGAGATCGTGTTCCTGA
TGTTCACCCTGAAGCAGGCCTTTCCAGCCGAGTACCTGC
CTCAA
SEQ ID NO: 124:
X₁X₂SX₃X₄X₅LQX₆ESVFQSGAHAYRIPALLYLPGQQSL
LAFAEQRASX₇X₈DEHAELIVX₉RRGDYDAX₁₀THQVQW
X₁₁AQEVVAQAX₁₂LX₁₃GHRSMNPCPLYDX₁₄QTGTLFL
FFIAIPX₁₅X₁₆VTEX₁₇QQLQTRANVTRLX₁₈X₁₉VTST
DHGRTWSSPRDLTDAAIGPX₂₀YREWSTFAVGPGHX₂₁LQ
LHDX₂₂X₂₃RSLVVPAYAYRKLHPX₂₄X₂₅X₂₆PIPSAF
X₂₇FLSHDHGRTWARGHFVX₂₈QDTX₂₉ECQVAEVX₃₀TG
EQRVVTLNARSX₃₁X₃₂X₃₃X₃₄RX₃₅QAQSX₃₆NX₃₇GL
DFQX₃₈X₃₉QX₄₀VKKLX₄₁EPPPX₄₂GX₄₃QGSVISFPS
PRSGPGSPAQX₄₄LLYTHPTHX₄₅X₄₆QRADLGAYLNPRP
PAPEAWSEPX₄₇LLAKGSX₄₈AYSDLQSMGTGPDGSPLFG
X₄₉LYEANDYEEIX₅₀FX₅₁MFTLKQAFPAEYLPQ
SEQ ID NO: 125:
X₁ASLPX₂LQX₃ESVFQSGAHAYRIPALLYLPGQQSLLAF
AEQRASKKDEHAELIVLRRGDYDAX₄THQVQWQAQEVVAQ
ARLDGHRSMNPCPLYDX₅QTGTLFLFFIAIPGQVTEQQQL
QTRANVTRLCX₆VTSTDHGRTWSSPRDLTDAAIGPAYREW
STFAVGPGHCLQLHDRARSLVVPAYAYRKLHPX₇QRPIPS
AFCFLSHDHGRTWARGHFVAQDTLECQVAEVETGEQRVVT
LNARSHLRX₈RVQAQSTNDGLDFQESQLVKKLVEPPPXGG
CQGSVISFPSPRSGPGSPAQWLLYTHPTHX₁₀X₁₁QRADL
GAYLNPRPPAPEAWSEPVLLAKGSX₁₂AYSDLQSMGTGPD
GSPLFGCLYEANDYEEIX₃FX₁₄MFTLKQAFPAEYLPQ
SEQ ID NO: 126:
GGGGS
SEQ ID NO: 127:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCYVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPTGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLYCLVKGFYPSDIAVEWESNGQPENNYKTTP
PVLDSDGSFFLYSKLTVDKSRWQQGNVFSCSVMHEALHNH
YTQKSLSLSPGK
SEQ ID NO: 128:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCYVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RFRVQAQSTNDGLDFQESQLVKKLVEPPPTGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLYCLVKGFYPSDIAVEWESNGQPENNYKTTP
PVLDSDGSFFLYSKLTVDKSRWQQGNVFSCSVMHEALHNH
YTQKSLSLSPGK
SEQ ID NO: 129:
QVQLQQPGAELVKPGASVKMSCKASGYTFTSYNMHWVKQT
PGRGLEWIGAIYPGNGDTSYNQKFKGKATLTADKSSSTAY
MQLSSLTSEDSAVYYCARSTYYGGDWYFNVWGAGTTVTVS
AASTKGPSVFPLAPSSKSTSGGTAALGCLVKDYFPEPVTV
SWNSGALTSGVHTFPAVLQSSGLYSLSSVVTVPSSSLGTQ
TYICNVNHKPSNTKVDKKVEPKSCDKTHTCPPCPAPELLG
GPSVFLFPPKPKDTLMISRTPEVTCVVVDVSHEDPEVKFN
WYVDGVEVHNAKTKPREEQYNSTYRVVSVLTVLHQDWLNG
KEYKCKVSNKALPAPIEKTISKAKGQPREPQVYTLPPSRE
EMTKNQVSLTCLVKGFYPSDIAVEWESNGQPENNYKTTPP
VLDSDGSFFLTSKLTVDKSRWQQGNVFSCSVMHEALHNHY
TQKSLSLSPGK
SEQ ID NO: 130:
EVQLVESGGGLVQPGRSLRLSCAASGFTFNDYAMHWVRQA
PGKGLEWVSTISWNSGSIGYADSVKGRFTISRDNAKKSLY
LQMNSLRAEDTALYYCAKDIQYGNYYYGMDVWGQGTTVTV
SSASTKGPSVFPLAPSSKSTSGGTAALGCLVKDYFPEPVT
VSWNSGALTSGVHTFPAVLQSSGLYSLSSVVTVPSSSLGT
QTYICNVNHKPSNTKVDKKVEPKSCDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGEYPSDIAVEWESNGQPENNYKTTP
PVLDSDGSFFLTSKLTVDKSRWQQGNVFSCSVMHEALHNH
YTQKSLSLSPGK
SEQ ID NO: 131:
X₁ASLPX₂LQX₃ESVFQSGAHAYRIPALLYLPGQQSLLAF
AEQRASKKDEHAELIVLRRGDYDAX₄THQVQWQAQEVVAQ
ARLDGHRSMNPCPLYDX₅QTGTLFLFFIAIPGQVTEQQQL
QTRANVTRLCX₆VTSTDHGRTWSSPRDLTDAAIGPAYREW
STFAVGPGHCLQLHDRARSLWPAYAYRKLHPX₇QRPIPSA
FCFLSHDHGRTWARGHFVAQDTLECQVAEVETGEQRVVTL
NARSHLRX₈RVQAQSTNDGLDFQESQLVKKLVEPPPX₉GC
QGSVISFPSPRSGPGSPAQWLLYTHPTHX₁₀X₁₁QRADLG
AYLNPRPPAPEAWSEPVLLAKGSX₁₂AYSDLQSMGTGPDG
SPLFGCLYEANDYEEIX₁₃FX₁₄MFTLKQAFPAEYLPQX₁₅
DKTHTCPPCPAPELLGGPSVFLFPPKPKDTLMISRTPEV
TCVVVDVSHEDPEVKFNWYVDGVEVHNAKTKPREEQYNST
YRVVSVLTVLHQDWLNGKEYKCKVSNKALPAPIEKTISKA
KGQPREPQVYTLPPSREEMTKNQVSLTCLVKGFYPSDIAV
EWESNGQPENNYKTTPPVLDSDGSFFLTSKLTVDKSRWQQ
GNVFSCSVMHEALHNHYTQKSLSLSPGK
SEQ ID NO: 132:
X₁X₂SX₃X₄X₅LQX₆ESVFQSGAHAYRIPALLYLPGQQSL
LAFAEQRASX₇X₈DEHAELIVX₉RRGDYDAX₁₀THQVQWX₁₁
AQEVVAQAX₁₂LX₁₃GHRSMNPCPLYDX₁₄QTGTLFLF
FIAIPX₁₅X₁₆VTEX₁₇QQLQTRANVTRLX₁₈X₁₉VTSTD
HGRTWSSPRDLTDAAIGPX₂₀YREWSTFAVGPGHX₂₁LQL
HDX₂₂X₂₃RSLWPAYAYRKLHPX₂₄X₂₅X₂₆PIPSAFX₂₇
FLSHDHGRTWARGHFVX₂₈QDTX₂₉ECQVAEVX₃₀TGEQR
VVTLNARSX₃₁X₃₂X₃₃X₃₄RX₃₅QAQSX₃₆NX₃₇GLDFQ
X₃₈X₃₉QX₄₀VKKLX₄₁EPPPX₄₂GX₄₃QGSVISFPSPRS
GPGSPAQX₄₄LLYTHPTHX₄₅X₄₆QRADLGAYLNPRPPAP
EAWSEPX₄₇LLAKGSX₄₈AYSDLQSMGTGPDGSPLFGX₄₉
LYEANDYEEIX₅₀FX₅₁MFTLKQAFPAEYLPQX₅₂DKTHT
CPPCPAPELLGGPSVFLFPPKPKDTLMISRTPEVTCVVVD
VSHEDPEVKFNWYVDGVEVHNAKTKPREEQYNSTYRVVSV
LTVLHQDWLNGKEYKCKVSNKALPAPIEKTISKAKGQPRE
PQVYTLPPSREEMTKNQVSLTCLVKGFYPSDIAVEWESNG
QPENNYKTTPPVLDSDGSFFLTSKLTVDKSRWQQGNVFSC
SVMHEALHNHYTQKSLSLSPGK
SEQ ID NO: 133:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCYVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQ
SEQ ID NO: 134:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCYVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQEPKSSDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLTSKLTVDKSRWQQGNVFSCSVMHEALHNH
YTQKSLSLSPGK
SEQ ID NO: 135:
DASLPYLQKESVFQSGAHAYRIPALLYLPGQQSLLAFAEQ
RASKKDEHAELIVLRRGDYDAGTHQVQWQAQEVVAQARLD
GHRSMNPCPLYDEQTGTLFLFFIAIPGQVTEQQQLQTRAN
VTRLCYVTSTDHGRTWSSPRDLTDAAIGPAYREWSTFAVG
PGHCLQLHDRARSLVVPAYAYRKLHPKQRPIPSAFCFLSH
DHGRTWARGHFVAQDTLECQVAEVETGEQRVVTLNARSHL
RARVQAQSTNDGLDFQESQLVKKLVEPPPQGCQGSVISFP
SPRSGPGSPAQWLLYTHPTHSWQRADLGAYLNPRPPAPEA
WSEPVLLAKGSAAYSDLQSMGTGPDGSPLFGCLYEANDYE
EIVFLMFTLKQAFPAEYLPQGGGGSGGGGSDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLTSKLTVDKSRWQQGNVFSCSVMHE
ALHNHYTQKSLSLSPGK

What is claimed is:

1. A fusion protein comprising:
(a) a sialidase enzyme; and
(b) an anti-CD20 immunoglobulin antigen-binding domain.

2. The fusion protein of claim 1, wherein the sialidase is a human sialidase.

3. The fusion protein of claim 1 or 2, wherein the sialidase is a recombinant mutant human sialidase.

4. The fusion protein of claim 3, wherein the sialidase comprises:
(a) a substitution of a proline residue at a position corresponding to position 5 of wild-type human Neu2 (P5);
(b) a substitution of a lysine residue at a position corresponding to position 9 of wild-type human Neu2 (K9);
(c) a substitution of a lysine residue at a position corresponding to position 44 of wild-type human Neu2 (K44);
(d) a substitution of a lysine residue at a position corresponding to position 45 of wild-type human Neu2 (K45);
(e) a substitution of a leucine residue at a position corresponding to position 54 of wild-type human Neu2 (L54);
(f) a substitution of a proline residue at a position corresponding to position 62 of wild-type human Neu2 (P62);
(g) a substitution of a glutamine residue at a position corresponding to position 69 of wild-type human Neu2 (Q69);
(h) a substitution of an arginine residue at a position corresponding to position 78 of wild-type human Neu2 (R78);
(i) a substitution of an aspartic acid residue at a position corresponding to position 80 of wild-type human Neu2 (D80);
(j) a substitution of an alanine residue at a position corresponding to position 93 of wild-type human Neu2 (A93);
(k) a substitution of a glycine residue at a position corresponding to position 107 of wild-type human Neu2 (G107);
(l) a substitution of a glutamine residue at a position corresponding to position 108 of wild-type human Neu2 (Q108);
(m) a substitution of a glutamine residue at a position corresponding to position 112 of wild-type human Neu2 (Q112);
(n) a substitution of a cysteine residue at a position corresponding to position 125 of wild-type human Neu2 (C125);
(o) a substitution of a glutamine residue at a position corresponding to position 126 of wild-type human Neu2 (Q126);
(p) a substitution of an alanine residue at a position corresponding to position 150 of wild-type human Neu2 (A150);
(q) a substitution of a cysteine residue at a position corresponding to position 164 of wild-type human Neu2 (C164);
(r) a substitution of an arginine residue at a position corresponding to position 170 of wild-type human Neu2 (R170);
(s) a substitution of an alanine residue at a position corresponding to position 171 of wild-type human Neu2 (A171);
(t) a substitution of a glutamine residue at a position corresponding to position 188 of wild-type human Neu2 (Q188);
(u) a substitution of an arginine residue at a position corresponding to position 189 of wild-type human Neu2 (R189);
(v) a substitution of an alanine residue at a position corresponding to position 213 of wild-type human Neu2 (A213);
(w) a substitution of a leucine residue at a position corresponding to position 217 of wild-type human Neu2 (L217);
(x) a substitution of a glutamic acid residue at a position corresponding to position 225 of wild-type human Neu2 (E225);
(y) a substitution of a histidine residue at a position corresponding to position 239 of wild-type human Neu2 (H239);
(z) a substitution of a leucine residue at a position corresponding to position 240 of wild-type human Neu2 (L240);
(aa) a substitution of an arginine residue at a position corresponding to position 241 of wild-type human Neu2 (R241);
(bb) a substitution of an alanine residue at a position corresponding to position 242 of wild-type human Neu2 (A242);
(cc) a substitution of a valine residue at a position corresponding to position 244 of wild-type human Neu2 (V244);
(dd) a substitution of a threonine residue at a position corresponding to position 249 of wild-type human Neu2 (T249);
(ee) a substitution of an aspartic acid residue at a position corresponding to position 251 of wild-type human Neu2 (D251);
(ff) a substitution of a glutamic acid residue at a position corresponding to position 257 of wild-type human Neu2 (E257);
(gg) a substitution of a serine residue at a position corresponding to position 258 of wild-type human Neu2 (S258);
(hh) a substitution of a leucine residue at a position corresponding to position 260 of wild-type human Neu2 (L260);
(ii) a substitution of a valine residue at a position corresponding to position 265 of wild-type human Neu2 (V265);
(jj) a substitution of a glutamine residue at a position corresponding to position 270 of wild-type human Neu2 (Q270);
(kk) a substitution of a tryptophan residue at a position corresponding to position 292 of wild-type human Neu2 (W292);
(ll) a substitution of a serine residue at a position corresponding to position 301 of wild-type human Neu2 (S301);
(mm) a substitution of a tryptophan residue at a position corresponding to position 302 of wild-type human Neu2 (W302);
(nn) a substitution of a valine residue at a position corresponding to position 363 of wild-type human Neu2 (V363); or
(oo) a substitution of a leucine residue at a position corresponding to position 365 of wild-type human Neu2 (L365);
or a combination of any of the foregoing substitutions.

5. The fusion protein of claim 3 or 4, wherein the sialidase comprises:
(a) a substitution of a proline residue at a position corresponding to position 5 of wild-type human Neu2 (P5);
(b) a substitution of a lysine residue at a position corresponding to position 9 of wild-type human Neu2 (K9);
(c) a substitution of a lysine residue at a position corresponding to position 44 of wild-type human Neu2 (K44);
(d) a substitution of a lysine residue at a position corresponding to position 45 of wild-type human Neu2 (K45);
(e) a substitution of a leucine residue at a position corresponding to position 54 of wild-type human Neu2 (L54);
(f) a substitution of a proline residue at a position corresponding to position 62 of wild-type human Neu2 (P62);
(g) a substitution of a glutamine residue at a position corresponding to position 69 of wild-type human Neu2 (Q69);
(h) a substitution of an arginine residue at a position corresponding to position 78 of wild-type human Neu2 (R78);
(i) a substitution of an alanine residue at a position corresponding to position 93 of wild-type human Neu2 (A93);
(j) a substitution of a glycine residue at a position corresponding to position 107 of wild-type human Neu2 (G107);
(k) a substitution of a glutamine residue at a position corresponding to position 108 of wild-type human Neu2 (Q108);
(l) a substitution of a glutamine residue at a position corresponding to position 112 of wild-type human Neu2 (Q112);
(m) a substitution of a cysteine residue at a position corresponding to position 125 of wild-type human Neu2 (C125);
(n) a substitution of a glutamine residue at a position corresponding to position 126 of wild-type human Neu2 (Q126);
(o) a substitution of an alanine residue at a position corresponding to position 150 of wild-type human Neu2 (A150);
(p) a substitution of a cysteine residue at a position corresponding to position 164 of wild-type human Neu2 (C164);
(q) a substitution of an alanine residue at a position corresponding to position 171 of wild-type human Neu2 (A171);
(r) a substitution of a leucine residue at a position corresponding to position 217 of wild-type human Neu2 (L217);
(s) a substitution of a threonine residue at a position corresponding to position 249 of wild-type human Neu2 (T249);
(t) a substitution of an aspartic acid residue at a position corresponding to position 251 of wild-type human Neu2 (D251);
(u) a substitution of a glutamine residue at a position corresponding to position 270 of wild-type human Neu2 (Q270);
(v) a substitution of a tryptophan residue at a position corresponding to position 292 of wild-type human Neu2 (W292);
(w) a substitution of a serine residue at a position corresponding to position 301 of wild-type human Neu2 (S301);
(x) a substitution of a tryptophan residue at a position corresponding to position 302 of wild-type human Neu2 (W302);
(y) a substitution of a valine residue at a position corresponding to position 363 of wild-type human Neu2 (V363); or
(z) a substitution of a leucine residue at a position corresponding to position 365 of wild-type human Neu2 (L365);
or a combination of any of the foregoing substitutions.

6. The fusion protein of claim 4 or 5, wherein the sialidase comprises a substitution of K9, P62, A93, Q216, A242, Q270, S301, W302, V363, or L365, or a combination of any of the foregoing substitutions.

7. The fusion protein of any one of claims 4-6, wherein the sialidase comprises a substitution of K9, P62, A93, Q270, S301, W302, V363, or L365, or a combination of any of the foregoing substitutions.

8. The fusion protein of any one of claims 4-7, wherein, in the sialidase:
(a) the proline residue at a position corresponding to position 5 of wild-type human Neu2 is substituted by histidine (P5H);
(b) the lysine residue at a position corresponding to position 9 of wild-type human Neu2 is substituted by aspartic acid (K9D);
(c) the lysine residue at a position corresponding to position 44 of wild-type human Neu2 is substituted by arginine (K44R) or glutamic acid (K44E);
(d) the lysine residue at a position corresponding to position 45 of wild-type human Neu2 is substituted by alanine (K45A), arginine (K45R), or glutamic acid (K45E);
(e) the leucine residue at a position corresponding to position 54 of wild-type human Neu2 is substituted by methionine (L54M);
(f) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T);
(g) the glutamine residue at a position corresponding to position 69 of wild-type human Neu2 is substituted by histidine (Q69H);
(h) the arginine residue at a position corresponding to position 78 of wild-type human Neu2 is substituted by lysine (R78K);
(i) the aspartic acid residue at a position corresponding to position 80 of wild-type human Neu2 is substituted by proline (D80P);
(j) the alanine residue at a position corresponding to position 93 of wild-type human Neu2 is substituted by glutamic acid (A93E) or lysine (A93K);
(k) the glycine residue at a position corresponding to position 107 of wild-type human Neu2 is substituted by aspartic acid (G107D);
(l) the glutamine residue at a position corresponding to position 108 of wild-type human Neu2 is substituted by histidine (Q108H);
(m) the glutamine residue at a position corresponding to position 112 of wild-type human Neu2 is substituted by arginine (Q112R) or lysine (Q112K);
(n) the cysteine residue at a position corresponding to position 125 of wild-type human Neu2 is substituted by leucine (C125L);
(o) the glutamine residue at a position corresponding to position 126 of wild-type human Neu2 is substituted by leucine (Q126L), glutamic acid (Q126E), phenylalanine (Q126F), histidine (Q126H), isoleucine (Q126I), or tyrosine (Q126Y);
(p) the alanine residue at a position corresponding to position 150 of wild-type human Neu2 is substituted by valine (A150V);
(q) the cysteine residue at a position corresponding to position 164 of wild-type human Neu2 is substituted by glycine (C164G);
(r) the arginine residue at a position corresponding to position 170 of wild-type human Neu2 is substituted by proline (R170P);
(s) the alanine residue at a position corresponding to position 171 of wild-type human Neu2 is substituted by glycine (A171G);
(t) the glutamine residue at a position corresponding to position 188 of wild-type human Neu2 is substituted by proline (Q188P);
(u) the arginine residue at a position corresponding to position 189 of wild-type human Neu2 is substituted by proline (R189P);
(v) the alanine residue at a position corresponding to position 213 of wild-type human Neu2 is substituted by cysteine (A213C), asparagine (A213N), serine (A213S) or threonine (A213 T);
(w) the leucine residue at a position corresponding to position 217 of wild-type human Neu2 is substituted by alanine (L217A) or valine (L217V);
(x) the threonine residue at a position corresponding to position 249 of wild-type human Neu2 is substituted by alanine (T249A);
(y) the aspartic acid residue at a position corresponding to position 251 of wild-type human Neu2 is substituted by glycine (D251G);
(z) the glutamic acid residue at a position corresponding to position 225 of wild-type human Neu2 is substituted by proline (E225P);
(aa) the histidine residue at a position corresponding to position 239 of wild-type human Neu2 is substituted by proline (H239P);
(bb) the leucine residue at a position corresponding to position 240 of wild-type human Neu2 is substituted by aspartic acid (L240D), asparagine (L240N), or tyrosine (L240Y);
(cc) the arginine residue at a position corresponding to position 241 of wild-type human Neu2 is substituted by alanine (R241A), aspartic acid (R241D), leucine (R241L), glutamine (R241Q), or tyrosine (R241Y);
(dd) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y);
(ee) the valine residue at a position corresponding to position 244 of wild-type human Neu2 is substituted by isoleucine (V244I) or proline (V244P);
(ff) the glutamic acid residue at a position corresponding to position 257 of wild-type human Neu2 is substituted by proline (E257P);
(gg) the serine residue at a position corresponding to position 258 is substituted by cysteine (S258C);
(hh) the leucine residue at a position corresponding to position 260 of wild-type human Neu2 is substituted by aspartic acid (L260D), phenylalanine (L260F), glutamine (L260Q), or threonine (L260T);
(ii) the valine residue at a position corresponding to position 265 of wild-type human Neu2 is substituted by phenylalanine (V265F);
(jj) the glutamine residue at a position corresponding to position 270 of wild-type human Neu2 is substituted by alanine (Q270A), histidine (Q270H), phenylalanine (Q270F), proline (Q270P), serine (Q270S), or threonine (Q270T);
(kk) the tryptophan residue at a position corresponding to position 292 of wild-type human Neu2 is substituted by arginine (W292R);
(ll) the serine residue at a position corresponding to position 301 of wild-type human Neu2 is substituted by alanine (S301A), aspartic acid (S301D), glutamic acid (S301E), phenylalanine (S301F), histidine (S301H), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y);
(mm) the tryptophan residue at a position corresponding to position 302 of wild-type human Neu2 is substituted by alanine (W302A), aspartic acid (W302D), phenylalanine (W302F), glycine (W302G), histidine (W302H), isoleucine (W302I), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y);
(nn) the valine residue at a position corresponding to position 363 of wild-type human Neu2 is substituted by arginine (V363R); or
(oo) the leucine residue at a position corresponding to position 365 of wild-type human Neu2 is substituted by glutamine (L365Q), histidine (L365H), isoleucine (L365I), lysine (L365K) or serine (L365S);
or the sialidase comprises a combination of any of the foregoing substitutions.

9. The fusion protein of any one of claims 4-8, wherein, in the sialidase:
(a) the proline residue at a position corresponding to position 5 of wild-type human Neu2 is substituted by histidine (P5H);
(b) the lysine residue at a position corresponding to position 9 of wild-type human Neu2 is substituted by aspartic acid (K9D);
(c) the lysine residue at a position corresponding to position 44 of wild-type human Neu2 is substituted by arginine (K44R) or glutamic acid (K44E);
(d) the lysine residue at a position corresponding to position 45 of wild-type human Neu2 is substituted by alanine (K45A), arginine (K45R), or glutamic acid (K45E);
(e) the leucine residue at a position corresponding to position 54 of wild-type human Neu2 is substituted by methionine (L54M);
(f) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T);
(g) the glutamine residue at a position corresponding to position 69 of wild-type human Neu2 is substituted by histidine (Q69H);
(h) the arginine residue at a position corresponding to position 78 of wild-type human Neu2 is substituted by lysine (R78K);
(i) the alanine residue at a position corresponding to position 93 of wild-type human Neu2 is substituted by glutamic acid (A93E) or lysine (A93K);
(j) the glycine residue at a position corresponding to position 107 of wild-type human Neu2 is substituted by aspartic acid (G107D);
(k) the glutamine residue at a position corresponding to position 108 of wild-type human Neu2 is substituted by histidine (Q108H);
(l) the glutamine residue at a position corresponding to position 112 of wild-type human Neu2 is substituted by arginine (Q112R) or lysine (Q112K);
(m) the cysteine residue at a position corresponding to position 125 of wild-type human Neu2 is substituted by leucine (C125L);
(n) the glutamine residue at a position corresponding to position 126 of wild-type human Neu2 is substituted by leucine (Q126L);
(o) the alanine residue at a position corresponding to position 150 of wild-type human Neu2 is substituted by valine (A150V);
(p) the cysteine residue at a position corresponding to position 164 of wild-type human Neu2 is substituted by glycine (C164G);
(q) the alanine residue at a position corresponding to position 171 of wild-type human Neu2 is substituted by glycine (A171G);
(r) the leucine residue at a position corresponding to position 217 of wild-type human Neu2 is substituted by alanine (L217A) or valine (L217V);
(s) the threonine residue at a position corresponding to position 249 of wild-type human Neu2 is substituted by alanine (T249A);
(t) the aspartic acid residue at a position corresponding to position 251 of wild-type human Neu2 is substituted by glycine (D251G);
(u) the glutamine residue at a position corresponding to position 270 of wild-type human Neu2 is substituted by alanine (Q270A), histidine (Q270H), phenylalanine (Q270F) or proline (Q270P);
(v) the tryptophan residue at a position corresponding to position 292 of wild-type human Neu2 is substituted by arginine (W292R);
(w) the serine residue at a position corresponding to position 301 of wild-type human Neu2 is substituted by arginine (S301R);
(x) the tryptophan residue at a position corresponding to position 302 of wild-type human Neu2 is substituted by lysine (W302K);
(y) the valine residue at a position corresponding to position 363 of wild-type human Neu2 is substituted by arginine (V363R); or
(z) the leucine residue at a position corresponding to position 365 of wild-type human Neu2 is substituted by glutamine (L365Q), histidine (L365H), isoleucine (L365I), lysine (L365K) or serine (L365S);
or the sialidase comprises a combination of any of the foregoing substitutions.

10. The fusion protein of claim 8 or 9, wherein the sialidase comprises a substitution selected from K9D, P62G, P62N, P62S, P62T, A93E, Q126Y, A242F, A242W, A242Y, Q270 A, Q270T, S301A, S301R, W302K, W302R, V363R, and L365I, or a combination of any of the foregoing substitutions.

11. The fusion protein of any one of claims 8-10, wherein the sialidase comprises a substitution selected from K9D, P62G, P62N, P62S, P62T, A93E, Q270A, S301R, W302K, V363R, and L365I, or a combination of any of the foregoing substitutions.

12. The fusion protein of any one of claims 1-11, wherein the sialidase comprises:
(a) a substitution or deletion of a methionine residue at a position corresponding to position 1 of wild-type human Neu2 (M1);
(b) a substitution of a valine residue at a position corresponding to position 6 of wild-type human Neu2 (V6);
(c) a substitution of an isoleucine residue at a position corresponding to position 187 of wild-type human Neu2 (I187); or
(d) a substitution of a cysteine residue at a position corresponding to position 332 of wild-type human Neu2 (C332);
or a combination of any of the foregoing substitutions.

13. The fusion protein of claim 12, wherein, in the sialidase:
(a) the methionine residue at a position corresponding to position 1 of wild-type human Neu2 is deleted (ΔM1), is substituted by alanine (M1A), or is substituted by aspartic acid (M1D);
(b) the valine residue at a position corresponding to position 6 of wild-type human Neu2 is substituted by tyrosine (V6Y);
(c) the isoleucine residue at a position corresponding to position 187 of wild-type human Neu2 is substituted by lysine (I187K); or
(d) the cysteine residue at a position corresponding to position 332 of wild-type human Neu2 is substituted by alanine (C332A);
or the sialidase comprises a combination of any of the foregoing substitutions.

14. The fusion protein of claim 13, wherein the sialidase comprises:
(a) the M1D, V6Y, P62G, A93E, I187K, and C332A substitutions;
(b) the M1D, V6Y, K9D, A93E, I187K, C332A, V363R, and L365I substitutions;
(c) the M1D, V6Y, P62N, I187K, and C332A substitutions;
(d) the M1D, V6Y, I187K, Q270A, S301R, W302K, and C332A substitutions;
(e) the M1D, V6Y, P62S, I187K, Q270A, S301R, W302K, and C332A substitutions;
(f) the M1D, V6Y, P62T, I187K, Q270A, S301R, W302K, and C332A substitutions;
(g) the M1D, V6Y, P62N, I187K, Q270A, S301R, W302K, and C332A substitutions;
(h) the M1D, V6Y, P62G, A93E, I187K, S301A, W302R, and C332A substitutions;
(i) the M1D, V6Y, P62G, A93E, Q126Y, I187K, Q270T, and C332A substitutions;
(j) the M1D, V6Y, P62G, A93E, Q126Y, I187K, and C332A substitutions; or
(k) the M1D, V6Y, P62G, A93E, Q126Y, I187K, A242F, Q270T, and C332A substitutions.

15. The fusion protein of claim 13 or 14, wherein the sialidase comprises:
(a) the M1D, V6Y, P62G, A93E, I187K, and C332A substitutions;
(b) the M1D, V6Y, K9D, A93E, I187K, C332A, V363R, and L365I substitutions;
(c) the M1D, V6Y, P62N, I187K, and C332A substitutions;
(d) the M1D, V6Y, I187K, Q270A, S301R, W302K, and C332A substitutions;
(e) the M1D, V6Y, P62S, I187K, Q270A, S301R, W302K, and C332A substitutions;
(f) the M1D, V6Y, P62T, I187K, Q270A, S301R, W302K, and C332A substitutions; or
(g) the M1D, V6Y, P62N, I187K, Q270A, S301R, W302K, and C332A substitutions.

16. The fusion protein of any one of claims 1-15, wherein the sialidase is selected from Neu1, Neu2, Neu3, and Neu4.

17. The fusion protein of claim 16, wherein the sialidase is Neu2.

18. The fusion protein of any one of claims 1-17, wherein the sialidase has a different substrate specificity than the corresponding wild-type sialidase.

19. The fusion protein of claim 18, wherein the sialidase can cleave α2,3, α2,6, and/or α2,8 linkages.

20. The fusion protein of claim 19, wherein the sialidase can cleave α2,3 and α2,8 linkages.

21. The fusion protein of any one of claims 1-20, wherein the sialidase comprises any one of SEQ ID NOs: 48-62, 102, 105, 108, or 133.

22. The fusion protein of any one of claims 1-21, wherein the sialidase comprises any one of SEQ ID NOs: 48-54.

23. The fusion protein of any one of claims 1-22, wherein the sialidase comprises a mutation set forth in any one of Tables 5-12 or 14-27.

24. The fusion protein of any one of claims 1-23, wherein the sialidase comprises a mutation set forth in any one of Tables 1-4.

25. The fusion protein of any one of claims 1-24, wherein the fusion protein further comprises an immunoglobulin Fc domain.

26. The fusion protein of claim 25, wherein the immunoglobulin Fc domain is derived from a human IgG1, IgG2, IgG3, IgG4, IgA1, IgA2, IgD, IgE, or IgM Fc domain.

27. The fusion protein of claim 26, wherein the immunoglobulin Fc domain is derived from a human IgG1, IgG2, IgG3, or IgG4 Fc domain.

28. The fusion protein of claim 27, wherein the immunoglobulin Fc domain is derived from a human IgG1 Fc domain.

29. The fusion protein of any one of claims 1-28, wherein the anti-CD20 immunoglobulin antigen-binding domain is associated with a second anti-CD20 immunoglobulin antigen-binding domain to produce an anti-CD20 antigen-binding site.

30. The fusion protein of any one of claims 1-29, wherein the anti-CD20 immunoglobulin antigen-binding domain is derived from an antibody selected from ofatumumab, rituximab, ocrelizumab, obinutuzumab, ibritumomab, veltuzumab, reditux, PRO131921, TRU-015, ubituximab, NCD1.2, blontuvetmab, FBTA05, mosunetuzumab, ocaratuzumab, ocrelizumab, and tositumomab.

31. The fusion protein of claim 30, wherein the anti-CD20 immunoglobulin antigen-binding domain is derived from ofatumumab or rituximab.

32. The fusion protein of any one of claims 1-31, wherein the sialidase and the immunoglobulin Fc domain and/or the anti-CD20 immunoglobulin antigen-binding domain are linked by a peptide bond or an amino acid linker.

33. The fusion protein of any one of claims 1-32, wherein the fusion protein comprises any one of SEQ ID NOs: 66-74, 78, 88-97, 103, 104, 106, 107, 109, 110, 113, 115, 117, 119, 121, 127, 128, 134, or 135.

34. The fusion protein of any one of claims 1-33, wherein the fusion protein comprises any one of SEQ ID NOs: 66-74, 78, or 88-97.

35. An antibody conjugate comprising the fusion protein of any one of claims 1-34.

36. The antibody conjugate of claim 35, wherein the antibody conjugate comprises a single sialidase.

37. The antibody conjugate of claim 35, wherein the antibody conjugate comprises two sialidases.

38. The antibody conjugate of claim 37, wherein the two sialidases are identical.

39. The antibody conjugate of any one of claims 35-38, wherein the antibody conjugate comprises a single anti-CD20 antigen-binding site.

40. The antibody conjugate of any one of claims 35-38, wherein the antibody conjugate comprises two anti-CD20 antigen-binding sites.

41. The antibody conjugate of claim 40, wherein the two anti-CD20 antigen-binding sites are identical.

42. The antibody conjugate of any one of claims 35-41, wherein the antibody conjugate has a molecular weight from about 135 kDa to about 165 kDa.

43. The antibody conjugate of any one of claims 35-41, wherein the antibody conjugate has a molecular weight from about 215 kDa to about 245 kDa.

44. The antibody conjugate of any one of claims 35-43, wherein the antibody conjugate comprises:
(a) a first polypeptide comprising an immunoglobulin light chain;
(b) a second polypeptide comprising an immunoglobulin heavy chain; and
(c) a third polypeptide comprising an immunoglobulin Fc domain and a sialidase;
wherein the first and second polypeptides are covalently linked together and the second and third polypeptides are linked together, and wherein the first polypeptide and the second polypeptide together define an anti-CD20 antigen-binding site.

45. The antibody conjugate of claim 44, wherein the third polypeptide comprises the sialidase and the immunoglobulin Fc domain in an N- to C-terminal orientation.

46. The antibody conjugate of claim 44 or 45, wherein the first polypeptide comprises SEQ ID NO: 66.

47. The antibody conjugate of claim 44 or 45, wherein the first polypeptide comprises any one of SEQ ID NOs: 77, 80, or 111.

48. The antibody conjugate of claim 44 or 45, wherein the first polypeptide comprises SEQ ID NO: 80.

49. The antibody conjugate of any one of claims 44-48, wherein the second polypeptide comprises any one of SEQ ID NOs: 67 or 129.

50. The antibody conjugate of any one of claims 44-48, wherein the second polypeptide comprises SEQ ID NO: 67.

51. The antibody conjugate of any one of claims 44-48, wherein the second polypeptide comprises any one of SEQ ID NOs: 81 or 130.

52. The antibody conjugate of any one of claims 44-48, wherein the second polypeptide comprises SEQ ID NO: 81.

53. The antibody conjugate of any one of claims 44-52, wherein the third polypeptide comprises any one of SEQ ID NOs: 68-74, 78, 88-97, 103, 104, 106, 107, 109, 110, 113, 115, 117, 127, 128, 134, or 135.

54. The antibody conjugate of any one of claims 44-52, wherein the third polypeptide comprises any one of SEQ ID NOs: 68-74, 78, or 88-97.

55. The antibody conjugate of any one of claims 35-43, wherein the fusion protein comprises:
(a) a first polypeptide comprising a first immunoglobulin light chain;
(b) a second polypeptide comprising a first immunoglobulin heavy chain and a first sialidase;
(c) a third polypeptide comprising a second immunoglobulin heavy chain and a second sialidase; and
(d) a fourth polypeptide comprising a second immunoglobulin light chain;
wherein the first and second polypeptides are covalently linked together, the third and fourth polypeptides are covalently linked together, and the second and third polypeptides are covalently linked together, and wherein the first polypeptide and the second polypeptide together define a first anti-CD20 antigen-binding site, and the third polypeptide and the fourth polypeptide together define a second anti-CD20 antigen-binding site.

56. The antibody conjugate of claim 55, wherein the second and third polypeptides comprise the first and second immunoglobulin heavy chain and the first and second sialidase, respectively, in an N- to C-terminal orientation.

57. The antibody conjugate of claim 55 or 56, wherein the first polypeptide and/or fourth polypeptide comprises any one of SEQ ID NOs: 77, 80, or 111.

58. The antibody conjugate of any one of claims 55-57, wherein the second and/or third polypeptide comprises any one of SEQ ID NOs: 119 or 121.

59. The antibody conjugate of any one of claims 35-43, wherein the fusion protein comprises:
(a) a first polypeptide comprising a first sialidase, a first immunoglobulin Fc domain, and a first single chain variable fragment (scFv); and
(b) a second polypeptide comprising a second sialidase, a second immunoglobulin Fc domain, and a second single chain variable fragment (scFv);
wherein the first and second polypeptides are covalently linked together, and wherein the first scFv defines a first anti-CD20 antigen-binding site, and the second scFv defines a second anti-CD20 antigen-binding site.

60. The antibody conjugate of claim 59, wherein the first polypeptide comprises the first sialidase, the first immunoglobulin Fc domain, and the first scFv in an N- to C-terminal orientation, and the second polypeptide comprises the second sialidase, the second immunoglobulin Fc domain, and the second scFv in an N- to C-terminal orientation.

61. The antibody conjugate of any one of claims 35-43, wherein the antibody conjugate comprises:
(a) a first polypeptide comprising an immunoglobulin light chain;
(b) a second polypeptide comprising an immunoglobulin heavy chain and a single chain variable fragment (scFv); and
(c) a third polypeptide comprising an immunoglobulin Fc domain and a sialidase;
wherein the first and second polypeptides are covalently linked together and the second and third polypeptides are covalently linked together, and wherein the immunoglobulin light chain and immunoglobulin heavy chain together define a first anti-CD20 antigen-binding site and the scFv defines a second anti-CD20 antigen-binding site.

62. The antibody conjugate of claim 61, wherein the second polypeptide comprises the immunoglobulin heavy chain and the scFv in an N- to C-terminal orientation, and the third polypeptide comprises the sialidase and the immunoglobulin Fc domain in an N- to C-terminal orientation.

63. An isolated nucleic acid comprising a nucleotide sequence encoding the fusion protein of any one of claims 1-34, or at least a portion of the antibody conjugate of any one of claims 35-62.

64. An expression vector comprising the nucleic acid of claim 63.

65. A host cell comprising the expression vector of claim 64.

66. A pharmaceutical composition comprising the fusion protein of any one of claims 1-34 or the antibody conjugate of any one of claims 35-62.

67. A method of treating cancer in a subject in need thereof, the method comprising administering to the subject an effective amount of the fusion protein of any one of claims 1-34, the antibody conjugate of any one of claims 35-62, or the pharmaceutical composition of claim 66.

68. The method of claim 67, wherein the cancer is selected from a lymphoma (e.g., a B-cell lymphoma, Hodgkin lymphoma, lymphoblastic lymphoma), a leukemia (e.g., hairy cell leukemia, B-cell chronic lymphocytic leukemia, acute myeloid leukemia (AML), acute lymphoblastic leukemia), and a melanoma.


SEQUENCE LISTING (152 sequences)

SEQ ID NO: 1 - protein, 380 aa, Homo sapiens
MASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 2 - protein, 385 aa, Mus musculus
MEDLRPMATCPVLQKETLFRTGVHAYRIPALLYLKKQKTLLAFAEKRASKTDEHAELIVLRRGSYNEATNRVKWQPEEVVTQAQLEGHRSMNPCPLYDKQTKTLFLFFIAVPGRVSEHHQLHTKVNVTRLCCVSSTDHGRTWSPIQDLTETTIGSTHQEWATFAVGPGHCLQLRNPAGSLLVPAYAYRKLHPAQKPTPFAFCFISLDHGHTWKLGNFVAENSLECQVAEVGTGAQRMVYLNARSFLGARVQAQSPNDGLDFQDNRVVSKLVEPPHGCHGSVVAFHNPISKPHALDTWLLYTHPTDSRNRTNLGVYLNQMPLDPTAWSEPTLLAMGICAYSDLQNMGQGPDGSPQFGCLYESGNYEEIIFLIFTLKQAFPTVFDAQ

SEQ ID NO: 3 - protein, 5 aa, Mus musculus
EDLRP

SEQ ID NO: 4 - protein, 6 aa, Mus musculus
MEDLRP

SEQ ID NO: 5 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 1143 bp, Salmonella typhimurium
acagtggaaaagtccgtggtgttcaaggccgagggcgagcacttcaccgaccagaaaggcaataccatcgtcggctctggcagcggcggcaccaccaagtactttagaatccccgccatgtgcaccaccagcaagggcaccattgtggtgttcgccgacgccagacacaacaccgccagcgatcagagcttcatcgataccgctgccgccagatctaccgatggcggcaagacctggaacaagaagatcgccatctacaacgaccgcgtgaacagcaagctgagcagagtgatggaccctacctgcatcgtggccaacatccagggcagagaaaccatcctggtcatggtcggaaagtggaacaacaacgataagacctggggcgcctacagagacaaggcccctgataccgattgggacctcgtgctgtacaagagcaccgatgacggcgtgaccttcagcaaggtggaaacaaacatccacgacatcgtgaccaagaacggcaccatctctgccatgctcggcggcgttggatctggcctgcaactgaatgatggcaagctggtgttccccgtgcagatggtccgaacaaagaatatcaccaccgtgctgaataccagcttcatctacagcaccgacggcatcacatggtccctgcctagcggctactgtgaaggctttggcagcgagaacaacatcatcgagttcaacgccagcctggtcaacaacatccggaacagcggcctgcggagaagcttcgagacaaaggacttcggaaagacgtggaccgagtttcctccaatggacaagaaggtggacaaccggaaccacggcgtgcagggcagcacaatcacaatccctagcggcaacaaactggtggccgctcactctagcgcccagaacaagaacaacgactacaccagaagcgacatcagcctgtacgcccacaacctgtacagcggcgaagtgaagctgatcgacgacttctaccccaaagtgggcaatgccagcggagccggctacagctgtctgagctaccggaaaaatgtggacaaagaaaccctgtacgtggtgtacgaggccaacggcagcatcgagtttcaggacctgagcagacatctgcccgtgatcaagagctacaac

SEQ ID NO: 7 - protein, 364 aa, Homo sapiens
ENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISV

SEQ ID NO: 8 - protein, 428 aa, Homo sapiens
MEEVTTCSFNSPLFRQEDDRGITYRIPALLYIPPTHTFLAFAEKRSTRRDEDALHLVLRRGLRIGQLVQWGPLKPLMEATLPGHRTMNPCPVWEQKSGCVFLFFICVRGHVTERQQIVSGRNAARLCFIYSQDAGCSWSEVRDLTEEVIGSELKHWATFAVGPGHGIQLQSGRLVIPAYTYYIPSWFFCFQLPCKTRPHSLMIYSDDLGVTWHHGRLIRPMVTVECEVAEVTGRAGHPVLYCSARTPNRCRAEALSTDHGEGFQRLALSRQLCEPPHGCQGSVVSFRPLEIPHRCQDSSSKDAPTIQQSSPGSSLRLEEEAGTPSESWLLYSHPTSRKQRVDLGIYLNQTPLEAACWSRPWILHCGPCGYSDLAALEEEGLFGCLFECGTKQECEQIAFRLFTHREILSHLQGDCTSPGRNPSQFKSN

SEQ ID NO: 9 - protein, 461 aa, Homo sapiens
MRPADLPPRPMEESPASSSAPTETEEPGSSAEVMEEVTTCSFNSPLFRQEDDRGITYRIPALLYIPPTHTFLAFAEKRSTRRDEDALHLVLRRGLRIGQLVQWGPLKPLMEATLPGHRTMNPCPVWEQKSGCVFLFFICVRGHVTERQQIVSGRNAARLCFIYSQDAGCSWSEVRDLTEEVIGSELKHWATFAVGPGHGIQLQSGRLVIPAYTYYIPSWFFCFQLPCKTRPHSLMIYSDDLGVTWHHGRLIRPMVTVECEVAEVTGRAGHPVLYCSARTPNRCRAEALSTDHGEGFQRLALSRQLCEPPHGCQGSVVSFRPLEIPHRCQDSSSKDAPTIQQSSPGSSLRLEEEAGTPSESWLLYSHPTSRKQRVDLGIYLNQTPLEAACWSRPWILHCGPCGYSDLAALEEEGLFGCLFECGTKQECEQIAFRLFTHREILSHLQGDCTSPGRNPSQFKSN

SEQ ID NO: 10 - protein, 484 aa, Homo sapiens
MGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 11 - protein, 496 aa, Homo sapiens
MMSSAAFPRWLSMGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 12 - protein, 5 aa, Homo sapiens
MASLP

SEQ ID NO: 13 - protein, 4 aa, Homo sapiens
ASLP

SEQ ID NO: 14 - protein, 8 aa, Salmonella typhimurium
TVEKSVVF

SEQ ID NO: 15 - protein, 12 aa, Homo sapiens
GDYDAPTHQVQW

SEQ ID NO: 16 - protein, 8 aa, Homo sapiens
SMDQGSTW

SEQ ID NO: 17 - protein, 8 aa, Salmonella typhimurium
STDGGKTW

SEQ ID NO: 18 - protein, 8 aa, Homo sapiens
PRPPAPEA

SEQ ID NO: 19 - protein, 8 aa, Homo sapiens
QTPLEAAC

SEQ ID NO: 20 - protein, 9 aa, Homo sapiens
NPRPPAPEA

SEQ ID NO: 21 - protein, 7 aa, Unknown
  Description of Unknown Recombinant mutant human sialidase substitution sequence
SQNDGES

SEQ ID NO: 22 - protein, 7 aa, Unknown
  Description of Unknown Recombinant mutant human sialidase substitution sequence
LSHSLST

SEQ ID NO: 23 - DNA, 1092 bp, Homo sapiens
gagaacgactttggactggtgcagcctctggtcaccatggaacagctgctgtgggtttccggcagacagatcggcagcgtggacaccttcagaatccctctgatcaccgccacacctagaggcaccctgctggcctttgccgaggccagaaagatgagcagctctgacgagggcgccaagtttattgccctgaggcggtctatggaccagggctctacatggtcccctaccgccttcatcgtgaacgatggcgacgtgcccgatggcctgaatctgggagctgtggtgtccgatgtggaaaccggcgtggtgttcctgttctacagcctgtgtgcccacaaggccggttgtcaggtggccagcacaatgctcgtgtggtccaaggacgacggcgtgtcctggtctacccctagaaacctgagcctggacatcggcaccgaagtgtttgctccaggacctggctctggcatccagaagcagagagagcccagaaagggcagactgatcgtgtgtggccacggcacccttgagagagatggcgttttctgcctgctgagcgacgatcatggcgcctcttggagatacggcagcggagtgtctggaatcccttacggccagcctaagcaagagaacgatttcaaccccgacgagtgccagccttacgagctgcctgatggcagcgtcgtgatcaacgcccggaaccagaacaactaccactgccactgccggatcgtgctgagaagctacgacgcctgcgataccctgcggcctagagatgtgaccttcgatcctgagctggtggaccctgttgttgccgctggtgccgtcgtgacatctagcggcatcgtgttcttcagcaaccctgctcaccccgagttcagagtgaatctgaccctgcggtggtccttcagcaatggcacaagctggcggaaagaaaccgtgcagctttggcctggacctagcggctactcttctctggctacactggaaggcagcatggacggcgaagaacaggcccctcagctgtacgtgctgtacgagaagggcagaaaccactacaccgagagcatcagcgtggccaagatcagcgtt

SEQ ID NO: 24 - DNA, 1140 bp, Homo sapiens
atggccagcctgcctgtgctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgcccctacacatcaggtgcagtggcaggctcaagaggtggtggctcaggctagactggacggccacagatctatgaacccctgtcctctgtacgatgcccagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccatccagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagctgtgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaa

SEQ ID NO: 25 - DNA, 1284 bp, Homo sapiens
atggaggaagtgaccacctgtagcttcaacagccctctgttccggcaagaggacgaccggggcatcacctacagaatccctgctctgctgtacatccctcctacacacacctttctggccttcgccgagaagcggagcaccagacgagatgaagatgccctgcacctggtgctgagaagaggcctgagaatcggacagctggtgcagtggggacctctgaagcctctgatggaagccacactgcccggccacagaaccatgaatccttgtcctgtgtgggagcagaaaagcggctgcgtgttcctgttcttcatctgcgtgcggggccacgtgaccgagagacagcaaatcgtgtccggcagaaacgccgccagactgtgcttcatctacagccaggatgccggctgctcttggagcgaagttcgggatctgaccgaagaagtgatcggcagcgagctgaagcactgggccacatttgctgttggccctggccacggaatccagctgcaatctggcagactggtcatccccgcctacacctactatatccccagctggttcttctgcttccaactgccttgcaagacccggcctcacagcctgatgatctacagcgacgatctgggcgtgacatggcaccacggcagactgatcagacccatggtcaccgtggaatgcgaggtggccgaagtgacaggcagagctggacaccctgtgctgtactgctctgccagaacacccaaccggtgtagagccgaggctctgtctacagatcacggcgagggctttcagagactggccctctctagacagctgtgcgaacctcctcatggctgtcagggcagcgtggtgtccttcagacctctggaaatccctcaccggtgccaggacagcagctctaaggatgcccctaccatccagcagtctagccctggcagcagcctgagactggaagaggaagccggaacacctagcgagagctggctgctgtactctcaccccaccagcagaaagcagagagtggacctgggcatctacctgaatcagacccctctggaagccgcctgttggagcagaccttggattctgcactgtggcccttgcggctactctgatctggccgctctggaagaagagggcctgttcggctgcctgtttgagtgcggcacaaagcaagagtgcgagcagatcgccttccggctgttcacccacagagagatcctgagccatctgcagggcgactgcacaagcccaggcagaaatcccagccagttcaagagcaac

SEQ ID NO: 26 - DNA, 1452 bp, Homo sapiens
atgggcgtgcccagaacacccagcagaaccgtgctgttcgagagagagaggaccggcctgacctacagagtgccttctctgctgcctgtgcctcctggacctacactgctggccttcgtggaacagagactgagccccgatgattctcacgcccacagactggtgctgagaagaggaacactggctggcggctctgttagatggggagcactgcatgtgctgggcacagctgctcttgccgagcacagatccatgaatccctgtcctgtgcacgacgccggaaccggcacagtgtttctgttctttatcgccgtgctgggccacacacctgaggccgttcaaattgccaccggcagaaatgccgccagactgtgttgtgtggcctccagagatgccggcctgtcttggggatctgccagagatctgaccgaggaagccattggcggagccgttcaggattgggccacatttgctgttggacctggacacggcgtgcagctgccaagtggtagactgctggtgcctgcctacacatacagagtggatcggagagagtgcttcggaaagatctgccggacaagccctcacagcttcgccttctactccgacgatcacggccggacttggagatgtggtggcctggtgcctaatctgagaagcggcgaatgtcaactggccgccgttgatggtggacaggctggcagcttcctgtactgcaacgccagatctcctctgggctctagagtgcaggccctgtctaccgatgagggcaccagttttctgcccgccgaaagagttgcctctctgcctgaaacagcctggggctgtcagggctctatcgtgggatttcctgctcctgctccaaacagaccccgggacgattcttggagtgtcggccctggatctccactgcagcctccattgcttggaccaggcgttcacgagccacctgaagaggctgccgttgatcctagaggcggacaagttcctggcggcccttttagcagactgcagccaagaggcgacggccctagacaacctggaccaagacctggcgtcagcggagatgttggctcttggacactggccctgcctatgccttttgccgctcctcctcagtctcctacctggctgctgtactctcaccctgttggcagacgggccagactgcacatgggcatcagactgtctcagagccctctggaccccagaagctggacagagccttgggtcatctatgagggccctagcggctacagcgatctggcctctattggcccagctcctgaaggcggactggtgttcgcttgtctgtatgagagcggcgccagaaccagctacgacgagatcagcttctgcaccttcagcctgcgcgaggtgctggaaaatgtgcccgcctctcctaagcctcctaacctgggcgataagcctagaggctgttgctggccatct

SEQ ID NO: 27 - protein, 47 aa, Homo sapiens
MTGERPSTALPDRRWGPRILGFWGGCRVWVFAAIFLLLSLAASWSKA

SEQ ID NO: 28 - protein, 22 aa, Unknown
  Description of Unknown N-terminal signal peptide
MDMRVPAQLLGLLLLWLPGARC

SEQ ID NO: 29 - protein, 4 aa, Unknown
  Description of Unknown C-terminal lysosomal signal motif
YGTL

SEQ ID NO: 30 - protein, 382 aa, Salmonella typhimurium
MTVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYN

SEQ ID NO: 31 - protein, 232 aa, Artificial Sequence
  Description of Artificial Sequence Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 33 - protein, 232 aa, Artificial Sequence
  Description of Artificial Sequence Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 1383 bp, Homo sapiens
atgagacctgcggacctgcccccgcgccccatggaagaatccccggcgtccagctctgccccgacagagacggaggagccggggtccagtgcagaggtcatggaagaagtgacaacatgctccttcaacagccctctgttccggcaggaagatgacagagggattacctaccggatcccagccctgctctacataccccccacccacaccttcctggcctttgcagagaagcgttctacgaggagagatgaggatgctctccacctggtgctgaggcgagggttgaggattgggcagttggtacagtgggggcccctgaagccactgatggaagccacactaccggggcatcggaccatgaacccctgtcctgtatgggagcagaagagtggttgtgtgttcctgttcttcatctgtgtgcggggccatgtcacagagcgtcaacagattgtgtcaggcaggaatgctgcccgcctttgcttcatctacagtcaggatgctggatgttcatggagtgaggtgagggacttgactgaggaggtcattggctcagagctgaagcactgggccacatttgctgtgggcccaggtcatggcatccagctgcagtcagggagactggtcatccctgcgtatacctactacatcccttcctggttcttttgcttccagctaccatgtaaaaccaggcctcattctctgatgatctacagtgatgacctaggggtcacatggcaccatggtagactcattaggcccatggttacagtagaatgtgaagtggcagaggtgactgggagggctggccaccctgtgctatattgcagtgcccggacaccaaacaggtgccgggcagaggcgctcagcactgaccatggtgaaggctttcagagactggccctgagtcgacagctctgtgagcccccacatggttgccaagggagtgtggtaagtttccggcccctggagatcccacataggtgccaggactctagcagcaaagatgcacccaccattcagcagagctctccaggcagttcactgaggctggaggaggaagctggaacaccgtcagaatcatggctcttgtactcacacccaaccagtaggaaacagagggttgacctaggtatctatctcaaccagacccccttggaggctgcctgctggtcccgcccctggatcttgcactgtgggccctgtggctactctgatctggctgctctggaggaggagggcttgtttgggtgtttgtttgaatgtgggaccaagcaagagtgtgagcagattgccttccgcctgtttacacaccgggagatcctgagtcacctgcagggggactgcaccagccctggtaggaacccaagccaattcaaaagcaat

SEQ ID NO: 35 - DNA, 1488 bp, Homo sapiens
atgatgagctctgcagccttcccaaggtggctgagcatgggggtccctcgtaccccttcacggacagtgctcttcgagcgggagaggacgggcctgacctaccgcgtgccctcgctgctccccgtgccccccgggcccaccctgctggcctttgtggagcagcggctcagccctgacgactcccacgcccaccgcctggtgctgaggaggggcacgctggccgggggctccgtgcggtggggtgccctgcacgtgctggggacagcagccctggcggagcaccggtccatgaacccctgccctgtgcacgatgctggcacgggcaccgtcttcctcttcttcatcgcggtgctgggccacacgcctgaggccgtgcagatcgccacgggaaggaacgccgcgcgcctctgctgtgtggccagccgtgacgccggcctctcgtggggcagcgcccgggacctcaccgaggaggccatcggtggtgccgtgcaggactgggccacattcgctgtgggtcccggccacggtgtgcagctgccctcaggccgcctgctggtacccgcctacacctaccgcgtggaccgccgagagtgttttggcaagatctgccggaccagccctcactccttcgccttctacagcgatgaccacggccgcacctggcgctgtggaggcctcgtgcccaacctgcgctcaggcgagtgccagctggcagcggtggacggtgggcaggccggcagcttcctctactgcaatgcccggagcccactgggcagccgtgtgcaggcgctcagcactgacgagggcacctccttcctgcccgcagagcgcgtggcttccctgcccgagactgcctggggctgccagggcagcatcgtgggcttcccagcccccgcccccaacaggccacgggatgacagttggtcagtgggccccgggagtcccctccagcctccactcctcggtcctggagtccacgaacccccagaggaggctgctgtagacccccgtggaggccaggtgcctggtgggcccttcagccgtctgcagcctcggggggatggccccaggcagcctggccccaggcctggggtcagtggggatgtggggtcctggaccctggcactccccatgccctttgctgccccgccccagagccccacgtggctgctgtactcccacccagtggggcgcagggctcggctacacatgggtatccgcctgagccagtccccgctggacccgcgcagctggacagagccctgggtgatctacgagggccccagcggctactccgacctggcgtccatcgggccggcccctgaggggggcctggtttttgcctgcctgtacgagagcggggccaggacctcctatgatgagatttccttttgtacattctccctgcgtgaggtcctggagaacgtgcccgccagccccaaaccgcccaaccttggggacaagcctcgggggtgctgctggccctcc

SEQ ID NO: 36 - protein, 781 aa, Vibrio cholerae
MRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 37 - DNA, 2424 bp, Vibrio cholerae
ttgtcaatcaagatgacttcacaacgaagaagagcatcgattcacaaggaaacagattctaatataaagggagtagatatgcgtttcaaaaacgtaaagaaaaccgctttaatgcttgcaatgttcggtatggcgacaagctcaaacgccgcactttttgactataacgcaacgggtgacactgagtttgacagtccagccaaacagggatggatgcaagacaacacgaataatggcagcggcgttttaaccaatgcagatggaatgcccgcttggttggtgcaaggtattggagggagagctcaatggacatattctctctctactaatcaacatgcccaagcatcaagtttcggttggcgaatgacgacagaaatgaaagtgctcagtggtggaatgatcacaaactactacgccaacggcactcagcgtgtcttacccatcatttcattagatagcagtggtaacttagttgttgagtttgaagggcaaactggacgcaccgttttggcaaccggcacagcagcaacggaatatcataaatttgaattggtattccttcctggaagtaacccatccgctagcttttacttcgatggcaaactcattcgtgacaacatccagccgactgcatcaaaacaaaatatgatcgtatgggggaatggctcatcaaatacggatggtgtcgccgcttatcgtgatattaagtttgaaattcaaggcgacgtcatcttcagaggcccagaccgtataccgtccattgtagcaagtagcgtaacaccaggggtggtaaccgcatttgcagagaaacgtgtggggggaggagatcccggtgctctgagtaataccaatgacataatcactcgtacctcacgagatggcggtataacttgggataccgagctcaacctcactgagcaaatcaatgtcagtgatgagtttgatttctccgatcctcggcctatctatgatccttcctccaatacggttcttgtctcttatgctcgatggccgaccgatgccgctcaaaacggagatcgaataaaaccatggatgccaaacggtattttttacagcgtctatgatgttgcatcagggaactggcaagcgcctatcgatgttaccgatcaggtgaaagaacgcagtttccaaatcgctggttggggtggttcagagctgtatcgccgaaataccagcctaaatagccagcaagactggcaatcaaacgctaagatccgaattgttgatggtgcagcgaaccagatacaagttgccgatggtagccgaaaatatgttgtcacactgagtattgatgaatcaggtggtctagtcgctaatctaaacggtgttagtgctccgattatcctgcaatctgaacacgcaaaggtacactctttccatgactacgaacttcaatattcggcgttaaaccacaccacaacgttattcgtggatggtcagcaaatcacaacttgggctggcgaagtatcgcaggagaacaacattcagtttggtaatgcggatgcccaaattgacggcagactgcatgtgcaaaaaattgttctcacacagcaaggccataacctcgtggagtttgatgctttctatttagcacagcaaacccctgaagtagagaaagaccttgaaaagcttggttggacaaaaattaaaacgggcaacaccatgagtttgtatggaaatgccagtgtcaacccaggaccgggtcatggcatcacccttactcgacaacaaaatatcagtggcagccaaaacggccgcttgatctacccagcgattgtgcttgatcgtttcttcttgaacgtcatgtctatttacagtgatgatggcggttcaaactggcaaaccggttcaacactccctatcccctttcgctggaagagttcgagtatcctagaaactctcgaacctagtgaagctgatatggttgaactccaaaacggtgatctactccttactgcacgccttgattttaaccaaatcgttaatggtgtgaactatagcccacgccagcaatttttgagtaaagatggtggaatcacgtggagcctacttgaggctaacaacgctaacgtctttagcaatatcagtactggtaccgttgatgcttctattactcggttcgagcaaagtgacggtagccatttcttactctttactaacccacaaggaaaccctgcggggacaaatggcaggcaaaatctaggcttatggtttagcttcgatgaaggggtgacatggaaaggaccaattcaacttgttaatggtgcatcggcatattctgatatttatcaattggattcggaaaatgcgattgtcattgttgaaacggataattcaaatatgcgaattcttcgtatgcctatcacattgctaaaacagaagctgaccttatcgcaaaactaa

SEQ ID NO: 38 - protein, 409 aa, Mus musculus
MVGADPTRPRGPLSYWAGRRGQGLAAIFLLLVSAAESEARAEDDFSLVQPLVTMEQLLWVSGKQIGSVDTFRIPLITATPRGTLLAFAEARKKSASDEGAKFIAMRRSTDQGSTWSSTAFIVDDGEASDGLNLGAVVNDVDTGIVFLIYTLCAHKVNCQVASTMLVWSKDDGISWSPPRNLSVDIGTEMFAPGPGSGIQKQREPGKGRLIVCGHGTLERDGVFCLLSDDHGASWHYGTGVSGIPFGQPKHDHDFNPDECQPYELPDGSVIINARNQNNYHCRCRIVLRSYDACDTLRPRDVTFDPELVDPVVAAGALATSSGIVFFSNPAHPEFRVNLTLRWSFSNGTSWLKERVQVWPGPSGYSSLTALENSTDGKKQPPQLFVLYEKGLNRYTESISMVKISVYGTL

SEQ ID NO: 39 - protein, 393 aa, Mus musculus
MTVQPSPWFSDLRPMATCPVLQKETLFRTGVHAYRIPALLYLKKQKTLLAFAEKRASKTDEHAELIVLRRGSYNEATNRVKWQPEEVVTQAQLEGHRSMNPCPLYDKQTKTLFLFFIAVPGRVSEHHQLHTKVNVTRLCCVSSTDHGRTWSPIQDLTETTIGSTHQEWATFAVGPGHCLQLRNPAGSLLVPAYAYRKLHPAQKPTPFAFCFISLDHGHTWKLGNFVAENSLECQVAEVGTGAQRMVYLNARSFLGARVQAQSPNDGLDFQDNRVVSKLVEPPHGCHGSVVAFHNPISKPHALDTWLLYTHPTDSRNRTNLGVYLNQMPLDPTAWSEPTLLAMGICAYSDLQNMGQGPDGSPQFGCLYESGNYEEIIFLIFTLKQAFPTVFDAQ

SEQ ID NO: 40 - protein, 418 aa, Mus musculus
MEEVPPYSLSSTLFQQEEQSGVTYRIPALLYLPPTHTFLAFAEKRTSVRDEDAACLVLRRGLMKGRSVQWGPQRLLMEATLPGHRTMNPCPVWEKNTGRVYLFFICVRGHVTERCQIVWGKNAARLCFLCSEDAGCSWGEVKDLTEEVIGSEVKRWATFAVGPGHGIQLHSGRLIIPAYAYYVSRWFLCFACSVKPHSLMIYSDDFGVTWHHGKFIEPQVTGECQVAEVAGTAGNPVLYCSARTPSRFRAEAFSTDSGGCFQKPTLNPQLHEPRTGCQGSVVSFRPLKMPNTYQDSIGKGAPATQKCPLLDSPLEVEKGAETPSATWLLYSHPTSKRKRINLGIYYNRNPLEVNCWSRPWILNRGPSGYSDLAVVEEQDLVACLFECGEKNEYERIDFCLFSDHEVLSCEDCTSPSSD

SEQ ID NO: 41 - protein, 501 aa, Mus musculus
METAGAPFCFHVDSLVPCSYWKVMGPTRVPRRTVLFQRERTGLTYRVPALLCVPPRPTLLAFAEQRLSPDDSHAHRLVLRRGTLTRGSVRWGTLSVLETAVLEEHRSMNPCPVLDEHSGTIFLFFIAVLGHTPEAVQIATGKNAARLCCVTSCDAGLTWGSVRDLTEEAIGAALQDWATFAVGPGHGVQLRSGRLLVPAYTYHVDRRECFGKICWTSPHSLAFYSDDHGISWHCGGLVPNLRSGECQLAAVDGDFLYCNARSPLGNRVQALSADEGTSFLPGELVPTLAETARGCQGSIVGFLAPPSIEPQDDRWTGSPRNTPHSPCFNLRVQESSGEGARGLLERWMPRLPLCYPQSRSPENHGLEPGSDGDKTSWTPECPMSSDSMLQSPTWLLYSHPAGRRARLHMGIYLSRSPLDPHSWTEPWVIYEGPSGYSDLAFLGPMPGASLVFACLFESGTRTSYEDISFCLFSLADVLENVPTGLEMLSLRDKAQGHCWPS

SEQ ID NO: 42 - DNA, 3850 bp, Mus musculus
gggtcacatgctgatggactaattggagtcgcggcagcgcgggctgcggcccccaaggggaggggtcggagtgacgtgcgcgcttttaaagggccgaggtcagctgacggcttgccaccggtgaccagttcctggacagggatcgccgggagctatggtgggggcagacccgaccagaccccggggaccgctgagctattgggcgggccgtcggggtcaggggctcgcagcgatcttcctgctcctggtgtccgcggcggaatccgaggccagggcagaggatgacttcagcctggtgcagccgctggtgaccatggagcagctgctgtgggtgagcgggaagcagatcggctctgtagacactttccgcatcccgctcatcacagccacccctcggggcacgctcctggccttcgctgaggccaggaaaaaatctgcatccgatgagggggccaagttcatcgccatgaggaggtccacggaccagggtagcacgtggtcctctacagccttcatcgtagacgatggggaggcctccgatggcctgaacctgggcgctgtggtgaacgatgtagacacagggatagtgttccttatctataccctctgtgctcacaaggtcaactgccaggtggcctctaccatgttggtttggagtaaggacgacggcatttcctggagcccaccccggaatctctctgtggatattggcacagagatgtttgcccctggacctggctcaggcattcagaaacagcgggagcctgggaagggccggctcattgtgtgtggacacgggacgctggagcgagatggggtcttctgtctcctcagtgatgaccacggtgcctcctggcactacggcactggagtgagcggcattccctttggccagcccaaacacgatcacgatttcaaccccgacgagtgccagccctacgagcttccagatggctcggtcatcatcaacgcccggaaccagaataactaccattgccgctgcaggatcgtcctccgcagctatgacgcctgtgacaccctcaggccccgggatgtgaccttcgaccctgagctcgtggaccctgtggtagctgcaggagcactagccaccagctccggcattgtcttcttctccaatccagcccaccctgagttccgagtgaacctgaccctgcgctggagtttcagcaatggtacatcctggcagaaggagagggtccaggtgtggccgggacccagcggctactcgtccctgacagccctggaaaacagcacggatggaaagaagcagcccccgcagctgttcgttctgtacgagaaaggcctgaaccggtacaccgagagcatctccatggtcaaaatcagcgtctacggcacgctctgagccccgtgcccaaaggacaccaagtcctggtcgctgacttcacagctctctggaccatctgcagagggtgcctgaaacacagctcttcctctgaactctgaccttttgcaacttctcatcaacagggaagtctcttcgttatgacttaacacccagcttcctctcggggcaggaagtccctccgtcaccaagagcacttttttccagtatgctggggatggcccctgtccattctcttccaggacaacggagctgtgcctttctgggacaggatgggggaggggctccccctggagagatgaacagatacgaactcagggaactgagaaggcccggtgtcctagggtacaaaggcaggtactagatgtgattgctgaaagtccccagggcagagtgtcctttcagagcaaggataagcacacctacgtgtgcacctttgattatttatgaatcgaaatatttgtaacttaaaatttttgatgcagaaaaagcgtttgtggagtctgtggttctgtctgctcacgccttcccaattgcctcctggagagacaggaaggcagctggaagaggagccgatgtacttactgggaagcagaaacccctagattccatcctggctgctgctgtttgcaagtgtcaaagatgggggggcgtgtttatattttatatttctaagatggggtggcataggaaatagggaacagatgtgtaaaaccagatgggaaggacagtctgtgagaaaggagcaagcagttgctgcaggtgtgggagagcaaagcccttctccacgtggaaagagcccagatggacgctaagcatgttgggcacctgtaaccccgcactcgctggactgacggtgtagctcagtggtggagctagtacttggaacgcctaagactctgggttcagtccttgggggggggggtatgtgtttattgagaggaaggtgtacgtactgtaggtcagaggacagcttactggagttgtctctctccttcacgctgtgagtcctgtggaatgacctcaggtgtcagagttgggggcaggtgcctttgccagctgagccatcttgctgtctctgctttaatttaaaaaaaaaaaaaaaaaagaatattaaggtctgagggattcgggctgcgttcatttcaattagagggtcatatttcttttgacatttcttctctaagaaatgttaagatcatttgttctgtgtgatagaggtatagctccattgtatgtcagcagtgagggatcctgtgcattttatccagagtttgtacggtgttctaggggctgctagtgcagcccagtgctaaacacttcagcatgcacaaggcctcaatcagtgcatgcatgtgcacacacacacagacacacacgtacacactgacacaggtacacaaatacacactggcccacatgtacacatcgactcacaggtacacagacccactttgacacacatatacacagacacaaacgcactggcacacacatatacacaggcacacatggatagatggacacacgtgtacacatacacacacacacagaaatacaaatgttcaggttttctaaaaaaaaaaaaattagagacgtgttgacttcatttttagcaaaaatcctgtcatgtatcttaaagtggattgaacccactatgtagcccaggctggcctccaaatgggcatccttctgcctcagtctcccgagggctaggataacaggagtatgccatcacacctggctaatagaaattttcaaaattgtttgtttgaaggtgactcttactatattgcctaactgatctccagttcgtgaaatcctcctgcctcagaaccaggactgtcaatataacccaccaagacaggccaacattcacaattgattgttagtttgtggtctgaatcaaggtcttatactgtagcccaggctagcccggaatacacgatatctccagtgcttcagatcctcagttctaactaagcatggccacatccatgtttaactgcaaatttgatgttaccatggtttggtttggtttggtttggtttggtttggtttggtttggttttttggccattttttttttctcatgctgaggccttgtgctctcaagttggggagacagcatggagggtagctgcaactgtaaccccagttccaggggacctgacaccctctggcctccacaagtattaggcacatctgtggtgcacagacatacaatcaggcaaaatattcatacacataaaataaaataatttaaaacaaaagcaaaaatcaggacctaagaaaaaaatctattcctgattcttttatgttttgtttgtattttatcaagacagggttgtttctctgtatagccctggctgtcttggaattcactctgtagaccaggctggcctcaaactcagaaatcctcctgcctttgccttccaagtgctggaattaaaggcatgcgccacc

SEQ ID NO: 43 - DNA, 1722 bp, Mus musculus
gacatgacccaaacggcccctggctgcaaggtaatatcggaagttgactaagaatggacgccccaccactgactgacccgccccctgagtctgagattggacttgtctctggatacagtcatactttgaggtactacaagttagaaactgttaggttactcagttcagtccatgacagtccaaccttctccatggttttccgatctcaggcccatggcgacctgccctgtcctgcagaaggagacactgttccgcacaggcgtccatgcttacagaatccctgctctgctctacctgaagaagcagaagaccctgctggcctttgcggaaaagcgagccagcaagacggatgagcacgcagagttgattgtcctgagaagaggaagctacaacgaagccaccaaccgtgtcaagtggcagcctgaggaagtggtgacccaagcccagctggaaggccaccgctccatgaatccatgtcccttgtatgacaagcaaacaaagaccctcttccttttcttcatcgctgtccctgggcgtgtatcagaacatcatcagctccacactaaggttaatgtcacacggctgtgctgtgtcagcagcactgaccatgggaggacctggagccccatccaggacctcacagagaccaccattggcagcactcatcaggaatgggccacatttgctgtgggtcctgggcattgtctgcagctgcggaacccagctgggagcctgctggtacctgcttatgcctaccggaaactgcaccctgctcagaagcctaccccctttgccttctgcttcatcagccttgaccatgggcacacatggaaactaggcaactttgtggctgaaaactcactggagtgccaggtggctgaggttggcactggagctcagaggatggtatatctcaatgctaggagcttcctgggagccagggtccaggcacaaagtcctaatgatggtctggatttccaggacaaccgggtagtgagtaagcttgtagagcccccccacgggtgtcatggaagtgtggttgccttccacaaccccatctctaagccacatgccttagacacatggcttctttatacacaccctacagactccaggaatagaaccaacctgggtgtgtacctaaaccagatgccactagatcccacagcctggtcagagcccaccctgctggccatgggcatctgtgcctactcagacttacagaacatggggcaaggccctgatggctccccacagtttgggtgtctgtatgaatcaggtaactatgaagagatcattttcctcatattcaccctgaagcaagctttccccactgtatttgatgcccagtgatctcagtgcacgtggcccaaagggcttccttgtgcttcaaaacacccatctctctttgcttccagcatcctctggactcttgagtccagctcttgggtaacttcctcaggaggatgcagagaatttggtctcttgactctctgcaggccttattgtttcagcctctggttctcttttcagcccagaaatcaaaggagcctggctttcctcagcctgttggcagggcaggtggggacagtatatatagaggctgccattctgcatgtcggttgtcactatgctagtttaacctgcctgtttccccatgcctagtgtttgaatgagtattaataaaatatccaacccagcccatttcttcctggaaaaaaa

SEQ ID NO: 44 - DNA, 3340 bp, Mus musculus
actgcgcggtgaaggggcgtggcctggccggggaggttgacacccagacgctgctctcagtcctctggcgcctgctccccagcgcattccttctgctcctgggatatttgtctcattactgccagttcttgcgcagcggtcactgggttcgtttcagcgtctgtggtttctgtcgctgttatccagtctccatcgccccagctcagcttcaggccttcttccgagactccacgggagagcccagagagcctccggagccgaagccatggaggaagtcccaccctactccctcagcagcaccctgttccagcaggaagaacagagtggggtgacctaccggatcccagccctgctgtaccttcctcccacccacaccttcctggcctttgcagagaagcggacctcagtcagagatgaggatgctgcctgcctggtgctcagacgagggctgatgaaggggcgctctgtacagtggggcccccaacggctactgatggaggccacattacctgggcatcgcaccatgaacccctgccctgtgtgggagaaaaatactggccgtgtgtacctgtttttcatctgtgtgcggggccatgttactgagaggtgccagattgtgtggggcaaaaatgccgcccgtctctgcttcctttgcagtgaagatgccggctgctcttggggtgaagtgaaagacttgaccgaggaggtcattggctcagaggtgaagcgctgggccacatttgctgtgggcccaggtcatggcatccagctacactcgggaaggctgatcatccccgcctatgcctactatgtctcacgttggtttctctgctttgcgtgttcagtcaagccccattccctgatgatctacagtgatgactttggagtcacatggcaccatggcaagttcattgagccccaggtgacaggggagtgccaagtggccgaagtggctgggacggctggtaaccctgtgctcactgcagtgcccgaacaccaagccgatttcgagcagaggcttttagtactgatagtggtggctgctttcagaagccaaccctgaacccacaactccatgagcctcgaaccggctgccaaggtagtgtagtgagcttccggcctttgaagatgccaaatacctatcaagactcaattggcaaaggtgctcccgctactcagaagtgccctctgctggacagtcctctggaggtggagaaaggagctgaaacaccatcagcaacatggctcttgtactcacatccaactagcaagaggaagaggattaacctaggcatctactacaaccggaaccccttggaggtgaactgctggtcccgcccgtggatcttgaaccgtgggcccagtggctactctgatctggctgttgtggaagaacaggacttggtggcgtgtttgtttgagtgtggggagaagaatgagtatgagcggattgacttctgtctgttttcagaccatgaggtcctgagctgtgaagactgtaccagccctagtagcgactaaagccaaatcaagacggatgagtgaggcccagcttcccacagaaaggaatggcagctacagccagggtaacagaggtctctgatgtctagagaaaactctaaaaactaataatctgctccttgaattttttcacttttcccttcaatgagcatggtgaaaattgtgccatatcttacataacgaggctcttgaactgggagtttgaatctcttctcttcccattaaaaggagaggccatgtgctcgcttcgcgttcgacaaagcctggattctgatcttgagtggaagccacaggcttgtcttttccaatggttcactgctcacctgagtattaggtgatgtgtaggtgccttggccagaagaaagatctgtgttgttgtatttttttaaatttatttatttactatatgtaagtacactgcagctgtcttcagacacaccagaagagggcgtcagatctcattagagatggttgtgagccaccatgtggttgctgggatttgaactcaggaccttcagaagagcagtcagtgctcttaactactgagccatctctcaagccccgcattgctgtatttttaataagaaaaatgcccttatccttccaataatgcctggagctgtacaaattctctgtcttagaagacttgagaaagcagaactgtaaggtcagatgctttctccagccttgatgctgtgttccaccttcccttcctcatccagaaaacagttactagggagaaaatgagaaacccatgccagctgcccttgatgatggttgataacggtgcttattgcttttgatgtcattacctctgttagagatgaatcagagtcagaggtccttagctgcatccacccatttccagggggacattctaacactgctgaacagtcagctaaaatgagagctgtgtgtcctagcctgattccaggttagtcatgatgcttcctggagctgggcttttatctaatcccaggagccatctaggggaggctcagagctagcaggtgatcttcctgagatggtttcaccgtgacaggtgaaccatgagcccttccaagcaaggccaaaggacaacattataggaaagatttctagtattaatatgccttttctctgtgtgtgtactgtcttgtagtgatgctatatagacaaatagatgatttcttattttttgtttgtttgtttgtttttttgtttttctgtagccctagctgtcctggaactcactttgtaaaccaggctggcctcgatctcagaaatccgcctgcctctgcctcccgagtgctgggattaaaggtgtgcaccaccacaccttaatgatgatcctataagtattcctaaaattatactagtaattattaactcctttataataggactgctattaaagccctcgctgatatgaaaactacagtgagaactctgccagtcttcacatgtcataattacttctgagatagaaagcaggcatttacaacttagaacacatttcttagagctgtaaaacaattaactagaggtcataaaagggaatgaaagatttattgtaggtgctaggacagaacataaaatattgactgggcttatctatatgaaacttcattgttaacttttacacaagaattatggtttttaactttcagtgaacctgcggagctagtgacagaagagaaatgtctagttagataactactcttaatggaaattcacataaacatctgttgccatcttctttttgaatttatgtttaaacttgtgaatgtttgaattagacactacgcgagcacatagaaaataaagaactaagcgtgaa

SEQ ID NO: 45 - DNA, 4608 bp, Mus musculus
ggacagtgtgcatcacggagcttgtggcccagactgtgcctggcagacccagaggacctaaggcttggctctagtggtggtcagcacagccctcggtggtctgcggagcctgatattgctttacgtaagggctgttctgctgtgcatctcctgtgtctgaagctattcgccatggagactgctggagctcccttctgcttccatgtggactccctggtaccttgctcctactggaaggttatggggcccacgcgtgttcccaggagaacggtgctcttccagagggaaaggacgggcctgacctaccgtgtgcctgcgttactctgtgtgcctcccaggcctactctgctggccttcgcggaacagcgacttagccctgatgactcccatgcccaccgcctggtgctacggaggggcacgctgaccaggggctcagtgcggtggggcactctgagtgtactggagactgcagtactggaggagcacaggtctatgaacccttgcccggtgctggatgagcactctggtaccatcttcctcttcttcattgccgtgctgggccacacaccggaggccgtgcaaatcgccactggcaagaacgctgctcgcctctgctgtgtgaccagctgtgacgctggcctcacctggggcagtgttcgagatctcactgaggaagccattggtgctgcattgcaggactgggccacctttgctgtgggtccgggccatggagttcagctgcgctcgggtcgcctgcttgttcctgcttacacctatcatgtggaccgacgggaatgttttggcaagatctgctggaccagtccccactccttggcattctacagtgatgatcatgggatctcctggcattgtggaggccttgtgcccaacctacgctctggagagtgccaactggctgcggtagatggagactttctctactgtaatgctcgaagccctctgggtaaccgtgtgcaggcactgagtgctgatgaaggcacgtccttcctaccaggggagctggtgcctacattggcagagacggctcgtggttgccagggtagcattgtgggcttcctagctccaccctcaatcgagcctcaggatgaccggtggacagggagtcctaggaacaccccacattccccatgcttcaatctcagagtacaggagtcttcgggggaaggtgccagaggtcttcttgaacgttggatgcccaggttgcctctctgctacccacagtcccggagcccagagaatcatggcctagagcctgggtcagatggagataagacatcctggactccggaatgtcctatgtcctctgattccatgcttcagagccccacatggctactatattcccacccagcagggcgtagagctcggctccacatgggaatctacctgagccgatcccccttggatccccacagctggacagagccctgggtgatctatgagggccccagtggctactctgaccttgcctttcttgggcctatgcctggggcatccctggtttttgcctgtctgtttgagagcgggaccaggacttcctatgaagacatttctttttgcttgttctcactggcggatgtcctggagaatgtgcccactggcttagagatgctaagtctcagggataaggctcaggggcattgctggccctcttgatggcctcaccctctcgtagccgcctggagaggaagggtagactatatagaggaggttaggggtaggtcagcatgatgctaggatggagagagctctgtcccctcgtggatggtggtggtgactcacccggggggccagctgctttctgagtgcaaatgagaaaaataaagagctgcgctgtgacttttctttccacatcaaagcttgggtgtcagtgctttagcttgatgctctgatcaccatgcaaatcttccaccggcgccttgctcagctttcatatcccaagggtgcctgggaggaaggcaacagggacagtggacatcactgcaccactttccacgaccctgtgtgccaacctcagccactttgaaacatgctgatgactgaggtctgttcactttcttaatttcaagcaggagaagcaggttggggagccagcctccccagctagaggggacagaacttgacttgagcaggggggtacctcctaggacctgctccatgtgcctacttctttacccttctctagagagggctcttgtcctgtcagagctgttttctcccttctcttgttttttctttttcaagactgtttctctgtgttagccctggctgtcctggatctcactctgtagatcaggctgaccttgagttcaaagctccatctgcctctacttctcacattactgtgattaaaggcatatactaccactgcctggtgcccttttgtatttcttattaaagtcctaatgtctgattataaaaacagtctgtgtgggctggagtgatggcttactcagtaaagcacttgccatggaatctgggcaatctgagtttcatttttagcatcctgtaaaaatcccaatttgatggtgtacttgtaatgtcagcatggagaggcagagataggtaagttccccaagactctttgaaccgacagcttggcctcactggcacattccaggtctcagtgagagaccctgcctcaaaatacaaagaaagagctgctgaagagtgggtcagagttgacctctgatctccggaagtatatgatacacacccgtgcatgcactcttccttacaaaataaaaagcaaaacaaaaccccaacaggtatatggccattttagaaaaattagaagatttagaaagctatacataaaaaaaaatgacctaaagaaaaatctttactgttctgggcactatccctatcaaaccactgtgttctttggccaagccttggggtggacactgttttgaggtgggtcctgttatctccactaggtagtggagttttgtgtcagactaactgggtcttaaagctgtctttaaggccatcaggagctactgacttgcctgcctcagcagagcatatcctgaaggtcggggttaagtctccttcccgagcgagttgccttccagtgggcccctggactcctaggtcctcagcgctcatcagctgccaaggactctgagggaatgtcctctgactgtggccccgaaaggtaggggagggggatgtgcttaggcttaggacagggtcctgtttcagtctgccttcactgttagtagcactgtgccacatggcacagactgggcgagctttaaaggaaggaggttgatattggttcccacttctggggatcatggttgagcagccttgtctgatgatggttgtcttgatggtagatcgtgaggtagttgatgaaggtatgacatggtgagaaactctgtgtgtgtgtgttattttctctgtgttctacctatacatctatctatgtatatatgtatctatctatctacctggaggctggagagatagcttagtggttaagaacatttgttgttcttgcatagtcctggatttaaattttcagcacccacatggcagctcacaacaacccataaatccagtttcagaggatccaacctctgatataccatgtcagccagagcagacacggctgaaggtggtttgatccccgtatggagaggtgacaattgggaagagagaaagatcaacttaaccatgcaaggaacaggaagttaaatactgaacagggaaggtaaaggcaggaagtagatgtagagggcaaatcaatgaaacccaaacatacccaaattacgctaaacacacactgacatgccaattaaaaggacaaattggctccactggcaaaaccaaaacagacactgaagatccaaacagtcacatgccaactaccgcggagggagacagacacagagaagaccgtgacagacacttggacactcttgagagtggatgtgcaggaagagagctctgccagtggagaagaaagcactcagaagaaagtgacagcagctgtaaatttgtattctgctaatgttatgttccaaagttgaaagcaaaattgtaccaattcataagaacaaacaggctgactctcagttgtgactgaacgtctctcagtaactgacggggcgagcaggccaaaggagagtcggctcagaagggtgcatagccacgccaaatcaaataagcaagtacaaccggcaggctctatttctagcacaaaggggtctgtgcctcattctgtgcttgggtcagagcttgggtctctcatttggatgtaagtggtgtagtggagaagcaggaaataatccggagcgcatattttgattttaacataagtgctgatttgggagggagttttgtcaaattgtgtttttacaatgtttttttttttttaaatgatgcttttttgtaaagtgtacaaatgtgatataagattggttctgctacattcagtttctataaaagtggttctaaaatattgtactgtcaatcatctcatgattattctactgtacacattactgactttgtatgtaataattaatattagaagaaaatataatttatttgaatataaaaaaaaaaaaaaaaaaa

SEQ ID NO: 46 - protein, 380 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (6)..(6): Phe, Trp, Tyr or Val
  MOD_RES (9)..(9): Lys or Asp
  MOD_RES (62)..(62): Pro, Asn, Asp, His, Glu, Gly, Ser or Thr
  MOD_RES (93)..(93): Ala, Glu, or Lys
  MOD_RES (187)..(187): Arg, Ile, or Lys
  MOD_RES (270)..(270): Gln, Ala, His, Phe, or Pro
  MOD_RES (301)..(301): Ser or Arg
  MOD_RES (302)..(302): Trp or Lys
  MOD_RES (332)..(332): Ala, Cys, Ser, or Val
  MOD_RES (363)..(363): Val or Arg
  MOD_RES (365)..(365): Leu, Gln, His, Ile, Lys, or Ser
  See specification as filed for detailed description of substitutions and preferred embodiments
XASLPXLQXESVFQSGAHAYRIPALLYLPGQQSLLAFAEQRASKKDEHAELIVLRRGDYDAXTHQVQWQAQEVVAQARLDGHRSMNPCPLYDXQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRARVQAQSTNDGLDFQESQLVKKLVEPPPXGCQGSVISFPSPRSGPGSPAQWLLYTHPTHXXQRADLGAYLNPRPPAPEAWSEPVLLAKGSXAYSDLQSMGTGPDGSPLFGCLYEANDYEEIXFXMFTLKQAFPAEYLPQ

SEQ ID NO: 47 - protein, 380 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (2)..(2): Ala or Lys
  MOD_RES (4)..(4): Asn or Leu
  MOD_RES (5)..(5): Pro or His
  MOD_RES (6)..(6): Phe, Trp, Tyr or Val
  MOD_RES (9)..(9): Lys or Asp
  MOD_RES (44)..(44): Lys, Arg, or Glu
  MOD_RES (45)..(45): Lys, Ala, Arg, or Glu
  MOD_RES (54)..(54): Leu or Met
  MOD_RES (62)..(62): Pro, Asn, Asp, His, Glu, Gly, Ser or Thr
  MOD_RES (69)..(69): Gln or His
  MOD_RES (78)..(78): Arg or Lys
  MOD_RES (93)..(93): Ala, Glu or Lys
  MOD_RES (107)..(107): Gly or Asp
  MOD_RES (108)..(108): Gln or His
  MOD_RES (112)..(112): Gln, Arg, or Lys
  MOD_RES (125)..(125): Ala, Cys, Ile, Ser, Val, or Leu
  MOD_RES (126)..(126): Gln or Leu
  MOD_RES (150)..(150): Ala or Val
  MOD_RES (164)..(164): Cys or Gly
  MOD_RES (171)..(171): Ala or Gly
  MOD_RES (187)..(187): Arg, Ile, or Lys
  MOD_RES (196)..(196): Ala, Cys, Leu, or Val
  MOD_RES (217)..(217): Leu, Ala, or Val
  MOD_RES (249)..(249): Thr or Ala
  MOD_RES (251)..(251): Asp or Gly
  MOD_RES (257)..(257): Glu or Lys
  MOD_RES (270)..(270): Gln, Ala, His, Phe, or Pro
  MOD_RES (272)..(272): Cys or Val
  MOD_RES (292)..(292): Trp or Arg
  MOD_RES (301)..(301): Ser or Arg
  MOD_RES (302)..(302): Trp or Lys
  MOD_RES (325)..(325): Lys or Val
  MOD_RES (332)..(332): Ala, Cys, Ser, or Val
  MOD_RES (352)..(352): Cys, Leu, or Val
  MOD_RES (363)..(363): Val or Arg
  MOD_RES (365)..(365): Leu, Gln, His, Ile, Lys, or Ser
  See specification as filed for detailed description of substitutions and preferred embodiments
XXSXXXLQXESVFQSGAHAYRIPALLYLPGQQSLLAFAEQRASXXDEHAELIVXRRGDYDAXTHQVQWXAQEVVAQAXLDGHRSMNPCPLYDXQTGTLFLFFIAIPXXVTEXQQLQTRANVTRLXXVTSTDHGRTWSSPRDLTDAAIGPXYREWSTFAVGPGHXLQLHDRXRSLVVPAYAYRKLHPXQRPIPSAFXFLSHDHGRTWARGHFVAQDTXECQVAEVETGEQRVVTLNARSHLRARVQAQSXNXGLDFQXSQLVKKLVEPPPXGXQGSVISFPSPRSGPGSPAQXLLYTHPTHXXQRADLGAYLNPRPPAPEAWSEPXLLAKGSXAYSDLQSMGTGPDGSPLFGXLYEANDYEEIXFXMFTLKQAFPAEYLPQ

SEQ ID NO: 48 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 49 - protein, 380 aa, Artificial Sequence
  Description of Artificial Sequence Synthetic polypeptide
DASLPYLQDESVFQSGAHAYRIPALLYLPGQQSLLAFAEQRASKKDEHAELIVLRRGDYDAPTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIRFIMFTLKQAFPAEYLPQ

SEQ ID NO: 50 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDANTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 51 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQ

SEQ ID NO: 52 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDASTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQ

SEQ ID NO: 53 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDATTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQ

SEQ ID NO: 54 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDANTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQ

SEQ ID NO: 55 - protein, 379 aa, Artificial Sequence
  Description of Artificial Sequence Synthetic polypeptide
ASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 56 - protein, 380 aa, Artificial Sequence
  Description of Artificial Sequence Synthetic polypeptide
MASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 57 - protein, 379 aa, Artificial Sequence
  Description of Artificial Sequence Synthetic polypeptide
ASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 58 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 59 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 60 - protein, 380 aa, Artificial Sequence
  Description of Artificial Sequence Synthetic polypeptide
AASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 61 - protein, 380 aa, Artificial Sequence
  Description of Artificial Sequence Synthetic polypeptide
MASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 62 - protein, 380 aa, Artificial Sequence
  Description of Artificial Sequence Synthetic polypeptide
AASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 63 - protein, 451 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 213 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 222 aa, Artificial Sequence
  Description of Artificial Sequence Synthetic polypeptide
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEP

SEQ ID NO: 66 - protein, 213 aa, Artificial Sequence; identical to SEQ ID NO: 64
  Description of Artificial Sequence Synthetic polypeptide

SEQ ID NO: 67 - protein, 451 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 69 - protein, 617 aa, Artificial Sequence
  Description of Artificial Sequence Synthetic polypeptide
DASLPYLQDESVFQSGAHAYRIPALLYLPGQQSLLAFAEQRASKKDEHAELIVLRRGDYDAPTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIRFIMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 70 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDANTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 71 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 72 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDASTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 73 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDATTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 74 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDANTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 75 - protein, 617 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (6)..(6): Phe, Trp, Tyr or Val
  MOD_RES (9)..(9): Lys or Asp
  MOD_RES (62)..(62): Pro, Asn, Asp, His, Glu, Gly, Ser or Thr
  MOD_RES (93)..(93): Ala, Glu, or Lys
  MOD_RES (187)..(187): Arg, Ile, or Lys
  MOD_RES (270)..(270): Gln, Ala, His, Phe, or Pro
  MOD_RES (301)..(301): Ser or Arg
  MOD_RES (302)..(302): Trp or Lys
  MOD_RES (332)..(332): Ala, Cys, Ser, or Val
  MOD_RES (363)..(363): Val or Arg
  MOD_RES (365)..(365): Leu, Gln, His, Ile, Lys, or Ser
  See specification as filed for detailed description of substitutions and preferred embodiments
XASLPXLQXESVFQSGAHAYRIPALLYLPGQQSLLAFAEQRASKKDEHAELIVLRRGDYDAXTHQVQWQAQEVVAQARLDGHRSMNPCPLYDXQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRARVQAQSTNDGLDFQESQLVKKLVEPPPXGCQGSVISFPSPRSGPGSPAQWLLYTHPTHXXQRADLGAYLNPRPPAPEAWSEPVLLAKGSXAYSDLQSMGTGPDGSPLFGCLYEANDYEEIXFXMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 76 - protein, 617 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (2)..(2): Ala or Lys
  MOD_RES (4)..(4): Asn or Leu
  MOD_RES (5)..(5): Pro or His
  MOD_RES (6)..(6): Phe, Trp, Tyr or Val
  MOD_RES (9)..(9): Lys or Asp
  MOD_RES (44)..(44): Lys, Arg, or Glu
  MOD_RES (45)..(45): Lys, Ala, Arg, or Glu
  MOD_RES (54)..(54): Leu or Met
  MOD_RES (62)..(62): Pro, Asn, Asp, His, Glu, Gly, Ser or Thr
  MOD_RES (69)..(69): Gln or His
  MOD_RES (78)..(78): Arg or Lys
  MOD_RES (93)..(93): Ala, Glu or Lys
  MOD_RES (107)..(107): Gly or Asp
  MOD_RES (108)..(108): Gln or His
  MOD_RES (112)..(112): Gln, Arg, or Lys
  MOD_RES (125)..(125): Ala, Cys, Ile, Ser, Val, or Leu
  MOD_RES (126)..(126): Gln or Leu
  MOD_RES (150)..(150): Ala or Val
  MOD_RES (164)..(164): Cys or Gly
  MOD_RES (171)..(171): Ala or Gly
  MOD_RES (187)..(187): Arg, Ile, or Lys
  MOD_RES (196)..(196): Ala, Cys, Leu, or Val
  MOD_RES (217)..(217): Leu, Ala, or Val
  MOD_RES (249)..(249): Thr or Ala
  MOD_RES (251)..(251): Asp or Gly
  MOD_RES (257)..(257): Glu or Lys
  MOD_RES (270)..(270): Gln, Ala, His, Phe, or Pro
  MOD_RES (272)..(272): Cys or Val
  MOD_RES (292)..(292): Trp or Arg
  MOD_RES (301)..(301): Ser or Arg
  MOD_RES (302)..(302): Trp or Lys
  MOD_RES (325)..(325): Lys or Val
  MOD_RES (332)..(332): Ala, Cys, Ser, or Val
  MOD_RES (352)..(352): Cys, Leu, or Val
  MOD_RES (363)..(363): Val or Arg
  MOD_RES (365)..(365): Leu, Gln, His, Ile, Lys, or Ser
  See specification as filed for detailed description of substitutions and preferred embodiments
XXSXXXLQXESVFQSGAHAYRIPALLYLPGQQSLLAFAEQRASXXDEHAELIVXRRGDYDAXTHQVQWXAQEVVAQAXLDGHRSMNPCPLYDXQTGTLFLFFIAIPXXVTEXQQLQTRANVTRLXXVTSTDHGRTWSSPRDLTDAAIGPXYREWSTFAVGPGHXLQLHDRXRSLVVPAYAYRKLHPXQRPIPSAFXFLSHDHGRTWARGHFVAQDTXECQVAEVETGEQRVVTLNARSHLRARVQAQSXNXGLDFQXSQLVKKLVEPPPXGXQGSVISFPSPRSGPGSPAQXLLYTHPTHXXQRADLGAYLNPRPPAPEAWSEPXLLAKGSXAYSDLQSMGTGPDGSPLFGXLYEANDYEEIXFXMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 77 - protein, 211 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 78 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 79 - DNA, 1851 bp, Artificial Sequence
  Description of Artificial Sequence Synthetic polynucleotide
gatgcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgcccctacacatcaggtgcagtggcaggctcaagaggtggtggctcaggctagactggacggccacagatctatgaacccctgtcctctgtacgatgcccagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgccgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaaggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcactagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 80 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 452 aa, Artificial Sequence
  Description of Artificial Sequence Synthetic polypeptide
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - DNA, 642 bp, Artificial Sequence
  Description of Artificial Sequence Synthetic polynucleotide
gagatcgtgctgacacagtctcccgccacactgtcactgtctccaggcgaaagagccacactgagctgtagagccagccagagcgtgtcctcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcccatcacctttggccagggcaccagactggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 83 - DNA, 1321 bp, Artificial Sequence
  Description of Artificial Sequence Synthetic polynucleotide
gcagcctggcagatctctgagactgtcttgtgccgccagcggcttcaccttcaacgactatgccatgcactgggtccgacaggcccctggcaaaggacttgaatgggtgtccaccatcagctggaacagcggctctatcggctacgccgattccgtgaagggcagattcaccatctccagagacaacgccaagaagtccctgtacctgcagatgaacagcctgagagccgaggacacagccctgtactactgcgccaaggacatccagtacggcaactactactacggcatggacgtgtggggccagggcacaacagtgacagtctcttctgctagcaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 84 - DNA, 1851 bp, Artificial Sequence
  Description of Artificial Sequence Synthetic polynucleotide
gatgcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaaggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcactagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 85 - DNA, 1140 bp, Artificial Sequence
  Description of Artificial Sequence Synthetic polynucleotide
gatgcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaa

SEQ ID NO: 86 - DNA, 641 bp, Artificial Sequence
  Description of Artificial Sequence Synthetic polynucleotide
cagatcgtgctgtctcagtctcctgccatcctgtctgctagccctggcgagaaagtgaccatgacctgtagagccagcagcagcgtgtcctacatccactggttccagcagaagcctggcagcagccctaagccttggatctacgccacaagcaatctggccagcggcgtgccagtcagattttctggctctggcagcggcaccagctacagcctgacaatctctagagtggaagccgaggacgccgccacctactattgtcagcagtggaccagcaatcctcctacctttggcggaggcaccaagctggaaatcaaggccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 87 - DNA, 1353 bp, Artificial Sequence
  Description of Artificial Sequence Synthetic polynucleotide
caggttcagctgcaacagcctggcgccgaacttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggcaccaccgtgacagtttctgctgctagcaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 88 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 89 - protein, 612 aa, Artificial Sequence
  Description of Artificial Sequence Synthetic polypeptide
DASLPYLQDESVFQSGAHAYRIPALLYLPGQQSLLAFAEQRASKKDEHAELIVLRRGDYDAPTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIRFIMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 90 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDANTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 91 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 92 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDASTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 93 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDATTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 94 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDANTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPAGCQGSVISFPSPRSGPGSPAQWLLYTHPTHRKQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 95 - protein, 617 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (6)..(6): Phe, Trp, Tyr or Val
  MOD_RES (9)..(9): Lys or Asp
  MOD_RES (62)..(62): Pro, Asn, Asp, His, Glu, Gly, Ser or Thr
  MOD_RES (93)..(93): Ala, Glu, or Lys
  MOD_RES (187)..(187): Arg, Ile, or Lys
  MOD_RES (270)..(270): Gln, Ala, His, Phe, or Pro
  MOD_RES (301)..(301): Ser or Arg
  MOD_RES (302)..(302): Trp or Lys
  MOD_RES (332)..(332): Ala, Cys, Ser, or Val
  MOD_RES (363)..(363): Val or Arg
  MOD_RES (365)..(365): Leu, Gln, His, Ile, Lys, or Ser
  MOD_RES (381)..(390): This region may encompass “GGGGSGGGGS” or “EPKSS”
  See specification as filed for detailed description of substitutions and preferred embodiments
XASLPXLQXESVFQSGAHAYRIPALLYLPGQQSLLAFAEQRASKKDEHAELIVLRRGDYDAXTHQVQWQAQEVVAQARLDGHRSMNPCPLYDXQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRARVQAQSTNDGLDFQESQLVKKLVEPPPXGCQGSVISFPSPRSGPGSPAQWLLYTHPTHXXQRADLGAYLNPRPPAPEAWSEPVLLAKGSXAYSDLQSMGTGPDGSPLFGCLYEANDYEEIXFXMFTLKQAFPAEYLPQXXXXXXXXXXDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 96 - protein, 617 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (2)..(2): Ala or Lys
  MOD_RES (4)..(4): Asn or Leu
  MOD_RES (5)..(5): Pro or His
  MOD_RES (6)..(6): Phe, Trp, Tyr or Val
  MOD_RES (9)..(9): Lys or Asp
  MOD_RES (44)..(44): Lys, Arg, or Glu
  MOD_RES (45)..(45): Lys, Ala, Arg, or Glu
  MOD_RES (54)..(54): Leu or Met
  MOD_RES (62)..(62): Pro, Asn, Asp, His, Glu, Gly, Ser or Thr
  MOD_RES (69)..(69): Gln or His
  MOD_RES (78)..(78): Arg or Lys
  MOD_RES (93)..(93): Ala, Glu or Lys
  MOD_RES (107)..(107): Gly or Asp
  MOD_RES (108)..(108): Gln or His
  MOD_RES (112)..(112): Gln, Arg, or Lys
  MOD_RES (125)..(125): Ala, Cys, Ile, Ser, Val, or Leu
  MOD_RES (126)..(126): Gln or Leu
  MOD_RES (150)..(150): Ala or Val
  MOD_RES (164)..(164): Cys or Gly
  MOD_RES (171)..(171): Ala or Gly
  MOD_RES (187)..(187): Arg, Ile, or Lys
  MOD_RES (196)..(196): Ala, Cys, Leu, or Val
  MOD_RES (217)..(217): Leu, Ala, or Val
  MOD_RES (249)..(249): Thr or Ala
  MOD_RES (251)..(251): Asp or Gly
  MOD_RES (257)..(257): Glu or Lys
  MOD_RES (270)..(270): Gln, Ala, His, Phe, or Pro
  MOD_RES (272)..(272): Cys or Val
  MOD_RES (292)..(292): Trp or Arg
  MOD_RES (301)..(301): Ser or Arg
  MOD_RES (302)..(302): Trp or Lys
  MOD_RES (325)..(325): Lys or Val
  MOD_RES (332)..(332): Ala, Cys, Ser, or Val
  MOD_RES (352)..(352): Cys, Leu, or Val
  MOD_RES (363)..(363): Val or Arg
  MOD_RES (365)..(365): Leu, Gln, His, Ile, Lys, or Ser
  MOD_RES (381)..(390): This region may encompass “GGGGSGGGGS” or “EPKSS”
  See specification as filed for detailed description of substitutions and preferred embodiments
XXSXXXLQXESVFQSGAHAYRIPALLYLPGQQSLLAFAEQRASXXDEHAELIVXRRGDYDAXTHQVQWXAQEVVAQAXLDGHRSMNPCPLYDXQTGTLFLFFIAIPXXVTEXQQLQTRANVTRLXXVTSTDHGRTWSSPRDLTDAAIGPXYREWSTFAVGPGHXLQLHDRXRSLVVPAYAYRKLHPXQRPIPSAFXFLSHDHGRTWARGHFVAQDTXECQVAEVETGEQRVVTLNARSHLRARVQAQSXNXGLDFQXSQLVKKLVEPPPXGXQGSVISFPSPRSGPGSPAQXLLYTHPTHXXQRADLGAYLNPRPPAPEAWSEPXLLAKGSXAYSDLQSMGTGPDGSPLFGXLYEANDYEEIXFXMFTLKQAFPAEYLPQXXXXXXXXXXDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 97 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 98 - protein, 10 aa, Artificial Sequence
  Description of Artificial Sequence Synthetic peptide
GGGGSGGGGS

SEQ ID NO: 99 - protein, 5 aa, Artificial Sequence
  Description of Artificial Sequence Synthetic peptide
EPKSS

SEQ ID NO: 100 - protein, 232 aa, Artificial Sequence
  Description of Artificial Sequence Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 101 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHARQRADLGAYLNPRPPAPEAWSEPVLLAKGSAAYSDLQSMGTGPDGSPLFGCLYEANDYEEIVFLMFTLKQAFPAEYLPQ

SEQ ID NO: 103 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHARQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 104 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHARQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 105 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 106 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 107 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 108 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 109 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 110 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 111 - protein, 211 aa, Artificial Sequence; identical to SEQ ID NO: 77
  Description of Artificial Sequence Synthetic polypeptide

SEQ ID NO: 112 - DNA, 633 bp, Artificial Sequence
  Description of Artificial Sequence Synthetic polynucleotide
gagatcgtgctgacacagtctcccgccacactgtcactgtctccaggcgaaagagccacactgagctgtagagccagccagagcgtgtcctcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcccatcacctttggccagggcaccagactggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacagg

SEQ ID NO: 113 - protein, 617 aa, Artificial Sequence; identical to SEQ ID NO: 68
  Description of Artificial Sequence Synthetic polypeptide

SEQ ID NO: 114 - DNA, 1848 bp, Artificial Sequence
  Description of Artificial Sequence Synthetic polynucleotide
gcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaaggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcactagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 115 - protein, 612 aa, Artificial Sequence; identical to SEQ ID NO: 88
  Description of Artificial Sequence Synthetic polypeptide

SEQ ID NO: 116 - DNA, 1833 bp, Artificial Sequence
  Description of Artificial Sequence Synthetic polynucleotide
gcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaagagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcactagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 117 - protein, 617 aa, Artificial Sequence
  Description of Artificial Sequence Synthetic polypeptide
DASLPYLQDESVFQSGAHAYRIPALLYLPGQQSLLAFAEQRASKKDEHAELIVLRRGDYDAPTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLANQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIRFRMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 118 - DNA, 1851 bp, Artificial Sequence
  Description of Artificial Sequence Synthetic polynucleotide
gatgcatctctgccttacctgcaggatgaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgcccctacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggcgaatcaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatccgtttccgtatgttcaccctgaagcaggcctttccagccgagtacctgcctcaaggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcactagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 119 - protein, 842 aa, Artificial Sequence
  Description of Artificial Sequence Synthetic polypeptide
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 120 - DNA, 2526 bp, Artificial Sequence
  Description of Artificial Sequence Synthetic polynucleotide
gaggtgcagctggttgaatctggcggaggactggtgcagcctggcagatctctgagactgtcttgtgccgccagcggcttcaccttcaacgactatgccatgcactgggtccgacaggcccctggcaaaggacttgaatgggtgtccaccatcagctggaacagcggctctatcggctacgccgattccgtgaagggcagattcaccatctccagagacaacgccaagaagtccctgtacctgcagatgaacagcctgagagccgaggacacagccctgtactactgcgccaaggacatccagtacggcaactactactacggcatggacgtgtggggccagggcacaacagtgacagtctcttctgctagcaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggcggaggtggaagtggcggaggcggatccgatgcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgtcaagtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccacagggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaa

SEQ ID NO: 121 - protein, 842 aa, Artificial Sequence
  Description of Artificial Sequence Synthetic polypeptide
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 122 - DNA, 2526 bp, Artificial Sequence
  Description of Artificial Sequence Synthetic polynucleotide
gaggtgcagctggttgaatctggcggaggactggtgcagcctggcagatctctgagactgtcttgtgccgccagcggcttcaccttcaacgactatgccatgcactgggtccgacaggcccctggcaaaggacttgaatgggtgtccaccatcagctggaacagcggctctatcggctacgccgattccgtgaagggcagattcaccatctccagagacaacgccaagaagtccctgtacctgcagatgaacagcctgagagccgaggacacagccctgtactactgcgccaaggacatccagtacggcaactactactacggcatggacgtgtggggccagggcacaacagtgacagtctcttctgctagcaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggcggaggtggaagtggcggaggcggatccgatgcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgttacgtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagagccagagtgcaggcccagagcacaaacgacggcctggatttccaagagagccagctggtcaagaaactggtggaacctcctccaaccggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaa

SEQ ID NO: 123 - DNA, 1140 bp, Artificial Sequence
  Description of Artificial Sequence Synthetic polynucleotide
gatgcatctctgccttacctgcagaaagaaagcgtgttccagtctggcgcccacgcctacagaattcccgctctgctgtatctgccaggccagcagtctctgctggctttcgctgaacagcgggccagcaagaaggatgagcacgccgaactgatcgtgctgcggagaggcgattacgacgccggcacacatcaggtgcagtggcaggctcaagaggtggtggctcaggctagactggacggccacagatctatgaacccctgtcctctgtacgatgaacagaccggcacactgtttctgttctttatcgctatccccggccaagtgaccgagcagcagcagctgcagacaagagccaacgtgaccagactgtgttacgtgacctccaccgaccacggcagaacctggtctagccctagagatctgaccgacgccgccatcggacctgcctatagagagtggtccaccttcgccgttggacctggacactgtctccagctgcacgacagggctagatctctggtggtgcctgcctacgcctatagaaagctgcaccccaaacagcggcctattcctagcgccttctgctttctgagccacgatcacggcaggacatgggccagaggacatttcgtggcccaggacacactggaatgccaggtggccgaagtggaaaccggcgagcagagagtcgtgaccctgaacgccagatctcacctgagattcagagtgcaggcccagagcacaaacgacggcctggatttccaagagagccagctggtcaagaaactggtggaacctcctccaaccggctgtcagggaagcgtgatcagctttccatctcctagaagcggccctggctctcctgctcagtggctgctgtatacacaccccacacacagctggcagagagccgatctgggcgcctacctgaatcctagacctcctgctcctgaggcttggagcgaacctgttctgctggccaagggcagcgctgcctacagcgatctgcagtctatgggcacaggccctgatggcagccctctgtttggctgtctgtacgaggccaacgactacgaagagatcgtgttcctgatgttcaccctgaagcaggcctttccagccgagtacctgcctcaa

SEQ ID NO: 124 - protein, 380 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (6)..(6): Phe, Trp, Tyr or Val
  MOD_RES (9)..(9): Lys or Asp
  MOD_RES (62)..(62): Pro, Asn, Asp, His, Glu, Gly, Ser or Thr
  MOD_RES (93)..(93): Ala, Glu, or Lys
  MOD_RES (126)..(126): Gln, Leu, Glu, Phe, His, Ile, Leu, or Tyr
  MOD_RES (187)..(187): Arg, Ile, or Lys
  MOD_RES (242)..(242): Ala, Cys, Phe, Gly, His, Ile, Lys, Leu, Met, Asn, Gln, Arg, Ser, Val, Trp, or Tyr
  MOD_RES (270)..(270): Gln, Ala, His, Phe, Pro, Ser, or Thr
  MOD_RES (301)..(301): Ser, Arg, Ala, Asp, Glu, Phe, Gly, His, Ile, Lys, Leu, Met, Asn, Pro, Gln, Thr, Val, Trp, or Tyr
  MOD_RES (302)..(302): Trp, Lys, Ala, Asp, Glu, Phe, Gly, His, Ile, Lys, Leu, Met, Asn, Pro, Gln, Arg, Ser, Thr, Val, or Tyr
  MOD_RES (332)..(332): Ala, Cys, Ser, or Val
  MOD_RES (363)..(363): Val or Arg
  MOD_RES (365)..(365): Leu, Gln, His, Ile, Lys, or Ser
  See specification as filed for detailed description of substitutions and preferred embodiments
XASLPXLQXESVFQSGAHAYRIPALLYLPGQQSLLAFAEQRASKKDEHAELIVLRRGDYDAXTHQVQWQAQEVVAQARLDGHRSMNPCPLYDXQTGTLFLFFIAIPGQVTEQQQLQTRANVTRLCXVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRXRVQAQSTNDGLDFQESQLVKKLVEPPPXGCQGSVISFPSPRSGPGSPAQWLLYTHPTHXXQRADLGAYLNPRPPAPEAWSEPVLLAKGSXAYSDLQSMGTGPDGSPLFGCLYEANDYEEIXFXMFTLKQAFPAEYLPQ

SEQ ID NO: 125 - protein, 380 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (2)..(2): Ala or Lys
  MOD_RES (4)..(4): Asn or Leu
  MOD_RES (5)..(5): Pro or His
  MOD_RES (6)..(6): Phe, Trp, Tyr or Val
  MOD_RES (9)..(9): Lys or Asp
  MOD_RES (44)..(44): Lys, Arg, or Glu
  MOD_RES (45)..(45): Lys, Ala, Arg, or Glu
  MOD_RES (54)..(54): Leu or Met
  MOD_RES (62)..(62): Pro, Asn, Asp, His, Glu, Gly, Ser or Thr
  MOD_RES (69)..(69): Gln or His
  MOD_RES (78)..(78): Arg or Lys
  MOD_RES (80)..(80): Asp or Pro
  MOD_RES (93)..(93): Ala, Glu or Lys
  MOD_RES (107)..(107): Gly or Asp
  MOD_RES (108)..(108): Gln or His
  MOD_RES (112)..(112): Gln, Arg, or Lys
  MOD_RES (125)..(125): Ala, Cys, Ile, Ser, Val, or Leu
  MOD_RES (126)..(126): Gln, Leu, Glu, Phe, His, Ile, Leu, or Tyr
  MOD_RES (150)..(150): Ala or Val
  MOD_RES (164)..(164): Cys or Gly
  MOD_RES (170)..(170): Arg or Pro
  MOD_RES (171)..(171): Ala or Gly
  MOD_RES (187)..(187): Arg, Ile, or Lys
  MOD_RES (188)..(188): Gln or Pro
  MOD_RES (189)..(189): Arg or Pro
  MOD_RES (196)..(196): Ala, Cys, Leu, or Val
  MOD_RES (213)..(213): Ala, Cys, Asn, Ser, or Thr
  MOD_RES (217)..(217): Leu, Ala, or Val
  MOD_RES (225)..(225): Glu or Pro
  MOD_RES (239)..(239): His or Pro
  MOD_RES (240)..(240): Leu, Asp, Asn, or Tyr
  MOD_RES (241)..(241): Arg, Ala, Asp, Leu, Gln, or Tyr
  MOD_RES (242)..(242): Ala, Cys, Phe, Gly, His, Ile, Lys, Leu, Met, Asn, Gln, Arg, Ser, Val, Trp, or Tyr
  MOD_RES (244)..(244): Val, Ile, or Lys
  MOD_RES (249)..(249): Thr or Ala
  MOD_RES (251)..(251): Asp or Gly
  MOD_RES (257)..(257): Glu, Lys, or Pro
  MOD_RES (258)..(258): Ser or Cys
  MOD_RES (260)..(260): Leu, Asp, Phe, Gln, or Thr
  MOD_RES (265)..(265): Val or Phe
  MOD_RES (270)..(270): Gln, Ala, His, Phe, Pro, Ser, or Thr
  MOD_RES (272)..(272): Cys or Val
  MOD_RES (292)..(292): Trp or Arg
  MOD_RES (301)..(301): Ser, Arg, Ala, Asp, Glu, Phe, Gly, His, Ile, Lys, Leu, Met, Asn, Pro, Gln, Thr, Val, Trp, or Tyr
  MOD_RES (302)..(302): Trp, Lys, Ala, Asp, Glu, Phe, Gly, His, Ile, Lys, Leu, Met, Asn, Pro, Gln, Arg, Ser, Thr, Val, or Tyr
  MOD_RES (325)..(325): Lys or Val
  MOD_RES (332)..(332): Ala, Cys, Ser, or Val
  MOD_RES (352)..(352): Cys, Leu, or Val
  MOD_RES (363)..(363): Val or Arg
  MOD_RES (365)..(365): Leu, Gln, His, Ile, Lys, or Ser
  See specification as filed for detailed description of substitutions and preferred embodiments
XXSXXXLQXESVFQSGAHAYRIPALLYLPGQQSLLAFAEQRASXXDEHAELIVXRRGDYDAXTHQVQWXAQEVVAQAXLXGHRSMNPCPLYDXQTGTLFLFFIAIPXXVTEXQQLQTRANVTRLXXVTSTDHGRTWSSPRDLTDAAIGPXYREWSTFAVGPGHXLQLHDXXRSLVVPAYAYRKLHPXXXPIPSAFXFLSHDHGRTWARGHFVXQDTXECQVAEVXTGEQRVVTLNARSXXXXRXQAQSXNXGLDFQXXQXVKKLXEPPPXGXQGSVISFPSPRSGPGSPAQXLLYTHPTHXXQRADLGAYLNPRPPAPEAWSEPXLLAKGSXAYSDLQSMGTGPDGSPLFGXLYEANDYEEIXFXMFTLKQAFPAEYLPQ

SEQ ID NO: 126 - protein, 5 aa, Artificial Sequence
  Description of Artificial Sequence Synthetic peptide
GGGGS

SEQ ID NO: 127 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRFRVQAQSTNDGLDFQESQLVKKLVEPPPTGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 451 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 130 - protein, 452 aa, Artificial Sequence
  Description of Artificial Sequence Synthetic polypeptide
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 131 - protein, 617 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (6)..(6): Phe, Trp, Tyr or Val
  MOD_RES (9)..(9): Lys or Asp
  MOD_RES (62)..(62): Pro, Asn, Asp, His, Glu, Gly, Ser or Thr
  MOD_RES (93)..(93): Ala, Glu, or Lys
  MOD_RES (126)..(126): Gln, Leu, Glu, Phe, His, Ile, Leu, or Tyr
  MOD_RES (187)..(187): Arg, Ile, or Lys
  MOD_RES (242)..(242): Ala, Cys, Phe, Gly, His, Ile, Lys, Leu, Met, Asn, Gln, Arg, Ser, Val, Trp, or Tyr
  MOD_RES (270)..(270): Gln, Ala, His, Phe, Pro, Ser, or Thr
  MOD_RES (301)..(301): Ser, Arg, Ala, Asp, Glu, Phe, Gly, His, Ile, Lys, Leu, Met, Asn, Pro, Gln, Thr, Val, Trp, or Tyr
  MOD_RES (302)..(302): Trp, Lys, Ala, Asp, Glu, Phe, Gly, His, Ile, Lys, Leu, Met, Asn, Pro, Gln, Arg, Ser, Thr, Val, or Tyr
  MOD_RES (332)..(332): Ala, Cys, Ser, or Val
  MOD_RES (363)..(363): Val or Arg
  MOD_RES (365)..(365): Leu, Gln, His, Ile, Lys, or Ser
  MOD_RES (381)..(390): This region may encompass “GGGGS” or “GGGGSGGGGS” or “EPKSS”
  See specification as filed for detailed description of substitutions and preferred embodiments
XASLPXLQXESVFQSGAHAYRIPALLYLPGQQSLLAFAEQRASKKDEHAELIVLRRGDYDAXTHQVQWQAQEVVAQARLDGHRSMNPCPLYDXQTGTLFLFFIAIPGQVTEQQQLQTRANVTRLCXVTSTDHGRTWSSPRDLTDAAIGPAYREWSTFAVGPGHCLQLHDRARSLVVPAYAYRKLHPXQRPIPSAFCFLSHDHGRTWARGHFVAQDTLECQVAEVETGEQRVVTLNARSHLRXRVQAQSTNDGLDFQESQLVKKLVEPPPXGCQGSVISFPSPRSGPGSPAQWLLYTHPTHXXQRADLGAYLNPRPPAPEAWSEPVLLAKGSXAYSDLQSMGTGPDGSPLFGCLYEANDYEEIXFXMFTLKQAFPAEYLPQXXXXXXXXXXDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 132 - protein, 617 aa, Artificial Sequence
  Description of Artificial Sequence Synthetic polypeptide
  MOD_RES (1)..(1): Ala, Arg, Asn, Asp, Gln, Glu, Gly, His, Leu, Lys, Met, Phe, Thr, Val, or not present
  MOD_RES (2)..(2): Ala or Lys
  MOD_RES (4)..(4): Asn or Leu
  MOD_RES (5)..(5): Pro or His
  MOD_RES (6)..(6): Phe, Trp, Tyr or Val
  MOD_RES (9)..(9): Lys or Asp
  MOD_RES (44)..(44): Lys, Arg, or Glu
  MOD_RES (45)..(45): Lys, Ala, Arg, or Glu
  MOD_RES (54)..(54): Leu or Met
  MOD_RES (62)..(62): Pro, Asn, Asp, His, Glu, Gly, Ser or Thr
  MOD_RES (69)..(69): Gln or His
  MOD_RES (78)..(78): Arg or Lys
  MOD_RES (80)..(80): Asp or Pro
  MOD_RES (93)..(93): Ala, Glu or Lys
  MOD_RES (107)..(107): Gly or Asp
  MOD_RES (108)..(108): Gln or His
  MOD_RES (112)..(112): Gln, Arg, or Lys
  MOD_RES (125)..(125): Ala, Cys, Ile, Ser, Val, or Leu
  MOD_RES (126)..(126): Gln, Leu, Glu, Phe, His, Ile, Leu, or Tyr
  MOD_RES (150)..(150): Ala or Val
  MOD_RES (164)..(164): Cys or Gly
  MOD_RES (170)..(170): Arg or Pro
  MOD_RES (171)..(171): Ala or Gly
  MOD_RES (187)..(187): Arg, Ile, or Lys
  MOD_RES (188)..(188): Gln or Pro
  MOD_RES (189)..(189): Arg or Pro
  MOD_RES (196)..(196): Ala, Cys, Leu, or Val
  MOD_RES (213)..(213): Ala, Cys, Asn, Ser, or Thr
  MOD_RES (217)..(217): Leu, Ala, or Val
  MOD_RES (225)..(225): Glu or Pro
  MOD_RES (239)..(239): His or Pro
  MOD_RES (240)..(240): Leu, Asp, Asn, or Tyr
  MOD_RES (241)..(241): Arg, Ala, Asp, Leu, Gln, or Tyr
  MOD_RES (242)..(242): Ala, Cys, Phe, Gly, His, Ile, Lys, Leu, Met, Asn, Gln, Arg, Ser, Val, Trp, or Tyr
  MOD_RES (244)..(244): Val, Ile, or Lys
  MOD_RES (249)..(249): Thr or Ala
  MOD_RES (251)..(251): Asp or Gly
  MOD_RES (257)..(257): Glu, Lys, or Pro
  MOD_RES (258)..(258): Ser or Cys
  MOD_RES (260)..(260): Leu, Asp, Phe, Gln, or Thr
  MOD_RES (265)..(265): Val or Phe
  MOD_RES (270)..(270): Gln, Ala, His, Phe, Pro, Ser, or Thr
  MOD_RES (272)..(272): Cys or Val
  MOD_RES (292)..(292): Trp or Arg
  MOD_RES (301)..(301): Ser, Arg, Ala, Asp, Glu, Phe, Gly, His, Ile, Lys, Leu, Met, Asn, Pro, Gln, Thr, Val, Trp, or Tyr
  MOD_RES (302)..(302): Trp, Lys, Ala, Asp, Glu, Phe, Gly, His, Ile, Lys, Leu, Met, Asn, Pro, Gln, Arg, Ser, Thr, Val, or Tyr
  MOD_RES (325)..(325): Lys or Val
  MOD_RES (332)..(332): Ala, Cys, Ser, or Val
  MOD_RES (352)..(352): Cys, Leu, or Val
  MOD_RES (363)..(363): Val or Arg
  MOD_RES (365)..(365): Leu, Gln, His, Ile, Lys, or Ser
  MOD_RES (381)..(390): This region may encompass “GGGGS” or “GGGGSGGGGS” or “EPKSS”
  See specification as filed for detailed description of substitutions and preferred embodiments
XXSXXXLQXESVFQSGAHAYRIPALLYLPGQQSLLAFAEQRASXXDEHAELIVXRRGDYDAXTHQVQWXAQEVVAQAXLXGHRSMNPCPLYDXQTGTLFLFFIAIPXXVTEXQQLQTRANVTRLXXVTSTDHGRTWSSPRDLTDAAIGPXYREWSTFAVGPGHXLQLHDXXRSLVVPAYAYRKLHPXXXPIPSAFXFLSHDHGRTWARGHFVXQDTXECQVAEVXTGEQRVVTLNARSXXXXRXQAQSXNXGLDFQXXQXVKKLXEPPPXGXQGSVISFPSPRSGPGSPAQXLLYTHPTHXXQRADLGAYLNPRPPAPEAWSEPXLLAKGSXAYSDLQSMGTGPDGSPLFGXLYEANDYEEIXFXMFTLKQAFPAEYLPQXXXXXXXXXXDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 133 - protein, 380 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQ

SEQ ID NO: 134 - protein, 612 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 135 - protein, 617 aa, Artificial Sequence
  Description of Artificial Sequence Synthetic polypeptide
DASLPYLQKESVFQSGAHAYRIPALLYLPGQQSLLAFAEQRASKKDEHAELIVLRRGDYDAGTHQVQWQAQEVVAQARLDGHRSMNPCPLYDEQTGTLFLFFIAIPGQVTEQQQLQTRANVTRLCYVTSTDHGRTWSSPRDLTDAAIGPAYREWSTFAVGPGHCLQLHDRARSLVVPAYAYRKLHPKQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSAAYSDLQSMGTGPDGSPLFGCLYEANDYEEIVFLMFTLKQAFPAEYLPQGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 136 - protein, 10 aa, Artificial Sequence
  Description of Artificial Sequence Synthetic 10xHis tag
HHHHHHHHHH

SEQ ID NO: 137 - protein, 15 aa, Artificial Sequence
  Description of Artificial Sequence Synthetic peptide
  MISC_FEATURE (1)..(15): This sequence may encompass 1-5 “Gly Gly Pro” repeating units
GGPGGPGGPGGPGGP

SEQ ID NO: 138 - protein, 25 aa, Artificial Sequence
  Description of Artificial Sequence Synthetic peptide
  MISC_FEATURE (1)..(25): This sequence may encompass 1-5 “Gly Gly Gly Gly Ser” repeating units
GGGGSGGGGSGGGGSGGGGSGGGGS

SEQ ID NO: 139 - protein, 6 aa, Artificial Sequence
  Description of Artificial Sequence Synthetic 6xHis tag
HHHHHH

SEQ ID NO: 140 - protein, 5 aa, Homo sapiens
LHPIQ

SEQ ID NO: 141 - protein, 10 aa, Homo sapiens
TFAVGPGHCL

SEQ ID NO: 142 - protein, 6 aa, Homo sapiens
LRARVQ

SEQ ID NO: 143 - protein, 6 aa, Homo sapiens
LRCRVQ

SEQ ID NO: 144 - protein, 6 aa, Homo sapiens
LRAKVQ

SEQ ID NO: 145 - protein, 6 aa, Homo sapiens
LRARIQ

SEQ ID NO: 146 - protein, 6 aa, Homo sapiens
LRARPQ

SEQ ID NO: 147 - protein, 6 aa, Homo sapiens
LRCRKQ

SEQ ID NO: 148 - protein, 6 aa, Homo sapiens
LRRRRQ

SEQ ID NO: 149 - protein, 6 aa, Homo sapiens
LRRRHQ

SEQ ID NO: 150 - protein, 6 aa, Homo sapiens
LRTRPQ

SEQ ID NO: 151 - protein, 6 aa, Homo sapiens
LRYRPQ

SEQ ID NO: 152 - protein, 6 aa, Homo sapiens
LRNRPQ